(19) United States
(12) Patent Application Publication
(10) Pub. No.: US 2026/0007772 A1
(43) Pub. Date: Jan. 8, 2026

(54) COMPOSITIONS AND METHODS FOR TREATING ALPHA-1 ANTITRYPSIN DEFICIENCY

(71) Applicant: Intellia Therapeutics, Inc., Cambridge, MA (US)

(72) Inventors: Laura Sepp-Lorenzino, Jenkintown, PA (US); Zachary W. Dymek, Grafton, MA (US)

(21) Appl. No.: 18/700,487

(22) PCT Filed: Oct. 14, 2022
(86) PCT No.: PCT/US2022/078140

§ 371 Date: Apr. 11, 2024

Related U.S. Application Data
(60) Provisional application No. 63/256,365, filed on Oct. 15, 2021.

(51) Int. Cl. A61K 48/00; A61K 9/127; A61K 9/51; A61P 3/00; C07K 14/81; C12N 9/22; C12N 15/11; C12N 15/86; C12N 15/88

(52) U.S. Cl. CPC A61K 48/005; A61K 9/127; A61K 9/5123; A61P 3/00; C07K 14/8125; C12N 9/226; C12N 15/111; C12N 15/86; C12N 15/88; C12N 2310/20; C12N 2750/14143; C12N 2830/50

(57) ABSTRACT

Compositions and methods for expressing alpha 1 antitrypsin (AAT) in a host cell are provided. Also provided are compositions and methods for treating subjects having alpha 1 antitrypsin deficiency (AATD).

### CROSS-REFERENCE TO RELATED APPLICATIONS

This application claims the benefit of U.S. Provisional Application No. 63/256,365, filed on Oct. 15, 2021, the disclosure of which is hereby incorporated by reference in its entirety.

### BACKGROUND

Alpha-1 antitrypsin (AAT or A1AT) or serum trypsin inhibitor is a type of serine protease inhibitor (also termed a serpin) encoded by the SERPINA1 gene. AAT is primarily synthesized and secreted by hepatocytes, and functions to inhibit the activity of neutrophil elastase in the lung. Without sufficient quantities of functioning AAT, neutrophil elastase is uncontrolled and damages alveoli in the lung. Thus, mutations in SERPINA1 that result in decreased levels of AAT, or decreased levels of properly functioning AAT, lead to lung pathology. Moreover, mutations in SERPINA1 that lead to production of misformed AAT can lead to liver pathology due to accumulation of AAT in hepatocytes. Thus, insufficient and improperly formed AAT caused by SERPINA1 mutation can lead to lung and liver pathology.

More than one hundred allelic variants have been described for the SERPINA1 gene. Variants are generally classified according to their effect on serum levels of AAT. For example, M alleles are normal variants associated with normal serum AAT levels, whereas Z and S alleles are mutant variants associated with decreased AAT levels. The presence of Z and S alleles is associated with al-antitrypsin deficiency (AATD or A1AD), a genetic disorder characterized by mutations in the SERPINA1 gene that leads to the production of abnormal AAT.

There are many forms and degrees of AATD. The “Z-variant” is the most common, causing severe clinical disease in both liver and lung. The Z-variant is characterized by a single nucleotide change in the 5′ end of the 5^{th} exon that results in a missense mutation of glutamic acid to lysine at amino acid position 342 (E342K). Symptoms arise in patients that are both homozygous (ZZ) and heterozygous (MZ or SZ) at the Z allele. The presence of one or two Z alleles results in SERPINA1 mRNA instability, and AAT protein polymerization and aggregation in liver hepatocytes. Patients having at least one Z allele have an increased incidence of liver cancer due to the accumulation of aggregated AAT protein in the liver. In addition to liver pathology, AATD characterized by at least one Z allele is also characterized by lung disease due to the decrease in AAT in the alveoli and the resulting decrease in inhibition of neutrophil elastase. The prevalence of the severe ZZ-form (i.e., homozygous expression of the Z-variant) is 1:2,000 in northern European populations, and 1:4,500 in the United States. The other common mutation is the S-variant, which results in a protein that is degraded intracellularly before secretion. Compared to the Z-variant, the S-variant causes milder reduction in serum AAT and lower risk for lung disease.

A need exists for methods and compositions that ameliorate the negative effects of AATD in both the liver and lung.

### SUMMARY

The present disclosure provides compositions and methods for expressing heterologous AAT at a human genomic locus, such as an albumin safe harbor site, thereby allowing secretion of heterologous AAT and alleviating the negative effects of AATD in the lung. The present disclosure also provides compositions and methods to knock out or reduce expression of the endogenous SERPINA1 gene thereby, thereby eliminating or reducing the production of mutant forms of AAT that are associated with liver symptoms in patients with AATD. Thus, in certain embodiments are compositions and methods for inserting heterologous AAT at a safe harbor site to restore AAT function in a cell or an organism and blocking expression of an endogenous SERPINA1 allele (e.g., by targeting it with a guide RNA or siRNA).

In certain aspects, provided herein are bidirectional nucleic acid constructs. In some embodiments, such constructs comprise: a) a first segment comprising a first alpha-1 antitrypsin (AAT) polypeptide coding sequence, wherein the codon usage of the first AAT polypeptide coding sequence is different from the codon usage of the SERPINA1 gene; and b) a second segment comprising a reverse complement of a second AAT polypeptide coding sequence wherein the codon usage of the second AAT polypeptide coding sequence is different from the codon usage of the first AAT polypeptide coding sequence and from the codon usage of the SERPINA1 gene. In some embodiments, the coding sequences of the first segment and the second segment are CpG depleted. In some embodiments, the bidirectional nucleic acid construct nucleotide sequence is CpG depleted. In certain embodiments, the construct does not comprise a promoter that drives the expression of either the first AAT polypeptide coding sequence or the second AAT polypeptide coding sequence. In some embodiments, the second segment is 3′ of the first segment. In certain embodiments, the construct does not comprise a homology arm.

As used herein, an AAT polypeptide coding sequence is a nucleotide sequence that encodes an active polypeptide that inhibits neutrophil elastase. For example, in some embodiments the AAT polypeptide coding sequence encodes a polypeptide comprising the sequence SEQ ID NO: 700 or 702.

In certain embodiments, wherein the first segment of the bidirectional nucleic acid construct is linked to the second segment of the bidirectional nucleic acid construct by a linker. In some embodiments, the linker is 5, 10, 20, 30, 40, 50, 60, 70, 80, 90, 100, 150, 200, 250, 300, 500, 1000, 1500, 2000 nucleotides in length. In certain embodiments, the linker is CpG depleted.

In some embodiments, each of the first segment and second segment of the bidirectional nucleic acid construct comprises a polyadenylation tail sequence, a polyadenylation signal sequence, or a polyadenylation site. In some embodiments, the construct comprises a splice acceptor site. In certain embodiments, the construct comprises a first splice acceptor site upstream of the first segment and a second (reverse) splice acceptor site downstream of the second segment. In certain embodiments, the splice acceptor site is a human splice acceptor site. In certain embodiments, the splice acceptor site is a murine splice acceptor site.

In certain embodiments, the bidirectional nucleic acid construct is double-stranded, optionally double-stranded DNA. In some embodiments, the construct is single-stranded, optionally single-stranded DNA.

In certain embodiments, the first AAT polypeptide coding sequence of the bidirectional nucleic acid construct or the second AAT polypeptide coding sequence of the bidirectional nucleic acid construct is codon-optimized. In certain embodiments, the construct comprises one or more of the following terminal structures: hairpin, loops, inverted terminal repeats (ITR), or toroid. In some embodiments, the terminal structure is CpG depleted. In some embodiments, the bidirectional nucleic acid construct nucleotide sequence is CpG depleted but the ITR is not CPG depleted.

In certain embodiments, the bidirectional nucleic acid construct comprises one, two, or three inverted terminal repeats (ITR). In some embodiments, the construct comprises no more than two ITRs.

In some embodiments, the AAT polypeptide coding sequences of the bidirectional nucleic acid construct have codon usage that prevents or reduces the ability of a SERPINA1 targeting siRNA, dsRNA or guide RNA to target it.

In certain embodiments, both the first AAT polypeptide coding sequence of the bidirectional nucleic acid construct and the second AAT polypeptide coding sequence of the bidirectional nucleic acid construct includes the use of a non-wild type codon within the a region (or one or more regions) of the sequence corresponding to bases 409-431, 409-410, 412-431, 415-418, 506-528, 506-525, 519-522, 527-528, 538-560, 538-557, 551-554, 559-560, 957-977, 970-976, 1403-1436, 1403-1425, 1410-1436, 1418-1424, 1423-1435, or any combination thereof of SEQ ID NO:703.

In some embodiments, both the first AAT polypeptide coding sequence of the bidirectional nucleic acid construct and the second AAT polypeptide coding sequence of the bidirectional nucleic acid construct includes at least one, at least 2, or at least 3 mismatches (e.g., from 1-10 mismatches, from 1-9 mismatches, from 1-8 mismatches, from 1-7 mismatches, from 1-6 mismatches, from 1-5 mismatches, from 1-4 mismatches, from 1-3 mismatches, from 1-2 mismatches, 1 mismatch, from 2-10 mismatches, from 2-9 mismatches, from 2-8 mismatches, from 2-7 mismatches, from 2-6 mismatches, from 2-5 mismatches, from 2-4 mismatches, from 1-3 mismatches, 2 mismatches, from 3-10 mismatches, from 3-9 mismatches, from 3-8 mismatches, from 3-7 mismatches, from 3-6 mismatches, from 3-5 mismatches, from 3-4 mismatches, 3 mismatches, from 4-10 mismatches, from 4-9 mismatches, from 4-8 mismatches, from 4-7 mismatches, from 4-6 mismatches, from 4-5 mismatches, 4 mismatches, from 5-10 mismatches, from 5-9 mismatches, from 5-8 mismatches, from 5-7 mismatches, from 5-6 mismatches, 5 mismatches, from 6-10 mismatches, from 6-9 mismatches, from 6-8 mismatches, from 6-7 mismatches, 6 mismatches, from 7-10 mismatches, from 7-9 mismatches, from 7-8 mismatches, 7 mismatches, from 8-10 mismatches, from 8-9 mismatches, or 8 mismatches) from a wild-type SERPINA1 gene sequence within the region (or one or more regions) of the AAT polypeptide coding sequence corresponding to bases 409-431, 409-410, 412-431, 415-418, 506-528, 506-525, 519-522, 527-528, 538-560, 538-557, 551-554, 559-560, 957-977, 970-976, 1403-1436, 1403-1425, 1410-1436, 1418-1424, 1423-1435, or any combination thereof of SEQ ID NO: 703.

In some embodiments, neither the first AAT polypeptide coding sequence of the bidirectional nucleic acid construct nor the second AAT polypeptide coding sequence of the bidirectional nucleic acid construct is targeted by an RNAi agent targeted to nucleotides 957-977, 1403-1425, or 1410-1436 of SEQ ID NO: 703.

In certain embodiments, neither the first AAT polypeptide coding sequence of the bidirectional nucleic acid construct nor the second AAT polypeptide coding sequence of the bidirectional nucleic acid construct is targeted by a SERPINA1 targeting guide RNA having a targeting sequence of SEQ ID NOs: 1129, 1130, or 1131.

In some embodiments, both the first AAT polypeptide coding sequence of the bidirectional nucleic acid construct and the second AAT polypeptide coding sequence of the bidirectional nucleic acid construct includes the use of a non-wild type codon within the region (or one or more regions) of the sequence corresponding to bases 409-431, 409-410, 412-431, 415-418, 506-528, 506-525, 519-522, 527-528, 538-560, 538-557, 551-554, 559-560, 957-977, 970-976, 1403-1436, 1403-1425, 1410-1436, 1418-1424, 1423-1435, or any combination thereof of SEQ ID NO: 703.

In certain embodiments, the first AAT polypeptide coding sequence of the bidirectional nucleic acid construct comprises a sequence selected from SEQ ID NOs: 771, 772, 781, 782. In some embodiments, the second AAT polypeptide coding sequence of the bidirectional nucleic acid construct comprises a sequence selected from SEQ ID NOs: 771, 772, 781, and 782. In certain embodiments, the nucleic acid sequence of the bidirectional nucleic acid construct is selected from: SEQ ID NOs: 770, 780, and 1564.

In certain aspects, provided herein is a method of introducing a SERPINA1 nucleic acid sequence into a cell or population of cells comprising administering to the cell or population of cells comprising administering to the cell or population of cells a bidirectional nucleic acid construct provided herein. In some embodiments, the method comprises administering to a cell or population of cells: i) a bidirectional nucleic acid construct provided herein, ii) an RNA-guided DNA binding agent; and iii) an albumin guide RNA (gRNA); thereby introducing the SERPINA1 nucleic acid to the cell or population of cells. In some embodiments, the albumin gRNA comprises a sequence chosen from: a) a sequence that is at least 95%, SEQ ID Nos: 2-33; b) at least 17, 18, 19, or 20 contiguous nucleotides of a sequence selected from the group consisting of SEQ ID NOs: 2-33; c) a sequence selected from the group consisting of SEQ ID NOs: 2-33. In some embodiments, the cell or population of cells includes a liver cell (e.g., a hepatocyte). In some embodiments, the cell or population of cells expresses functional AAT at a level that is increased by at least about 10%, 20%, 30%, 40%, 50%, 60%, 70%, 80%, 90%, 100%, or more, as compared to a level before administration.

In certain aspects, provided herein is a method of increasing alpha-1 antitrypsin (AAT) secretion from a liver cell or population of cells comprising administering to the cell or population of cells comprising administering to the liver cell or population of liver cells a bidirectional nucleic acid construct provided herein. In some embodiments, the method comprises administering to a liver cell or population of cells: i) a bidirectional nucleic acid construct provided herein; ii) an RNA-guided DNA binding agent; and iii) an albumin guide RNA (gRNA); thereby increasing AAT secretion from the liver cell or the population of liver cells. In some embodiments the albumin gRNA comprises a sequence chosen from: a) a sequence that is at least 95%, SEQ ID Nos: 2-33; b) at least 17, 18, 19, or 20 contiguous nucleotides of a sequence selected from the group consisting of SEQ ID NOs: 2-33; c) a sequence selected from the group consisting of SEQ ID NOs: 2-33. In certain embodiments, the liver cell is a hepatocyte. In some embodiments, the cell or population of cells expresses functional AAT at a level that is increased by at least about 10%, 20%, 30%, 40%, 50%, 60%, 70%, 80%, 90%, 100%, or more, as compared to a level before administration.

In certain aspects, provided herein is a method of expressing alpha-1 antitrypsin (AAT) in a subject (e.g., a subject in need thereof), the method comprising administering to the subject a bidirectional nucleic acid construct provided herein. In certain embodiments, the method comprises administering to the subject: i) a bidirectional nucleic acid construct provided herein; ii) an RNA-guided DNA binding agent; and iii) an albumin guide RNA (gRNA); thereby expressing AAT in a subject. In some embodiments, the albumin guide RNA comprises a sequence chosen from: a) a sequence that is at least 95% identical to a sequence selected from the group consisting of SEQ ID Nos: 2-33; b) at least 17, 18, 19, or 20 contiguous nucleotides of a sequence selected from the group consisting of SEQ ID NOs: 2-33; and c) a sequence selected from the group consisting of SEQ ID NOs: 2-33.

In certain aspects, provided herein is a method of treating alpha-1 antitrypsin deficiency (AATD) in a subject (e.g., a subject in need thereof), the method comprising administering to the subject a bidirectional nucleic acid construct provided herein. In certain embodiments, the method comprises administering to the subject: i) a bidirectional nucleic acid construct provided herein; ii) an RNA-guided DNA binding agent; and iii) an albumin guide RNA (gRNA); thereby treating AATD in the subject. In some embodiments, the albumin guide RNA comprises a sequence chosen from: a) a sequence that is at least 95% identical to a sequence selected from the group consisting of SEQ ID Nos: 2-33; b) at least 17, 18, 19, or 20 contiguous nucleotides of a sequence selected from the group consisting of SEQ ID NOs: 2-33; and c) a sequence selected from the group consisting of SEQ ID NOs: 2-33.

In certain embodiments of the methods provided herein the subject's level of functional AAT is increased to at least about 500 μg/ml. In some embodiments, the subject's level of functional AAT is increased by at least about 10%, 20%, 30%, 40%, 50%, 60%, 70%, 80%, 90%, 100%, or more, as compared to the subject's level of functional AAT before administration. In some embodiments, the level of AAT is measured in serum or plasma. In certain embodiments, the level of AAT in serum is at least 500 μg/ml, at least 500 μg/ml, at least 571 μg/ml at least 750 μg/ml, at least 1000 μg/ml, 500-4000 μg/ml, 500-3500 μg/ml, 750-3500 μg/ml, 1000-3500 μg/ml, 1000-3000 μg/ml, or 1000-2700 μg/ml. In some embodiments, the level is measured at least 8 weeks, at least 9 weeks, at least 10 weeks, at least 11 weeks, or at least 12 weeks after the administration of the bidirectional nucleic acid construct. In certain embodiments, the level of functional AAT in the subject is maintained for at least a year following administration.

In certain embodiments of the methods provided herein, the subject has impaired liver or lung function. In some embodiments, administration delays progression of emphysema in the subject.

In certain embodiments, the methods provided herein further comprise reducing expression of the endogenous SERPINA1 gene without significantly reducing expression of the AAT polypeptide coding sequences of the bidirectional nucleic acid construct. In some embodiments, the method comprises administration of an endogenous SERPINA1 gene targeted nucleic acid agent. In some embodiments, the endogenous SERPINA1 gene targeted nucleic acid agent is an siRNA, a dsRNA, or a guide RNA. In certain embodiments, the endogenous SERPINA1 gene targeted nucleic acid agent is selected from an RNAi agent targeted to nucleotides 957-977, 1403-1425, or 1410-1436 of SEQ ID NO: 703, and a guide RNA targeted the endogenous SERPINA1 gene at a position corresponding to nucleotides 412-431, 506-525, or 538-557 of SEQ ID NO: 703.

In some embodiments, the methods provided herein further comprise inducing a double-stranded break (DSB) within the endogenous SERPINA1 gene. In some embodiments, the method comprises inducing a double-strand break (DSB) is induced within the endogenous SERPINA1 gene at a position corresponding to nucleotides 412-431, 506-525, or 538-557 of SEQ ID NO: 703. In certain embodiments, the method further comprises modifying the endogenous SERPINA1 gene. In some embodiments, the DSB is induced within the endogenous SERPINA1 gene or the endogenous SERPINA1 gene is modified after contacting the cell or population of cells or administering to the subject the bidirectional nucleic acid construct.

In some embodiments of the methods provided herein, the endogenous SERPINA1 gene targeted nucleic acid agent is a SERPINA1 guide RNA that is at least partially complementary to a target sequence present in exon 2, 3, 4, or 5 of the endogenous human SERPINA1 gene and that targets neither the first AAT polypeptide coding sequence nor the second AAT polypeptide coding sequences. In some embodiments, the endogenous SERPINA1 gene targeted nucleic acid agent is a SERPINA1 guide RNA that is at least partially complementary to a target sequence within the endogenous SERPINA1 gene at a position corresponding to nucleotides 412-431, 506-525, or 538-557 of SEQ ID NO: 703. In some embodiments, the SERPINA1 guide RNA comprises: a guide sequence selected from SEQ ID NOs: 1129-1131; a guide sequence that is at least 95% identical to SEQ ID NOs: 1129-1131; or 17, 18, 19, or 20 consecutive nucleotides of a sequence chosen from SEQ ID 15 NOs: 1129-1131.

In certain embodiments of the methods provided herein, the administration step is performed in vivo. In some embodiments, the nucleic acid construct is administered in a nucleic acid vector or a lipid nanoparticle. In some embodiments, the RNA-guided DNA binding agent or albumin gRNA is delivered or administered in a nucleic acid vector or lipid nanoparticle.

In certain embodiments provided herein, the RNA-guided DNA binding agent or SERPINA1 gRNA is delivered or administered in a nucleic acid vector or lipid nanoparticle. In some embodiments, the nucleic acid vector is a viral vector. In some embodiments, the viral vector is selected from an adeno associate viral (AAV) vector, adenovirus vector, retrovirus vector, and lentivirus vector. In some embodiments, the AAV vector is selected from the group consisting of AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof.

In certain embodiments of the methods provided herein, the RNA-guided DNA binding agent is a class 2 Cas nuclease. In some embodiments, the Cas nuclease is a Cas9 nuclease. In some embodiments, the Cas9 nuclease is an S. pyogenes Cas9 nuclease. In some embodiments, the Cas nuclease is cleavase.

In certain aspects, provided herein is a vector comprising a bidirectional nucleic acid construct provided herein. In some embodiments, the vector is an adeno-associated virus (AAV) vector. In certain embodiments, the AAV comprises a single-stranded genome (ssAAV) or a self-complementary genome (scAAV). In some embodiments, the AAV vector is selected from the group consisting of AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof. In some embodiments, the vector does not comprise a homology arm. In some embodiments, the vector is CpG depleted.

In certain aspects, provided herein is a lipid nanoparticle comprising a bidirectional nucleic acid construct provided herein.

In certain aspects, provided herein is a host cell comprising a bidirectional nucleic acid construct provided herein. In some embodiments, the host cell is a liver cell (e.g., a hepatocyte). In some embodiments, the host cell is a non-dividing cell type. In certain embodiments, the host cell expresses the AAT polypeptide encoded by the bidirectional construct.

In certain aspects, provided herein is a method of reducing endogenous alpha-1 antitrypsin (AAT) expression in a subject comprising a bidirectional nucleic acid construct provided herein (e.g., comprising in the genome of one or more of the subject's cells, such as their liver cells). In some embodiments, the method comprising administering to the subject: an RNA-guided DNA binding agent; and an endogenous SERPINA1 gene targeted nucleic acid agent that reducing expression of the endogenous SERPINA1 gene without significantly reducing expression of the AAT polypeptide coding sequences of the bidirectional nucleic acid construct.

In some embodiments of the methods provided herein, the endogenous SERPINA1 gene targeted nucleic acid agent is an siRNA, a dsRNA, or a guide RNA. In some embodiments, the endogenous SERPINA1 gene targeted nucleic acid agent is selected from an RNAi agent targeted to nucleotides 957-977, 1403-1425, or 1410-1436 of SEQ ID NO: 703, and a guide RNA targeted the endogenous SERPINA1 gene at a position corresponding to nucleotides 412-431, 506-525, or 538-557 of SEQ ID NO: 703.

In some embodiments, the method comprises inducing a double-stranded break (DSB) within the endogenous SERPINA1 gene. In certain embodiments, the method comprises inducing a double-strand break (DSB) is induced within the endogenous SERPINA1 gene at a position corresponding to nucleotides 412-431, 506-525, or 538-557 of SEQ ID NO: 703. In some embodiments, the method comprises modifying the endogenous SERPINA1 gene.

In certain embodiments, the SERPINA1 gene targeted nucleic acid agent is a SERPINA1 guide RNA that is at least partially complementary to a target sequence present in exon 2, 3, 4, or 5 of the endogenous human SERPINA1 gene and that targets neither the first AAT polypeptide coding sequence nor the second AAT polypeptide coding sequences. In some embodiments, the SERPINA1 gene targeted nucleic acid agent is a SERPINA1 guide RNA that is at least partially complementary to a target sequence within the endogenous SERPINA1 gene at a position corresponding to nucleotides 412-431, 506-525, or 538-557 of SEQ ID NO: 703. In some embodiments, the SERPINA1 guide RNA comprises: a guide sequence selected from SEQ ID NOs: 1129-1131; a guide sequence that is at least 95% identical to SEQ ID NOs: 1129-1131; or 17, 18, 19, or 20 consecutive nucleotides of a sequence chosen from SEQ ID NOs: 1129-1131.

In certain embodiments, the methods provided herein further comprise reducing expression of the endogenous SERPINA1 gene without significantly reducing expression of the AAT polypeptide coding sequences of the bidirectional nucleic acid construct.

In some embodiments of the methods provided herein, the subject has elevated liver enzymes. In some embodiments, the subject has at least 2×, at least 2.5× at least 3×, at least 3.5×, at least 4×, at least 4.5×, or at least 5×, upper limit of normal (ULN) of one or more liver enzymes. In some embodiments, the one or more liver enzymes is selected from alanine aminotransferase (ALT), and aspartate aminotransferase (AST). In certain embodiments, the method results in clinically relevant reduction of liver enzymes. In some embodiments, treatment results in reduction of the elevated liver enzymes to within 2×, 2.5×, 3×, 3.5×, 4×, 4.5×, or 5×ULN. In some embodiments, the method results in the treatment or prevention of liver fibrosis in the subject.

In certain embodiments, guide RNAs are used for the targeted insertion of a bidirectional nucleic acid construct provided herein into a human safe harbor site, such as intron 1 of an albumin safe harbor site. Also provided herein are donor constructs (e.g., a bidirectional nucleic acid construct provided herein), comprising a sequence encoding AAT, for use in targeted insertion into a human safe harbor site, such as intron 1 of an albumin safe harbor site. In some embodiments, the bidirectional nucleic acid construct provided herein can be used with any one or more gene editing systems (e.g., CRISPR/Cas system; zinc finger nuclease (ZFN) system; transcription activator-like effector nuclease (TALEN) system).

In some embodiments, the present disclosure provides a method of introducing a SERPINA1 nucleic acid to a cell or population of cells, comprising administering: i) a bidirectional nucleic acid construct provided herein; ii) an RNA-guided DNA binding agent; and iii) an albumin guide RNA (gRNA) comprising a sequence chosen from: a) a sequence that is at least 95%, 90%, 85%, 80%, or 75% identical to a sequence selected from the group consisting of SEQ ID NOs: 2-33; b) at least 17, 18, 19, or 20 contiguous nucleotides of a sequence selected from the group consisting of SEQ ID NOs: 2-33; c) a sequence that is at least 95%, 90%, 85%, 80%, or 75% identical to a sequence selected from the group consisting of SEQ ID NOs: 2-33; and d) a sequence that is complementary to 15 consecutive nucleotides+/−5 nucleotides of the genomic coordinates listed for SEQ ID NOs: 2-33, thereby introducing the SERPINA1 nucleic acid to the cell or population of cells.

In some embodiments, the present disclosure provides a method of expressing AAT in a subject in need thereof, comprising administering: i) a bidirectional nucleic acid construct provided herein; ii) an RNA-guided DNA binding agent; and iii) an albumin guide RNA (gRNA) comprising a sequence chosen from: a) a sequence that is at least 95%, 90%, 85%, 80%, or 75% identical to a sequence selected from the group consisting of SEQ ID Nos: 2-33; b) at least 17, 18, 19, or 20 contiguous nucleotides of a sequence selected from the group consisting of SEQ ID NOs: 2-33; c) a sequence that is at least 95%, 90%, 85%, 80%, or 75% identical to a sequence selected from the group consisting of SEQ ID NOs: 2-33; and d) a sequence that is complementary to 15 consecutive nucleotides+/−5 nucleotides of the genomic coordinates listed for SEQ ID NOs: 2-33, thereby expressing AAT in a subject in need thereof.

In some embodiments, the present disclosure provides a method of treating alpha-1 antitrypsin deficiency (AATD) in a subject in need of AAT protein, comprising administering: i) a bidirectional nucleic acid construct provided herein; ii) an RNA-guided DNA binding agent; and iii) an albumin guide RNA (gRNA) comprising a sequence chosen from: a) a sequence that is at least 95%, 90%, 85%, 80%, or 75% identical to a sequence selected from the group consisting of SEQ ID Nos: 2-33; b) at least 17, 18, 19, or 20 contiguous nucleotides of a sequence selected from the group consisting of SEQ ID NOs: 2-33; c) a sequence that is at least 95%, 90%, 85%, 80%, or 75% identical to a sequence selected from the group consisting of SEQ ID NOs: 2-33; and d) a sequence that is complementary to 15 consecutive nucleotides+/−5 nucleotides of the genomic coordinates listed for SEQ ID NOs: 2-33, thereby treating AATD in the subject.

In some embodiments, the present disclosure provides a method of increasing AAT secretion from a liver cell or population of cells, comprising administering: i) a bidirectional nucleic acid construct provided herein; ii) an RNA-guided DNA binding agent; and iii) an albumin guide RNA (gRNA) comprising a sequence chosen from: a) a sequence that is at least 95%, 90%, 85%, 80%, or 75% identical to a sequence selected from the group consisting of SEQ ID Nos: 2-33; b) at least 17, 18, 19, or 20 contiguous nucleotides of a sequence selected from the group consisting of SEQ ID NOs: 2-33; c) a sequence that is at least 95%, 90%, 85%, 80%, or 75% identical to a sequence selected from the group consisting of SEQ ID NOs: 2-33; and d) a sequence that is complementary to 15 consecutive nucleotides+/−5 nucleotides of the genomic coordinates listed for SEQ ID NOs: 2-33, thereby increasing AAT secretion from the liver cell or the population of cells.

In some embodiments, the bidirectional nucleic acid construct, RNA-guided DNA binding agent, albumin gRNA, and SERPINA1 gRNA are delivered or administered sequentially, in any order or in any combination.

In some embodiments, the bidirectional nucleic acid construct, RNA-guided DNA binding agent, albumin gRNA, and SERPINA1 gRNA, individually or in any combination, are delivered or administered simultaneously.

In some embodiments, the RNA-guided DNA binding agent, or RNA-guided DNA binding agent and albumin gRNA in combination, is delivered or administered prior to administering the bidirectional nucleic acid construct.

In some embodiments, the bidirectional nucleic acid construct is delivered or administered prior to delivering or administering the albumin gRNA or RNA-guided DNA binding agent

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the percent editing via indel formation in hSERPINA1 PIZ variant transgene in mouse liver after administration of LNP formulated guide RNAs G000409, G000414, or G000415 targeted to human SERPINA1.

FIGS. 2A and 2B show hA1AT serum levels (A) in μg/ml and (B) relative to control treated (% TSS) in hSERPINA1 PIZ variant transgene in mouse liver after administration of LNP formulated guide RNAs G000409, G000414, or G000415 targeted to human SERPINA1.

FIG. 3 shows A1AT protein expression (ng/ml) in primary mouse hepatocytes (PMH) after administration of various bidirectional constructs encoding human A1AT with various codon usages in AAV vectors.

FIGS. 4A and 4B show (A) serum hA1AT and (B) serum ALT activity levels in wild type (NGS) mice or in the PIZ transgenic mouse after administration of bidirectional constructs encoding hSERPINA1 or nanoluc in an AAV vector.

FIG. 5 shows A1AT protein expression in primary mouse hepatocytes (PMH) administration of various bidirectional constructs encoding human A1AT with various codon usages in AAV vectors.

FIGS. 6A-6C show results from a dose response study after administration of various bidirectional constructs (A) Construct 7, (B) Construct 8, and (C) Construct 9, each encoding human A1AT with various codon usages in AAV vectors.

FIG. 7 shows the percent editing (indel formation) in the cynomolgus albumin locus on Day 14 after treatment with G009860 and Construct 1, or treatment with vehicle.

FIG. 8 shows percent editing (indel formation) in cSERPINA1 on Day 259 of the study, 14 days after treatment with G014418, a cynomolgus specific SERPINA1 guide, or treatment with vehicle.

FIGS. 9A and 9B serum (A) hA1AT and (B) cA1AT assessed at the time points indicated. Bidirectional Construct 1 was administered on Day 1. Cynomolgus specific SERPINA1 guide G014418 was administered at Day 244 (indicated with arrow).

FIG. 10 shows percent editing (indel formation) in the cynomolgus albumin locus on Day 14 after treatment with G009860 and Construct 7 or Construct 8, or treatment with vehicle.

FIG. 11 shows circulating hA1AT levels in cynomolgus monkeys after treatment on Day 1 with G009860 and Construct 7 or Construct 8, or treatment with vehicle, at the indicated time points. The shaded area indicates normal levels of hA1AT in circulation (about 1000-2700 μg/ml or 20-53 μM).

FIGS. 12A and 12B show expression of AAT from expression constructs Alb-A1AT and Native-A1AT (FIG. 12A) and the percent inhibition of neutrophil elastase (FIG. 12B).

FIGS. 13A and 13B show hA1AT protein levels as measured by ELISA at Day 28 (pre-dose), and at Day 32 (post-dose) (FIG. 13A) and the percent knockdown of AAT following dosing of either siRNA2 or siRNA3 (FIG. 13B).

FIG. 14 shows serum hA1AT levels at one week and two weeks post dose. Asterisk (*) indicates 4 animals per group.

### DETAILED DESCRIPTION

Reference will now be made in detail to certain embodiments of the invention, examples of which are illustrated in the accompanying drawings. While the present teachings are described in conjunction with various embodiments, it is not intended to limit the invention to those embodiments. On the contrary, the present teachings encompass various alternatives, modifications, and equivalents, as will be appreciated by those of skill in the art.

Before describing the present teachings in detail, it is to be understood that the disclosure is not limited to specific compositions or process steps, as such may vary. It should be noted that, as used in this specification and the appended embodiments, the singular form “a,” “an,” and “the” include plural references unless the context dictates otherwise. Thus, for example, reference to “a conjugate” includes a plurality of conjugates and reference to “a cell” includes a plurality of cells and the like. As used herein, the term “include” and its grammatical variants are intended to be non-limiting, such that recitation of items in a list is not to the exclusion of other like items that can be substituted or added to the listed items.

Numeric ranges are inclusive of the numbers defining the range. Measured and measurable values are understood to be approximate, taking into account significant digits and the error associated with the measurement. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the embodiments).

The term “or” is used in an inclusive sense, i.e., equivalent to “and/or,” unless the context clearly indicates otherwise.

The term “about,” when used before a list, modifies each member of the list. The term “about” or “approximately” means an acceptable error for a particular value as determined by one of ordinary skill in the art, which depends in part on how the value is measured or determined.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 17 nucleotides of a 20 nucleotide nucleic acid molecule” means that 17, 18, 19, or 20 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified. As used herein, ranges include both the upper and lower limit.

As used herein, it is understood that when the maximum amount of a value is represented by 100% (e.g., 100% inhibition) that the value is limited by the method of detection. For example, 100% inhibition is understood as inhibition to a level below the level of detection of the assay.

The section headings used herein are for organizational purposes only and are not to be construed as limiting the desired subject matter in any way. In the event that any material incorporated by reference contradicts any term defined in this specification or any other express content of this specification, this specification controls.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with optional substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion, see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleosides and one or more nucleoside analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA,” “gRNA,” and simply “guide” are used herein interchangeably to refer to either a guide that comprises a guide sequence, e.g. either a crRNA (also known as CRISPR RNA), or the combination of a crRNA and a trRNA (also known as tracrRNA crRNA (also known as CRISPR RNA), or the combination of a crRNA and a trRNA (also known as tracrRNA). The crRNA and trRNA may be associated as a single RNA molecule (single guide RNA, sgRNA) or, for example, in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences. Guide RNAs, such as sgRNAs or dgRNAs, can include modified RNAs as described herein.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 15, 16, 17, 18, 19, or 20 contiguous nucleotides of an albumin guide sequence selected from SEQ ID NOs: 2-33 or SERPINA1 guide sequence selected from SEQ ID Nos: 1000-1131. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, or 100%. For example, in some embodiments, the guide sequence comprises a sequence with about 75%, 80%, 85%, 90%, 95%, or 100% identity to at least 15, 16, 17, 18, 19, or 20 contiguous nucleotides of an albumin guide sequence selected from SEQ ID NOs: 2-33 or SERPINA1 guide sequence selected from SEQ ID Nos: 1000-1131. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 15, 16, 17, 18, 19, 20 or more base pairs. In some embodiments, the guide sequence and the target region may contain 1-4 mismatches where the guide sequence comprises at least 15, 16, 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse complement), as a nucleic acid substrate for an RNA-guided DNA binding agent is a double stranded nucleic acid. Accordingly, where a guide sequence is said to be “complementary to a target sequence,” it is to be understood that the guide sequence may direct a guide RNA to bind to the sense or antisense strand (e.g. reverse complement) of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA-binding agent” means a polypeptide or complex of polypeptides having RNA and DNA binding activity, or a DNA-binding subunit of such a complex, wherein the DNA binding activity is sequence-specific and depends on the sequence of the RNA. The term RNA-guided DNA binding-agent also includes nucleic acids encoding such polypeptides. Exemplary RNA-guided DNA-binding agents include Cas cleavases/nickases. Exemplary RNA-guided DNA-binding agents may include inactivated forms thereof (“dCas DNA-binding agents”), e.g. if those agents are modified to permit DNA cleavage, e.g. via fusion with a FokI cleavase domain. “Cas nuclease,” as used herein, encompasses Cas cleavases and Cas nickases. Cas cleavases and Cas nickases include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA-binding agents, in which cleavase/nickase activity is inactivated”), if those agents are modified to permit DNA cleavage. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015) also contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015). As used herein, delivery of an RNA-guided DNA-binding agent (e.g. a Cas nuclease, a Cas9 nuclease, or an S. pyogenes Cas9 nuclease) includes delivery of the polypeptide or mRNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm inteace provided by the EBI at the www.ebi.ac.uk web server is generally appropriate.

As used herein, a first sequence is considered to be “X % complementary to” a second sequence if X % of the bases of the first sequence base pairs with the second sequence. For example, a first sequence 5′AAGA3′ is 100% complementary to a second sequence 3′TTCT5′, and the second sequence is 100% complementary to the first sequence. In some embodiments, a first sequence 5′AAGA3′ is 100% complementary to a second sequence 3′TTCTGTGA5′, whereas the second sequence is 50% complementary to the first sequence.

As used herein, “CpG depleted” and the like are understood as modification of a nucleotide sequence to reduce, or preferably eliminate, the presence of CpG dinucleotides. CpG depletion in a coding sequence without changing the encoded amino acid sequence can be readily accomplished by alternative codon usage. As used herein, a CpG depleted coding sequence of an A1AT protein contains no more than 3 CpG dinucleotides (i.e., 3, 2, 1, or 0 CpG dinucleotides), preferably the coding sequence for an A1AT protein contains no CpG dinucleotides. It is understood that other portions of expression constructs may be selected or designed to have a minimal number of CpG dinucleotides (see, e.g., Wright J F, Mol Ther. 2020).

As used herein, “use of a non-wild type codon” is understood as modification of a coding sequence without changing the encoded amino acid sequence can be readily accomplished by alternative codon usage. As used herein, use of a non-wild type codon includes alternate codon usage for at least 10%, 20%, 30%, or 40% of the wild type codons with non-wild type codons within a defined region. As some regions defined herein may include codons that are partially within the region, the partial codon sequence is compared against the wild type sequence. If the partial codon includes a change from the wild type sequence within the defined region, the codon is considered to use a non-wild type codon. If the partial codon does not include a change from the wild type sequence within the defined region, the codon is considered to have wild-type codon usage.

As used herein, “mRNA” is used herein to refer to a polynucleotide that is entirely or predominantly RNA or modified RNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Exemplary guide sequences useful in the guide RNA compositions and methods described herein are shown in Table 1, Table 2, and throughout the application.

As used herein, “indels” refer to insertion/deletion mutations consisting of a number of nucleotides that are either inserted or deleted at the site of double-stranded breaks (DSBs) in a target nucleic acid.

As used herein, “heterologous alpha-1 antitrypsin” is used interchangeably with “heterologous AAT” or “heterologous A1AT” or “AAT/A1AT transgene,” which is the gene product of a SERPINA1 gene that is heterologous with respect to its insertion site. In some embodiments, the SERPINA1 gene is exogenous. The human wild-type AAT protein sequence is available at NCBI NP_000286; gene sequence is available at NCBI NM_000295. The human wild-type AAT cDNA has been sequenced (see, e.g., Long et al., “Complete sequence of the cDNA for human alpha 1-antitrypsin and the gene for the S variant,” Biochemistry 1984) and encodes a precursor molecule containing a signal peptide and a mature AAT peptide. Domains of the peptide responsible for intracellular targeting, carbohydrate attachment, catalytic function, protease inhibitory activity, etc., have been characterized (see, e.g., Kalsheker, “Alpha 1-antitrypsin: structure, function and molecular biology of the gene,” Biosci Rep. 1989; Matamala et al., “Identification of Novel Short C-Terminal Transcripts of Human SERPINA1 Gene,” PLoS One 2017; Niemann et al., “Isolation and serine protease inhibitory activity of the 44-residue, C-terminal fragment of alpha 1-antitrypsin from human placenta,” Matrix 1992). As used herein, heterologous AAT encompasses precursor AAT, mature AAT, and variants and fragments thereof, e.g., functional fragments, e.g., fragments that retain protease inhibitory activity (e.g., at least 60%, 70%, 80%, 85%, 90%, 92%, 94%, 96%, 98%, 99%, or 100%, compared to wild-type AAT, e.g., as assayed by a commercially available protease inhibition assay or human neutrophil elastase (HNE) inhibition assay). In some embodiments, the functional fragment is naturally occurring, e.g., a short C-terminal fragment. In some embodiments, the functional fragment is genetically engineered, e.g., a hyperactive functional fragment. Examples of the AAT protein sequence are described herein (e.g. SEQ ID NO: 700 or SEQ ID NO: 702). As used herein, heterologous AAT also encompasses a variant of AAT, e.g., a variant that possesses increased protease inhibitor activity as compared to wild type AAT. As used herein, heterologous AAT also encompasses a variant that is 80%, 85%, 90%, 93%, 95%, 97%, 99% identical to SEQ ID NO: 700, having functional activity—e.g., at least 60%, 70%, 80%, 85%, 90%, 92%, 94%, 96%, 98%, 99%, 100%, or more, activity as compared to wild type AAT, e.g., as assayed by HNE inhibition. As used herein, heterologous AAT also encompasses a fragment that possesses functional activity—e.g., at least 80%, 85%, 90%, 92%, 94%, 96%, 98%, 99%, 100%, or more, activity as compared to wild type AAT, e.g., as assayed by HNE inhibition. As used herein, heterologous AAT refers to an AAT, e.g. a functional AAT, useful in treating AATD, which may be wild-type AAT or a variant thereof useful in treating AATD.

As used herein, a “heterologous gene” refers to a gene that has been introduced as an exogenous source to a site within a host cell genome (e.g., at a genomic locus such as a safe harbor locus, including an albumin intron 1 site). A polypeptide expressed from such heterologous gene is referred to as a “heterologous polypeptide.” The heterologous gene can be naturally-occurring or engineered, and can be wild type or a variant. The heterologous gene may include nucleotide sequences other than the sequence that encodes the heterologous polypeptide. The heterologous gene can be a gene that occurs naturally in the host genome, as a wild type or a variant (e.g., mutant). For example, although the host cell contains the gene of interest (as a wild type or as a variant), the same gene or variant thereof can be introduced as an exogenous source for, e.g., expression at a locus that is highly expressed. The heterologous gene can also be a gene that is not naturally occurring in the host genome, or that expresses a heterologous polypeptide that does not naturally occur in the host genome. “Heterologous gene,” “exogenous gene,” and “transgene” are used interchangeably. In some embodiments, the heterologous gene or transgene includes an exogenous nucleic acid sequence, e.g. a nucleic acid sequence is not endogenous to the recipient cell. In certain embodiments, the heterologous gene can include an AAT nucleic acid sequence that does not naturally occur in the recipient cell. An AAT polypeptide coding sequence is a nucleic acid sequence that encodes for active polypeptide that inhibits elastase. For example, heterologous AAT may be heterologous with respect to its insertion site and with respect to its recipient cell.

As used herein, “mutant SERPINA1” or “mutant SERPINA1 allele” refers to a SERPINA1 sequence having a change in the nucleotide sequence of SERPINA1 compared to the wildtype sequence (NCBI Gene ID: 5265; NCBI NM_000295; Ensembl: Ensembl:ENSG00000197249). In some embodiments, a mutant SERPINA1 allele encodes a non-functional or non-secreted AAT protein.

As used herein, “AATD” or “A1AD” refers to alpha-1 antitrypsin deficiency. AATD comprises diseases and disorders caused by a variety of different genetic mutations in SERPINA1. AATD may refer to a disease where decreased levels of functional AAT are expressed (e.g., less than 100%, 90%, 80%, 70%, 60%, 50%, 40%, 30%, 20%, 10%, or 5% AAT gene or protein expression as compared to a control sample, e.g., by nephelometry or immunoturbidimetry, e.g., AAT less than about 100 mg/dL, 90 mg/dL, 80 mg/dL, 70 mg/dL, 60 mg/dL, 50 mg/dL, 40 mg/dL, 30 mg/dL, 20 mg/dL, 10 mg/dL, or 5 mg/dL in serum), functional AAT is not expressed, or a mutant or non-functional AAT is expressed (e.g., forms aggregates or is not capable of being secreted or has decreased protease inhibitor activity). See, e.g., Greulich and Vogelmeier, Ther Adv Respir Dis 2016; Stoller and Aboussouan, Lancet, 2005. In some embodiments, AATD refers to a disease where AAT is aggregated or accumulated intracellularly, e.g., in a hepatocyte, and not secreted, e.g., into circulation where it may be delivered to the lungs to function as a protease inhibitor. In some embodiments, AATD may be detected by PASD staining of liver tissue sections, e.g., to measure aggregation. In some embodiments, AATD may be detected by decreased inhibition of neutrophil elastase, e.g., in the lung.

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, a “nucleic acid therapeutic agent” is understood as a therapeutic agent comprising a sufficient length of nucleotides to specifically hybridize to a target sequence in a target nucleic acid in a cell such that the hybridization reduces levels of a protein encoded by the target nucleic acid, e.g., by inhibiting translation or promoting sequence specific degradation of the target nucleic acid, or causing a change in the DNA encoding the protein resulting in a reduction of mRNA or protein expression. Exemplary nucleic acid therapeutic agents include RNAi agents, including Dicer Substrate (ds)RNAi agents, or antisense oligonucleotide agents; or RNA-guided DNA binding agents including CISPR, TALEN, or zinc finger nuclease (ZFN).

The terms “iRNA”, “RNAi agent,” “iRNA agent,”, “RNA interference agent”, “siRNA”, “siRNA agent” as used interchangeably herein, refer to an agent that contains RNA as that term is defined herein, and which mediates the targeted cleavage of an RNA transcript, e.g., via an RNA-induced silencing complex (RISC) pathway. iRNA directs the sequence-specific degradation of mRNA through a process known as RNA interference (RNAi). In general, an “iRNA” includes ribonucleotides with chemical modifications. Such modifications may include all types of modifications disclosed herein or known in the art. Any such modifications, as used in a dsRNA molecule, are encompassed by “iRNA” for the purposes of this specification and claims. The RNAi agent may or may not be processed by Dicer prior to entering the RISC pathway. That is, an RNAi agent is a nucleic acid therapeutic that acts by reducing the expression of a target gene, thereby reducing the expression of the polypeptide encoded by the target gene. Exemplary iRNA agents targeted to SERPINA1 are provided, for example, in WO2018098117, WO2015003113, and WO2015195628A2.

As used herein, a “nucleic acid therapeutic agent that reduces expression of SERPINA1” and the like as used herein is understood as a nucleic acid therapeutic agent that reduces levels of SERPINA1 RNA, A1AT protein encoded by SERPINA1, or both of SERPINA1 RNA and protein encoded by SERPINA1. In some embodiments, the nucleic acid therapeutic agent that reduces expression of SERPINA1 is a therapeutic agent that promotes the degradation of an mRNA encoding SERPINA1 or inhibits the translation of an mRNA encoding SERPINA1. Such agents include, but are not limited to, nucleic acid therapeutics, e.g., RNAi interference agents and antisense oligonucleotide agents. Such agents can typically inhibit expression of both endogenous wild type and mutant SERPINA1. In certain embodiments, expression of endogenous SERPINA1 may be inhibited while expression of a heterologous SERPINA1 is not inhibited due to the design of the heterologous coding sequence. As used herein, “normal” or “healthy” individuals include those individuals that do not have the AATD-associated alleles—e.g., AATD-associated alleles are ZZ, MZ, or SZ.

As used herein, “treatment” refers to any administration or application of a therapeutic for disease or disorder in a subject, and includes inhibiting the disease, arresting its development, relieving one or more symptoms of the disease, curing the disease, or preventing reoccurrence of one or more symptoms of the disease. AATD may be associated with lung disease or liver disease; wheezing or shortness of breath; increased risk of lung infections; chronic obstructive pulmonary disease (COPD); bronchitis, asthma, dyspnea; cirrhosis; neonatal jaundice; panniculitis; chronic cough or phlegm; recurring chest colds; yellowing of the skin or the white part of the eyes; swelling of the belly or legs. For example, treatment of AATD may comprise alleviating symptoms of AATD, e.g., liver or lung symptoms. In some embodiments, treatment refers to increasing serum AAT levels, e.g., to protective levels. In some embodiments, treatment refers to increasing serum AAT levels, e.g., within the normal range. In some embodiments, treatment refers to increasing serum AAT levels, e.g., above 40, 50, 60, 70, 80, 90, or 100 mg/dL, e.g., as measured using nephelometry or immunoturbidimetry and a purified standard. In some embodiments, treatment refers to improvement in baseline serum AAT as compared to control, e.g., before and after treatment. In some embodiments, treatment refers to an improvement in histologic grading of AATD associated liver disease, e.g., by 1, 2, 3, or more points, as compared to control, e.g., before and after treatment. In some embodiments, treatment refers to improvement in Ishak fibrosis score as compared to control, e.g., before and after treatment. In some embodiments, treatment refers to improvement in genotype serum level, AAT lung function, spirometry test, chest X-ray of lung, CT scan of lung, blood testing of liver function, or ultrasound of liver.

As used herein, “knockdown” refers to a decrease in expression of a particular gene product (e.g., protein, mRNA, or both). Knockdown of a protein can be measured by, for example, detecting protein secreted by tissue or population of cells (e.g., in serum or cell media) or by detecting total cellular amount of the protein from a tissue or cell population of interest. Methods for measuring knockdown of mRNA are known, and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues). In some embodiments, the methods of the disclosure “knockdown” endogenous AAT in one or more cells (e.g., in a population of cells including in vivo populations such as those found in tissues). Relevant cells include cells that are capable of producing AAT. In some embodiments, the methods provided herein knockdown an endogenous mutant SERPINA1 allele, or an endogenous wildtype SERPINA1 allele (e.g., in a heterozygous MZ individual).

As used herein, “knockout” refers to a loss of expression of a particular protein in a cell. Knockout can be measured either by detecting the amount of protein secretion from a tissue or population of cells (e.g., in serum or cell media) or by detecting total cellular amount of a protein a tissue or a population of cells. Relevant cells include cells that are capable of producing AAT. In some embodiments, the methods provided herein “knockout” endogenous AAT in one or more cells (e.g., in a population of cells including in vivo populations such as those found in tissues). In some embodiments, the methods of the of the disclosure knockout an endogenous mutant SERPINA1 allele, or an endogenous wildtype SERPINA1 allele (e.g., in a heterozygous MZ individual). In some embodiments, a knockout is the complete loss of expression of endogenous AAT protein in a cell.

As used herein, “polypeptide” refers to a wild-type or variant protein (e.g., mutant, fragment, fusion, or combinations thereof). A variant polypeptide may possess at least or about 5%, 10%, 15%, 20%, 30%, 35%, 40%, 45%, 50%, 55%, 60%, 65%, 70%, 75%, 80%, 85%, 90%, 95%, or 100% functional activity of the wild-type polypeptide. In some embodiments, the variant is at least 70%, 75%, 80%, 85%, 90%, 92%, 93%, 94%, 95%, 96%, 97%, 98%, or 99% identical to the sequence of the wild-type polypeptide. In some embodiments, a variant polypeptide may be a hyperactive variant. In certain instances, the variant possesses between about 80% and about 120%, 140%, 160%, 180%, 200% of the functional activity of the wild-type polypeptide.

As used herein, a “bidirectional nucleic acid construct” (interchangeably referred to herein as “bidirectional construct”) comprises at least two nucleic acid segments, wherein one segment (the first segment) comprises a coding sequence that encodes a polypeptide of interest (the coding sequence may be referred to herein as “transgene” or a first transgene), while the other segment (the second segment) comprises a sequence wherein the complement of the sequence encodes a polypeptide of interest, or a second transgene. That is, the at least two segments can encode identical or different polypeptides. When the two segments encode the identical polypeptide, the coding sequence of the first segment need not be identical to the complement of the sequence of the second segment. In some embodiments, the sequence of the second segment is a reverse complement of the coding sequence of the first segment. A bidirectional construct can be single-stranded or double-stranded. The bidirectional construct disclosed herein encompasses a construct that is capable of expressing any polypeptide of interest.

As used herein, a “reverse complement” refers to a sequence that is a complement sequence of a reference sequence, wherein the complement sequence is written in the reverse orientation. For example, for a hypothetical sequence 5′ CTGGACCGA 3′ (SEQ ID NO: 500), the “perfect” complement sequence is 3′ GACCTGGCT 5′ (SEQ ID NO: 501), and the “perfect” reverse complement is written 5′ TCGGTCCAG 3′ (SEQ ID NO: 502). A reverse complement sequence need not be “perfect” and may still encode the same polypeptide or a similar polypeptide as the reference sequence. Due to codon usage redundancy, a reverse complement can diverge from a reference sequence that encodes the same polypeptide. As used herein, “reverse complement” also includes sequences that are, e.g., 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence.

In some embodiments, a bidirectional nucleic acid construct comprises a first segment that comprises a coding sequence that encodes a first polypeptide (a first transgene), and a second segment that comprises a sequence wherein the complement of the sequence encodes a second polypeptide (a second transgene). In some embodiments, the first and the second polypeptides are at least 50%, 55%, 60%, 65%, 70%, 75%, 80%, 85%, 90%, 91%, 92%, 93%, 94%, 95%, 96%, 97%, 98%, 99%, or 100% identical. In some embodiments, the first and the second polypeptides comprise an amino acid sequence that is at least 80%, 85%, 90%, 91%, 92%, 93%, 94%, 95%, 96%, 97%, 98%, 99%, or 100% identical, e.g. across 50, 100, 200, 500, 1000 or more amino acid residues.

A “safe harbor” locus is a locus within the genome wherein a gene may be inserted without significant deleterious effects on the host cell, e.g. hepatocyte, e.g., without causing apoptosis, necrosis, or senescence, or without causing more than 5%, 10%, 15%, 20%, 25%, 30%, or 40% apoptosis, necrosis, or senescence as compared to a control cell. See, e.g., Hsin et al., “Hepatocyte death in liver inflammation, fibrosis, and tumorigenesis,” 2017. In some embodiments, a safe harbor locus allows overexpression of an exogenous gene without significant deleterious effects on the host cell, e.g. hepatocyte, without causing apoptosis, necrosis, or senescence, or without causing more than 5%, 10%, 15%, 20%, 25%, 30%, or 40% apoptosis, necrosis, or senescence as compared to a control cell. In some embodiments, a desirable safe harbor locus may be one in which expression of the inserted gene sequence is not perturbed by read-through expression from neighboring genes. The safe harbor may be within an albumin gene, such as a human albumin gene. The safe harbor may be within an albumin intron 1 region, e.g., human albumin intron 1. The safe harbor may be a human safe harbor, e.g., for a liver tissue or hepatocyte host cell. In some embodiments, a safe harbor allows overexpression of an exogenous gene without significant deleterious effects on the host cell or cell population, such as hepatocytes or liver cells, e.g. without causing apoptosis, necrosis, or senescence, or without causing more than 5%, 10%, 15%, 20%, 25%, 30%, or 40% apoptosis, necrosis, or senescence as compared to a control cell.

In some embodiments, the gene may be inserted into a safe harbor locus and use the safe harbor locus's endogenous signal sequence, e.g., the albumin signal sequence encoded by exon 1. For example, an AAT coding sequence may be inserted into human albumin intron 1 such that it is downstream of and fuses to the signal sequence of human albumin exon 1.

In some embodiments, the gene may comprise its own signal sequence, may be inserted into the safe harbor locus, and may further use the safe harbor locus's endogenous signal sequence. For example, an AAT coding sequence comprising an AAT signal sequence may be inserted into human albumin intron 1 such that it is downstream of and fuses to the signal sequence of human albumin encoded by exon 1.

In some embodiments, the gene may comprise its own signal sequence and an internal ribosomal entry site (IRES), may be inserted into the safe harbor locus, and may further use the safe harbor locus's endogenous signal sequence. For example, an AAT coding sequence comprising an AAT signal sequence and an IRES sequence may be inserted into human albumin intron 1 such that it is downstream of and fuses to the signal sequence of human albumin encoded by exon 1.

In some embodiments, the gene may comprise its own signal sequence and IRES, may be inserted into the safe harbor locus, and does not use the safe harbor locus's endogenous signal sequence. For example, an AAT coding sequence comprising an AAT signal sequence and an IRES sequence may be inserted into human albumin intron 1 such that it does not fuse to the signal sequence of human albumin encoded by exon 1. In these embodiments, the protein is translated from the IRES site and is not chimeric (e.g., albumin signal peptide fused to AAT protein), which may be advantageously non- or low-immunogenic. In some embodiments, the protein is not secreted or transported extracellularly.

In some embodiments, the gene may be inserted into the safe harbor locus and may comprise an IRES and does not use any signal sequence. For example, an AAT coding sequence comprising an IRES sequence and no AAT signal sequence may be inserted into human albumin intron 1 such that it does not fuse to the signal sequence of human albumin encoded by exon 1. In some embodiments, the proteins is translated from the IRES site without the need for any signal sequence. In some embodiments, the proteins is not transported extracellularly.

As used herein, a cell that is not undergoing mitotic cell division is referred to as a “non-dividing” cell. A “non-dividing” cell encompasses cell types that never or rarely undergo mitotic cell division, e.g., many types of neurons. A “non-dividing” cell also encompasses cells that are capable of, but not undergoing or about to undergo, mitotic cell division, e.g., a quiescent cell. Liver cells, for example, retain the ability to divide (e.g., when injured or resected), but do not typically divide. During mitotic cell division, homologous recombination is a mechanism by which the genome is protected and double-stranded breaks are repaired. In some embodiments, a “non-dividing” cell refers to a cell in which homologous recombination (HR) is not the primary mechanism by which double-stranded DNA breaks are repaired in the cell, e.g., as compared to a control dividing cell. In some embodiments, a “non-dividing” cell refers to a cell in which non-homologous end joining (NHEJ) is the primary mechanism by which double-stranded DNA breaks are repaired in the cell, e.g., as compared to a control dividing cell.

Non-dividing cell types have been described in the literature, e.g. by active NHEJ double-stranded DNA break repair mechanisms. See, e.g. Iyama, DNA Repair (Amst.) 2013, 12(8): 620-636. In some embodiments, the host cell includes, but is not limited to, a liver cell, a muscle cell, or a neuronal cell. In some embodiments, the host cell is a hepatocyte, such as a mouse, cynomolgus, or human hepatocyte. In some embodiments, the host cell is a myocyte, such as a mouse, cynomolgus, or human myocyte. In some embodiments, provided herein is a host cell, described above, that comprises the bidirectional construct disclosed herein. In some embodiments the host cell expresses the transgene polypeptide encoded by the bidirectional construct disclosed herein. In some embodiments, provided herein is a host cell made by a method disclosed herein. In certain embodiments, the host cell is made by administering or delivering to a host cell a bidirectional nucleic acid construct described herein, and a gene editing system such as a ZFN, TALEN, or CRISPR/Cas9 system.

### II. Compositions

A. Compositions Comprising Safe Harbor Albumin Guide RNA (gRNAs) or SERPINA1 Guide RNA (gRNAs)

Provided herein are albumin guide RNA compositions, AAT template compositions, and methods useful for inserting and expressing a heterologous AAT gene (e.g., a functional or wild-type AAT) within a genomic locus such as a safe harbor gene of a host cell. In particular, as exemplified herein, targeting and inserting a heterologous AAT gene at the albumin locus (e.g., at intron 1) allows the use of albumin's endogenous promoter to drive robust expression of the heterologous AAT gene. The present disclosure is based, in part, on the identification of albumin guide RNAs that specifically target sites within intron 1 of the albumin gene, SERPINA1 nucleic acid sequences with alternative codon usage, and guide RNAs that bind to endogenous SERPINA1 nucleic acids but not the SERPINA1 nucleic acids with alternative codon usage. As shown in the Examples and further described herein, expression of the AAT transgene is unaffected by simultaneous or non-simultaneous administrating of gRNAs (or siRNAs) that specifically target endogenous SERPINA1 nucleic acids.

In some embodiments, disclosed herein are compositions useful for introducing or inserting a heterologous AAT gene (e.g., a functional or wild-type AAT) within a locus such as an albumin locus (e.g., intron 1) of a host cell, e.g., using an albumin guide RNA disclosed herein with an RNA-guided DNA binding agent (e.g., Cas nuclease), and a construct (e.g., donor construct or template) comprising a heterologous AAT nucleic acid (“AAT transgene”). In some embodiments, disclosed herein are compositions useful for expressing a heterologous AAT gene at an albumin locus of a host cell, e.g., using an albumin guide RNA disclosed herein with an RNA-guided DNA binding agent and a construct (e.g., donor) comprising a heterologous AAT nucleic acid. In some embodiments, disclosed herein are compositions useful for expressing a heterologous AAT at an albumin locus of a host cell, e.g., using an albumin guide RNA disclosed herein with an RNA-guided DNA binding agent and a bidirectional construct comprising a heterologous AAT nucleic acid. In some embodiments, disclosed herein are compositions useful for inducing a break (e.g., double-stranded break (DSB) or single-stranded break (SSB or nick)) within the albumin gene of a host cell, e.g., using an albumin guide RNA disclosed herein with an RNA-guided DNA binding agent (e.g., a CRISPR/Cas system). The compositions may be used in vitro or in vivo for, e.g., treating AATD.

In some embodiments, the albumin guide RNAs disclosed herein comprise a guide sequence that binds, or is capable of binding, within an intron of an albumin locus. In some embodiments, the albumin guide RNAs disclosed herein bind within a region of intron 1 of the human albumin gene of SEQ ID NO: 1. It will be appreciated that not every base of the albumin guide sequence must bind within the recited regions. For example, in some embodiments, 15, 16, 17, 18, 19, 20, or more, bases of the albumin guide RNA sequence bind within the recited regions. For example, in some embodiments, 15, 16, 17, 18, 19, 20, or more contiguous bases of the guide RNA sequence bind with the recited regions.

In some embodiments, the albumin guide RNAs disclosed herein mediate a target-specific cutting by an RNA-guided DNA binding agent (e.g., Cas nuclease) at a site within intron 1 of human albumin (SEQ ID NO: 1). It will be appreciated that, in some embodiments, the guide RNAs comprise guide sequences that bind to, or are capable of binding to, said regions.

In some embodiments, the albumin guide RNAs disclosed herein comprise a guide sequence that is at least 95% identical or 90% identical to a sequence selected from the group consisting of SEQ ID NOs: 2-33.

In some embodiments, the albumin guide RNAs disclosed herein comprise a guide sequence having at least 15, 16, 17, 18, 19, or 20 contiguous nucleotides of a sequence selected from the group consisting of SEQ ID NOs: 2-33.

In some embodiments, the albumin guide RNA (gRNA) comprises a guide sequence chosen from: a) a sequence that is at least 95%, 90%, 85%, 80%, or 75% identical to a sequence selected from the group consisting of SEQ ID NOs: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides+/−5 nucleotides of the genomic coordinates listed for SEQ ID NOs: 2-33. In some embodiments, the albumin guide RNA comprises a sequence selected from the group consisting of SEQ ID NO: 2, 8, 13, 19, 28, 29, 31, 32, 33. See Table 1.

Human albumin intron 1:
(SEQ ID NO: 1)
GTAAGAAATCCATTTTTCTATTGTTCAACTTTTATTCTATTTTCCCAGTA
AAATAAAGTTTTAGTAAACTCTGCATCTTTAAAGAATTATTTTGGCATTT
ATTTCTAAAATGGCATAGTATTTTGTATTTGTGAAGTCTTACAAGGTTAT
CTTATTAATAAAATTCAAACATCCTAGGTAAAAAAAAAAAAAGGTCAGAA
TTGTTTAGTGACTGTAATTTTCTTTTGCGCACTAAGGAAAGTGCAAAGTA
ACTTAGAGTGACTGAAACTTCACAGAATAGGGTTGAAGATTGAATTCATA
ACTATCCCAAAGACCTATCCATTGCACTATGCTTTATTTAAAAACCACAA
AACCTGTGCTGTTGATCTCATAAATAGAACTTGTATTTATATTTATTTTC
ATTTTAGTCTGTCTTCTTGGTTGCTGTTGATAGACACTAAAAGAGTATTA
GATATTATCTAAGTTTGAATATAAGGCTATAAATATTTAATAATTTTTAA
AATAGTATTCTTGGTAATTGAATTATTCTTCTGTTTAAAGGCAGAAGAAA
TAATTGAACATCATCCTGAGTTTTTCTGTAGGAATCAGAGCCCAATATTT
TGAAACAAATGCATAATCTAAGTCAAATGGAAAGAAATATAAAAAGTAAC
ATTATTACTTCTTGTTTTCTTCAGTATTTAACAATCCTTTTTTTTCTTCC
CTTGCCCAG

TABLE 1
Albumin targeted human guide RNA sequences
and chromosomal coordinates

| | | | SEQ |
|---|---|---|---|
| Guide | | | ID |
| ID | Guide Sequence | Genomic Coordinates | NO: |
| G009844 | GAGCAACCUCACUCUUGUCU | chr4:73405113-73405133 | 2 |
| G009851 | AUGCAUUUGUUUCAAAAUAU | chr4:73405000-73405020 | 3 |
| G009852 | UGCAUUUGUUUCAAAAUAUU | chr4:73404999-73405019 | 4 |
| G009857 | AUUUAUGAGAUCAACAGCAC | chr4:73404761-73404781 | 5 |
| G009858 | GAUCAACAGCACAGGUUUUG | chr4:73404753-73404773 | 6 |
| G009859 | UUAAAUAAAGCAUAGUGCAA | chr4:73404727-73404747 | 7 |
| G009860 | UAAAGCAUAGUGCAAUGGAU | chr4:73404722-73404742 | 8 |
| G009861 | UAGUGCAAUGGAUAGGUCUU | chr4:73404715-73404735 | 9 |
| G009866 | UACUAAAACUUUAUUUUACU | chr4:73404452-73404472 | 10 |
| G009867 | AAAGUUGAACAAUAGAAAAA | chr4:73404418-73404438 | 11 |
| G009868 | AAUGCAUAAUCUAAGUCAAA | chr4:73405013-73405033 | 12 |
| G009874 | UAAUAAAAUUCAAACAUCCU | chr4:73404561-73404581 | 13 |
| G012747 | GCAUCUUUAAAGAAUUAUUU | chr4:73404478-73404498 | 14 |
| G012748 | UUUGGCAUUUAUUUCUAAAA | chr4:73404496-73404516 | 15 |
| G012749 | UGUAUUUGUGAAGUCUUACA | chr4:73404529-73404549 | 16 |
| G012750 | UCCUAGGUAAAAAAAAAAAA | chr4:73404577-73404597 | 17 |
| G012751 | UAAUUUUCUUUUGCGCACUA | chr4:73404620-73404640 | 18 |
| G012752 | UGACUGAAACUUCACAGAAU | chr4:73404664-73404684 | 19 |
| G012753 | GACUGAAACUUCACAGAAUA | chr4:73404665-73404685 | 20 |
| G012754 | UUCAUUUUAGUCUGUCUUCU | chr4:73404803-73404823 | 21 |
| G012755 | AUUAUCUAAGUUUGAAUAUA | chr4:73404859-73404879 | 22 |
| G012756 | AAUUUUUAAAAUAGUAUUCU | chr4:73404897-73404917 | 23 |
| G012757 | UGAAUUAUUCUUCUGUUUAA | chr4:73404924-73404944 | 24 |
| G012758 | AUCAUCCUGAGUUUUUCUGU | chr4:73404965-73404985 | 25 |
| G012759 | UUACUAAAACUUUAUUUUAC | chr4:73404453-73404473 | 26 |
| G012760 | ACCUUUUUUUUUUUUUACCU | chr4:73404581-73404601 | 27 |
| G012761 | AGUGCAAUGGAUAGGUCUUU | chr4:73404714-73404734 | 28 |
| G012762 | UGAUUCCUACAGAAAAACUC | chr4:73404973-73404993 | 29 |
| G012763 | UGGGCAAGGGAAGAAAAAAA | chr4:73405094-73405114 | 30 |
| G012764 | CCUCACUCUUGUCUGGGCAA | chr4:73405107-73405127 | 31 |
| G012765 | ACCUCACUCUUGUCUGGGCA | chr4:73405108-73405128 | 32 |
| G012766 | UGAGCAACCUCACUCUUGUC | chr4:73405114-73405134 | 33 |

The albumin guide RNAs disclosed herein mediate a target-specific cutting resulting in a double-stranded break (DSB). The albumin guide RNAs disclosed herein mediate a target-specific cutting resulting in a single-stranded break (SSB or nick).

In some embodiments, the albumin guide RNAs disclosed herein bind to a region upstream of a protospacer adjacent motif (PAM). As would be understood by those of skill in the art, the PAM sequence occurs on the strand opposite to the strand that contains the target sequence. That is, the PAM sequence is on the complement strand of the target strand (the strand that contains the target sequence to which the guide RNA binds). In some embodiments, the PAM is selected from the group consisting of NGG, NNGRRT, NNGRR(N), NNAGAAW, NNNNG(A/C)TT, and NNNNRYAC. In some embodiments, the PAM is NGG.

In some embodiments, the guide RNA sequences provided herein are complementary to a sequence adjacent to a PAM sequence.

In some embodiments, the guide RNA sequence comprises a sequence that is complementary to a sequence within a genomic region selected from the tables herein according to coordinates in human reference genome hg38. In some embodiments, the guide RNA sequence comprises a sequence that is complementary to a sequence that comprises 15, 16, 17, 18, 19, or 20 consecutive nucleotides from within a genomic region selected from the tables herein. In some embodiments, the guide RNA sequence comprises a sequence that is complementary to a sequence that comprises 15, 16, 17, 18, 19, or 20 consecutive nucleotides spanning a genomic region selected from the tables herein.

The guide RNAs disclosed herein mediate a target-specific cutting resulting in a double-stranded break (DSB). The guide RNAs disclosed herein mediate a target-specific cutting resulting in a single-stranded break (SSB or nick).

In some embodiments, the albumin guide RNAs disclosed herein mediates target-specific cutting by an RNA-guided DNA binding agent (e.g., a Cas nuclease, as disclosed herein), wherein a resultant cut site allows insertion of a heterologous AAT nucleic acid (e.g., a functional or wild-type AAT) within intron 1 of an albumin gene. In some embodiments, the guide RNA or cut site allows between 25 and 30%, 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95% insertion of a heterologous AAT gene. In some embodiments, the guide RNA or cut site allows 25-90%, 25-80%, 25-70%, 25-50%, 35-80%, or 35-70% insertion of a heterologous AAT gene. In some embodiments, the guide RNA or cut site allows at least 25%, at least 30%, at least 40%, at least 50%, at least 60%, at least 70%, at least 80%, at least 90% insertion of a heterologous AAT nucleic acid. Insertion rates can be measured in vitro or in vivo. For example, in some embodiments, rate of insertion can be determined by detecting and measuring the inserted heterologous AAT nucleic acid within a population of cells, and calculating a percentage of the population that contains the inserted heterologous AAT nucleic acid. Methods of measuring insertion rates are known and available in the art. Such methods include, e.g., sequencing of the insertion site or sequencing mRNA isolated from a tissue or cell population of interest.

In some embodiments, the guide RNA allows between 50 and 55%, 55 and 60%, 60 and 65%, 65 and 70%, 70 and 75%, 75 and 80%, 80 and 85%, 85 and 90%, 90 and 95%, 95 and 99% or more increased expression or secretion of a heterologous AAT gene. In some embodiments, the RNA allows at least 50%, 60%, 70%, 80%, 90% or 100% of the lower limit of normal of AAT expression. In certain embodiments, the level expressed is a combination of endogenous protein and heterologous protein. For example, in some embodiments, increased expression or secretion can be determined by detecting and measuring the AAT polypeptide level and comparing the level against the AAT polypeptide level before, e.g., treating the cells or administration to a subject. Increased expression or secretion of a heterologous AAT gene can be measured in vitro or in vivo. In some embodiments, secretion or expression of AAT is measured either by detecting protein secreted by tissue or population of cells (e.g., in serum or cell media) or by detecting total cellular amount of the protein from a tissue or cell population of interest, using, e.g., an enzyme-linked immunosorbent assay (ELISA), HPLC, mass spectrometry (e.g., liquid mass spectrometry (e.g., LC-MS, LC-MS/MS), or western blot assay with culture media or cell or tissue (e.g., liver) extract. In some embodiments, secretion or expression of AAT is measured in primary human hepatocytes, e.g. media or cellular samples. In some embodiments, secretion of AAT is measured in HUH7 cells, e.g. media samples. In some embodiments, the cell used is HUH7 cells. In some embodiments, the amount of AAT is compared to the amount of glyceraldehyde 3-phosphate dehydrogenase GAPDH (a housekeeping gene) to control for changes in cell number. In some embodiments, AAT may be assessed by PASD staining of liver tissue sections, e.g., to measure aggregation. In some embodiments, AAT may be assessed by measuring inhibition of neutrophil elastase, e.g., in the lung.

In some embodiments, the guide RNA allows between 50 and 55%, 55 and 60%, 60 and 65%, 65 and 70%, 70 and 75%, 75 and 80%, 80 and 85%, 85 and 90%, 90 and 95%, 95 and 99% or more increased activity that results from expression of a heterologous AAT gene (e.g., a functional or wild-type AAT). In some embodiments, the guide RNA allows at least 50%, 60%, 70%, 80%, 90% or 100% activity level of the lower limit of normal of AAT in a subject not suffering from AATD. In certain embodiments, the activity is a combination of endogenous protein and heterologous protein. For example, increased activity can be determined by detecting and measuring the protease inhibitor activity level and comparing the level against a level of activity before, e.g., treating the cells or administration to a subject. Such methods are available and known in the art. See, e.g., Mullins et al., “Standardized automated assay for functional alpha 1-antitrypsin,” 1984; Eckfeldt et al., “Automated assay for alpha-1-antitiypsin with N-a-benzoyl-DL-arginine-p-nitroanilide astrypsin substrate and standardized with p-nitrophenyl-p′-guanidinobenzoateastitrant fortrypsinactivesites,” 1982.

In some embodiments, the target sequence or region within intron 1 of a human albumin locus (of SEQ ID NO: 1) may be complementary to the guide sequence of the albumin guide RNA. In some embodiments, the degree of complementarity or identity between a guide sequence of a guide RNA and its corresponding target sequence may be at least 80%, 85%, 90%, or 95%; or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is about 20, or 20. In some embodiments, the target sequence and the guide sequence of the gRNA may contain 1-4 mismatches where the guide sequence is about 20, or 20 nucleotides.

As described and exemplified herein, the albumin guide RNAs can be used to insert and express a heterologous AAT gene (e.g., a functional or wild-type AAT) at intron 1 of an albumin gene, in combination with a SERPINA1 guide RNA to knockdown or knockout an endogenous SERPINA1 gene (e.g., a mutant SERPINA1 gene). Thus, in some embodiments, the present disclosure includes compositions comprising one or more SERPINA1 guide RNA (gRNA) comprising guide sequences that direct an RNA-guided DNA binding agent (e.g., Cas9) to a target DNA sequence in SERPINA1. The gRNA may comprise one or more of the guide sequences shown in Table 2. In some embodiments, provided herein are one or more SERPINA1 guide RNAs comprising a guide sequence of any one of SEQ ID NOs: 1000-1131.

In one aspect, the disclosure provides a SERPINA1 gRNA that comprises a guide sequence that is at least 95% identical or 90% identical to a sequence selected from SEQ ID NOs: 1000-1131.

In other embodiments, the composition comprises at least two SERPINA1 gRNA's comprising guide sequences selected from any two or more of the guide sequences of SEQ ID NOs: 1000-1131. In some embodiments, the composition comprises at least two gRNA's that each are at least 95% identical or 90%, identical to any of the nucleic acids of SEQ ID NOs: 1000-1131.

The SERPINA1 guide RNA compositions provided herein are designed to recognize a target sequence in the SERPINA1 gene. For example, the SERPINA1 target sequence may be recognized and cleaved by the provided RNA-guided DNA binding agent. In some embodiments, a Cas protein may be directed by a SERPINA1 guide RNA to a target sequence of the SERPINA1 gene, where the guide sequence of the guide RNA hybridizes with the target sequence and the Cas protein cleaves the target sequence.

In some embodiments, the selection of the one or more SERPINA1 guide RNAs is determined based on target sequences within the SERPINA1 gene.

Without being bound by any particular theory, mutations in critical regions of the gene may be less tolerable than mutations in non-critical regions of the gene, thus the location of a DSB is an important factor in the amount or type of protein knockdown or knockout that may result. In some embodiments, a SERPINA1 gRNA complementary or having complementarity to a target sequence within SERPINA1 is used to direct the Cas protein to a particular location in the SERPINA1 gene. In some embodiments, SERPINA1 gRNAs are designed to have guide sequences that are complementary or have complementarity to target sequences in exons 2, 3, 4, or 5 of SERPINA1.

In some embodiments, SERPINA1 gRNAs are designed to be complementary or have complementarity to target sequences in exons of SERPINA1 that code for the N-terminal region of AAT.

TABLE 2
SERPINA1 targeted and control guide sequence nomenclature, chromosomal
coordinates, and sequence

| SEQ | | | | |
|---|---|---|---|---|
| ID | | | Human Chromosomal | |
| No | Guide ID | Description | coordinates (hg38) | Guide Sequences |
| 1000 | CR001261 | Control 1 | Chr1:55039269- | GCCAGACUCCAAGUUCUGCC |
| | | | 55039291 | |
| 1001 | CR001262 | Control 2 | Chr1:55039155- | UAAGGCCAGUGGAAAGAAUU |
| | | | 55039177 | |
| 1002 | CR001263 | Control 3 | Chr1:55039180- | GGCAGCGAGGAGUCCACAGU |
| | | | 55039202 | |
| 1003 | CR001264 | Control 4 | Chr1:55039149- | UCUUUCCACUGGCCUUAACC |
| | | | 55039171 | |
| 1004 | CR001367 | Exon 2 | Chr14:94383211- | CAAUGCCGUCUUCUGUCUCG |
| | | | 94383233 | |
| 1005 | CR001368 | Exon 2 | Chr14:94383210- | AAUGCCGUCUUCUGUCUCGU |
| | | | 94383232 | |
| 1006 | CR001369 | Exon 2 | Chr14:94383209- | AUGCCGUCUUCUGUCUCGUG |
| | | | 94383231 | |
| 1007 | CR001370 | Exon 2 | Chr14:94383206- | AUGCCCCACGAGACAGAAGA |
| | | | 94383228 | |
| 1008 | CR001371 | Exon 2 | Chr14:94383195- | CUCGUGGGGCAUCCUCCUGC |
| | | | 94383217 | |
| 1009 | CR001372 | Exon 2 | Chr14:94383152- | GGAUCCUCAGCCAGGGAGAC |
| | | | 94383174 | |
| 1010 | CR001373 | Exon 2 | Chr14:94383146- | UCCCUGGCUGAGGAUCCCCA |
| | | | 94383168 | |
| 1011 | CR001374 | Exon 2 | Chr14:94383145- | UCCCUGGGGAUCCUCAGCCA |
| | | | 94383167 | |
| 1012 | CR001375 | Exon 2 | Chr14:94383144- | CUCCCUGGGGAUCCUCAGCC |
| | | | 94383166 | |
| 1013 | CR001376 | Exon 2 | Chr14:94383115- | GUGGGAUGUAUCUGUCUUCU |
| | | | 94383137 | |
| 1014 | CR001377 | Exon 2 | Chr14:94383114- | GGUGGGAUGUAUCUGUCUUC |
| | | | 94383136 | |
| 1015 | CR001378 | Exon 2 | Chr14:94383105- | AGAUACAUCCCACCAUGAUC |
| | | | 94383127 | |
| 1016 | CR001379 | Exon 2 | Chr14:94383097- | UGGGUGAUCCUGAUCAUGGU |
| | | | 94383119 | |
| 1017 | CR001380 | Exon 2 | Chr14:94383096- | UUGGGUGAUCCUGAUCAUGG |
| | | | 94383118 | |
| 1018 | CR001381 | Exon 2 | Chr14:94383093- | AGGUUGGGUGAUCCUGAUCA |
| | | | 94383115 | |
| 1019 | CR001382 | Exon 2 | Chr14:94383078- | GGGUGAUCUUGUUGAAGGUU |
| | | | 94383100 | |
| 1020 | CR001383 | Exon 2 | Chr14:94383077- | GGGGUGAUCUUGUUGAAGGU |
| | | | 94383099 | |
| 1021 | CR001384 | Exon 2 | Chr14:94383069- | CAACAAGAUCACCCCCAACC |
| | | | 94383091 | |
| 1022 | CR001385 | Exon 2 | Chr14:94383057- | AGGCGAACUCAGCCAGGUUG |
| | | | 94383079 | |
| 1023 | CR001386 | Exon 2 | Chr14:94383055- | GAAGGCGAACUCAGCCAGGU |
| | | | 94383077 | |
| 1024 | CR001387 | Exon 2 | Chr14:94383051- | GGCUGAAGGCGAACUCAGCC |
| | | | 94383073 | |
| 1025 | CR001388 | Exon 2 | Chr14:94383037- | CAGCUGGCGGUAUAGGCUGA |
| | | | 94383059 | |
| 1026 | CR001389 | Exon 2 | Chr14:94383036- | CUUCAGCCUAUACCGCCAGC |
| | | | 94383058 | |
| 1027 | CR001390 | Exon 2 | Chr14:94383030- | GGUGUGCCAGCUGGCGGUAU |
| | | | 94383052 | |
| 1028 | CR001391 | Exon 2 | Chr14:94383021- | UGUUGGACUGGUGUGCCAGC |
| | | | 94383043 | |
| 1029 | CR001392 | Exon 2 | Chr14:94383009- | AGAUAUUGGUGCUGUUGGAC |
| | | | 94383031 | |
| 1030 | CR001393 | Exon 2 | Chr14:94383004- | GAAGAAGAUAUUGGUGCUGU |
| | | | 94383026 | |
| 1031 | CR001394 | Exon 2 | Chr14:94382995- | CACUGGGGAGAAGAAGAUAU |
| | | | 94383017 | |
| 1032 | CR001395 | Exon 2 | Chr14:94382980- | GGCUGUAGCGAUGCUCACUG |
| | | | 94383002 | |
| 1033 | CR001396 | Exon 2 | Chr14:94382979- | AGGCUGUAGCGAUGCUCACU |
| | | | 94383001 | |
| 1034 | CR001397 | Exon 2 | Chr14:94382978- | AAGGCUGUAGCGAUGCUCAC |
| | | | 94383000 | |
| 1035 | CR001398 | Exon 2 | Chr14:94382928- | UGACACUCACGAUGAAAUCC |
| | | | 94382950 | |
| 1036 | CR001399 | Exon 2 | Chr14:94382925- | CACUCACGAUGAAAUCCUGG |
| | | | 94382947 | |
| 1037 | CR001400 | Exon 2 | Chr14:94382924- | ACUCACGAUGAAAUCCUGGA |
| | | | 94382946 | |
| 1038 | CR001401 | Exon 2 | Chr14:94382910- | GGUUGAAAUUCAGGCCCUCC |
| | | | 94382932 | |
| 1039 | CR001402 | Exon 2 | Chr14:94382904- | GGGCCUGAAUUUCAACCUCA |
| | | | 94382926 | |
| 1040 | CR001403 | Exon 2 | Chr14:94382895- | UUUCAACCUCACGGAGAUUC |
| | | | 94382917 | |
| 1041 | CR001404 | Exon 2 | Chr14:94382892- | CAACCUCACGGAGAUUCCGG |
| | | | 94382914 | |
| 1042 | CR001405 | Exon 2 | Chr14:94382889- | GAGCCUCCGGAAUCUCCGUG |
| | | | 94382911 | |
| 1043 | CR001406 | Exon 2 | Chr14:94382876- | CCGGAGGCUCAGAUCCAUGA |
| | | | 94382898 | |
| 1044 | CR001407 | Exon 2 | Chr14:94382850- | UGAGGGUACGGAGGAGUUCC |
| | | | 94382872 | |
| 1045 | CR001408 | Exon 2 | Chr14:94382841- | CUGGCUGGUUGAGGGUACGG |
| | | | 94382863 | |
| 1046 | CR001409 | Exon 2 | Chr14:94382833- | CUGGCUGUCUGGCUGGUUGA |
| | | | 94382855 | |
| 1047 | CR001410 | Exon 2 | Chr14:94382810- | CUCCAGCUGACCACCGGCAA |
| | | | 94382832 | |
| 1048 | CR001411 | Exon 2 | Chr14:94382808- | GGCCAUUGCCGGUGGUCAGC |
| | | | 94382830 | |
| 1049 | CR001412 | Exon 2 | Chr14:94382800- | GAGGAACAGGCCAUUGCCGG |
| | | | 94382822 | |
| 1050 | CR001413 | Exon 2 | Chr14:94382797- | GCUGAGGAACAGGCCAUUGC |
| | | | 94382819 | |
| 1051 | CR001414 | Exon 2 | Chr14:94382793- | CAAUGGCCUGUUCCUCAGCG |
| | | | 94382815 | |
| 1052 | CR001415 | Exon 2 | Chr14:94382792- | AAUGGCCUGUUCCUCAGCGA |
| | | | 94382814 | |
| 1053 | CR001416 | Exon 2 | Chr14:94382787- | UCAGGCCCUCGCUGAGGAAC |
| | | | 94382809 | |
| 1054 | CR001417 | Exon 2 | Chr14:94382781- | CUAGCUUCAGGCCCUCGCUG |
| | | | 94382803 | |
| 1055 | CR001418 | Exon 2 | Chr14:94382778- | CAGCGAGGGCCUGAAGCUAG |
| | | | 94382800 | |
| 1056 | CR001419 | Exon 2 | Chr14:94382769- | AAAACUUAUCCACUAGCUUC |
| | | | 94382791 | |
| 1057 | CR001420 | Exon 2 | Chr14:94382766- | GAAGCUAGUGGAUAAGUUUU |
| | | | 94382788 | |
| 1058 | CR001421 | Exon 2 | Chr14:94382763- | GCUAGUGGAUAAGUUUUUGG |
| | | | 94382785 | |
| 1059 | CR001422 | Exon 2 | Chr14:94382724- | UGACAGUGAAGGCUUCUGAG |
| | | | 94382746 | |
| 1060 | CR001423 | Exon 2 | Chr14:94382716- | AAGCCUUCACUGUCAACUUC |
| | | | 94382738 | |
| 1061 | CR001424 | Exon 2 | Chr14:94382715- | AGCCUUCACUGUCAACUUCG |
| | | | 94382737 | |
| 1062 | CR001425 | Exon 2 | Chr14:94382713- | GUCCCCGAAGUUGACAGUGA |
| | | | 94382735 | |
| 1063 | CR001426 | Exon 2 | Chr14:94382703- | CAACUUCGGGGACACCGAAG |
| | | | 94382725 | |
| 1064 | CR001427 | Exon 2 | Chr14:94382689- | GAUCUGUUUCUUGGCCUCUU |
| | | | 94382711 | |
| 1065 | CR001428 | Exon 2 | Chr14:94382680- | GUAAUCGUUGAUCUGUUUCU |
| | | | 94382702 | |
| 1066 | CR001429 | Exon 2 | Chr14:94382676- | GAAACAGAUCAACGAUUACG |
| | | | 94382698 | |
| 1067 | CR001430 | Exon 2 | Chr14:94382670- | GAUCAACGAUUACGUGGAGA |
| | | | 94382692 | |
| 1068 | CR001431 | Exon 2 | Chr14:94382669- | AUCAACGAUUACGUGGAGAA |
| | | | 94382691 | |
| 1069 | CR001432 | Exon 2 | Chr14:94382660- | UACGUGGAGAAGGGUACUCA |
| | | | 94382682 | |
| 1070 | CR001433 | Exon 2 | Chr14:94382659- | ACGUGGAGAAGGGUACUCAA |
| | | | 94382681 | |
| 1071 | CR001434 | Exon 2 | Chr14:94382643- | UCAAGGGAAAAUUGUGGAUU |
| | | | 94382665 | |
| 1072 | CR001435 | Exon 2 | Chr14:94382637- | GAAAAUUGUGGAUUUGGUCA |
| | | | 94382659 | |
| 1073 | CR001436 | Exon 2 | Chr14:94382607- | CAGAGACACAGUUUUUGCUC |
| | | | 94382629 | |
| 1074 | CR001437 | Exon 3 | Chr14:94381127- | UCCCCUCUCUCCAGGCAAAU |
| | | | 94381149 | |
| 1075 | CR001438 | Exon 3 | Chr14:94381098- | CUCGGUGUCCUUGACUUCAA |
| | | | 94381120 | |
| 1076 | CR001439 | Exon 3 | Chr14:94381097- | CUUUGAAGUCAAGGACACCG |
| | | | 94381119 | |
| 1077 | CR001440 | Exon 3 | Chr14:94381080- | CACGUGGAAGUCCUCUUCCU |
| | | | 94381102 | |
| 1078 | CR001441 | Exon 3 | Chr14:94381079- | CGAGGAAGAGGACUUCCACG |
| | | | 94381101 | |
| 1079 | CR001442 | Exon 3 | Chr14:94381073- | AGAGGACUUCCACGUGGACC |
| | | | 94381095 | |
| 1080 | CR001443 | Exon 3 | Chr14:94381064- | CGGUGGUCACCUGGUCCACG |
| | | | 94381086 | |
| 1081 | CR001444 | Exon 3 | Chr14:94381058- | GGACCAGGUGACCACCGUGA |
| | | | 94381080 | |
| 1082 | CR001445 | Exon 3 | Chr14:94381055- | GCACCUUCACGGUGGUCACC |
| | | | 94381077 | |
| 1083 | CR001446 | Exon 3 | Chr14:94381047- | CAUCAUAGGCACCUUCACGG |
| | | | 94381069 | |
| 1084 | CR001447 | Exon 3 | Chr14:94381036- | GUGCCUAUGAUGAAGCGUUU |
| | | | 94381058 | |
| 1085 | CR001448 | Exon 3 | Chr14:94381033- | AUGCCUAAACGCUUCAUCAU |
| | | | 94381055 | |
| 1086 | CR001449 | Exon 3 | Chr14:94381001- | UGGACAGCUUCUUACAGUGC |
| | | | 94381023 | |
| 1087 | CR001450 | Exon 3 | Chr14:94380995- | CUGUAAGAAGCUGUCCAGCU |
| | | | 94381017 | |
| 1088 | CR001451 | Exon 3 | Chr14:94380974- | GGUGCUGCUGAUGAAAUACC |
| | | | 94380996 | |
| 1089 | CR001452 | Exon 3 | Chr14:94380973- | GUGCUGCUGAUGAAAUACCU |
| | | | 94380995 | |
| 1090 | CR001453 | Exon 3 | Chr14:94380956- | AGAUGGCGGUGGCAUUGCCC |
| | | | 94380978 | |
| 1091 | CR001454 | Exon 3 | Chr14:94380945- | AGGCAGGAAGAAGAUGGCGG |
| | | | 94380967 | |
| 1092 | CR001474 | Exon 5 | Chr14:94378611- | GGUCAGCACAGCCUUAUGCA |
| | | | 94378633 | |
| 1093 | CR001475 | Exon 5 | Chr14:94378581- | AGAAAGGGACUGAAGCUGCU |
| | | | 94378603 | |
| 1094 | CR001476 | Exon 5 | Chr14:94378580- | GAAAGGGACUGAAGCUGCUG |
| | | | 94378602 | |
| 1095 | CR001477 | Exon 5 | Chr14:94378565- | UGCUGGGGCCAUGUUUUUAG |
| | | | 94378587 | |
| 1096 | CR001478 | Exon 5 | Chr14:94378557- | GGGUAUGGCCUCUAAAAACA |
| | | | 94378579 | |
| 1097 | CR001483 | Exon 5 | Chr14:94378526- | UGUUGAACUUGACCUCGGGG |
| | | | 94378548 | |
| 1098 | CR001484 | Exon 5 | Chr14:94378521- | GGGUUUGUUGAACUUGACCU |
| | | | 94378543 | |
| 1099 | CR003190 | Exon 2 | Chr14:94383131- | UUCUGGGCAGCAUCUCCCUG |
| | | | 94383153 | |
| 1100 | CR003191 | Exon 2 | Chr14:94383129- | UCUUCUGGGCAGCAUCUCCC |
| | | | 94383151 | |
| 1101 | CR003196 | Exon 2 | Chr14:94383024- | UGGACUGGUGUGCCAGCUGG |
| | | | 94383046 | |
| 1102 | CR003204 | Exon 2 | Chr14:94382961- | AGCCUUUGCAAUGCUCUCCC |
| | | | 94382983 | |
| 1103 | CR003205 | Exon 2 | Chr14:94382935- | UUCAUCGUGAGUGUCAGCCU |
| | | | 94382957 | |
| 1104 | CR003206 | Exon 2 | Chr14:94382901- | UCUCCGUGAGGUUGAAAUUC |
| | | | 94382923 | |
| 1105 | CR003207 | Exon 2 | Chr14:94382822- | GUCAGCUGGAGCUGGCUGUC |
| | | | 94382844 | |
| 1106 | CR003208 | Exon 2 | Chr14:94382816- | AGCCAGCUCCAGCUGACCAC |
| | | | 94382838 | |
| 1107 | CR003217 | Exon 3 | Chr14:94380942- | AUCAGGCAGGAAGAAGAUGG |
| | | | 94380964 | |
| 1108 | CR003218 | Exon 3 | Chr14:94380938- | CAUCUUCUUCCUGCCUGAUG |
| | | | 94380960 | |
| 1109 | CR003219 | Exon 3 | Chr14:94380937- | AUCUUCUUCCUGCCUGAUGA |
| | | | 94380959 | |
| 1110 | CR003220 | Exon 3 | Chr14:94380881- | CGAUAUCAUCACCAAGUUCC |
| | | | 94380903 | |
| 1111 | CR003221 | Exon 4 | Chr14:94379554- | CAGAUCAUAGGUUCCAGUAA |
| | | | 94379576 | |
| 1112 | CR003222 | Exon 4 | Chr14:94379507- | AUCACUAAGGUCUUCAGCAA |
| | | | 94379529 | |
| 1113 | CR003223 | Exon 4 | Chr14:94379506- | UCACUAAGGUCUUCAGCAAU |
| | | | 94379528 | |
| 1114 | CR003224 | Exon 4 | Chr14:94379505- | CACUAAGGUCUUCAGCAAUG |
| | | | 94379527 | |
| 1115 | CR003225 | Exon 4 | Chr14:94379453- | CUCACCUUGGAGAGCUUCAG |
| | | | 94379475 | |
| 1116 | CR003226 | Exon 4 | Chr14:94379452- | UCUCACCUUGGAGAGCUUCA |
| | | | 94379474 | |
| 1117 | CR003227 | Exon 4 | Chr14:94379451- | AUCUCACCUUGGAGAGCUUC |
| | | | 94379473 | |
| 1118 | CR003235 | Exon 5 | Chr14:94378525- | UUGUUGAACUUGACCUCGGG |
| | | | 94378547 | |
| 1119 | CR003236 | Exon 5 | Chr14:94378524- | UUUGUUGAACUUGACCUCGG |
| | | | 94378546 | |
| 1120 | CR003237 | Exon 5 | Chr14:94378523- | GUUUGUUGAACUUGACCUCG |
| | | | 94378545 | |
| 1121 | CR003238 | Exon 5 | Chr14:94378522- | GGUUUGUUGAACUUGACCUC |
| | | | 94378544 | |
| 1122 | CR003240 | Exon 5 | Chr14:94378501- | UCAAUCAUUAAGAAGACAAA |
| | | | 94378523 | |
| 1123 | CR003241 | Exon 5 | Chr14:94378500- | UUCAAUCAUUAAGAAGACAA |
| | | | 94378522 | |
| 1124 | CR003242 | Exon 5 | Chr14:94378472- | UACCAAGUCUCCCCUCUUCA |
| | | | 94378494 | |
| 1125 | CR003243 | Exon 5 | Chr14:94378471- | ACCAAGUCUCCCCUCUUCAU |
| | | | 94378493 | |
| 1126 | CR003244 | Exon 5 | Chr14:94378463- | UCCCCUCUUCAUGGGAAAAG |
| | | | 94378485 | |
| 1127 | CR003245 | Exon 5 | Chr14:94378461- | CACCACUUUUCCCAUGAAGA |
| | | | 94378483 | |
| 1128 | CR003246 | Exon 5 | Chr14:94378460- | UCACCACUUUUCCCAUGAAG |
| | | | 94378482 | |
| 1129 | GR000409 | Exon 2 | chr14:94382932- | ACUCACGAUGAAAUCCUGGA |
| | | | 94382952 | |
| 1130 | GR000414 | Exon 2 | chr14:94382900- | CAACCUCACGGAGAUUCCGG |
| | | | 94382920 | |
| 1131 | GR000415 | Exon 2 | chr14:94383026- | UGUUGGACUGGUGUGCCAGC |
| | | | 94383046 | |

Each of the albumin guide sequences and SERPINA1 guide sequences described herein may further comprise additional nucleotides to form a crRNA or guide RNA, e.g., with the following exemplary nucleotide sequence following the guide sequence at its 3′ end: GUUUUAGAGCUAUGCUGUUUUG (SEQ ID NO: 900) in 5′ to 3′ orientation. In the case of a sgRNA, the above guide sequences (the albumin guide sequences and SERPINA1 guide sequences shown in Table 1 at SEQ ID NOs:2-33 and Table 2 at SEQ ID Nos: 1000-1131, respectively) may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 901) in 5′ to 3′ orientation.

In the case of a sgRNA, the guide sequences may be integrated into the following modified motif: 15 mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage between nucleotide residues; and wherein the N's are collectively the nucleotide sequence of a guide sequence.

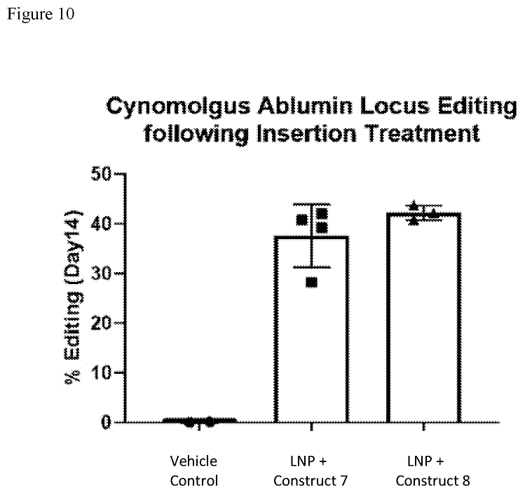

In the case of a sgRNA, the guide sequences may further comprise a SpyCas9 sgRNA sequence. An example of a SpyCas9 sgRNA sequence is shown below (SEQ ID NO: 902: GUUUUAGAGCUAGAAAUAGCAAGUUAAAAUAAGGCUAGUCCGUUAUCAACUU GAAAAAGUGGCACCGAGUCGGUGC—“Exemplary SpyCas9 sgRNA-1”), included at the 3′ end of the guide sequence, and provided with the domains as shown in the table below. LS is lower stem. B is bulge. US is upper stem. H1 and H2 are hairpin 1 and hairpin 2, respectively. Collectively H1 and H2 are referred to as the hairpin region. A model of the structure is provided in FIG. 10A of WO2019237069 which is incorporated herein by reference.

The nucleotide sequence of Exemplary SpyCas9 sgRNA-1 may serve as a template sequence for specific chemical modifications, sequence substitutions and truncations. In certain embodiments, the gRNA is an sgRNA or a dgRNA, for example, and it optionally comprises a chemical modification. In some embodiments, the modified sgRNA comprises a guide sequence and a SpyCas9 sgRNA sequence, e.g., Exemplary SpyCas9 sgRNA-1. A gRNA, such as an sgRNA, may include modifications on the 5′ end of the guide sequence and/or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof. In certain embodiments, the modified nucleotide includes a 2′-OMe modified nucleotide. In certain embodiments, the modified nucleotide includes a PS linkage. In certain embodiments, the modified nucleotide includes a 2′-OMe modified nucleotide and a PS linkage.

In certain embodiments, using SEQ ID NO: 201 (“Exemplary SpyCas9 sgRNA-1”) as an example, the Exemplary SpyCas9 sgRNA-1 further includes one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 4-8 nucleotides, preferably 4-6 nucleotides; and
      a. one or more of positions H1-1, H1-2, or H1-3 is deleted or substituted relative to Exemplary SpyCas9 sgRNA-1 (SEQ ID NO: 201) or
      b. one or more of positions H1-6 through H1-10 is substituted relative to Exemplary SpyCas9 sgRNA-1 (SEQ ID NO: 902); or
   3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 902); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 201); or
C. a substitution relative to Exemplary SpyCas9 sgRNA-1 (SEQ ID NO: 902) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. an Exemplary SpyCas9 sgRNA-1 (SEQ ID NO: 902) with an upper stem region, wherein the upper stem modification comprises a modification to any one or more of US1-US12 in the upper stem region, wherein
   1. the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof; or
   2. the modified nucleotide optionally includes a 2′-OMe modified.

In certain embodiments, Exemplary SpyCas9 sgRNA-1, or an sgRNA, such as an sgRNA comprising an Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In certain embodiments, the hairpin region includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the upper stem region includes one or more modified nucleotides. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a modified nucleotide. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a substituted nucleotide, i.e., sequence substituted nucleotide, wherein the pyrimidine is substituted for a purine. In certain embodiments, when the pyrimidine forms a Watson-Crick base pair in the single guide, the Watson-Crick based nucleotide of the substituted pyrimidine nucleotide is substituted to maintain Watson-Crick base pairing.

Exemplary spyCas9 sgRNA-1 (SEQ ID NO: 902)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |

| | | | | |
|---|---|---|---|---|
| LS1-LS6 | B1-B2 | US1-US12 | B2-B6 | LS7-LS12 |

| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
|---|---|---|---|---|---|---|---|---|---|---|---|---|---|---|---|---|---|---|---|---|---|---|---|---|---|---|---|---|---|
| A | A | G | G | C | U | A | G | U | C | C | G | U | U | A | U | C | A | A | C | U | U | G | A | A | A | A | A | G | U |

| | |
|---|---|
| Nexus | H1-1 through H1-12 |

| 61 | 62 | 63 | 64 | 65 | 66 | 67 | 68 | 69 | 70 | 71 | 72 | 73 | 74 | 75 | 76 |
|---|---|---|---|---|---|---|---|---|---|---|---|---|---|---|---|
| G | G | C | A | C | C | G | A | G | U | C | G | G | U | G | C |

| | |
|---|---|
| N | H2-1 through H2-15 |

TABLE 3
Human sgRNA and modification patterns

| | | SEQ | | SEQ |
|---|---|---|---|---|
| Guide | | ID | | ID |
| ID | Full Sequence | NO: | Full Sequence Modified | NO: |
| G009844 | GAGCAACCUCACUCUUGUCUGUUUU | 34 | mG*mA*mG*CAACCUCACUCUUGUCUGU | 66 |
| | AGAGCUAGAAAUAGCAAGUUAAAAU | | UUUAGAmGmCmUmAmGmAmAmAmUm | |
| | AAGGCUAGUCCGUUAUCAACUUGAA | | AmGmCAAGUUAAAAUAAGGCUAGUCC | |
| | AAAGUGGCACCGAGUCGGUGCUUUU | | GUUAUCAmAmCmUmUmGmAmAmAmAm | |
| | | | AmGmUmGmGmCmAmCmCmGmAmGmUm | |
| | | | CmGmGmUmGmCmU*mU*mU*mU | |
| G009851 | AUGCAUUUGUUUCAAAAUAUGUUUU | 35 | mA*mU*mG*CAUUUGUUUCAAAAUAUG | 67 |
| | AGAGCUAGAAAUAGCAAGUUAAAAU | | UUUUAGAmGmCmUmAmGmAmAmAmUm | |
| | AAGGCUAGUCCGUUAUCAACUUGAA | | AmGmCAAGUUAAAAUAAGGCUAGUCCG | |
| | AAAGUGGCACCGAGUCGGUGCUUUU | | UUAUCAmAmCmUmUmGmAmAmAmAmAm | |
| | | | GmUmGmGmCmAmCmCmGmAmGmUmCm | |
| | | | GmGmUmGmCmU*mU*mU*mU | |
| G009852 | UGCAUUUGUUUCAAAAUAUUGUUUU | 36 | mU*mG*mC*AUUUGUUUCAAAAUAUUGU | 68 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G009857 | AUUUAUGAGAUCAACAGCACGUUUU | 37 | mA*mU*mU*UAUGAGAUCAACAGCACGU | 69 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGm | |
| | | | UmGmGmCmAmCmCmGmAmGmUmCmGmG | |
| | | | mUmGmCmU*mU*mU*mU | |
| G009858 | GAUCAACAGCACAGGUUUUGGUUUU | 38 | mG*mA*mU*CAACAGCACAGGUUUUGGU | 70 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGm | |
| | | | UmGmGmCmAmCmCmGmAmGmUmCmGm | |
| | | | GmUmGmCmU*mU*mU*mU | |
| G009859 | UUAAAUAAAGCAUAGUGCAAGUUUU | 39 | mU*mU*mA*AAUAAAGCAUAGUGCAAGU | 71 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G009860 | UAAAGCAUAGUGCAAUGGAUGUUUU | 40 | mU*mA*mA*AGCAUAGUGCAAUGGAUGU | 72 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G009861 | UAGUGCAAUGGAUAGGUCUUGUUUU | 41 | mU*mA*mG*UGCAAUGGAUAGGUCUUGU | 73 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G009866 | UACUAAAACUUUAUUUUACUGUUUU | 42 | mU*mA*mC*UAAAACUUUAUUUUACUGU | 74 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G009867 | AAAGUUGAACAAUAGAAAAAGUUUU | 43 | mA*mA*mA*GUUGAACAAUAGAAAAAGU | 75 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G009868 | AAUGCAUAAUCUAAGUCAAAGUUUU | 44 | mA*mA*mU*GCAUAAUCUAAGUCAAAGU | 76 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G009874 | UAAUAAAAUUCAAACAUCCUGUUUU | 45 | mU*mA*mA*UAAAAUUCAAACAUCCUGU | 77 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012747 | GCAUCUUUAAAGAAUUAUUUGUUUU | 46 | mG*mC*mA*UCUUUAAAGAAUUAUUUGU | 78 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012748 | UUUGGCAUUUAUUUCUAAAAGUUUU | 47 | mU*mU*mU*GGCAUUUAUUUCUAAAAGU | 79 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012749 | UGUAUUUGUGAAGUCUUACAGUUUU | 48 | mU*mG*mU*AUUUGUGAAGUCUUACAGU | 80 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012750 | UCCUAGGUAAAAAAAAAAAAGUUUU | 49 | mU*mC*mC*UAGGUAAAAAAAAAAAAGU | 81 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012751 | UAAUUUUCUUUUGCGCACUAGUUUU | 50 | mU*mA*mA*UUUUCUUUUGCGCACUAGU | 82 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012752 | UGACUGAAACUUCACAGAAUGUUUU | 51 | mU*mG*mA*CUGAAACUUCACAGAAUGU | 83 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012753 | GACUGAAACUUCACAGAAUAGUUUU | 52 | mG*mA*mC*UGAAACUUCACAGAAUAGU | 84 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012754 | UUCAUUUUAGUCUGUCUUCUGUUUU | 53 | mU*mU*mC*AUUUUAGUCUGUCUUCUGU | 85 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012755 | AUUAUCUAAGUUUGAAUAUAGUUUU | 54 | mA*mU*mU*AUCUAAGUUUGAAUAUAGU | 86 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012756 | AAUUUUUAAAAUAGUAUUCUGUUUU | 55 | mA*mA*mU*UUUUAAAAUAGUAUUCUGU | 87 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012757 | UGAAUUAUUCUUCUGUUUAAGUUUU | 56 | mU*mG*mA*AUUAUUCUUCUGUUUAAGU | 88 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012758 | AUCAUCCUGAGUUUUUCUGUGUUUU | 57 | mA*mU*mC*AUCCUGAGUUUUUCUGUGU | 89 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012759 | UUACUAAAACUUUAUUUUACGUUUU | 58 | mU*mU*mA*CUAAAACUUUAUUUUACGU | 90 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012760 | ACCUUUUUUUUUUUUUACCUGUUUU | 59 | mA*mC*mC*UUUUUUUUUUUUUACCUGU | 91 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012761 | AGUGCAAUGGAUAGGUCUUUGUUUU | 60 | mA*mG*mU*GCAAUGGAUAGGUCUUUGU | 92 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012762 | UGAUUCCUACAGAAAAACUCGUUUU | 61 | mU*mG*mA*UUCCUACAGAAAAACUCGU | 93 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012763 | UGGGCAAGGGAAGAAAAAAAGUUUU | 62 | mU*mG*mG*GCAAGGGAAGAAAAAAAGU | 94 |
| | AGAGCUAGAAAUAGCAAGUUAAAAU | | UUUAGAmGmCmUmAmGmAmAmAmUmAm | |
| | AAGGCUAGUCCGUUAUCAACUUGAA | | GmCAAGUUAAAAUAAGGCUAGUCCGUUA | |
| | AAAGUGGCACCGAGUCGGUGCUUUU | | UCAmAmCmUmUmGmAmAmAmAmAmGmU | |
| | | | mGmGmCmAmCmCmGmAmGmUmCmGmGm | |
| | | | UmGmCmU*mU*mU*mU | |
| G012764 | CCUCACUCUUGUCUGGGCAAGUUUU | 63 | mC*mC*mU*CACUCUUGUCUGGGCAAGUU | 95 |
| | AGAGCUAGAAAUAGCAAGUUAAAAU | | UUAGAmGmCmUmAmGmAmAmAmUmAmG | |
| | AAGGCUAGUCCGUUAUCAACUUGAA | | mCAAGUUAAAAUAAGGCUAGUCCGUUAU | |
| | AAAGUGGCACCGAGUCGGUGCUUUU | | CAmAmCmUmUmGmAmAmAmAmAmGmU | |
| | | | mGmGmCmAmCmCmGmAmGmUmCmGmGm | |
| | | | UmGmCmU*mU*mU*mU | |
| G012765 | ACCUCACUCUUGUCUGGGCAGUUUU | 64 | mA*mC*mC*UCACUCUUGUCUGGGCAGUU | 96 |
| | AGAGCUAGAAAUAGCAAGUUAAAAU | | UUAGAmGmCmUmAmGmAmAmAmUmAmG | |
| | AAGGCUAGUCCGUUAUCAACUUGAA | | mCAAGUUAAAAUAAGGCUAGUCCGUUAU | |
| | AAAGUGGCACCGAGUCGGUGCUUUU | | CAmAmCmUmUmGmAmAmAmAmAmGmU | |
| | | | mGmGmCmAmCmCmGmAmGmUmCmGmGm | |
| | | | UmGmCmU*mU*mU*mU | |
| G012766 | UGAGCAACCUCACUCUUGUCGUUUU | 65 | mU*mG*mA*GCAACCUCACUCUUGUCGUU | 97 |
| | AGAGCUAGAAAUAGCAAGUUAAAAU | | UUAGAmGmCmUmAmGmAmAmAmUmAmG | |
| | AAGGCUAGUCCGUUAUCAACUUGAA | | mCAAGUUAAAAUAAGGCUAGUCCGUUAU | |
| | AAAGUGGCACCGAGUCGGUGCUUUU | | CAmAmCmUmUmGmAmAmAmAmAmGmU | |
| | | | mGmGmCmAmCmCmGmAmGmUmCmGmGm | |
| | | | UmGmCmU*mU*mU*mU | |

TABLE 4
Mouse albumin guide RNA

| | | | SEQ |
|---|---|---|---|
| | | | ID |
| Guide ID | Guide Sequence | Mouse Genomic Coordinates (mm10) | NO: |
| G000551 | AUUUGCAUCUGAGAACCCUU | chr5:90461148-90461168 | 98 |
| G000552 | AUCGGGAACUGGCAUCUUCA | chr5:90461590-90461610 | 99 |
| G000553 | GUUACAGGAAAAUCUGAAGG | chr5:90461569-90461589 | 100 |
| G000554 | GAUCGGGAACUGGCAUCUUC | chr5:90461589-90461609 | 101 |
| G000555 | UGCAUCUGAGAACCCUUAGG | chr5:90461151-90461171 | 102 |
| G000666 | CACUCUUGUCUGUGGAAACA | chr5:90461709-90461729 | 103 |
| G000667 | AUCGUUACAGGAAAAUCUGA | chr5:90461572-90461592 | 104 |
| G000668 | GCAUCUUCAGGGAGUAGCUU | chr5:90461601-90461621 | 105 |
| G000669 | CAAUCUUUAAAUAUGUUGUG | chr5:90461674-90461694 | 106 |
| G000670 | UCACUCUUGUCUGUGGAAAC | chr5:90461710-90461730 | 107 |
| G011722 | UGCUUGUAUUUUUCUAGUAA | chr5:90461039-90461059 | 108 |
| G011723 | GUAAAUAUCUACUAAGACAA | chr5:90461425-90461445 | 109 |
| G011724 | UUUUUCUAGUAAUGGAAGCC | chr5:90461047-90461067 | 110 |
| G011725 | UUAUAUUAUUGAUAUAUUUU | chr5:90461174-90461194 | 111 |
| G011726 | GCACAGAUAUAAACACUUAA | chr5:90461480-90461500 | 112 |
| G011727 | CACAGAUAUAAACACUUAAC | chr5:90461481-90461501 | 113 |
| G011728 | GGUUUUAAAAAUAAUAAUGU | chr5:90461502-90461522 | 114 |
| G011729 | UCAGAUUUUCCUGUAACGAU | chr5:90461572-90461592 | 115 |
| G011730 | CAGAUUUUCCUGUAACGAUC | chr5:90461573-90461593 | 116 |
| G011731 | CAAUGGUAAAUAAGAAAUAA | chr5:90461408-90461428 | 117 |
| G013018 | GGAAAAUCUGAAGGUGGCAA | chr5:90461563-90461583 | 118 |
| G013019 | GGCGAUCUCACUCUUGUCUG | chr5:90461717-90461737 | 119 |

TABLE 5
Mouse albumin guide sgRNA and modification pattern

| Guide | | SEQ ID | | SEQ ID |
|---|---|---|---|---|
| ID | Full Sequence | NO: | Full Sequence Modified | NO: |
| G000551 | AUUUGCAUCUGAGAACCCUU | 120 | mA*mU*mU*UGCAUCUGA | 142 |
| | GUUUUAGAGCUAGAAAUAGC | | GAACCCUUGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G000552 | AUCGGGAACUGGCAUCUUCA | 121 | mA*mU*mC*GGGAACUGG | 143 |
| | GUUUUAGAGCUAGAAAUAGC | | CAUCUUCAGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G000553 | GUUACAGGAAAAUCUGAAGG | 122 | mG*mU*mU*ACAGGAAAA | 144 |
| | GUUUUAGAGCUAGAAAUAGC | | UCUGAAGGGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G000554 | GAUCGGGAACUGGCAUCUUC | 123 | mG*mA*mU*CGGGAACUG | 145 |
| | GUUUUAGAGCUAGAAAUAGC | | GCAUCUUCGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G000555 | UGCAUCUGAGAACCCUUAGG | 124 | mU*mG*mC*AUCUGAGAA | 146 |
| | GUUUUAGAGCUAGAAAUAGC | | CCCUUAGGGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G000666 | CACUCUUGUCUGUGGAAACA | 125 | mC*mA*mC*UCUUGUCUG | 147 |
| | GUUUUAGAGCUAGAAAUAGC | | UGGAAACAGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G000667 | AUCGUUACAGGAAAAUCUGA | 126 | mA*mU*mC*GUUACAGGA | 148 |
| | GUUUUAGAGCUAGAAAUAGC | | AAAUCUGAGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G000668 | GCAUCUUCAGGGAGUAGCUU | 127 | mG*mC*mA*UCUUCAGGG | 149 |
| | GUUUUAGAGCUAGAAAUAGC | | AGUAGCUUGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G000669 | CAAUCUUUAAAUAUGUUGUG | 128 | mC*mA*mA*UCUUUAAAU | 150 |
| | GUUUUAGAGCUAGAAAUAGC | | AUGUUGUGGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G000670 | UCACUCUUGUCUGUGGAAAC | 129 | mU*mC*mA*CUCUUGUCU | 151 |
| | GUUUUAGAGCUAGAAAUAGC | | GUGGAAACGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011722 | UGCUUGUAUUUUUCUAGUAA | 130 | mU*mG*mC*UUGUAUUUU | 152 |
| | GUUUUAGAGCUAGAAAUAGC | | UCUAGUAAGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011723 | GUAAAUAUCUACUAAGACAA | 131 | mG*mU*mA*AAUAUCUAC | 153 |
| | GUUUUAGAGCUAGAAAUAGC | | UAAGACAAGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011724 | UUUUUCUAGUAAUGGAAGCC | 132 | mU*mU*mU*UUCUAGUAA | 154 |
| | GUUUUAGAGCUAGAAAUAGC | | UGGAAGCCGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011725 | UUAUAUUAUUGAUAUAUUUU | 133 | mU*mU*mA*UAUUAUUGA | 155 |
| | GUUUUAGAGCUAGAAAUAGC | | UAUAUUUUGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011726 | GCACAGAUAUAAACACUUAA | 134 | mG*mC*mA*CAGAUAUAA | 156 |
| | GUUUUAGAGCUAGAAAUAGC | | ACACUUAAGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011727 | CACAGAUAUAAACACUUAAC | 135 | mC*mA*mC*AGAUAUAAA | 157 |
| | GUUUUAGAGCUAGAAAUAGC | | CACUUAACGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011728 | GGUUUUAAAAAUAAUAAUGU | 136 | mG*mG*mU*UUUAAAAAU | 158 |
| | GUUUUAGAGCUAGAAAUAGC | | AAUAAUGUGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011729 | UCAGAUUUUCCUGUAACGAU | 137 | mU*mC*mA*GAUUUUCCU | 159 |
| | GUUUUAGAGCUAGAAAUAGC | | GUAACGAUGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011730 | CAGAUUUUCCUGUAACGAUC | 138 | mC*mA*mG*AUUUUCCUG | 160 |
| | GUUUUAGAGCUAGAAAUAGC | | UAACGAUCGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G011731 | CAAUGGUAAAUAAGAAAUAA | 139 | mC*mA*mA*UGGUAAAUA | 161 |
| | GUUUUAGAGCUAGAAAUAGC | | AGAAAUAAGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G013018 | GGAAAAUCUGAAGGUGGCAA | 140 | mG*mG*mA*AAAUCUGAA | 162 |
| | GUUUUAGAGCUAGAAAUAGC | | GGUGGCAAGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |
| G013019 | GGCGAUCUCACUCUUGUCUG | 141 | mG*mG*mC*GAUCUCACU | 163 |
| | GUUUUAGAGCUAGAAAUAGC | | CUUGUCUGGUUUUAGAm | |
| | AAGUUAAAAUAAGGCUAGUC | | GmCmUmAmGmAmAmAmU | |
| | CGUUAUCAACUUGAAAAAGU | | mAmGmCAAGUUAAAAUA | |
| | GGCACCGAGUCGGUGCUUUU | | AGGCUAGUCCGUUAUCAm | |
| | | | AmCmUmUmGmAmAmAmA | |
| | | | mAmGmUmGmGmCmAmCm | |
| | | | CmGmAmGmUmCmGmGmU | |
| | | | mGmCmU*mU*mU*mU | |

TABLE 6
Cyno albumin guide RNA

| | | | SEQ |
|---|---|---|---|
| | | | ID |
| Guide ID | Guide Sequence | Cyno Genomic Coordinates (mf5) | NO: |
| G009844 | GAGCAACCUCACUCUUGUCU | chr5:61198711-61198731 | 2* |
| G009845 | AGCAACCUCACUCUUGUCUG | chr5:61198712-61198732 | 165 |
| G009846 | ACCUCACUCUUGUCUGGGGA | chr5:61198716-61198736 | 166 |
| G009847 | CCUCACUCUUGUCUGGGGAA | chr5:61198717-61198737 | 167 |
| G009848 | CUCACUCUUGUCUGGGGAAG | chr5:61198718-61198738 | 168 |
| G009849 | GGGGAAGGGGAGAAAAAAAA | chr5:61198731-61198751 | 169 |
| G009850 | GGGAAGGGGAGAAAAAAAAA | chr5:61198732-61198752 | 170 |
| G009851 | AUGCAUUUGUUUCAAAAUAU | chr5:61198825-61198845 | 3* |
| G009852 | UGCAUUUGUUUCAAAAUAUU | chr5:61198826-61198846 | 4* |
| G009853 | UGAUUCCUACAGAAAAAGUC | chr5:61198852-61198872 | 173 |
| G009854 | UACAGAAAAAGUCAGGAUAA | chr5:61198859-61198879 | 174 |
| G009855 | UUUCUUCUGCCUUUAAACAG | chr5:61198889-61198909 | 175 |
| G009856 | UUAUAGUUUUAUAUUCAAAC | chr5:61198957-61198977 | 176 |
| G009857 | AUUUAUGAGAUCAACAGCAC | chr5:61199062-61199082 | 5* |
| G009858 | GAUCAACAGCACAGGUUUUG | chr5:61199070-61199090 | 6* |
| G009859 | UUAAAUAAAGCAUAGUGCAA | chr5:61199096-61199116 | 7* |
| G009860 | UAAAGCAUAGUGCAAUGGAU | chr5:61199101-61199121 | 8* |
| G009861 | UAGUGCAAUGGAUAGGUCUU | chr5:61199108-61199128 | 9* |
| G009862 | AGUGCAAUGGAUAGGUCUUA | chr5:61199109-61199129 | 182 |
| G009863 | UUACUUUGCACUUUCCUUAG | chr5:61199186-61199206 | 183 |
| G009864 | UACUUUGCACUUUCCUUAGU | chr5:61199187-61199207 | 184 |
| G009865 | UCUGACCUUUUAUUUUACCU | chr5:61199238-61199258 | 185 |
| G009866 | UACUAAAACUUUAUUUUACU | chr5:61199367-61199387 | 10* |
| G009867 | AAAGUUGAACAAUAGAAAAA | chr5:61199401-61199421 | 11* |
| G009868 | AAUGCAUAAUCUAAGUCAAA | chr5:61198812-61198832 | 12* |
| G009869 | AUUAUCCUGACUUUUUCUGU | chr5:61198860-61198880 | 189 |
| G009870 | UGAAUUAUUCCUCUGUUUAA | chr5:61198901-61198921 | 190 |
| G009871 | UAAUUUUCUUUUGCCCACUA | chr5:61199203-61199223 | 191 |
| G009872 | AAAAGGUCAGAAUUGUUUAG | chr5:61199229-61199249 | 192 |
| G009873 | AACAUCCUAGGUAAAAUAAA | chr5:61199246-61199266 | 193 |
| G009874 | UAAUAAAAUUCAAACAUCCU | chr5:61199258-61199278 | 13 |
| G009875 | UUGUCAUGUAUUUCUAAAAU | chr5:61199322-61199342 | 195 |
| G009876 | UUUGUCAUGUAUUUCUAAAA | chr5:61199323-61199343 | 196 |

SEQ ID NOs marked with an “*” above indicate that the indicated gRNA is applicable to both cyno and human.

TABLE 7
Cyno sgRNA and modification patterns

| | | SEQ | | SEQ |
|---|---|---|---|---|
| Guide | | ID | | ID |
| ID | Full Sequence | NO: | Full Sequence Modified | NO: |
| G009844 | GAGCAACCUCACUCUUGUCU | 34* | mG*mA*mG*CAACCUCACUCUUGUCUGUUUUAG | 66* |
| | GUUUUAGAGCUAGAAAUAGC | | AmGmCmUmAmGmAmAmAmUmAmGmCAAGUUA | |
| | AAGUUAAAAUAAGGCUAGUC | | AAAUAAGGCUAGUCCGUUAUCAmAmCmUmUm | |
| | CGUUAUCAACUUGAAAAAGU | | GmAmAmAmAmAmGmUmGmGmCmAmCmCmGm | |
| | GGCACCGAGUCGGUGCUUUU | | AmGmUmCmGmGmUmGmCmU*mU*mU*mU | |
| G009845 | AGCAACCUCACUCUUGUCUG | 198 | mA*mG*mC*AACCUCACUCUUGUCUGGUUUUAG | 231 |
| | GUUUUAGAGCUAGAAAUAGC | | AmGmCmUmAmGmAmAmAmUmAmGmCAAGUUA | |
| | AAGUUAAAAUAAGGCUAGUC | | AAAUAAGGCUAGUCCGUUAUCAmAmCmUmUm | |
| | CGUUAUCAACUUGAAAAAGU | | GmAmAmAmAmAmGmUmGmGmCmAmCmCmGm | |
| | GGCACCGAGUCGGUGCUUUU | | AmGmUmCmGmGmUmGmCmU*mU*mU*mU | |
| G009846 | ACCUCACUCUUGUCUGGGGA | 199 | mA*mC*mC*UCACUCUUGUCUGGGGAGUUUU | 232 |
| | GUUUUAGAGCUAGAAAUAGC | | AGAmGmCmUmAmGmAmAmAmUmAmGmCAA | |
| | AAGUUAAAAUAAGGCUAGUC | | GUUAAAAUAAGGCUAGUCCGUUAUCAmAmCm | |
| | CGUUAUCAACUUGAAAAAGU | | UmUmGmAmAmAmAmAmGmUmGmGmCmAmCm | |
| | GGCACCGAGUCGGUGCUUUU | | CmGmAmGmUmCmGmGmUmGmCmU*mU*mU*mU | |
| G009847 | CCUCACUCUUGUCUGGGGAA | 200 | mC*mC*mU*CACUCUUGUCUGGGGAAGUUUUA | 233 |
| | GUUUUAGAGCUAGAAAUAGC | | GAmGmCmUmAmGmAmAmAmUmAmGmCAAGU | |
| | AAGUUAAAAUAAGGCUAGUC | | UAAAAUAAGGCUAGUCCGUUAUCAmAmCmUm | |
| | CGUUAUCAACUUGAAAAAGU | | UmGmAmAmAmAmAmGmUmGmGmCmAmCmCm | |
| | GGCACCGAGUCGGUGCUUUU | | GmAmGmUmCmGmGmUmGmCmU*mU*mU*mU | |
| G009848 | CUCACUCUUGUCUGGGGAAG | 201 | mC*mU*mC*ACUCUUGUCUGGGGAAGGUUUU | 234 |
| | GUUUUAGAGCUAGAAAUAGC | | AGAmGmCmUmAmGmAmAmAmUmAmGmCAA | |
| | AAGUUAAAAUAAGGCUAGUC | | GUUAAAAUAAGGCUAGUCCGUUAUCAmAmCm | |
| | CGUUAUCAACUUGAAAAAGU | | UmUmGmAmAmAmAmAmGmUmGmGmCmAmCm | |
| | GGCACCGAGUCGGUGCUUUU | | CmGmAmGmUmCmGmGmUmGmCmU*mU*mU*mU | |
| G009849 | GGGGAAGGGGAGAAAAAAAA | 202 | mG*mG*mG*GAAGGGGAGAAAAAAAAGUUUUAG | 235 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009850 | GGGAAGGGGAGAAAAAAAAA | 203 | mG*mG*mG*AAGGGGAGAAAAAAAAAGUUUUAG | 236 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmUmU*mU*mU | |
| G009851 | AUGCAUUUGUUUCAAAAUAU | 35* | mA*mU*mG*CAUUUGUUUCAAAAUAUGUUUUAG | 67* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009852 | UGCAUUUGUUUCAAAAUAUU | 36* | mU*mG*mC*AUUUGUUUCAAAAUAUUGUUUUAG | 68* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009853 | UGAUUCCUACAGAAAAAGUC | 206 | mU*mG*mA*UUCCUACAGAAAAAGUCGUUUUAG | 239 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009854 | UACAGAAAAAGUCAGGAUAA | 207 | mU*mA*mC*AGAAAAAGUCAGGAUAAGUUUUAG | 240 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009855 | UUUCUUCUGCCUUUAAACAG | 208 | mU*mU*mU*CUUCUGCCUUUAAACAGGUUUUAG | 241 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009856 | UUAUAGUUUUAUAUUCAAAC | 209 | mU*mU*mA*UAGUUUUAUAUUCAAACGUUUUAG | 242 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009857 | AUUUAUGAGAUCAACAGCAC | 37* | mA*mU*mU*UAUGAGAUCAACAGCACGUUUUAG | 69* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009858 | GAUCAACAGCACAGGUUUUG | 38* | mG*mA*mU*CAACAGCACAGGUUUUGGUUUUAG | 70* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009859 | UUAAAUAAAGCAUAGUGCAA | 39* | mU*mU*mA*AAUAAAGCAUAGUGCAAGUUUUAG | 71* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009860 | UAAAGCAUAGUGCAAUGGAU | 40* | mU*mA*mA*AGCAUAGUGCAAUGGAUGUUUUAG | 72* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009861 | UAGUGCAAUGGAUAGGUCUU | 41* | mU*mA*mG*UGCAAUGGAUAGGUCUUGUUUUAG | 73* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009862 | AGUGCAAUGGAUAGGUCUUA | 215 | mA*mG*mU*GCAAUGGAUAGGUCUUAGUUUUAG | 248 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009863 | UUACUUUGCACUUUCCUUAG | 216 | mU*mU*mA*CUUUGCACUUUCCUUAGGUUUUAG | 249 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009864 | UACUUUGCACUUUCCUUAGU | 217 | mU*mA*mC*UUUGCACUUUCCUUAGUGUUUUAG | 250 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009865 | UCUGACCUUUUAUUUUACCU | 218 | mU*mC*mU*GACCUUUUAUUUUACCUGUUUUAG | 251 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009866 | UACUAAAACUUUAUUUUACU | 42* | mU*mA*mC*UAAAACUUUAUUUUACUGUUUUAG | 74* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009867 | AAAGUUGAACAAUAGAAAAA | 43* | mA*mA*mA*GUUGAACAAUAGAAAAAGUUUUAG | 75* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009868 | AAUGCAUAAUCUAAGUCAAA | 44* | mA*mA*mU*GCAUAAUCUAAGUCAAAGUUUUAG | 76* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009869 | AUUAUCCUGACUUUUUCUGU | 222 | mA*mU*mU*AUCCUGACUUUUUCUGUGUUUUAG | 255 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009870 | UGAAUUAUUCCUCUGUUUAA | 223 | mU*mG*mA*AUUAUUCCUCUGUUUAAGUUUUAG | 256 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009871 | UAAUUUUCUUUUGCCCACUA | 224 | mU*mA*mA*UUUUCUUUUGCCCACUAGUUUUAG | 257 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUm | |
| | CGUUAUCAACUUGAAAAAGU | | GmAmAmAmAmAmGmUmGmGmCmAmCmCmGm | |
| | GGCACCGAGUCGGUGCUUUU | | AmGmUmCmGmGmUmGmCmU*mU*mU*mU | |
| G009872 | AAAAGGUCAGAAUUGUUUAG | 225 | mA*mA*mA*AGGUCAGAAUUGUUUAGGUUUUAG | 258 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009873 | AACAUCCUAGGUAAAAUAAA | 226 | mA*mA*mC*AUCCUAGGUAAAAUAAAGUUUUAG | 259 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009874 | UAAUAAAAUUCAAACAUCCU | 45* | mU*mA*mA*UAAAAUUCAAACAUCCUGUUUUAG | 77* |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| G009875 | UUGUCAUGUAUUUCUAAAAU | 228 | mU*mU*mG*UCAUGUAUUUCUAAAAUGUUUUAG | 261 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |
| | UUUGUCAUGUAUUUCUAAAA | 229 | mU*mU*mU*GUCAUGUAUUUCUAAAAGUUUUAG | 262 |
| | GUUUUAGAGCUAGAAAUAGC | | AmGmCmUmAmGmAmAmAmUmAmGmCAAGUUAA | |
| | AAGUUAAAAUAAGGCUAGUC | | AAUAAGGCUAGUCCGUUAUCAmAmCmUmUmGm | |
| G009876 | CGUUAUCAACUUGAAAAAGU | | AmAmAmAmAmGmUmGmGmCmAmCmCmGmAmGm | |
| | GGCACCGAGUCGGUGCUUUU | | UmCmGmGmUmGmCmU*mU*mU*mU | |

SEQ ID NOs marked with an “*” above indicate that the indicated sgRNA is applicable to both cyno and human.

TABLE 8
sgRNA and Modifications

| Guide | Target site | Unmodified | Modified |
|---|---|---|---|
| G000409 | ACUCACGAUGA | ACUCACGAUGAAA | mA*mC*mU*CACGAUGAAAUCCUGGAGUU |
| | AAUCCUGGA | UCCUGGAGUUUUA | UUAGAmGmCmUmAmGmAmAmAmUmAmG |
| | SEQ ID NO: 1129 | GAGCUAGAAAUAG | mCAAGUUAAAAUAAGGCUAGUCCGUUAU |
| | | CAAGUUAAAAUAA | CAmAmCmUmUmGmAmAmAmAmAmGmUm |
| | | GGCUAGUCCGUUA | GmGmCmAmCmCmGmAmGmUmCmGmGmU |
| | | UCAACUUGAAAAA | mGmCmU*mU*mU*mU |
| | | GUGGCACCGAGUC | (SEQ ID NO: 1133) |
| | | GGUGCUUUU | |
| | | (SEQ ID NO: 1132) | |
| G000414 | CAACCUCACGG | CAACCUCACGGAG | mC*mA*mA*CCUCACGGAGAUUCCGGGUU |
| | AGAUUCCGG | AUUCCGGGUUUUA | UUAGAmGmCmUmAmGmAmAmAmUmAmG |
| | (SEQ ID NO: 1130) | GAGCUAGAAAUAG | mCAAGUUAAAAUAAGGCUAGUCCGUUAU |
| | | CAAGUUAAAAUAA | CAmAmCmUmUmGmAmAmAmAmAmGmUm |
| | | GGCUAGUCCGUUA | GmGmCmAmCmCmGmAmGmUmCmGmGmU |
| | | UCAACUUGAAAAA | mGmCmU*mU*mU*mU |
| | | GUGGCACCGAGUC | (SEQ ID NO: 1135) |
| | | GGUGCUUUU | |
| | | (SEQ ID NO: 1134) | |
| G000415 | UGUUGGACUGG | UGUUGGACUGGUG | mU*mG*mU*UGGACUGGUGUGCCAGCGUU |
| | UGUGCCAGC | UGCCAGCGUUUUA | UUAGAmGmCmUmAmGmAmAmAmUmAmG |
| | (SEQ ID NO: 1131) | GAGCUAGAAAUAG | mCAAGUUAAAAUAAGGCUAGUCCGUUAU |
| | | CAAGUUAAAAUAA | CAmAmCmUmUmGmAmAmAmAmAmGmUm |
| | | GGCUAGUCCGUUA | GmGmCmAmCmCmGmAmGmUmCmGmGmU |
| | | UCAACUUGAAAAA | mGmCmU*mU*mU*mU |
| | | GUGGCACCGAGUC | (SEQ ID NO: 1137) |
| | | GGUGCUUUU | |
| | | (SEQ ID NO: 1136) | |

SEQ ID NOs marked with an “*” above indicate that the indicated sgRNA is applicable to both cynomolgus and human.

The albumin or SERPINA1 guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, the sgRNA comprises one or more linkages between nucleotides that is not a phosphodiester linkage.

In each of the composition, use, and method embodiments described herein, the guide RNA may comprise two RNA molecules as a “dual guide RNA” or “dgRNA”. The dgRNA comprises a first RNA molecule comprising a crRNA comprising, e.g., a guide sequence shown in Table 1 or Table 2, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA (albumin gRNA or SERPINA1 gRNA) may comprise a single RNA molecule as a “single guide RNA” or “sgRNA”. The sgRNA may comprise a crRNA (or a portion thereof) comprising a guide sequence shown in Table 1 or Table 2 covalently linked to a trRNA. The sgRNA may comprise 15, 16, 17, 18, 19, or 20 contiguous nucleotides of a guide sequence shown in Table 1 or Table 2. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond. In some embodiments, the guide RNA comprises a sgRNA shown in any one of SEQ ID No: 34-67 or 120-163. In some embodiments, the guide RNA comprises a sgRNA comprising any one of the guide sequences of SEQ ID No: 2-33, 98-119, 165-170, 172, 174-176, 182-185, 189-193, 195-193, 195, or 196 and the nucleotides of SEQ ID No: 901 or 902, wherein the nucleotides of SEQ ID No: 901 or 902 are on the 3′ end of the guide sequence, and wherein the sgRNA may be modified as shown in Tables 9, 11, or 13 or SEQ ID NO: 300.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

C. Modified gRNAs and mRNAs

In some embodiments, the gRNA disclosed herein (e.g., albumin or SERPINA1 gRNA) is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar or backbone modification); and (vii) modification or replacement of the sugar (an exemplary sugar modification).

Chemical modifications such as those listed above can be combined to provide modified gRNAs or mRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some 25 embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′—C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or internal nucleosides may be modified, or the entire sgRNA may be chemically modified. Certain embodiments comprise a 5′ end modification. Certain embodiments comprise a 3′ end modification.

In some embodiments, the guide RNAs disclosed herein comprise one of the modification patterns disclosed in WO2018/107028 A1, filed Dec. 8, 2017, titled “Chemically Modified Guide RNAs,” the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, WO2017004279, US2018187186, US2019048338, the contents of which are hereby incorporated by reference in their entirety.

In some embodiments, the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, and wherein the totality of N's comprise an albumin intron 1 guide sequence as described in Table 1; and SERPINA1 guide sequences as described in Table 2. For example, encompassed herein is SEQ ID NO: 300, where the N's are replaced with any of the guide sequences disclosed herein in Table 1 (SEQ ID Nos: 2-33) or Table 2 (SEQ ID Nos: 1000-1131).

Any of the modifications described below may be present in the gRNAs and mRNAs described herein.

The terms “mA,” “mC,” “mU,” or “mG” may be used to denote a nucleotide that has been modified with 2′-O-Me.

Modification of 2′-O-methyl can be depicted as follows:

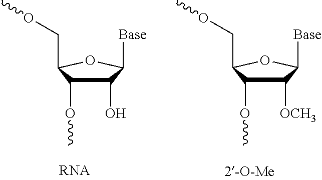

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

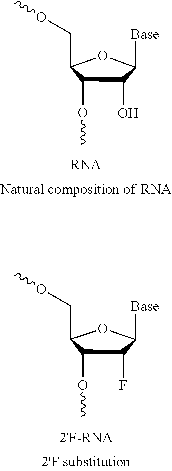

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S— into a nonbridging phosphate oxygen, generating a PS bond in lieu of a phosphodiester bond:

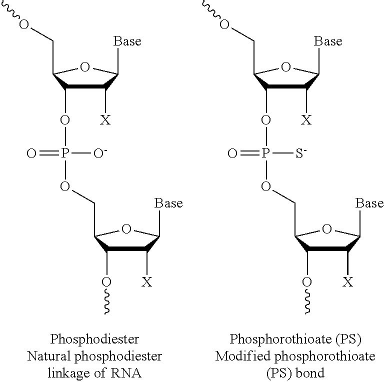

Abasic nucleotides refer to those which lack nitrogenous bases. The figure below depicts an oligonucleotide with an abasic (also known as apurinic) site that lacks a base:

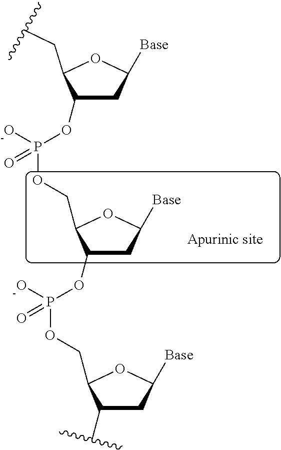

Inverted bases refer to those with linkages that are inverted from the normal 5′ to 3′ linkage (i.e., either a 5′ to 5′ linkage or a 3′ to 3′ linkage). For example:

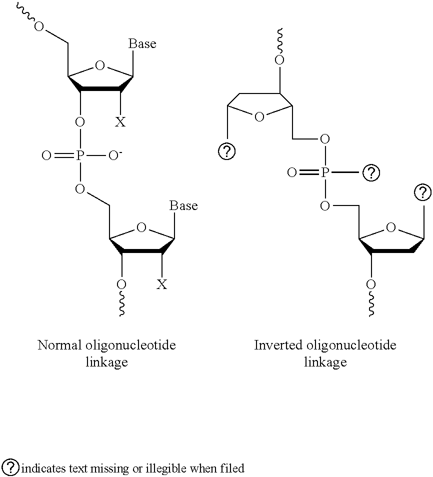

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted 10 abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic 15 nucleotide, PS bond, or other nucleotide modification well known in the art to increase stability or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, any of the guide RNAs disclosed herein comprises a modified sgRNA. In some embodiments, the sgRNA comprises the modification pattern shown in SEQ ID NO: 200, where N is any natural or non-natural nucleotide, and where the totality of the N's comprise a guide sequence (e.g., as shown in Table 1 or Table 2) that directs a nuclease to a target sequence (e.g., in human albumin intron 1 or SERPINA1).

As noted above, in some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered. As described below, the mRNA comprising a Cas nuclease may comprise a Cas9 nuclease, such as an S. pyogenes Cas9 nuclease having cleavase, nickase, or site-specific DNA binding activity. In some embodiments, the ORF encoding an RNA-guided DNA nuclease is a “modified RNA-guided DNA binding agent ORF” or simply a “modified ORF,” which is used as shorthand to indicate that the ORF is modified.

Cas9 ORFs, including modified Cas9 ORFs, are provided herein and are known in the art. As one example, the Cas9 ORF can be codon optimized, such that coding sequence includes one or more alternative codons for one or more amino acids. An “alternative codon” as used herein refers to variations in codon usage for a given amino acid, and may or may not be a preferred or optimized codon (codon optimized) for a given expression system. Preferred codon usage, or codons that are well-tolerated in a given system of expression, is known in the art. The Cas9 coding sequences, Cas9 mRNAs, and Cas9 protein sequences of WO2013/176772, WO2014/065596, WO2016/106121, and WO2019/067910 are hereby incorporated by reference. In particular, the ORFs and Cas9 amino acid sequences of the table at paragraph [0449] WO2019/067910, and the Cas9 mRNAs and ORFs of paragraphs [0214]-[0234] of WO2019/067910 are hereby incorporated by reference.

In some embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

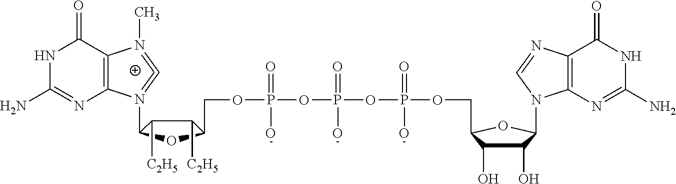

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

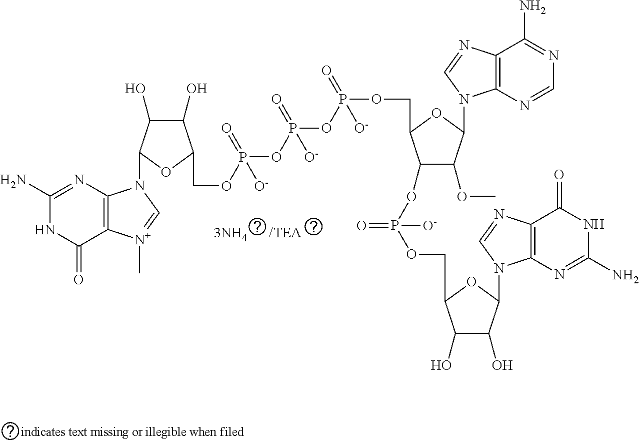 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides.

### D. Donor Constructs

The compositions and methods described herein include the use of a nucleic acid construct that comprises a sequence encoding a heterologous AAT gene (e.g., a functional or wild-type AAT) to be inserted into a cut site created by a guide RNA of the present disclosure and an RNA-guided DNA binding agent. In certain embodiments, the donor construct is a bidirectional nucleic acid construct provided herein. As used herein, such a construct is sometimes referred to as a “donor construct/template”. In some embodiments, the construct is a DNA construct. Methods of designing and making various functional/structural modifications to donor constructs are known in the art. In some embodiments, the construct may comprise any one or more of a polyadenylation tail sequence, a polyadenylation signal sequence, splice acceptor site, or selectable marker. In some embodiments, the polyadenylation tail sequence is encoded, e.g., as a “poly-A” stretch, at the 3′ end of the coding sequence. Methods of designing a suitable polyadenylation tail sequence or polyadenylation signal sequence are well known in the art. For example, the polyadenylation signal sequence AAUAAA (SEQ ID NO: 800) is commonly used in mammalian systems, although variants such as UAUAAA (SEQ ID NO: 801) or AU/GUAAA (SEQ ID NO: 802) have been identified. See, e.g., NJ Proudfoot, Genes & Dev. 25(17):1770-82, 2011.

In embodiments, the donor construct is a bidirectional nucleic acid construct. In some embodiments, such constructs comprise: a) a first segment comprising a first alpha-1 antitrypsin (AAT) polypeptide coding sequence, wherein the codon usage of the first AAT polypeptide coding sequence is different from the codon usage of the SERPINA1 gene; and b) a second segment comprising a reverse complement of a second AAT polypeptide coding sequence wherein the codon usage of the second AAT polypeptide coding sequence is different from the codon usage of the first AAT polypeptide coding sequence, from the codon usage of the SERPINA1 gene. In some embodiments, the coding sequences of the first segment and the second segment are CpG depleted. In certain embodiments, the construct does not comprise a promoter that drives the expression of either the first AAT polypeptide coding sequence or the second AAT polypeptide coding sequence. In some embodiments, the second segment is 3′ of the first segment. In certain embodiments, the construct does not comprise a homology arm.

In some embodiments, the AAT polypeptide coding sequences of the bidirectional nucleic acid construct have codon usage that prevents or reduces the ability of a SERPINA1 targeting siRNA, dsRNA or guide RNA to target it.

In certain embodiments, both the first AAT polypeptide coding sequence of the bidirectional nucleic acid construct and the second AAT polypeptide coding sequence of the bidirectional nucleic acid construct includes the use of a non-wild type codon within the a region (or one or more regions) of the sequence corresponding to bases 409-431, 409-410, 412-431, 415-418, 506-528, 506-525, 519-522, 527-528, 538-560, 538-557, 551-554, 559-560, 957-977, 970-976, 1403-1436, 1403-1425, 1410-1436, 1418-1424, 1423-1435, or any combination thereof of SEQ ID NO:703.

In some embodiments, both the first AAT polypeptide coding sequence of the bidirectional nucleic acid construct and the second AAT polypeptide coding sequence of the bidirectional nucleic acid construct includes at least one, at least 2, or at least 3 mismatches (e.g., from 1-10 mismatches, from 1-9 mismatches, from 1-8 mismatches, from 1-7 mismatches, from 1-6 mismatches, from 1-5 mismatches, from 1-4 mismatches, from 1-3 mismatches, from 1-2 mismatches, 1 mismatch, from 2-10 mismatches, from 2-9 mismatches, from 2-8 mismatches, from 2-7 mismatches, from 2-6 mismatches, from 2-5 mismatches, from 2-4 mismatches, from 1-3 mismatches, 2 mismatches, from 3-10 mismatches, from 3-9 mismatches, from 3-8 mismatches, from 3-7 mismatches, from 3-6 mismatches, from 3-5 mismatches, from 3-4 mismatches, 3 mismatches, from 4-10 mismatches, from 4-9 mismatches, from 4-8 mismatches, from 4-7 mismatches, from 4-6 mismatches, from 4-5 mismatches, 4 mismatches, from 5-10 mismatches, from 5-9 mismatches, from 5-8 mismatches, from 5-7 mismatches, from 5-6 mismatches, 5 mismatches, from 6-10 mismatches, from 6-9 mismatches, from 6-8 mismatches, from 6-7 mismatches, 6 mismatches, from 7-10 mismatches, from 7-9 mismatches, from 7-8 mismatches, 7 mismatches, from 8-10 mismatches, from 8-9 mismatches, or 8 mismatches) from a wild-type SERPINA1 gene sequence within the region (or one or more regions) of the AAT polypeptide coding sequence corresponding to bases 409-431, 409-410, 412-431, 415-418, 506-528, 506-525, 519-522, 527-528, 538-560, 538-557, 551-554, 559-560, 957-977, 970-976, 1403-1436, 1403-1425, 1410-1436, 1418-1424, 1423-1435, or any combination thereof of SEQ ID NO: 703.

In some embodiments, neither the first AAT polypeptide coding sequence of the bidirectional nucleic acid construct nor the second AAT polypeptide coding sequence of the bidirectional nucleic acid construct is targeted by an RNAi agent targeted to nucleotides 957-977, 1403-1425, or 1410-1436 of SEQ ID NO: 703.

In certain embodiments, neither the first AAT polypeptide coding sequence of the bidirectional nucleic acid construct nor the second AAT polypeptide coding sequence of the bidirectional nucleic acid construct is targeted by a SERPINA1 targeting guide RNA having a targeting sequence of SEQ ID NOs: 1129, 1130, or 1131.

In some embodiments, both the first AAT polypeptide coding sequence of the bidirectional nucleic acid construct and the second AAT polypeptide coding sequence of the bidirectional nucleic acid construct includes the use of a non-wild type codon within the region (or one or more regions) of the sequence corresponding to bases 409-431, 409-410, 412-431, 415-418, 506-528, 506-525, 519-522, 527-528, 538-560, 538-557, 551-554, 559-560, 957-977, 970-976, 1403-1436, 1403-1425, 1410-1436, 1418-1424, 1423-1435, or any combination thereof of SEQ ID NO:703.

In certain embodiments, the first AAT polypeptide coding sequence of the bidirectional nucleic acid construct comprises a sequence selected from SEQ ID NOs: 711, 712, 721, 722, 731, 732, 741, 742, 751, 752, 761, 762, 771, 772, 781, 782, 791, 792, 796, and 797. In some embodiments, the second AAT polypeptide coding sequence of the bidirectional nucleic acid construct comprises a sequence selected from SEQ ID NOs: 711, 712, 721, 722, 731, 732, 741, 742, 751, 752, 761, 762, 771, 772, 781, 782, 791, 792, 796, and 797. In certain embodiments, the nucleic acid sequence of the bidirectional nucleic acid construct is selected from: SEQ ID NOs: 711, 712, 721, 722, 731, 732, 741, 742, 751, 752, 761, 762, 771, 772, 781, 782, 791, 792, 796, and 797.

The length of the construct can vary, depending on the size of the gene to be inserted, and can be, for example, from 200 base pairs (bp) to about 5000 bp, such as about 200 bp to about 2000 bp, such as about 500 bp to about 1500 bp. In some embodiments, the length of the DNA donor template is about 200 bp, or is about 500 bp, or is about 800 bp, or is about 1000 base pairs, or is about 1500 base pairs. In other embodiments, the length of the donor template is at least 200 bp, or is at least 500 bp, or is at least 800 bp, or is at least 1000 bp, or is at least 1500 bp, or at least 2000, or at least 2500, or at least 3000, or at least 3500, or at least 4000, or at least 4500, or at least 5000.

The construct can be DNA or RNA, single-stranded, double-stranded or partially single- and partially double-stranded and can be introduced into a host cell in linear or circular (e.g., minicircle) form. See, e.g., U.S. Patent Publication Nos. 2010/0047805, 2011/0281361, 2011/0207221. If introduced in linear form, the ends of the donor sequence can be protected (e.g., from exonucleolytic degradation) by methods known to those of skill in the art. For example, one or more dideoxynucleotide residues are added to the 3′ terminus of a linear molecule or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters and genes encoding antibiotic resistance. A construct may omit viral elements. Moreover, donor constructs can be introduced as naked nucleic acid, as nucleic acid complexed with an agent such as a liposome or poloxamer, or can be delivered by viruses (e.g., adenovirus, AAV, herpesvirus, retrovirus, lentivirus).

In some embodiments, the construct may be inserted so that its expression is driven by the endogenous promoter at the insertion site (e.g., the endogenous albumin promoter when the donor is integrated into the host cell's albumin locus). In such cases, the transgene may lack control elements (e.g., promoter or enhancer) that drive its expression (e.g., a promoterless construct). Nonetheless, it will be apparent that in other cases the construct may comprise a promoter or enhancer, for example a constitutive promoter or an inducible or tissue specific (e.g., liver- or platelet-specific) promoter that drives expression of the functional protein upon integration. The construct may comprise a sequence encoding a heterologous AAT protein downstream of and operably linked to a signal sequence encoding a signal peptide. In some embodiments, the signal peptide is a signal peptide from a hepatocyte secreted protein. In some embodiments, the signal peptide is an AAT signal peptide. In some embodiments, the signal peptide is an albumin signal peptide. In some embodiments, the signal peptide is an Factor IX signal peptide. The construct may comprise a sequence encoding a heterologous AAT protein downstream of and operably linked to a signal sequence encoding an AAT signal peptide, e.g. SEQ ID NO: 700. The construct may comprise a sequence encoding a heterologous AAT protein downstream of and operably linked to a signal sequence encoding a heterologous signal peptide. In various embodiments, the methods comprise a sequence encoding a heterologous AAT protein downstream of and operably linked to a signal sequence encoding an albumin signal peptide. In some embodiments, the nucleic acid construct works in homology-independent insertion of a nucleic acid that encodes an AAT protein. In some embodiments, the nucleic acid construct works in non-dividing cells, e.g., cells in which NHEJ, not HR, is the primary mechanism by which double-stranded DNA breaks are repaired. The nucleic acid may be a homology-independent donor construct.

In some embodiments, the donor construct comprises a heterologous AAT gene that encodes a functional AAT protein. In some embodiments, the functional AAT protein is a human wild-type AAT protein sequence according to SEQ ID NO: 700. In some embodiments, the functional AAT protein is a human wild-type AAT protein sequence according to SEQ ID NO: 702. Nucleic acid encoding AAT are also exemplified and disclosed herein. In some embodiments, the construct comprises a heterologous AAT gene that encodes a functional variant of AAT, e.g., a variant that possesses increased protease inhibitor activity as compared to wild type AAT. In some embodiments, the construct comprises a heterologous AAT gene that encodes a functional variant that is 80%, 85%, 90%, 93%, 95%, 97%, 99% identical to SEQ ID NO: 700, having a functional activity that is at least 80%, 85%, 90%, 92%, 94%, 96%, 98%, 99%, 100%, or more, activity as compared to wild type AAT. In some embodiments, the construct comprises a heterologous AAT gene that encodes a functional variant that is 80%, 85%, 90%, 93%, 95%, 97%, 99% identical to SEQ ID NO: 702, having a functional activity that is at least 80%, 85%, 90%, 92%, 94%, 96%, 98%, 99%, 100%, or more, activity as compared to wild type AAT. In some embodiments, the construct comprises a heterologous AAT gene that encodes a fragment of AAT protein that possesses functional activity that is at least 80%, 85%, 90%, 92%, 94%, 96%, 98%, 99%, 100%, or more, activity as compared to wild type AAT.

Also described herein are bidirectional nucleic acid constructs that allow enhanced insertion and expression of a heterologous AAT gene. Briefly, various bidirectional constructs disclosed herein comprise at least two nucleic acid segments, wherein one segment (the first segment) comprises a coding sequence that encodes a heterologous AAT (sometimes interchangeably referred to herein as “transgene”), while the other segment (the second segment) comprises a sequence wherein the complement of the sequence encodes a heterologous AAT. The bidirectional constructs may comprise at least two nucleic acid segments in cis, wherein one segment (the first segment) comprises a coding sequence that encodes a heterologous AAT in one orientation, while the other segment (the second segment) comprises a sequence wherein its complement encodes a heterologous AAT in the other orientation. That is, first segment is a complement of the second segment but is not a perfect complement; the complement of the second segment is the reverse complement of the first segment but is not a perfect reverse complement; and both encode a heterologous AAT). A bidirectional construct may comprise a first coding sequence that encodes a heterologous AAT linked to a splice acceptor and a second coding sequence wherein the complement encodes a heterologous AAT in the other orientation, also linked to a splice acceptor. When used in combination with a gene editing system (e.g., CRISPR/Cas system; zinc finger nuclease (ZFN) system; transcription activator-like effector nuclease (TALEN) system) as described herein, the bidirectionality of the nucleic acid constructs allows the construct to be inserted in either direction (is not limited to insertion in one direction) within a target insertion site, allowing the expression of a heterologous AAT from either a) a coding sequence of one segment or 2) a complement of the other segment, thereby enhancing insertion and expression efficiency, as exemplified herein. Various known gene editing systems can be used in the practice of the present disclosure, including, e.g., CRISPR/Cas system; zinc finger nuclease (ZFN) system; transcription activator-like effector nuclease (TALEN) system.

The bidirectional constructs disclosed herein can be modified to include any suitable structural feature as needed for any particular use or that confers one or more desired function. In some embodiments, the bidirectional nucleic acid construct disclosed herein does not comprise a homology arm. In some embodiments, the bidirectional nucleic acid construct disclosed herein is a homology-independent donor construct. In some embodiments, owing in part to the bidirectional function of the nucleic acid construct, the bidirectional construct can be inserted into a genomic locus in either direction (orientation) as described herein to allow for efficient insertion or expression of a polypeptide of interest (e.g., a heterologous AAT).

In some embodiments, the bidirectional nucleic acid construct does not comprise a promoter that drives the expression of a heterologous AAT gene. For example, the expression of the polypeptide is driven by a promoter of the host cell (e.g., the endogenous albumin promoter when the transgene is integrated into a host cell's albumin locus). In some embodiments, the bidirectional nucleic acid construct includes a first segment and a second segment, each having a splice acceptor upstream of a transgene. In certain embodiments, the splice acceptor is compatible with the splice donor sequence of the host cell's safe harbor site, e.g. the splice donor of intron 1 of a human albumin gene.

In some embodiments, the bidirectional nucleic acid construct comprises a first segment comprising a coding sequence for heterologous AAT and a second segment comprising a reverse complement of a coding sequence of heterologous AAT. Thus, the coding sequence in the first segment is capable of expressing heterologous AAT, while the complement of the reverse complement in the second segment is also capable of expressing heterologous AAT. As used herein, “coding sequence” when referring to the second segment comprising a reverse complement sequence refers to the complementary (coding) strand of the second segment (i.e., the complement coding sequence of the reverse complement sequence in the second segment).

The coding sequence that encodes a heterologous AAT in the first segment is less than 100% complementary to the reverse complement of a coding sequence that also encodes heterologous AAT. That is, in some embodiments, the first segment comprises a coding sequence (1) for heterologous AAT, and the second segment is a reverse complement of a coding sequence (2) for heterologous AAT, wherein the coding sequence (1) is not identical to the coding sequence (2). For example, coding sequence (1) or coding sequence (2) that encodes for heterologous AAT can be codon optimized, such that coding sequence (1) and the reverse complement of coding sequence (2) possess less than 100% complementarity. In some embodiments, the coding sequence of the second segment encodes heterologous AAT using one or more alternative codons for one or more amino acids of the same (i.e., same amino acid sequence) heterologous AAT encoded by the coding sequence in the first segment. An “alternative codon” as used herein refers to variations in codon usage for a given amino acid, and may or may not be a preferred or optimized codon (codon optimized) for a given expression system. Preferred codon usage, or codons that are well-tolerated in a given system of expression is known in the art.

In some embodiments, the second segment comprises a reverse complement sequence that adopts different codon usage from that of the coding sequence of the first segment in order to reduce hairpin formation. Such a reverse complement forms base pairs with fewer than all nucleotides of the coding sequence in the first segment, yet it optionally encodes the same polypeptide. In such cases, the coding sequence, e.g. for Polypeptide A, of the first segment may be homologous to, but not identical to, the coding sequence, e.g. for Polypeptide A of the second half of the bidirectional construct. In some embodiments, the second segment comprises a reverse complement sequence that is not substantially complementary (e.g., not more than 70% complementary) to the coding sequence in the first segment. In some embodiments, the second segment comprises a reverse complement sequence that is highly complementary (e.g., at least 90% complementary) to the coding sequence in the first segment. In some embodiments, the second segment comprises a reverse complement sequence having at least about 30%, about 35%, about 40%, about 45%, about 50%, about 55%, about 60%, about 65%, about 70%, about 75%, about 80%, about 85%, about 90%, about 95%, about 97%, or about 99% complementarity to the coding sequence in the first segment.

In some embodiments, the first segment and the second segment are CpG depleted.

A coding sequence that encodes a polypeptide may optionally comprise one or more additional sequences, such as sequences encoding amino- or carboxy-terminal amino acid sequences such as a signal sequence, label sequence, or heterologous functional sequence (e.g. nuclear localization sequence (NLS)) linked to the polypeptide. A coding sequence that encodes a polypeptide may optionally comprise sequences encoding one or more amino-terminal signal peptide sequences. Each of these additional sequences can be the same or different in the first segment and second segment of the construct.

The bidirectional construct described herein can be used to express AAT as described herein.

In some embodiments, the bidirectional nucleic acid construct is linear. For example, the first and second segments are joined in a linear manner through a linker sequence. In some embodiments, the 5′ end of the second segment that comprises a reverse complement sequence is linked to the 3′ end of the first segment. In some embodiments, the 5′ end of the first segment is linked to the 3′ end of the second segment that comprises a reverse complement sequence. In some embodiments, the linker sequence is about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 500, 1000, 1500, 2000 or more nucleotides in length. As would be appreciated by those of skill in the art, other structural elements in addition to, or instead of a linker sequence, can be inserted between the first and second segments.

The constructs disclosed herein can be modified to include any suitable structural feature as needed for any particular use or that confers one or more desired function. In some embodiments, the bidirectional nucleic acid construct disclosed herein does not comprise a homology arm. In some embodiments, owing in part to the bidirectional function of the nucleic acid construct, the bidirectional construct can be inserted into a genomic locus in either direction as described herein to allow for efficient insertion or expression of a polypeptide of interest.

In some embodiments, one or both of the first and second segment comprises a polyadenylation tail sequence or a polyadenylation signal sequence or site downstream of an open reading frame. In some embodiments, the polyadenylation tail sequence is encoded, e.g., as a “poly-A” stretch, at the 3′ end of the first or second segment. In some embodiments, a polyadenylation tail sequence is provided co-transcriptionally as a result of a polyadenylation signal sequence or site that is encoded at or near the 3′ end of the first or second segment. Methods of designing a suitable polyadenylation tail sequence or polyadenylation signal sequence are well known in the art. Suitable splice acceptor sequences are disclosed and exemplified herein, including mouse albumin and human FIX splice acceptor sites. In some embodiments, the polyadenylation signal sequence AAUAAA (SEQ ID NO: 800) is commonly used in mammalian systems, although variants such as UAUAAA (SEQ ID NO: 801) or AU/GUAAA (SEQ ID NO: 802) have been identified. See, e.g., NJ Proudfoot, Genes & Dev. 25(17):1770-82, 2011. In some embodiments, a polyA tail sequence is included.

In some embodiments, the constructs disclosed herein can be DNA or RNA, single-stranded, double-stranded, or partially single- and partially double-stranded. For example, the constructs can be single- or double-stranded DNA. In some embodiments, the nucleic acid can be modified (e.g., using nucleoside analogs), as described herein.

In some embodiments, the constructs disclosed herein comprise a splice acceptor site on either or both ends of the construct, e.g., 5′ of an open reading frame in the first or second segments, or 5′ of one or both transgene sequences. In some embodiments, the splice acceptor site comprises NAG. In further embodiments, the splice acceptor site consists of NAG. In some embodiments, the splice acceptor is an albumin splice acceptor, e.g., an albumin splice acceptor used in the splicing together of exons 1 and 2 of albumin. In some embodiments, the splice acceptor is derived from the human albumin gene. In some embodiments, the splice acceptor is derived from the mouse albumin gene. In some embodiments, the splice acceptor is a mouse albumin splice acceptor, e.g., the mouse albumin splice acceptor used in the splicing together of exons 1 and 2 of albumin. In some embodiments, the splice acceptor is derived from the human albumin gene. Additional suitable splice acceptor sites useful in eukaryotes, including artificial splice acceptors are known and can be derived from the art. See, e.g., Shapiro, et al., 1987, Nucleic Acids Res., 15, 7155-7174, Burset, et al., 2001, Nucleic Acids Res., 29, 255-259.

In some embodiments, the constructs disclosed herein can be modified on either or both ends to include one or more suitable structural features as needed, or to confer one or more functional benefit. For example, structural modifications can vary depending on the method(s) used to deliver the constructs disclosed herein to a host cell—e.g., use of viral vector delivery or packaging into lipid nanoparticles for delivery. Such modifications include, without limitation, e.g., terminal structures such as inverted terminal repeats (ITR), hairpin, loops, and other structures such as toroid. In some embodiments, the constructs disclosed herein comprise one, two, or three ITRs. In some embodiments, the constructs disclosed herein comprise no more than two ITRs. Various methods of structural modifications are known in the art.

In some embodiments, one or both ends of the construct can be protected (e.g., from exonucleolytic degradation) by methods known in the art. For example, one or more dideoxynucleotide residues are added to the 3′ terminus of a linear molecule or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

In some embodiments, the constructs disclosed herein can be introduced into a cell as part of a vector having additional sequences such as, for example, replication origins, promoters and genes encoding antibiotic resistance. In some embodiments, the constructs can be introduced as naked nucleic acid, as nucleic acid complexed with an agent such as a liposome, polymer, or poloxamer, or can be delivered by viral vectors (e.g., adenovirus, AAV, herpesvirus, retrovirus, lentivirus).

In some embodiments, although not required for expression, the constructs disclosed herein may also include transcriptional or translational regulatory sequences, for example, promoters, enhancers, insulators, internal ribosome entry sites, sequences encoding peptides, or polyadenylation signals.

In some embodiments, the constructs comprising a coding sequence for a polypeptide of interest may include one or more of the following modifications: codon optimization (e.g., to human codons) or addition of one or more glycosylation sites. See, e.g., McIntosh et al. (2013) Blood (17):3335-44.

In some embodiments, constructs comprising alternative coding sequences can be designed to be resistant to reduction of expression by nucleic acid therapeutic agents. Nucleic acid therapeutic agents targeted to the SERPINA1 gene are provided herein. Potent gRNAs include G000409, G000414, and G000415 targeted to nucleotides 506-525, 538-557, and 412-431, respectively. RNAi agents targeted to SERPINA1 are known in the art, see, e.g., WO2018098117, WO2015003113, and WO2015195628 directed to iRNA agents targeted to SERPINA1. Potent RNAi agents provided in those applications are targeted to nucleotides 1403-1425, 1410-1436, and 957-997 of GenBank Accession No. NM_001127700.2 (in the version available on the date that the instant application is filed). Provided herein are methods for testing resistance of coding sequences and expression constructs to nucleic acid therapeutic agents. Also, methods of targeting of nucleic acid therapeutics to their target sites, and therefore methods of disrupting targeting of nucleic acid therapeutics to specific target sites are known in the art. Disruption of targeting for guide RNAs can include providing mismatches between the targeting sequence and in the PAM in the guide and the complementary sequence in the expression construct. The core sequence, located at positions +4 to +7 upstream of the PAM is particularly sensitive to mismatch with S. pyogenes Cas9 (see, e.g., Zheng et al., Sci Rep, 207), Disruption of targeting for RNAi agents can include providing mismatches between the antisense strand and the complementary sequence in the expression construct. The seed region of an RNAi agent, i.e., the hexamer or heptamer seed at positions 2-7 or 2-8 of the antisense strand of the siRNA, is particularly sensitive to mismatches (see, e.g., Birmingham et al., Nature Methods, 2006). As the standard of care for AATD relies on supplementation of AAT protein by infusion of ATT from serum, expression of AAT from the a bidirectional construct may be sufficient to treat the disease. However, as the liver pathology is, at least, in part, due to the accumulation of misfolded proteins, upon the development of liver damage, a nucleic acid therapeutic agent could be used to reduce the expression of from the endogenous SERPINA1 gene, without reducing, or substantially reducing (e.g., no more than 5% reduction, no more than 10% reduction) expression of the heterologous AAT from a bidirectional construct for expression of a heterologous AAT where both heterologous coding sequences are resistant to, i.e., not targeted by nucleic acid therapeutics. The bidirectional constructs herein are designed to be resistant to exemplary nucleic acid therapeutic agents known in the art and demonstrated to have robust activity. However, at the time of filing of the instant application, none of the agents have received approval from a regulatory authority for use in treatment of a human subject. It is also possible that other nucleic acid therapeutics targeted to SERPINA1 will be developed. Provided with the strategies and methods provided herein, one of skill in the art can design further bidirectional constructs to be resistant to newly developed nucleic acid therapeutics targeted to SERPINA1.

Thus, provided herein is a use of a nucleic acid therapeutic targeted to an endogenous SERPIINA1 gene in a method for treating AATD in a subject with one or more symptoms of liver damage associated with AATD, wherein the subject was previously treated with a bidirectional construct encoding a heterologous AAT, wherein both coding sequences within the bidirectional construct include non-wild type codon usage, wherein the coding sequences in the bidirectional construct are not targeted by the nucleic acid therapeutic targeted to the endogenous SERPINA1 gene, so that nucleic acid therapeutic agent reduces the expression of from the endogenous SERPINA1 gene, without reducing, or substantially reducing (e.g., no more than 5% reduction, no more than 10% reduction) expression of the heterologous AAT from a bidirectional construct.

### E. Gene Editing System

Various known gene editing systems can be used for targeted insertion of a bidirectional nucleic acid construct described herein, including, e.g., CRISPR/Cas system; zinc finger nuclease (ZFN) system; and transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases have been, and additional nucleases are being, for example developed based on the Argonaute system (e.g., from T. thermophilus, known as ‘TtAgo’, see Swarts et al (2014) Nature 507(7491): 258-261), which also may have the potential for uses in genome editing and gene therapy.

It will be appreciated that for methods that use the guide RNAs for a Cas nuclease, such as a Cas9 nuclease disclosed herein, the methods include the use of the CRISPR/Cas system (and any of the donor construct disclosed herein that comprises a sequence encoding a heterologous AAT). It will also be appreciated that the present disclosure contemplates methods of targeted insertion and expression of a heterologous AAT using the bidirectional constructs disclosed herein, which can be performed with or without the albumin guide RNAs disclosed herein (e.g., using a ZFN system to cause a break in a target DNA sequence, creating a site for insertion of the bidirectional construct).

In some embodiments, a CRISPR/Cas system (e.g., a guide RNA and RNA-guided DNA binding agent) can be used to create a site of insertion at a desired locus within a host genome, at which site a donor construct (e.g., bidirectional construct) comprising a sequence encoding a heterologous AAT disclosed herein can be inserted to express a heterologous AAT. In some embodiments, the heterologous AAT transgene may be heterologous with respect to its insertion site, for example inserted to a safe harbor locus, as described herein. In some embodiments, a guide RNA described herein (SEQ ID NO: 2-33) that targets a human albumin locus (e.g., intron 1) can be used according to the present methods with an RNA-guided DNA binding agent (e.g., Cas nuclease) to create a site of insertion, at which site a donor construct (e.g., bidirectional construct) comprising a sequence encoding a heterologous AAT can be inserted to express a heterologous AAT. The guide RNAs comprising guide sequences for targeted insertion of a heterologous AAT gene into intron 1 of the human albumin locus are exemplified and described herein (see, e.g., Table 1).

Methods of using various RNA-guided DNA-binding agents, e.g., a nuclease, such as a Cas nuclease, e.g., Cas9, are also well known in the art. It will be appreciated that, depending on the context, the RNA-guided DNA-binding agent can be provided as a nucleic acid (e.g., DNA or mRNA) or as a protein. In some embodiments, the present method can be practiced in a host cell that already expresses an RNA-guided DNA-binding agent.

In some embodiments, the RNA-guided DNA-binding agent, such as a Cas9 nuclease, has cleavase activity, which can also be referred to as double-strand endonuclease activity. In some embodiments, the RNA-guided DNA-binding agent, such as a Cas9 nuclease, has nickase activity, which can also be referred to as single-strand endonuclease activity. In some embodiments, the RNA-guided DNA-binding agent comprises a Cas nuclease. Examples of Cas9 nucleases include those of the type II CRISPR systems of S. pyogenes, S. aureus, and other prokaryotes (see, e.g., the list in the next paragraph), and mutant (e.g., engineered or other variant) versions thereof. See, e.g., US2016/0312198 A1; US 2016/0312199 A1.

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA-binding agent is called a ribonucleoprotein complex (RNP). In some embodiments, the RNA-guided DNA-binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—AOQ7Q2 (CPF1_FRATN)).

In some embodiments, a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. It may also be inserted within the RNA-guided DNA-binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 600) or PKKKRRV (SEQ ID NO: 601). In some embodiments, the NLS may be a bipartite sequence, such as the NLS of nucleoplasmin, KRPAATKKAGQAKKKK (SEQ ID NO: 602). In a specific embodiment, a single PKKKRKV (SEQ ID NO: 600) NLS may be linked at the C-terminus of the RNA-guided DNA-binding agent. One or more linkers are optionally included at the fusion site.

### III. Delivery Methods

The guide RNA (albumin gRNA; SERPINA1 gRNA), RNA-guided DNA binding agents (e.g., Cas nuclease), and nucleic acid constructs (e.g., bidirectional construct) disclosed herein can be delivered to a host cell or subject, in vivo or ex vivo, using various known and suitable methods available in the art. The guide RNA, RNA-guided DNA binding agents, and nucleic acid constructs can be delivered individually or together in any combination, using the same or different delivery methods as appropriate.

Conventional viral and non-viral based gene delivery methods can be used to introduce the guide RNA disclosed herein as well as the RNA-guided DNA binding agent and donor construct in cells (e.g., mammalian cells) and target tissues. As further provided herein, non-viral vector delivery systems nucleic acids such as non-viral vectors, plasmid vectors, and, e.g naked nucleic acid, and nucleic acid complexed with a delivery vehicle such as a liposome, lipid nanoparticle (LNP), or poloxamer. Viral vector delivery systems include DNA and RNA viruses.

Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by AmaxaBiosystems (Cologne, Germany), Maxcyte, Inc. (Rockville, Md.), BTX Molecular Delivery Systems (Holliston, Ma.) and Copernicus Therapeutics Inc., (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™). The preparation of lipid:nucleic acid complexes, including targeted liposomes such as immunolipid complexes, is well known in the art, and as described herein.

Various delivery systems (e.g., vectors, liposomes, LNPs) containing the guide RNAs, RNA-guided DNA binding agent, and donor construct, singly or in combination, can also be administered to an organism for delivery to cells in vivo or administered to a cell or cell culture ex vivo. Administration is by any of the routes normally used for introducing a molecule into ultimate contact with blood, fluid, or cells including, but not limited to, injection, infusion, topical application and electroporation. Suitable methods of administering such nucleic acids are available and well known to those of skill in the art.

In certain embodiments, the present disclosure provides DNA or RNA vectors encoding any one or more of the compositions disclosed herein—e.g., a guide RNA (albumin gRNA; or SERPINA1 gRNA) comprising any one or more of the guide sequences described herein; a construct (e.g., bidirectional construct) comprising a sequence encoding heterologous AAT; or a sequence encoding an RNA-guided DNA binding agent. In certain embodiments, the composition comprises DNA or RNA vectors encoding any one or more of the compositions described herein, or in any combination. In some embodiments, the vectors further comprise, e.g., promoters, enhancers, and regulatory sequences. In some embodiments, the vector that comprises a bidirectional construct comprising a sequence that encodes a heterologous AAT does not comprise a promoter that drives heterologous AAT expression. In some embodiments, the vector that comprises a guide RNA comprising any one or more of the guide sequences described herein (albumin gRNA; or SERPINA1 gRNA) also comprises one or more nucleotide sequence(s) encoding a crRNA, a trRNA, or a crRNA and trRNA, as disclosed herein.

In some embodiments, the vector comprises a nucleotide sequence encoding a guide RNA (albumin gRNA; or SERPINA1 gRNA) described herein. In some embodiments, the vector comprises one copy of a guide RNA. In other embodiments, the vector comprises more than one copy of a guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a complex with an RNA-guided DNA nuclease, such as a Cas RNP complex. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA (sgRNA). In other embodiments, the crRNA and the trRNA may be driven by their corresponding promoters on the same vector. In yet other embodiments, the crRNA and the trRNA may be encoded by different vectors.

In some embodiments, the nucleotide sequence encoding the guide RNA (albumin gRNA; or SERPINA1 gRNA) may be located on the same vector comprising the nucleotide sequence encoding an RNA-guided DNA binding agent such as a Cas protein. In some embodiments, one or more albumin gRNA or one or more SERPINA1 gRNA may be located on the same vector. In some embodiments, one or more albumin gRNA or one or more SERPINA1 gRNA may be located on the same vector with the nucleotide sequence encoding an RNA-guided DNA binding agent such as a Cas protein. In some embodiments, expression of the guide RNA and of the RNA-guided DNA binding agent such as a Cas protein may be driven by their own corresponding promoters. In some embodiments, expression of the guide RNA may be driven by the same promoter that drives expression of the RNA-guided DNA binding agent such as a Cas protein. In some embodiments, the guide RNA and the RNA-guided DNA binding agent such as a Cas protein transcript may be contained within a single transcript. For example, the guide RNA may be within an untranslated region (UTR) of the RNA-guided DNA binding agent such as a Cas protein transcript. In some embodiments, the guide RNA may be within the 5′ UTR of the transcript. In other embodiments, the guide RNA may be within the 3′ UTR of the transcript. In some embodiments, the intracellular half-life of the transcript may be reduced by containing the guide RNA within its 3′ UTR and thereby shortening the length of its 3′ UTR. In additional embodiments, the guide RNA may be within an intron of the transcript. In some embodiments, suitable splice sites may be added at the intron within which the guide RNA is located such that the guide RNA is properly spliced out of the transcript. In some embodiments, expression of the RNA-guided DNA binding agent such as a Cas protein and the guide RNA from the same vector in close temporal proximity may facilitate more efficient formation of the CRISPR RNP complex.

In some embodiments, the nucleotide sequence encoding the guide RNA (albumin gRNA; or SERPINA1 gRNA) or RNA-guided DNA binding agent may be located on the same vector comprising the construct that comprises a heterologous AAT gene. In some embodiments, proximity of the construct comprising the AAT gene and the guide RNA (or the RNA-guided DNA binding agent) on the same vector may facilitate more efficient insertion of the construct into a site of insertion created by the guide RNA/RNA-guided DNA binding agent.

In some embodiments, the vector comprises one or more nucleotide sequence(s) encoding a sgRNA (albumin gRNA; or SERPINA1 gRNA) and an mRNA encoding an RNA-guided DNA binding agent, which can be a Cas protein, such as Cas9 or Cpf1. In some embodiments, the vector comprises one or more nucleotide sequence(s) encoding a crRNA, a trRNA, and an mRNA encoding an RNA-guided DNA binding agent, which can be a Cas protein, such as, Cas9 or Cpf1. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the vector comprises a donor construct (e.g., the bidirectional nucleic acid construct) comprising a sequence that encodes a heterologous AAT, as disclosed herein. In some embodiments, in addition to the donor construct (e.g., bidirectional nucleic acid construct) disclosed herein, the vector may further comprise nucleic acids that encode the albumin guide RNAs described herein or nucleic acid encoding an RNA-guided DNA-binding agent (e.g., a Cas nuclease such as Cas9). In some embodiments, a nucleic acid encoding an albumin guide RNA or a nucleic acid encoding an RNA-guided DNA-binding agent are each or both on a separate vector from a vector that comprises the donor construct (e.g., bidirectional construct) disclosed herein. In any of the embodiments, the vector may include other sequences that include, but are not limited to, promoters, enhancers, regulatory sequences, as described herein. In some embodiments, the promoter does not drive the expression of the heterologous AAT of the donor construct (e.g., bidirectional construct). In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease (e.g., Cas9). In some embodiments, the vector comprises one or more nucleotide sequence(s) encoding a crRNA, a trRNA, and an mRNA encoding an RNA-guided DNA nuclease, which can be a Cas nuclease, such as, Cas9. In some embodiments, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the vector may be circular. In other embodiments, the vector may be linear. In some embodiments, the vector may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

In some embodiments, the vector may be a viral vector. In some embodiments, the viral vector may be genetically modified from its wild type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a host may be reduced. In some embodiments, viral genes (such as, e.g., integrase) that promote integration of the viral sequence into a host genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a host cell along with the vector system described herein. In other embodiments, the virus may be helper-free. For example, the virus may be capable of amplifying and packaging the vectors without a helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In other embodiments, the viral vector may a lentivirus vector.

In some embodiments, “AAV” refers all serotypes, subtypes, and naturally-occurring AAV as well as recombinant AAV. “AAV” may be used to refer to the virus itself or a derivative thereof. The term “AAV” includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV. In certain embodiments, the term “AAV” includes AAV3B, AAVhu.37, AAV9, AAV-DJ, AAV2/8, AAVrh10, AAVLK03, and AAV8. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest (e.g., AAT). The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV capside sequence. In general, the heterologous nucleic acid sequence (the transgene) is flanked by at least one, at least two, or at least three AAV inverted terminal repeat sequences (ITRs). An AAV vector may either be single-stranded (ssAAV) or self-complementary (scAAV). In certain embodiments, one or more regions of the AAV vector may be CpG depleted. In certain embodiments, the ITR are not CpG depleted. In certain embodiments, the ITR are CpG depleted.

In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding an RNA-guided DNA binding agent such as a Cas protein (e.g., Cas9), while a second AAV vector may contain one or more guide sequences.

In some embodiments, the vector system may be capable of driving expression of one or more coding sequences in a cell. In some embodiments, the vector does not comprise a promoter that drives expression of one or more coding sequences once it is integrated in a cell (e.g., uses the host cell's endogenous promoter such as when inserted at intron 1 of an albumin locus, as exemplified herein). In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding an RNA-guided DNA binding agent such as a Cas protein (e.g., Cas9) described herein. In some embodiments, the nuclease encoded by the vector may be a Cas protein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter.

In some embodiments, the vector may comprise any one or more of the constructs comprising a heterologous AAT gene described herein. In some embodiments, the heterologous AAT gene may be operably linked to at least one transcriptional or translational control sequence. In some embodiments, the heterologous AAT gene may be operably linked to at least one promoter. In some embodiments, the heterologous gene is not linked to a promoter that drives the expression of the heterologous gene.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In some embodiments, the promoter may be a tissue-specific promoter, e.g., a promoter specific for expression in the liver.

In some embodiments, the compositions comprise a vector system. In some embodiments, the vector system may comprise one single vector. In other embodiments, the vector system may comprise two vectors. In additional embodiments, the vector system may comprise three vectors. When different guide RNAs are used for multiplexing, or when multiple copies of the guide RNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

The vector comprising: one or more guide RNA (albumin gRNA or SERPINA1 gRNA), RNA-binding DNA binding agent, or donor construct comprising a sequence encoding a heterologous AAT protein, individually or in any combination, may be delivered by liposome, a nanoparticle, an exosome, or a microvesicle. The vector may also be delivered by a lipid nanoparticle (LNP). One or more guide RNA (albumin gRNA or SERPINA1 gRNA), RNA-binding DNA binding agent (e.g. mRNA), or donor construct comprising a sequence encoding a heterologous AAT protein, individually or in any combination, may be delivered by liposome, a nanoparticle, an exosome, or a microvesicle. One or more guide RNA (albumin gRNA or SERPINA1 gRNA), RNA-binding DNA binding agent (e.g. mRNA), or donor construct comprising a sequence encoding a heterologous AAT protein, individually or in any combination, may be delivered by LNP.

Lipid nanoparticles (LNPs) are a well-known means for delivery of nucleotide and protein cargo, and may be used for delivery of any of the guide RNAs (e.g., albumin gRNA; or SERPINA1 gRNA), RNA-guided DNA binding agent, or donor construct (e.g., bidirectional construct) disclosed herein. In some embodiments, the LNPs deliver the compositions in the form of nucleic acid (e.g., DNA or mRNA), or protein (e.g., Cas nuclease), or nucleic acid together with protein, as appropriate.

In some embodiments, provided herein is a method for delivering any of the guide RNAs described herein (albumin gRNA; or SERPINA1 gRNA) or donor construct (e.g., bidirectional construct) disclosed herein, alone or in combination, to a host cell or subject, wherein any one or more of the components is associated with an LNP. In some embodiments, the method further comprises an RNA-guided DNA binding agent (e.g., Cas9 or a sequence encoding Cas9).

In some embodiments, provided herein is a composition comprising any of the guide RNAs described herein (albumin gRNA; or SERPINA1 gRNA) or donor construct (e.g., bidirectional construct) disclosed herein, alone or in combination, with an LNP. In some embodiments, the composition further comprises an RNA-guided DNA binding agent (e.g., Cas9 or a nucleic acid sequence encoding Cas9).

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, LNPs associated with the bidirectional construct disclosed herein are for use in preparing a medicament for treating a disease or disorder. The disease or disorder may be a disease associated with al-antitrypsin deficiency (AATD).

In some embodiments, any of the guide RNAs described herein, RNA-guided DNA binding agents described herein, or donor construct (e.g., bidirectional construct) disclosed herein, alone or in combination, whether naked or as part of a vector, is formulated in or administered via a lipid nanoparticle; see e.g., WO/2017/173054, the contents of which are hereby incorporated by reference in their entirety.

It will be apparent that any one or more guide RNA disclosed herein (albumin gRNA; or SERPINA1 gRNA), an RNA-guided DNA binding agent (e.g., Cas nuclease or a nucleic acid encoding a Cas nuclease), and a donor construct (e.g., bidirectional construct) comprising a sequence encoding a heterologous AAT can be delivered using the same or different systems. For example, the guide RNA, RNA-guided DNA binding agent (e.g., Cas nuclease), and construct can be carried by the same vector (e.g., AAV). Alternatively, the RNA-guided DNA binding agent such as a Cas nuclease (as a protein or mRNA) or gRNA (albumin gRNA; or SERPINA1 gRNA) can be carried by a plasmid or LNP, while the donor construct can be carried by a vector such as AAV. The use of any of the variety of combinations will be guided by, e.g., the practicality and efficiency of their use. Furthermore, the different delivery systems can be administered by the same or different routes (e.g. by infusion; by injection, such as intramuscular injection, tail vein injection, or other intravenous injection; by intraperitoneal administration or intramuscular injection).

The different delivery systems can be delivered in vitro or in vivo simultaneously or in any sequential order. In some embodiments, the donor construct, guide RNA (albumin gRNA; or SERPINA1 gRNA), and Cas nuclease can be delivered in vitro or in vivo simultaneously, e.g., in one vector, two vectors, three vectors, individual vectors, one LNP, two LNPs, three LNPs, individual LNPs, or a combination thereof. In some embodiments, the donor construct can be delivered in vivo or in vitro, as a vector or associated with a LNP, prior to (e.g., about 1, 2, 3, 4, 5, 6, 7, 8, 9, 10, 11, 12, 13, 14, or more days) delivering the albumin guide RNA or Cas nuclease, as a vector or associated with a LNP singly or together as a ribonucleoprotein (RNP). In some embodiments, the donor construct is delivered in a single administration. In some embodiments, the donor construct can be delivered in multiple administrations. As a further example, the albumin guide RNA and Cas nuclease, as a vector or mRNA or associated with a LNP singly or together as a ribonucleoprotein (RNP), can be delivered in vivo or in vitro, prior to delivering the construct, as a vector or associated with a LNP. In some embodiments, the albumin guide RNA is delivered in a single administration. In some embodiments, the albumin guide RNA can be delivered in multiple administrations. Similarly, the SERPINA1 guide RNA and the Cas nuclease, as a vector or mRNA or associated with a LNP singly or together as a ribonucleoprotein (RNP).

In some embodiments, the present disclosure also provides pharmaceutical formulations for administering any of the guide RNAs (albumin gRNA; or SERPINA1 gRNA) disclosed herein. In some embodiments, the pharmaceutical formulation includes an RNA-guided DNA binding agent (e.g., Cas nuclease) and a donor construct comprising a coding sequence of a heterologous AAT, as disclosed herein. Pharmaceutical formulations suitable for delivery into a subject (e.g., human subject) are well known in the art.

### IV. Methods of Use

The gene encoding AAT is located on chromosome 14q32.1 and part of the Protease Inhibitor (Pi) locus. Normal AAT may be referred to as PiM. The PiZ mutation can cause liver or lung symptoms, including in homozygous (ZZ) and heterozygous (MZ or SZ) individuals. The PiS mutation can cause milder reduction in serum AAT and lower risk for lung disease. Numerous other allelic mutations are known in the art. See, e.g., Greulich et al. “Alpha-1-antitrypsin deficiency: increasing awareness and improving diagnosis,” Ther Adv Respir Dis. 2016.

AATD may be diagnosed by methods known in the art, e.g., by the presence of one or more physiologic symptoms, blood tests, or genetic tests for one or more of the 150+ known AAT mutations reported to date. See, e.g., id. Examples of blood or tests include, but are not limited to, assaying for serum AAT levels, detecting mutations by polymerase chain reaction (PCR) or next generation sequencing (NGS), isoelectric focusing (IEF) with or without immunoblotting, AAT gene locus sequencing, and serum separator cards (lateral flow assay to detect the Z protein).

In some embodiments, AAT serum levels may be considered normal within the 150-350 mg/dL range using immunodiffusion methods (which may overestimate serum levels). In these embodiments, a level of 80 mg/dL may be regarded as protective, e.g., decreased risk of one or more symptoms, e.g., emphysema, despite being lower than the normal range.

In some embodiments, AAT serum levels may be considered normal within the 90-200 mg/dL range using nephelometry or immunoturbidimetry and a purified standard. In these embodiments, a level of 50 mg/dL may be regarded as protective, e.g., decreased risk of decreased risk of one or more symptoms, e.g., emphysema, despite being lower than the normal range.

In some embodiments, AAT serum levels of less than about 130 mg/dL, 125 mg/dL, 120 mg/dL, 115 mg/dL, 110 mg/dL, 105 mg/dL, or 100 mg/dL indicate low likelihood of a homozygous AAT mutation and further genetic testing may not be necessary. In some embodiments, AAT serum levels of about 104 mg/dL indicate low likelihood of homozygous PiS, and 113 mg/dL indicates low likelihood of homozygous PiZ. In some embodiments, AAT serum levels may provide limited exclusion information for heterozygous carriers, and further genetic testing may be necessary, because AAT serum levels of about 150 mg/dL indicate low likelihood of heterozygous carrier PiMZ, and AAT serum levels of about 220 mg/dL indicate low likelihood of heterozygous carrier piMS.

Examples of detectable physiologic symptoms include, but are not limited to, lung disease or liver disease; wheezing or shortness of breath; increased risk of lung infections; chronic obstructive pulmonary disease (COPD); bronchitis, asthma, dyspnea; cirrhosis; neonatal jaundice; panniculitis; chronic cough or phlegm; recurring chest colds; yellowing of the skin or the white part of the eyes; swelling of the belly or legs. In some embodiments, individuals may be subject to blood or genetic tests if they are COPD patients, nonresponsive asthmatic patients, patients with bronchiectasis of unknown etiology, individuals with cryptogenic cirrhosis/liver disease, granulomatosis with polyangiitis, necrotizing panniculitis, or first-degree relatives of patients/carriers with AATD. In some embodiments, pulmonary function testing (PFT), functional residual capacity (RFC), or lung density loss at total lung capacity (TLC) may be performed.

In some embodiments, subjects to be treated include individuals with AAT serum below the normal range. In some embodiments, subjects to be treated include individuals with any allelic mutation combination, e.g., ZZ, MZ, MS. In some embodiments, subjects to be treated include individuals with post-bronchodilator FEV1 of at least 30%, 40%, 50%, 60% of predicted normal value. In some embodiments, subjects to be treated include individuals eligible for bronchoscopy. In some embodiments, subjects to be treated include individuals with adequate hepatic and renal function, nonsmokers, individuals who have not had lung or liver lobectomy, transplant, individuals who have not had lung volume reduction surgery, individuals who have not had acute respiratory tract infection or COPD exacerbation immediately prior to treatment, or individuals who do not have unstable cor pulmonale.

As described herein, the present disclosure provides compositions and methods for expressing heterologous AAT (e.g., a functional or wild-type AAT) at a human safe harbor site, such as an albumin safe harbor site to allow secretion of the protein. In some embodiments, the methods thereby alleviate the negative effects of AATD in the lung. The present disclosure also provides compositions and methods to knock out the endogenous SERPINA1 gene thereby eliminating the production of mutant forms of AAT associated with AAT protein polymerization and aggregation in liver hepatocytes, which lead to liver symptoms in patients with AATD. See WO/2018/119182, incorporated by reference in its entirety. Accordingly, the compositions and methods disclosed herein treat AATD by alleviating the negative effects of the disorder in the lung as well as in the liver.

AAT is primarily synthesized and secreted by hepatocytes, and functions to inhibit the activity of neutrophil elastase in the lung. Without sufficient quantities of functioning AAT, neutrophil elastase is uncontrolled and damages alveoli in the lung. Thus, mutations in SERPINA1 that result in decreased levels of AAT, or decreased levels of properly functioning AAT, lead to lung pathology, including, e.g., chronic obstructive pulmonary disease (COPD), bronchitis, or asthma.

The albumin gRNAs, donor construct (e.g., bidirectional construct comprising a sequence encoding a functional heterologous AAT), and RNA-guided DNA binding agents described herein are useful for introducing a heterologous AAT nucleic acid to a host cell, in vivo or in vitro. In some embodiments, the albumin gRNAs, donor construct (e.g., bidirectional construct comprising a sequence encoding a heterologous AAT), and RNA-guided DNA binding agents described herein are useful for expressing a functional heterologous AAT in a host cell, or in a subject in need thereof. In some embodiments, the albumin gRNAs, donor construct (e.g., bidirectional construct comprising a sequence encoding a heterologous AAT), and RNA-guided DNA binding agents described herein are useful for treating AATD in a subject in need thereof. In some embodiments, treatment of AATD by expressing heterologous AAT at an albumin locus enhances secretion of functional (e.g., wild type) AAT, and alleviates one or more symptoms of AATD, e.g., negative effects on the lungs. For example, heterologous AAT expression may alleviate lung disease or liver disease; wheezing or shortness of breath; increased risk of lung infections; COPD; bronchitis, asthma, dyspnea; cirrhosis; neonatal jaundice; panniculitis; chronic cough or phlegm; recurring chest colds; yellowing of the skin or the white part of the eyes; swelling of the belly or legs. Administration of any one or more of the albumin gRNAs, donor construct (e.g., bidirectional construct comprising a sequence encoding heterologous AAT), and RNA-guided DNA binding agents described herein leads to an increase in functional (e.g., wild type) AAT gene expression, AAT protein levels (e.g. circulating, serum, or plasma levels) or AAT activity levels (e.g., trypsin inhibition) (e.g., greater than 10%, 20%, 30%, 40%, 50%, 60%, 70%, 80%, or 90% AAT gene expression or protein levels as compared to an untreated control, e.g., by nephelometry or immunoturbidimetry, e.g., AAT greater than about 40 mg/dL, 45 mg/dL, 50 mg/dL, 60 mg/dL, 70 mg/dL, 80 mg/dL, 90 mg/dL, 100 mg/dL, or 110 mg/dL in serum). In some embodiments, the effectiveness of the treatment can be assessed by measuring serum or plasma AAT activity, wherein an increase in the subject's serum or plasma level or activity of AAT indicates effectiveness of the treatment. In some embodiments, the effectiveness of the treatment can be assessed by measuring serum or plasma AAT protein or activity levels, wherein an increase in the subject's serum or plasma level or activity of AAT indicates effectiveness of the treatment. In some embodiments, effectiveness of the treatment can be assessed by PASD staining of liver tissue sections, e.g., to measure aggregation. In some embodiments, effectiveness of the treatment can be assessed by measuring inhibition of neutrophil elastase, e.g., in the lung. In some embodiments, effectiveness of the treatment can be assessed by genotype serum level, AAT lung function, spirometry test, chest X-ray of lung, CT scan of lung, blood testing of liver function, or ultrasound of liver.

In some embodiments, treatment refers to increasing serum AAT levels, e.g., to protective levels. In some embodiments, treatment refers to increasing serum AAT levels, e.g., within the normal range. In some embodiments, treatment refers to increasing serum AAT levels, e.g., above 40, 50, 60, 70, 80, 90, or 100 mg/dL, e.g., as measured using nephelometry or immunoturbidimetry and a purified standard.

In some embodiments, treatment refers to increasing serum AAT levels, e.g., to protective levels. In some embodiments, treatment refers to increasing serum AAT levels, e.g., within the normal range. In some embodiments, treatment refers to increasing serum AAT levels, e.g., above 40, 50, 60, 70, 80, 90, or 100 mg/dL, e.g., as measured using nephelometry or immunoturbidimetry and a purified standard. In some embodiments, treatment refers to improvement in baseline serum AAT as compared to control, e.g., before and after treatment. In some embodiments, treatment refers to an improvement in histologic grading of AATD associated liver disease, e.g., by 1, 2, 3, or more points, as compared to control, e.g., before and after treatment. In some embodiments, treatment refers to improvement in Ishak fibrosis score as compared to control, e.g., before and after treatment.

In normal or healthy individuals (e.g., individuals that do not possess the ZZ, MZ, or SZ allele), AAT levels vary between about 500 μg/ml to about 3000 μg/ml in the serum. Clinically, the level of circulating AAT can be measured by enzymologic or immunologic assay (e.g., ELISA), which methods are well known in the art. See, e.g., Stoller, J. and Aboussouan, L. (2005) Alpha1-antitrypsin deficiency. Lancet 365: 2225-2236; Kanakoudi F, Drossou V, Tzimouli V, et al: Serum concentrations of 10 acute-phase proteins in healthy term and pre-term infants from birth to age 6 months. Clin Chem 1995; 41:605-608; Morse J O: Alpha-1-antitrypsin deficiency. N Engl J Med 1978; 299:1045-1048, 1099-1105; Cox D W: Alpha-1-antitrypsin deficiency. In The Metabolic and Molecular Basis of Inherited Disease. Vol 3. Seventh edition. Edited by CR Scriver, AL Beaudet, WS Sly, D Valle. New York, McGraw-Hill Book Company, 1995, pp 4125-4158.

Accordingly, in some embodiments, the compositions and methods disclosed herein are useful for increasing serum or plasma levels of AAT (e.g., functional AAT or wild type AAT) in a subject having AATD (e.g., individuals that possess the ZZ, MZ, or SZ allele) or at risk of developing AATD (e.g., individuals that possess the ZZ, MZ, or SZ allele) to about 500 μg/ml, or more. In some embodiments, the compositions and methods disclosed herein are useful for increasing AAT protein levels to about 1500 μg/ml. In some embodiments, the compositions and methods disclosed herein are useful for increasing AAT protein levels to about 1000 μg/ml to about 1500 μg/ml, about 1500 μg/ml to about 2000 μg/ml, about 2000 μg/ml to about 2500 μg/ml, about 2500 μg/ml to about 3000 μg/ml, or more. For example, the compositions and methods disclosed herein are useful for increasing serum or plasma levels of AAT in a subject having an AATD to about 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, g/ml, or more.

In some embodiments, the compositions and methods disclosed herein are useful for increasing serum or plasma levels of AAT in a subject having AATD (e.g., individuals that possess the ZZ, MZ, or SZ allele) or at risk of developing AATD (e.g., individuals that possess the ZZ, MZ, or SZ allele) by about 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more, as compared to the subject's serum or plasma level of AAT before administration.

In some embodiments, the compositions and methods disclosed herein are useful for increasing heterologous functional AAT protein or AAT activity in a host cell by about 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more, as compared to an AAT level before administration to the host cell, e.g. a normal level. In some embodiments, the cell is a liver cell.

In some embodiments, the cell (host cell) or population of cells is capable of expressing AAT, e.g., cells that originate from tissue of any one or more of liver, lung, gastric organ, kidney, stomach, proximal and distal small intestine, pancreas, adrenal glands, or brain.

In some embodiments, the method comprises administering a guide RNA and an RNA-guided DNA binding agent (such as an mRNA encoding a Cas9 nuclease) in an LNP. In further embodiments, the method comprises administering an AAV nucleic acid construct encoding a AAT protein, such as an bidirectional AAT construct. CRISPR/Cas9 LNP, comprising guide RNA and an mRNA encoding a Cas9, can be administered intravenously. AAV AAT donor construct can be administered intravenously. Exemplary dosing of CRISPR/Cas9 LNP includes about 0.1, 0.25, 0.3, 0.5, 1, 2, 3, 4, 5, 6, 8, or 10 mpk (RNA). The units mg/kg and mpk are being used interchangeably herein. Exemplary dosing of AAV comprising a nucleic acid encoding a AAT protein includes an MOI of about 10¹¹, 10¹², 10¹³, and 10¹⁴ vg/kg, optionally the MOI may be about 1×10¹³ to 1×10¹⁴ vg/kg.

In some embodiments, the method comprises expressing a therapeutically effective amount of the AAT protein. In some embodiments, the method comprises achieving a therapeutically effective level of circulating AAT activity in an individual. In particular embodiments, the method comprises achieving AAT activity of at least about 5% to about 50% of normal. The method may comprise achieving AAT activity of at least about 50% to about 150% of normal. In certain embodiments, the method comprises achieving an increase in AAT activity over the patient's baseline AAT activity of at least about 1% to about 50% of normal AAT activity, or at least about 5% to about 50% of normal AAT activity, or at least about 50% to about 150% of normal AAT activity.

In some embodiments, the method further comprises achieving a durable effect, e.g. at least 1 year. In some embodiments, the method further comprises achieving the therapeutic effect in a durable and sustained manner, e.g. at least 1 year. In some embodiments, the level of circulating AAT activity or level is stable for at least 1 year. In some embodiments a steady-state activity or level of AAT protein is achieved by at least 7 days, at least 14 days, or at least 28 days. In additional embodiments, the method comprises maintaining AAT activity or levels after a single dose for at least 1 year.

In additional embodiments involving insertion into the albumin locus, the individual's circulating albumin levels are normal. The method may comprise maintaining the individual's circulating albumin levels within ±5%, ±10%, ±15%, ±20%, or ±50% of normal circulating albumin levels. In certain embodiments, the individual's albumin levels are unchanged as compared to the albumin levels of untreated individuals by at least week 4, week 8, week 12, or week 20. In certain embodiments, the individual's albumin levels transiently drop then return to normal levels. In particular, the methods may comprise detecting no significant alterations in levels of plasma albumin.

In some embodiments, the methods provided herein comprise a method or use of modifying (e.g., creating a double strand break in) an albumin gene, such as a human albumin gene, comprising, administering or delivering to a host cell or population of host cells any one or more of the gRNAs, donor construct (e.g., bidirectional construct comprising a sequence encoding AAT), and RNA-guided DNA binding agents (e.g., Cas nuclease) described herein. In some embodiments, the method comprises a method or use of modifying (e.g., creating a double strand break in) an albumin intron 1 region, such as a human albumin intron 1, comprising, administering or delivering to a host cell or population of host cells any one or more of the gRNAs, donor construct (e.g., bidirectional construct comprising a sequence encoding AAT), and RNA-guided DNA binding agents (e.g., Cas nuclease) described herein. In some embodiments, the method comprises a method or use of modifying (e.g., creating a double strand break in) a human safe harbor, such as liver tissue or hepatocyte host cell, comprising, administering or delivering to a host cell or population of host cells any one or more of the gRNAs, donor construct (e.g., bidirectional construct comprising a sequence encoding AAT), and RNA-guided DNA binding agents (e.g., Cas nuclease) described herein. Insertion within a safe harbor locus, such as an albumin locus, allows overexpression of the SERPINA1 gene without significant deleterious effects on the host cell or cell population, such as liver cells.

In some embodiments, the present disclosure provides a method or use of modifying (e.g., creating a double strand break in) intron 1 of a human albumin locus comprising, administering or delivering to a host cell any one or more of the albumin gRNAs, donor construct (e.g., bidirectional construct comprising a sequence encoding a heterologous AAT), and RNA-guided DNA binding agents (e.g., Cas nuclease) described herein. In some embodiments, the albumin guide RNA comprises a guide sequence that contains at least 15, 16, 17, 18, 19, or 20 contiguous nucleotides that are capable of binding to a region within intron 1 of a human albumin locus (SEQ ID NO: 1). In some embodiments, the albumin guide RNA comprises at least 15, 16, 17, 18, 19, or 20 contiguous nucleotides of a sequence selected from the group consisting of SEQ ID NOs: 2-33. In some embodiments, the albumin guide RNA comprises a sequence that is at least 95% identical or 90% identical to a sequence selected from the group consisting of SEQ ID NOs: 2-33. In some embodiments, the albumin gRNA comprises a guide sequence comprising a sequence of any one of SEQ ID NOs.: 4, 13, 17, 19, 27, 28, 30, or 31. In some embodiments, the administration is in vitro. In some embodiments, the administration is in vivo. In some embodiments, the donor construct is a bidirectional construct that comprises a sequence encoding a heterologous AAT. In some embodiments, the host cell is a liver cell.

In some embodiments, the present disclosure provides a method or use of introducing a bidirectional nucleic acid construct provided herein to a host cell comprising, administering or delivering any one or more of the albumin gRNAs, donor construct (e.g., a bidirectional nucleic acid construct provided herein), and RNA-guided DNA binding agents (e.g., Cas nuclease) described herein. In some embodiments, the albumin gRNA comprises a guide sequence that contains at least 15, 16, 17, 18, 19, or 20 contiguous nucleotides that are capable of binding to a region within intron 1 of a human albumin locus (SEQ ID NO: 1). In some embodiments, the albumin guide RNA comprises at least 15, 16, 17, 18, 19, or 20 contiguous nucleotides of a sequence selected from the group consisting of SEQ ID NOs: 2-33. In some embodiments, the albumin guide RNA comprises a sequence that is at least 95% identical or 90% identical to a sequence selected from the group consisting of SEQ ID NOs: 2-33. In some embodiments, the albumin gRNA comprises a guide sequence comprising a sequence of any one of SEQ ID NOs.: 4, 13, 17, 19, 27, 28, 30, or 31. In some embodiments, the albumin gRNA comprising a sequence chosen from: a) a sequence that is at least 95%, 90%, 85%, 80%, or 75% identical to a sequence selected from the group consisting of SEQ ID NOs: 2, 8, 13, 19, 28, 29, 31, 32, or 33; b) at least 17, 18, 19, or 20 contiguous nucleotides of a sequence selected from the group consisting of SEQ ID NOs: 2, 8, 13, 19, 28, 29, 31, 32, or 33; c) a sequence selected from the group consisting of SEQ ID NOs: 34, 40, 45, 51, 60, 61, 63, 64, 65, 66, 72, 77, 83, 92, 93, 95, 96, or 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides+/−5 nucleotides of the genomic coordinates listed for SEQ ID NOs: 2-33. In some embodiments, the host cell is a liver cell.

In some embodiments, the present disclosure provides a method or use of expressing a heterologous AAT (e.g., functional or wild type AAT) in a host cell comprising, administering or delivering any one or more of the albumin gRNAs, a bidirectional nucleic acid construct provided herein, and RNA-guided DNA binding agents (e.g., Cas nuclease) described herein. In some embodiments, the subject in need thereof is between birth and 2 years of age; between 2 to 12 years of age; or between 12 to 21 years of age. In some embodiments, the albumin gRNA comprises a guide sequence that contains at least 15, 16, 17, 18, 19, or 20 contiguous nucleotides that are capable of binding to a region within intron 1 of a human albumin locus (SEQ ID NO: 1). In some embodiments, the albumin gRNA comprises at least 15, 16, 17, 18, 19, or 20 contiguous nucleotides of a sequence selected from the group consisting of SEQ ID NOs: 2-33. In some embodiments, the albumin gRNA comprises a sequence that is at least 95% identical or 90% identical to a sequence selected from the group consisting of SEQ ID NOs: 2-33. In some embodiments, the albumin gRNA comprises a guide sequence comprising a sequence of any one of SEQ ID NOs: 4, 13, 17, 19, 27, 28, 30, or 31. In some embodiments, the albumin gRNA comprising a sequence chosen from: a) a sequence that is at least 95%, 90%, 85%, 80%, or 75% identical to a sequence selected from the group consisting of SEQ ID Nos: 2, 8, 13, 19, 28, 29, 31, 32, or 33; b) at least 17, 18, 19, or 20 contiguous nucleotides of a sequence selected from the group consisting of SEQ ID NOs: 2, 8, 13, 19, 28, 29, 31, 32, or 33; c) a sequence selected from the group consisting of SEQ ID NOs: 34, 40, 45, 51, 60, 61, 63, 64, 65, 66, 72, 77, 83, 92, 93, 95, 96, or 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 5, 6, 7, 8, 9, 10, 11, 12, 13, 14, 15, 16, 17, 18, 19, 20, 21, 22, 23, 24, or 25 consecutive nucleotides within or spanning the genomic coordinates listed for SEQ ID NOs: 2-33. In some embodiments, the administration is in vitro. In some embodiments, the administration is in vivo. In some embodiments, the host cell is a liver cell.

In some embodiments, the present disclosure provides a method or use of treating AATD comprising, administering or delivering a bidirectional nucleic acid construct provided herein, and RNA-guided DNA binding agents (e.g., Cas nuclease) described herein to a subject in need thereof. In some embodiments, the albumin gRNA comprises a guide sequence that contains at least 15, 16, 17, 18, 19, or 20 contiguous nucleotides that are capable of binding to a region within intron 1 of a mouse or a human albumin locus (SEQ ID NO: 1). In some embodiments, the albumin gRNA comprises at least 15, 16, 17, 18, 19, or 20 contiguous nucleotides of a sequence selected from the group consisting of SEQ ID NOs: 2-33. In some embodiments, the albumin gRNA comprises a sequence that is at least 95% identical or 90% identical to a sequence selected from the group consisting of SEQ ID NOs: 2-33. In some embodiments, the albumin gRNA comprises a guide sequence comprising a sequence of any one of SEQ ID NO: 4, 13, 17, 19, 27, 28, 30, or 31. In some embodiments, the albumin gRNA comprising a sequence chosen from: a) a sequence that is at least 95%, 90%, 85%, 80%, or 75% identical to a sequence selected from the group consisting of SEQ ID Nos: 2, 8, 13, 19, 28, 29, 31, 32, 33; b) at least 17, 18, 19, or 20 contiguous nucleotides of a sequence selected from the group consisting of SEQ ID NOs: 2, 8, 13, 19, 28, 29, 31, 32, 33; c) a sequence selected from the group consisting of SEQ ID NOs: 34, 40, 45, 51, 60, 61, 63, 64, 65, 66, 72, 77, 83, 92, 93, 95, 96, and 97; d) a sequence that is at least 95%, 90%, 85%, 80%, or 75% identical to a sequence selected from the group consisting of SEQ ID NOs: 2-33; e) at least 17, 18, 19, or 20 contiguous nucleotides of a sequence selected from the group consisting of SEQ ID NOs: 2-33; f) a sequence selected from the group consisting of SEQ ID NOs: 34-97; and g) a sequence that is complementary to 15 consecutive nucleotides+/−5 nucleotides of the genomic coordinates listed for SEQ ID NOs: 2-33. In some embodiments, the host cell is a liver cell.

In some embodiments, the present disclosure provides a method or use of increasing functional AAT secretion from a liver cell comprising, administering or delivering any one or more of the albumin gRNAs, a bidirectional nucleic acid construct provided herein, and RNA-guided DNA binding agents (e.g., Cas nuclease) described herein. In some embodiments, the albumin gRNA comprises a guide sequence that contains at least 15, 16, 17, 18, 19, or 20 contiguous nucleotides that are capable of binding to a region within intron 1 of a mouse or a human albumin locus (SEQ ID NO: 1). In some embodiments, the albumin gRNA comprises at least 15, 16, 17, 18, 19, or 20 contiguous nucleotides of a sequence selected from the group consisting of SEQ ID NOs: 2-33. In some embodiments, the albumin gRNA comprises a sequence that is at least 95% identical or 90% identical to a sequence selected from the group consisting of SEQ ID NOs: 2-33. In some embodiments, the albumin gRNA comprises a guide sequence comprising a sequence of any one of SEQ ID NO.: 4, 13, 17, 19, 27, 28, 30, or 31. In some embodiments, the administration is in vitro. In some embodiments, the administration is in vivo. In some embodiments, the host cell is a liver cell.

As described herein, the bidirectional nucleic acid construct provided herein, albumin gRNA, and RNA-guided DNA binding agent can be delivered using any suitable delivery system and method known in the art. The compositions can be delivered in vitro or in vivo simultaneously or in any sequential order. In some embodiments, the bidirectional nucleic acid construct provided herein, albumin gRNA, and Cas nuclease can be delivered in vitro or in vivo simultaneously, e.g., in one vector, two vectors, individual vectors, one LNP, two LNPs, individual LNPs, or a combination thereof. In some embodiments, the bidirectional nucleic acid construct provided herein can be delivered in vivo or in vitro, as a vector or associated with a LNP, prior to (e.g., about 1, 2, 3, 4, 5, 6, 7, 8, 9, 10, 11, 12, 13, 14, or more days) delivering the albumin gRNA or Cas nuclease, as a vector or associated with a LNP singly or together as a ribonucleoprotein (RNP). As a further example, the guide RNA and Cas nuclease, as a vector or associated with a LNP singly or together as a ribonucleoprotein (RNP), can be delivered in vivo or in vitro, prior to (e.g., about 1, 2, 3, 4, 5, 6, 7, 8, 9, 10, 11, 12, 13, 14, or more days) delivering the construct, as a vector or associated with a LNP. In some embodiments, the guide RNA and Cas nuclease are associated with an LNP and delivered to the host cell prior to delivering the bidirectional nucleic acid construct provided herein.

In some embodiments, the bidirectional nucleic acid construct provided herein comprises a sequence encoding a heterologous AAT, wherein the AAT sequence is wild type AAT, e.g., SEQ ID NO: 700 or 702. In some embodiments, the sequence encodes a functional variant of AAT. For example, the variant possesses increased trypsin inhibition activity than wild type AAT. In some embodiments, the sequence encodes an AAT variant that is 80%, 85%, 90%, 93%, 95%, 97%, 99% identical to SEQ ID NO: 702, having at least 80%, 85%, 90%, 92%, 94%, 96%, 98%, 99%, 100%, or more, activity as compared to wild type AAT. In some embodiments, the sequence encodes a functional fragment of AAT, wherein the fragment possesses at least 80%, 85%, 90%, 92%, 94%, 96%, 98%, 99%, 100%, or more, activity as compared to wild type AAT.

In some embodiments, the bidirectional nucleic acid construct provided herein is administered in a nucleic acid vector, such as an AAV vector, e.g., AAV8. In some embodiments, the donor construct does not comprise a homology arm.

In some embodiments, the subject is a mammal. In some embodiments, the subject is human.

In some embodiments, the bidirectional nucleic acid construct provided herein, albumin gRNA, and RNA-guided DNA binding agent are administered intravenously. In some embodiments, the bidirectional nucleic acid construct provided herein, albumin gRNA, and RNA-guided DNA binding agent are administered into the hepatic circulation.

In some embodiments, a single administration of a bidirectional nucleic acid construct provided herein, albumin gRNA, and RNA-guided DNA binding agent is sufficient to increase expression and secretion of AAT to a desirable level. In other embodiments, more than one administration of a composition comprising a bidirectional nucleic acid construct provided herein, albumin gRNA, and RNA-guided DNA binding agent may be beneficial to maximize therapeutic effects.

In some embodiments, multiple administrations of bidirectional nucleic acid construct provided herein, albumin gRNA, and RNA-guided DNA binding agent are used to increase expression and secretion of AAT to a desirable level or maximize editing via cumulative effects. In some embodiments, multiple administrations of an albumin guide RNA are used to increase expression and secretion of AAT to a desirable level or maximize editing via cumulative effects. In some embodiments, multiple administrations of a Cas nuclease are used to increase expression and secretion of AAT to a desirable level or maximize editing via cumulative effects.

In some embodiments, a method of treating AATD further includes administering a SERPINA1 guide RNA comprising any one or more of the guide sequences of SEQ ID Nos: 1000-1131. In some embodiments, SERPINA1 gRNAs comprising any one or more of the guide sequences of SEQ ID Nos: 1000-1131 administered to treat AATD. The SERPINA1 guide RNAs may be administered together with a Cas protein or an mRNA or vector encoding a Cas protein, such as, for example, Cas9.

In some embodiments, a method of treating AATD includes reducing or preventing the accumulation of AAT (e.g., mutant, non-functional AAT) in the serum, liver, liver tissue, liver cells, or hepatocytes of a subject is provided comprising administering a SERPINA1 guide RNA comprising any one or more of the guide sequences of SEQ ID NOs: 1000-1131. In some embodiments, SERPINA1 gRNAs comprising any one or more of the guide sequences of SEQ ID NOs: 1000-1131 are administered to reduce or prevent the accumulation of AAT (e.g., mutant, non-functional AAT) in the liver, liver tissue, liver cells, or hepatocytes. The gRNAs may be administered together with an RNA-guided DNA binding agent such as a Cas protein or an mRNA or vector encoding a Cas protein, such as, for example, Cas9.

In some embodiments, the SERPINA1 gRNAs comprising the guide sequences of Table 2 together with a Cas protein induce DSBs, and non-homologous ending joining (NHEJ) during repair leads to a mutation in the SERPINA1 gene. In some embodiments, NHEJ leads to a deletion or insertion of a nucleotide(s), which induces a frame shift or nonsense mutation in the SERPINA1 gene. In some embodiments, the gRNAs comprising the guide sequences of Table 2 together with a Cas protein induce DSBs, and NHEJ repair mediates insertion of the template nucleic acid construct. In some embodiments, insertion of the template nucleic acid increases secreted AAT protein levels. In some embodiments, insertion of the template nucleic acid increases secreted heterologous AAT protein levels. In some embodiments, insertion of the template nucleic acid increases blood, serum, or plasma AAT protein levels.

In some embodiments, administering the SERPINA1 guide RNAs disclosed herein reduces levels of endogenous alpha-1 antitrypsin (AAT) produced by the subject, and therefore prevents accumulation and aggregation of AAT in the liver.

In some embodiments, a single administration of the SERPINA1 guide RNA disclosed herein is sufficient to knock down expression of the endogenous protein. In some embodiments, a single administration of the SERPINA1 guide RNA disclosed herein is sufficient to knock down or knock out expression of the endogenous protein. In other embodiments, more than one administration of the SERPINA1 guide RNA disclosed herein may be beneficial to maximize editing via cumulative effects.

In some embodiments, endogenous AAT protein expression is reduced by administration of a nucleic acid therapeutic other than a guide RNA. In certain embodiments, the nucleic acid is an RNAi agent. Exemplary iRNA agents targeted to SERPINA1 are provided, for example, in WO2018098117, WO2015003113, and WO2015195628A2. Potent RNAi agents have been described targeting nucleotides 957-977, 1418-1424, and 1423-1435. Methods of making RNAi agents and their use for reducing expression of endogenous AAT protein in a subject and of treating AATD are provided in the cited publications and known in the art.

In some embodiments, administering the insertion guide RNAs disclosed herein increases levels of circulating alpha-1 antitrypsin (AAT) produced by the subject, and therefore prevents damage associated with high neutrophil elastase activity.

In some embodiments, a single administration or multiple administrations of an insertion guide RNA disclosed herein is sufficient to increase expression of a functional AAT protein. In some embodiments, a single administration or multiple administrations of the insertion guide RNA disclosed herein is sufficient to supplement or restore expression of the AAT protein activity. In some embodiments, the insertion guide RNA results in increased AAT serum levels, e.g., to protective levels (e.g., at or above 80 mg/dL as measured by immunodiffusion, at or above 50 mg/dL as measured using nephelometry or immunoturbidimetry and a purified standard). In some embodiments, the insertion guide RNA results in increased AAT serum levels, e.g., to normal levels (e.g., 150-350 mg/dL as measured by immunodiffusion, 90-200 mg/dL as measured using nephelometry or immunoturbidimetry and a purified standard). In some embodiments, the insertion guide RNA results in improvement in histologic grading of AATD associated liver disease, e.g., by 1, 2, 3, or more points, as compared to control, e.g., before and after treatment. In some embodiments, the insertion guide RNA results in improvement in Ishak fibrosis score as compared to control, e.g., before and after treatment. In some embodiments, a single administration improves lung disease measures, e.g., as assayed by pulmonary function testing (PFT), functional residual capacity (RFC), or lung density loss at total lung capacity (TLC). In other embodiments, more than one administration of the insertion guide RNA disclosed herein may be beneficial to maximize editing via cumulative effects.

In some embodiments, the efficacy of treatment with the compositions provided herein is seen at 1 year, 2 years, 3 years, 4 years, 5 years, or 10 years after delivery.

In some embodiments, treatment slow or halts lung disease progression associated with AATD. In some embodiments, lung disease is measured by changes in lung structure, lung function, or symptoms in the subject. In some embodiments, efficacy of treatment is measured by increased survival time of the subject.

In some embodiments, efficacy of treatment is measured by the slowing of development of pulmonary indications. In some embodiments, efficacy of treatment is measured by the slowing of development of pulmonary indications. In some embodiments, efficacy of treatment is measured by slowing progression in any one or more COPD, emphysema, or dyspnea. In some embodiments, efficacy of treatment is measured by improvement or stabilization in any one or more of cough, sputum production, or wheezing.

In some embodiments, treatment slows or halts liver disease progression. In some embodiments, treatment improves liver disease measures. In some embodiments, liver disease is measured by changes in liver structure, liver function, or symptoms in the subject.

In some embodiments, efficacy of treatment is measured by the ability to delay or avoid a liver transplantation in the subject. In some embodiments, efficacy of treatment is measured by increased survival time of the subject.

In some embodiments, efficacy of treatment is measured by reduction in liver enzymes in blood. In some embodiments, the liver enzymes are alanine transaminase (ALT) or aspartate transaminase (AST).

In some embodiments, efficacy of treatment is measured by the slowing of development of scar tissue or decrease in scar tissue in the liver based on biopsy results.

In some embodiments, efficacy of treatment is measured using patient-reported results such as fatigue, weakness, itching, loss of appetite, loss of appetite, weight loss, nausea, or bloating. In some embodiments, efficacy of treatment is measured by decreases in edema, ascites, or jaundice. In some embodiments, efficacy of treatment is measured by decreases in portal hypertension. In some embodiments, efficacy of treatment is measured by decreases in rates of liver cancer.

In some embodiments, efficacy of treatment is measured using imaging methods. In some embodiments, the imaging methods are ultrasound, computerized tomography, magnetic resonance imagery, or elastography.

In some embodiments, the serum or liver AAT levels (e.g., mutant, non-functional AAT) are reduced by 70-95%, 80-95%, 85-95%, 80-99%, or 85-99% as compared to serum or liver AAT levels (e.g., mutant, non-functional AAT) before administration of the composition.

In some embodiments, the percent editing of the SERPINA1 gene is 70-99%. In some embodiments, the percent editing is 70-95%, 80-95%, 85-95%, 80-99%, or 85-99%.

In some embodiments, the use of any one or more guide RNAs (albumin gRNA; or SERPINA1 gRNA) comprising any one or more of the guide sequences in Table 1 or Table 2, or Table 3 (e.g., in a composition provided herein) is provided for the preparation of a medicament for treating a human subject having AATD.

In some embodiments, the present disclosure provides combination therapies comprising any one or more of the gRNAs comprising any one or more of the guide sequences disclosed in Table 1 or Table 2 together with an augmentation therapy suitable for alleviating the lung symptoms of AATD. In some embodiments, the augmentation therapy for lung disease is intravenous therapy with AAT purified from human plasma, as described in Turner, BioDrugs 2013 December; 27(6):547-58. In some embodiments, the augmentation therapy is with Prolastin®, Zemaira®, Aralast®, or Kamada®.

In some embodiments, the combination therapy comprises any one or more of the gRNAs comprising any one or more of the guide sequences disclosed in Table 1 with a bidirectional construct comprising a first alpha-1 antitrypsin (AAT) polypeptide coding sequence and second alpha-1 antitrypsin (AAT) polypeptide coding sequence, together with a siRNA that targets a wild type ATT sequence. In some embodiments, the siRNA is any siRNA capable of further reducing or eliminating the expression of wild type or mutant AAT. In some embodiments, the siRNA is administered after any one or more of the gRNAs comprising any one or more of the guide sequences disclosed in Table 1 and the bidirectional construct. In some embodiments, the siRNA is administered on a regular basis following treatment with any of the gRNA compositions of Table 1 in and the bidirectional constructs provided herein

In some embodiments, the combination therapy comprises any one or more of the gRNAs comprising any one or more of the guide sequences disclosed in Table 1 with a bidirectional construct comprising a first alpha-1 antitrypsin (AAT) polypeptide coding sequence and second alpha-1 antitrypsin (AAT) polypeptide coding sequence together with one or more treatment for smoking cessation, preventive vaccinations, bronchodilators, supplemental oxygen when indicated, and physical rehabilitation in a program similar to that designed for patients with smoking-related COPD.

This description and exemplary embodiments should not be taken as limiting. For the purposes of this specification and appended embodiments, unless otherwise indicated, all numbers expressing quantities, percentages, or proportions, and other numerical values used in the specification and embodiments, are to be understood as being modified in all instances by the term “about,” to the extent they are not already so modified. Accordingly, unless indicated to the contrary, the numerical parameters set forth in the following specification and attached embodiments are approximations that may vary depending upon the desired properties sought to be obtained. At the very least, and not as an attempt to limit the application of the doctrine of equivalents to the scope of the embodiments, each numerical parameter should at least be construed in light of the number of reported significant digits and by applying ordinary rounding techniques.

Human AAT Protein Sequence
NCBI Ref: NP_000286:
(SEQ ID NO: 700)
MPSSVSWGILLLAGLCCLVPVSLAEDPQGDAAQKTDTSHHDQDHPTFNKITPNLAEF
AFSLYRQLAHQSNSTNIFFSPVSIATAFAMLSLGTKADTHDEILEGLNFNLTEIPEAQIH
EGFQELLRTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEVKDTEEED
FHVDQVTTVKVPMMKRLGMFNIQHCKKLSSWVLLMKYLGNATAIFFLPDEGKLQH
LENELTHDIITKFLENEDRRSASLHLPKLSITGTYDLKSVLGQLGITKVFSNGADLSGV
TEEAPLKLSKAVHKAVLTIDEKGTEAAGAMFLEAIPMSIPPEVKFNKPFVFLMIEQNT
KSPLFMGKVVNPTQK
Human AAT Nucleotide Sequence
NCBI Ref: NM_000295):
(SEQ ID NO: 701)
ACAATGACTCCTTTCGGTAAGTGCAGTGGAAGCTGTACACTGCCCAGGCAAAGC
GTCCGGGCAGCGTAGGCGGGCGACTCAGATCCCAGCCAGTGGACTTAGCCCCTG
TTTGCTCCTCCGATAACTGGGGTGACCTTGGTTAATATTCACCAGCAGCCTCCCC
CGTTGCCCCTCTGGATCCACTGCTTAAATACGGACGAGGACAGGGCCCTGTCTCC
TCAGCTTCAGGCACCACCACTGACCTGGGACAGTGAATCGACAATGCCGTCTTCT
GTCTCGTGGGGCATCCTCCTGCTGGCAGGCCTGTGCTGCCTGGTCCCTGTCTCCCT
GGCTGAGGATCCCCAGGGAGATGCTGCCCAGAAGACAGATACATCCCACCATGA
TCAGGATCACCCAACCTTCAACAAGATCACCCCCAACCTGGCTGAGTTCGCCTTC
AGCCTATACCGCCAGCTGGCACACCAGTCCAACAGCACCAATATCTTCTTCTCCC
CAGTGAGCATCGCTACAGCCTTTGCAATGCTCTCCCTGGGGACCAAGGCTGACAC
TCACGATGAAATCCTGGAGGGCCTGAATTTCAACCTCACGGAGATTCCGGAGGC
TCAGATCCATGAAGGCTTCCAGGAACTCCTCCGTACCCTCAACCAGCCAGACAGC
CAGCTCCAGCTGACCACCGGCAATGGCCTGTTCCTCAGCGAGGGCCTGAAGCTA
GTGGATAAGTTTTTGGAGGATGTTAAAAAGTTGTACCACTCAGAAGCCTTCACTG
TCAACTTCGGGGACACCGAAGAGGCCAAGAAACAGATCAACGATTACGTGGAGA
AGGGTACTCAAGGGAAAATTGTGGATTTGGTCAAGGAGCTTGACAGAGACACAG
TTTTTGCTCTGGTGAATTACATCTTCTTTAAAGGCAAATGGGAGAGACCCTTTGA
AGTCAAGGACACCGAGGAAGAGGACTTCCACGTGGACCAGGTGACCACCGTGAA
GGTGCCTATGATGAAGCGTTTAGGCATGTTTAACATCCAGCACTGTAAGAAGCTG
TCCAGCTGGGTGCTGCTGATGAAATACCTGGGCAATGCCACCGCCATCTTCTTCC
TGCCTGATGAGGGGAAACTACAGCACCTGGAAAATGAACTCACCCACGATATCA
TCACCAAGTTCCTGGAAAATGAAGACAGAAGGTCTGCCAGCTTACATTTACCCA
AACTGTCCATTACTGGAACCTATGATCTGAAGAGCGTCCTGGGTCAACTGGGCAT
CACTAAGGTCTTCAGCAATGGGGCTGACCTCTCCGGGGTCACAGAGGAGGCACC
CCTGAAGCTCTCCAAGGCCGTGCATAAGGCTGTGCTGACCATCGACGAGAAAGG
GACTGAAGCTGCTGGGGCCATGTTTTTAGAGGCCATACCCATGTCTATCCCCCCC
GAGGTCAAGTTCAACAAACCCTTTGTCTTCTTAATGATTGAACAAAATACCAAGT
CTCCCCTCTTCATGGGAAAAGTGGTGAATCCCACCCAAAAATAACTGCCTCTCGC
TCCTCAACCCCTCCCCTCCATCCCTGGCCCCCTCCCTGGATGACATTAAAGAAGG
GTTGAGCTGGTCCCTGCCTGCATGTGACTGTAAATCCCTCCCATGTTTTCTCTGAG
TCTCCCTTTGCCTGCTGAGGCTGTATGTGGGCTCCAGGTAACAGTGCTGTCTTCG
GGCCCCCTGAACTGTGTTCATGGAGCATCTGGCTGGGTAGGCACATGCTGGGCTT
GAATCCAGGGGGGACTGAATCCTCAGCTTACGGACCTGGGCCCATCTGTTTCTGG
AGGGCTCCAGTCTTCCTTGTCCTGTCTTGGAGTCCCCAAGAAGGAATCACAGGGG
AGGAACCAGATACCAGCCATGACCCCAGGCTCCACCAAGCATCTTCATGTCCCCC
TGCTCATCCCCCACTCCCCCCCACCCAGAGTTGCTCATCCTGCCAGGGCTGGCTG
TGCCCACCCCAAGGCTGCCCTCCTGGGGGCCCCAGAACTGCCTGATCGTGCCGTG
GCCCAGTTTTGTGGCATCTGCAGCAACACAAGAGAGAGGACAATGTCCTCCTCTT
GACCCGCTGTCACCTAACCAGACTCGGGCCCTGCACCTCTCAGGCACTTCTGGAA
AATGACTGAGGCAGATTCTTCCTGAAGCCCATTCTCCATGGGGCAACAAGGACA
CCTATTCTGTCCTTGTCCTTCCATCGCTGCCCCAGAAAGCCTCACATATCTCCGTT
TAGAATCAGGTCCCTTCTCCCCAGATGAAGAGGAGGGTCTCTGCTTTGTTTTCTCT
ATCTCCTCCTCAGACTTGACCAGGCCCAGCAGGCCCCAGAAGACCATTACCCTAT
ATCCCTTCTCCTCCCTAGTCACATGGCCATAGGCCTGCTGATGGCTCAGGAAGGC
CATTGCAAGGACTCCTCAGCTATGGGAGAGGAAGCACATCACCCATTGACCCCC
GCAACCCCTCCCTTTCCTCCTCTGAGTCCCGACTGGGGCCACATGCAGCCTGACT
TCTTTGTGCCTGTTGCTGTCCCTGCAGTCTTCAGAGGGCCACCGCAGCTCCAGTG
CCACGGCAGGAGGCTGTTCCTGAATAGCCCCTGTGGTAAGGGCCAGGAGAGTCC
TTCCATCCTCCAAGGCCCTGCTAAAGGACACAGCAGCCAGGAAGTCCCCTGGGC
CCCTAGCTGAAGGACAGCCTGCTCCCTCCGTCTCTACCAGGAATGGCCTTGTCCT
ATGGAAGGCACTGCCCCATCCCAAACTAATCTAGGAATCACTGTCTAACCACTCA
CTGTCATGAATGTGTACTTAAAGGATGAGGTTGAGTCATACCAAATAGTGATTTC
GATAGTTCAAAATGGTGAAATTAGCAATTCTACATGATTCAGTCTAATCAATGGA
TACCGACTGTTTCCCACACAAGTCTCCTGTTCTCTTAAGCTTACTCACTGACAGCC
TTTCACTCTCCACAAATACATTAAAGATATGGCCATCACCAAGCCCCCTAGGATG
ACACCAGACCTGAGAGTCTGAAGACCTGGATCCAAGTTCTGACTTTTCCCCCTGA
CAGCTGTGTGACCTTCGTGAAGTCGCCAAACCTCTCTGAGCCCCAGTCATTGCTA
GTAAGACCTGCCTTTGAGTTGGTATGATGTTCAAGTTAGATAACAAAATGTTTAT
ACCCATTAGAACAGAGAATAAATAGAACTACATTTCTTGCA
Alpha 1-antitrypsin polypeptide encoded by P00450
(SEQ ID NO: 702):
EDPQGDAAQKTDTSHHDQDHPTFNKITPNLAEFAFSLYRQLAHQSNSTNIFFSPVSIA
TAFAMLSLGTKADTHDEILEGLNFNLTEIPEAQIHEGFQELLRTLNQPDSQLQLTTGN
GLFLSEGLKLVDKFLEDVKKLYHSEAFTVNFGDTEEAKKQINDYVEKGTQGKIVDLV
KELDRDTVFALVNYIFFKGKWERPFEVKDTEEEDFHVDQVTTVKVPMMKRLGMFNI
QHCKKLSSWVLLMKYLGNATAIFFLPDEGKLQHLENELTHDIITKFLENEDRRSASL
HLPKLSITGTYDLKSVLGQLGITKVFSNGADLSGVTEEAPLKLSKAVHKAVLTIDEKG
TEAAGAMFLEAIPMSIPPEVKFNKPFVFLMIEQNTKSPLFMGKVVNPTQK
Human AAT Nucleotide Sequence
NCBI Ref: NM_001127700.2):
(SEQ ID NO: 703)
AGAGTCCTGAGCTGAACCAAGAAGGAGGAGGGGGTCGGGCCTCCGAGGAAGGC
CTAGCCGCTGCTGCTGCCAGGAATTCCAGGTTGGAGGGGCGGCAACCTCCTGCC
AGCCTTCAGGCCACTCTCCTGTGCCTGCCAGAAGAGACAGAGCTTGAGGAGAGC
TTGAGGAGAGCAGGAAAGGTGGGACATTGCTGCTGCTGCTCACTCAGTTCCACA
GGACAATGCCGTCTTCTGTCTCGTGGGGCATCCTCCTGCTGGCAGGCCTGTGCTG
CCTGGTCCCTGTCTCCCTGGCTGAGGATCCCCAGGGAGATGCTGCCCAGAAGACA
GATACATCCCACCATGATCAGGATCACCCAACCTTCAACAAGATCACCCCCAACC
TGGCTGAGTTCGCCTTCAGCCTATACCGCCAGCTGGCACACCAGTCCAACAGCAC
CAATATCTTCTTCTCCCCAGTGAGCATCGCTACAGCCTTTGCAATGCTCTCCCTGG
GGACCAAGGCTGACACTCACGATGAAATCCTGGAGGGCCTGAATTTCAACCTCA
CGGAGATTCCGGAGGCTCAGATCCATGAAGGCTTCCAGGAACTCCTCCGTACCCT
CAACCAGCCAGACAGCCAGCTCCAGCTGACCACCGGCAATGGCCTGTTCCTCAG
CGAGGGCCTGAAGCTAGTGGATAAGTTTTTGGAGGATGTTAAAAAGTTGTACCA
CTCAGAAGCCTTCACTGTCAACTTCGGGGACACCGAAGAGGCCAAGAAACAGAT
CAACGATTACGTGGAGAAGGGTACTCAAGGGAAAATTGTGGATTTGGTCAAGGA
GCTTGACAGAGACACAGTTTTTGCTCTGGTGAATTACATCTTCTTTAAAGGCAAA
TGGGAGAGACCCTTTGAAGTCAAGGACACCGAGGAAGAGGACTTCCACGTGGAC
CAGGTGACCACCGTGAAGGTGCCTATGATGAAGCGTTTAGGCATGTTTAACATCC
AGCACTGTAAGAAGCTGTCCAGCTGGGTGCTGCTGATGAAATACCTGGGCAATG
CCACCGCCATCTTCTTCCTGCCTGATGAGGGGAAACTACAGCACCTGGAAAATGA
ACTCACCCACGATATCATCACCAAGTTCCTGGAAAATGAAGACAGAAGGTCTGC
CAGCTTACATTTACCCAAACTGTCCATTACTGGAACCTATGATCTGAAGAGCGTC
CTGGGTCAACTGGGCATCACTAAGGTCTTCAGCAATGGGGCTGACCTCTCCGGGG
TCACAGAGGAGGCACCCCTGAAGCTCTCCAAGGCCGTGCATAAGGCTGTGCTGA
CCATCGACGAGAAAGGGACTGAAGCTGCTGGGGCCATGTTTTTAGAGGCCATAC
CCATGTCTATCCCCCCCGAGGTCAAGTTCAACAAACCCTTTGTCTTCTTAATGATT
GAACAAAATACCAAGTCTCCCCTCTTCATGGGAAAAGTGGTGAATCCCACCCAA
AAATAACTGCCTCTCGCTCCTCAACCCCTCCCCTCCATCCCTGGCCCCCTCCCTGG
ATGACATTAAAGAAGGGTTGAGCTGGTCCCTGCCTGCATGTGACTGTAAATCCCT
CCCATGTTTTCTCTGAGTCTCCCTTTGCCTGCTGAGGCTGTATGTGGGCTCCAGGT
AACAGTGCTGTCTTCGGGCCCCCTGAACTGTGTTCATGGAGCATCTGGCTGGGTA
GGCACATGCTGGGCTTGAATCCAGGGGGGACTGAATCCTCAGCTTACGGACCTG
GGCCCATCTGTTTCTGGAGGGCTCCAGTCTTCCTTGTCCTGTCTTGGAGTCCCCAA
GAAGGAATCACAGGGGAGGAACCAGATACCAGCCATGACCCCAGGCTCCACCA
AGCATCTTCATGTCCCCCTGCTCATCCCCCACTCCCCCCCACCCAGAGTTGCTCAT
CCTGCCAGGGCTGGCTGTGCCCACCCCAAGGCTGCCCTCCTGGGGGCCCCAGAA
CTGCCTGATCGTGCCGTGGCCCAGTTTTGTGGCATCTGCAGCAACACAAGAGAGA
GGACAATGTCCTCCTCTTGACCCGCTGTCACCTAACCAGACTCGGGCCCTGCACC
TCTCAGGCACTTCTGGAAAATGACTGAGGCAGATTCTTCCTGAAGCCCATTCTCC
ATGGGGCAACAAGGACACCTATTCTGTCCTTGTCCTTCCATCGCTGCCCCAGAAA
GCCTCACATATCTCCGTTTAGAATCAGGTCCCTTCTCCCCAGATGAAGAGGAGGG
TCTCTGCTTTGTTTTCTCTATCTCCTCCTCAGACTTGACCAGGCCCAGCAGGCCCC
AGAAGACCATTACCCTATATCCCTTCTCCTCCCTAGTCACATGGCCATAGGCCTG
CTGATGGCTCAGGAAGGCCATTGCAAGGACTCCTCAGCTATGGGAGAGGAAGCA
CATCACCCATTGACCCCCGCAACCCCTCCCTTTCCTCCTCTGAGTCCCGACTGGG
GCCACATGCAGCCTGACTTCTTTGTGCCTGTTGCTGTCCCTGCAGTCTTCAGAGG
GCCACCGCAGCTCCAGTGCCACGGCAGGAGGCTGTTCCTGAATAGCCCCTGTGGT
AAGGGCCAGGAGAGTCCTTCCATCCTCCAAGGCCCTGCTAAAGGACACAGCAGC
CAGGAAGTCCCCTGGGCCCCTAGCTGAAGGACAGCCTGCTCCCTCCGTCTCTACC
AGGAATGGCCTTGTCCTATGGAAGGCACTGCCCCATCCCAAACTAATCTAGGAAT
CACTGTCTAACCACTCACTGTCATGAATGTGTACTTAAAGGATGAGGTTGAGTCA
TACCAAATAGTGATTTCGATAGTTCAAAATGGTGAAATTAGCAATTCTACATGAT
TCAGTCTAATCAATGGATACCGACTGTTTCCCACACAAGTCTCCTGTTCTCTTAAG
CTTACTCACTGACAGCCTTTCACTCTCCACAAATACATTAAAGATATGGCCATCA
CCAAGCCCCCTAGGATGACACCAGACCTGAGAGTCTGAAGACCTGGATCCAAGT
TCTGACTTTTCCCCCTGACAGCTGTGTGACCTTCGTGAAGTCGCCAAACCTCTCTG
AGCCCCAGTCATTGCTAGTAAGACCTGCCTTTGAGTTGGTATGATGTTCAAGTTA
GATAACAAAATGTTTATACCCATTAGAACAGAGAATAAATAGAACTACATTTCTT
GCA
Human AAT Protein Signal Sequence
(SEQ ID NO: 705)
MPSSVSWGILLLAGLCCLVPVSLA

TABLE 9A

| Construct | Description | Annotation | Sequence |
|---|---|---|---|
| 1 | Full | SEQ ID NO: | aagtggtggcctaactacggctacactagaagaacagtatttggtatctgcgctctgctgaagccagttaccttcggaaaaagagttggtagctcttgatc |
| | Sequence | 710 | cggcaaacaaaccaccgctggtagcggtggtttttttgtttgcaagcagcagattacgcgcagaaaaaaaggatctcaagaagatcctttgatcttttcta |
| | | | cggggtctgacgctcagtggaacgaaaactcacgttaagggattttggtcatgagattatcaaaaaggatcttcacctagatccttttaaattaaaaatg |
| | | | aagttttaaatcaagcccaatctgaataatgttacaaccaattaaccaattctgattagaaaaactcatcgagcatcaaatgaaactgcaatttattcata |
| | | | tcaggattatcaataccatatttttgaaaaagccgtttctgtaatgaaggagaaaactcaccgaggcagttccataggatggcaagatcctggtatcggt |
| | | | ctgcgattccgactcgtccaacatcaatacaacctattaatttcccctcgtcaaaaataaggttatcaagtgagaaatcaccatgagtgacgactgaatcc |
| | | | ggtgagaatggcaaaagtttatgcatttctttccagacttgttcaacaggccagccattacgctcgtcatcaaaatcactcgcatcaaccaaaccgttattc |
| | | | attcgtgattgcgcctgagcgagacgaaatacgcgatcgctgttaaaaggacaattacaaacaggaatcgaatgcaaccggcgcaggaacactgccag |
| | | | cgcatcaacaatattttcacctgaatcaggatattcttctaatacctggaatgctgtttttccggggatcgcagtggtgagtaaccatgcatcatcaggagt |
| | | | acggataaaatgcttgatggtcggaagaggcataaattccgtcagccagtttagtctgaccatctcatctgtaacatcattggcaacgctacctttgccat |
| | | | gtttcagaaacaactctggcgcatcgggcttcccatacaagcgatagattgtcgcacctgattgcccgacattatcgcgagcccatttatacccatataaa |
| | | | tcagcatccatgttggaatttaatcgcggcctcgacgtttcccgttgaatatggctcataacaccccttgtattactgtttatgtaagcagacagttttattgt |
| | | | tcatgatgatatatttttatcttgtgcaatgtaacatcagagattttgagacacgggccagagctgcatcgcgcgtttcggtgatgacggtgaaaacctctg |
| | | | acacatgcagctcccggagacggtcacagcttgtctgtaagcggatgccgggagcagacaagcccgtcagggcgcgtcagcgggtgttggcgggtgtc |
| | | | ggggctggcttaactatgcggcatcagagcagattgtactgagagtgcaccatatgcggtgtgaaataccgcacagatgcgtaaggagaaaataccgc |
| | | | atcaggcgccattcgccattcaggctgcgcaactgttgggaagggcgatcggtgcgggcctcttcgctattacgccagctggcgaaagggggatgtgctg |
| | | | caaggcgattaagttgggtaacgccagggttttcccagtcacgacgttgtaaaacgacggccagagaattcTTGGCCACTCCCTCTCTGCGCG |
| | | | CTCGCTCGCTCACTGAGGCCGGGCGACCAAAGGTCGCCCGACGCCCGGGCTTTGCCCGGGCGGCCTCAGTGAGCGA |
| | | | GCGAGCGCGCAGAGAGGGAGTGGCCAACTCCATCACTAGGGGTTCCTAGATCTACTAGTtaggtcagtgaagagaagaac |
| | | | aaaaagcagcatattacagttagttgtcttcatcaatctttaaatatgttgtgtggtttttctctccctgtttccacagttGAGGACCCCCAGGGCGA |
| | | | CGCCGCCCAGAAGACCGACACCAGCCACCACGACCAGGACCACCCCACCTTCAACAAGATCACCCCCAACCTGGCCG |
| | | | AGTTCGCCTTCAGCCTGTACAGGCAGCTGGCCCACCAGAGCAACAGCACCAACATCTTCTTCAGCCCCGTGAGCATC |
| | | | GCCACCGCCTTCGCCATGCTGAGCCTGGGCACCAAGGCCGACACCCACGACGAGATCCTGGAGGGCCTGAACTTCA |
| | | | ACCTGACCGAGATCCCCGAGGCCCAGATCCACGAGGGCTTCCAGGAGCTGCTGAGGACCCTGAACCAGCCCGACAG |
| | | | CCAGCTGCAGCTGACCACCGGCAACGGCCTGTTCCTGAGCGAGGGCCTGAAGCTGGTGGACAAGTTCCTGGAGGAC |
| | | | GTGAAGAAGCTGTACCACAGCGAGGCCTTCACCGTGAACTTCGGCGACACCGAGGAGGCCAAGAAGCAGATCAAC |
| | | | GACTACGTGGAGAAGGGCACCCAGGGCAAGATCGTGGACCTGGTGAAGGAGCTGGACAGGGACACCGTGTTCGCC |
| | | | CTGGTGAACTACATCTTCTTCAAGGGCAAGTGGGAGAGGCCCTTCGAGGTGAAGGACACCGAGGAGGAGGACTTC |
| | | | CACGTGGACCAGGTGACCACCGTGAAGGTGCCCATGATGAAGAGGCTGGGCATGTTCAACATCCAGCACTGCAAGA |
| | | | AGCTGAGCAGCTGGGTGCTGCTGATGAAGTACCTGGGCAACGCCACCGCCATCTTCTTCCTGCCCGACGAGGGCAA |
| | | | GCTGCAGCACCTGGAGAACGAGCTGACCCACGACATCATCACCAAGTTCCTGGAGAACGAGGACAGGAGGAGCGC |
| | | | CAGCCTGCACCTGCCCAAGCTGAGCATCACCGGCACCTACGACCTGAAGAGCGTGCTGGGCCAGCTGGGCATCACC |
| | | | AAGGTGTTCAGCAACGGCGCCGACCTGAGCGGCGTGACCGAGGAGGCCCCCCTGAAGCTGAGCAAGGCCGTGCAC |
| | | | AAGGCCGTGCTGACCATCGACGAGAAGGGCACCGAGGCCGCCGGCGCCATGTTCCTGGAGGCCATCCCCATGAGC |
| | | | ATCCCCCCCGAGGTGAAGTTCAACAAGCCCTTCGTGTTCCTGATGATCGAGCAGAACACCAAGAGCCCCCTGTTCAT |
| | | | GGGCAAGGTGGTGAACCCCACCCAGAAGTAACAGACATGATAAGATACATTGATGAGTTTGGACAAACCACAACTA |
| | | | GAATGCAGTGAAAAAAATGCTTTATTTGTGAAATTTGTGATGCTATTGCTTTATTTGTAACCATTATAAGCTGCAATA |
| | | | AACAAGTTAACAACAACAATTGCATTCATTTTATGTTTCAGGTTCAGGGGGAGGTGTGGGAGGTTTTTTggggatacccc |
| | | | ctagagccccagctggttctttccgcctcagaagCCATAGAGCCCACCGCATCCCCAGCATGCCTGCTATTGTCTTCCCAATCCTC |
| | | | CCCCTTGCTGTCCTGCCCCACCCCACCCCCCAGAATAGAATGACACCTACTCAGACAATGCGATGCAATTTCCTCATTT |
| | | | TATTAGGAAAGGACAGTGGGAGTGGCACCTTCCAGGGTCAAGGAAGGCACGGGGGAGGGGCAAACAACAGATGG |
| | | | CTGGCAACTAGAAGGCACAGTCGaggttaTTTTTGGGTGGGATTCACCACTTTTCCCATGAAGAGGGGAGACTTGGTA |
| | | | TTTTGTTCAATCATTAAGAAGACAAAGGGTTTGTTGAACTTGACCTCGGGGGGGATAGACATGGGTATGGCCTCTAA |
| | | | AAACATGGCCCCAGCAGCTTCAGTCCCTTTCTCGTCGATGGTCAGCACAGCCTTATGCACGGCCTTGGAGAGCTTCA |
| | | | GGGGTGCCTCCTCTGTGACCCCGGAGAGGTCAGCCCCATTGCTGAAGACCTTAGTGATGCCCAGTTGACCCAGGAC |
| | | | GCTCTTCAGATCATAGGTTCCAGTAATGGACAGTTTGGGTAAATGTAAGCTGGCAGACCTTCTGTCTTCATTTTCCAG |
| | | | GAACTTGGTGATGATATCGTGGGTGAGTTCATTTTCCAGGTGCTGTAGTTTCCCCTCATCAGGCAGGAAGAAGATGG |
| | | | CGGTGGCATTGCCCAGGTATTTCATCAGCAGCACCCAGCTGGACAGCTTCTTACAGTGCTGGATGTTAAACATGCCT |
| | | | AAACGCTTCATCATAGGCACCTTCACGGTGGTCACCTGGTCCACGTGGAAGTCCTCTTCCTCGGTGTCCTTGACTTCA |
| | | | AAGGGTCTCTCCCATTTGCCTTTAAAGAAGATGTAATTCACCAGAGCAAAAACTGTGTCTCTGTCAAGCTCCTTGACC |
| | | | AAATCCACAATTTTCCCTTGAGTACCCTTCTCCACGTAATCGTTGATCTGTTTCTTGGCCTCTTCGGTGTCCCCGAAGT |
| | | | TGACAGTGAAGGCTTCTGAGTGGTACAACTTTTTAACATCCTCCAAAAACTTATCCACTAGCTTCAGGCCCTCGCTGA |
| | | | GGAACAGGCCATTGCCGGTGGTCAGCTGGAGCTGGCTGTCTGGCTGGTTGAGGGTACGGAGGAGTTCCTGGAAGC |
| | | | CTTCATGGATCTGAGCCTCCGGAATCTCCGTGAGGTTGAAATTCAGGCCCTCCAGGATTTCATCGTGAGTGTCAGCC |
| | | | TTGGTCCCCAGGGAGAGCATTGCAAAGGCTGTAGCGATGCTCACTGGGGAGAAGAAGATATTGGTGCTGTTGGACT |
| | | | GGTGTGCCAGCTGGCGGTATAGGCTGAAGGCGAACTCAGCCAGGTTGGGGGTGATCTTGTTGAAGGTTGGGTGAT |
| | | | CCTGATCATGGTGGGATGTATCTGTCTTCTGGGCAGCATCTCCCTGGGGATCCTCaactgtggaaacagggagagaaaaacc |
| | | | acacaacatatttaaagattgatgaagacaactaactgtaatatgctgctttttgttcttctcttcactgacctaACTAGTAGATCTAGGAACCCC |
| | | | TAGTGATGGAGTTGGCCACTCCCTCTCTGCGCGCTCGCTCGCTCACTGAGGCCGCCCGGGCAAAGCCCGGGCGTCG |
| | | | GGCGACCTTTGGTCGCCCGGCCTCAGTGAGCGAGCGAGCGCGCAGAGAGGGAGTGGCCAAacgcgtggtgtaatcatggt |
| | | | catagctgtttcctgtgtgaaattgttatccgctcacaattccacacaacatacgagccggaagcataaagtgtaaagcctggggtgcctaatgagtgag |
| | | | ctaactcacattaattgcgttgcgctcactgcccgctttccagtcgggaaacctgtcgtgccagctgcattaatgaatcggccaacgcgcggggagaggc |
| | | | ggtttgcgtattgggcgctcttccgcttcctcgctcactgactcgctgcgctcggtcgttcggctgcggcgagcggtatcagctcactcaaaggcggtaata |
| | | | cggttatccacagaatcaggggataacgcaggaaagaacatgtgagcaaaaggccagcaaaaggccaggaaccgtaaaaaggccgcgttgctggcg |
| | | | tttttccataggctccgcccccctgacgagcatcacaaaaatcgacgctcaagtcagaggtggcgaaacccgacaggactataaagataccaggcgttt |
| | | | ccccctggaagctccctcgtgcgctctcctgttccgaccctgccgcttaccggatacctgtccgcctttctcccttcgggaagcgtggcgctttctcatagctc |
| | | | acgctgtaggtatctcagttcggtgtaggtcgttcgctccaagctgggctgtgtgcacgaaccccccgttcagcccgaccgctgcgccttatccggtaacta |
| | | | tcgtcttgagtccaacccggtaagacacgacttatcgccactggcagcagccactggtaacaggattagcagagcgaggtatgtaggcggtgctacaga |
| | | | gttcttg |
| | SERPINA1 | A1AT w/o | GAGGACCCCCAGGGCGACGCCGCCCAGAAGACCGACACCAGCCACCACGACCAGGACCACCCCACCTTCAACAAGA |
| | copy 1 | SP | TCACCCCCAACCTGGCCGAGTTCGCCTTCAGCCTGTACAGGCAGCTGGCCCACCAGAGCAACAGCACCAACATCTTC |
| | | (alternate | TTCAGCCCCGTGAGCATCGCCACCGCCTTCGCCATGCTGAGCCTGGGCACCAAGGCCGACACCCACGACGAGATCCT |
| | | codon usage | GGAGGGCCTGAACTTCAACCTGACCGAGATCCCCGAGGCCCAGATCCACGAGGGCTTCCAGGAGCTGCTGAGGACC |
| | | 1) | CTGAACCAGCCCGACAGCCAGCTGCAGCTGACCACCGGCAACGGCCTGTTCCTGAGCGAGGGCCTGAAGCTGGTGG |
| | | (SEQ ID NO: | ACAAGTTCCTGGAGGACGTGAAGAAGCTGTACCACAGCGAGGCCTTCACCGTGAACTTCGGCGACACCGAGGAGG |
| | | 711) | CCAAGAAGCAGATCAACGACTACGTGGAGAAGGGCACCCAGGGCAAGATCGTGGACCTGGTGAAGGAGCTGGAC |
| | | | AGGGACACCGTGTTCGCCCTGGTGAACTACATCTTCTTCAAGGGCAAGTGGGAGAGGCCCTTCGAGGTGAAGGACA |
| | | | CCGAGGAGGAGGACTTCCACGTGGACCAGGTGACCACCGTGAAGGTGCCCATGATGAAGAGGCTGGGCATGTTCA |
| | | | ACATCCAGCACTGCAAGAAGCTGAGCAGCTGGGTGCTGCTGATGAAGTACCTGGGCAACGCCACCGCCATCTTCTTC |
| | | | CTGCCCGACGAGGGCAAGCTGCAGCACCTGGAGAACGAGCTGACCCACGACATCATCACCAAGTTCCTGGAGAACG |
| | | | AGGACAGGAGGAGCGCCAGCCTGCACCTGCCCAAGCTGAGCATCACCGGCACCTACGACCTGAAGAGCGTGCTGG |
| | | | GCCAGCTGGGCATCACCAAGGTGTTCAGCAACGGCGCCGACCTGAGCGGCGTGACCGAGGAGGCCCCCCTGAAGC |
| | | | TGAGCAAGGCCGTGCACAAGGCCGTGCTGACCATCGACGAGAAGGGCACCGAGGCCGCCGGCGCCATGTTCCTGG |
| | | | AGGCCATCCCCATGAGCATCCCCCCCGAGGTGAAGTTCAACAAGCCCTTCGTGTTCCTGATGATCGAGCAGAACACC |
| | | | AAGAGCCCCCTGTTCATGGGCAAGGTGGTGAACCCCACCCAGAAGTAA |
| | SERPINA1 | A1AT w/o | GAGGATCCCCAGGGAGATGCTGCCCAGAAGACAGATACATCCCACCATGATCAGGATCACCCAACCTTCAACAAGA |
| | copy 2 (rev | SP | TCACCCCCAACCTGGCTGAGTTCGCCTTCAGCCTATACCGCCAGCTGGCACACCAGTCCAACAGCACCAATATCTTCT |
| | comp) | (SEQ ID NO: | TCTCCCCAGTGAGCATCGCTACAGCCTTTGCAATGCTCTCCCTGGGGACCAAGGCTGACACTCACGATGAAATCCTG |
| | | 712) | GAGGGCCTGAATTTCAACCTCACGGAGATTCCGGAGGCTCAGATCCATGAAGGCTTCCAGGAACTCCTCCGTACCCT |
| | | | CAACCAGCCAGACAGCCAGCTCCAGCTGACCACCGGCAATGGCCTGTTCCTCAGCGAGGGCCTGAAGCTAGTGGAT |
| | | | AAGTTTTTGGAGGATGTTAAAAAGTTGTACCACTCAGAAGCCTTCACTGTCAACTTCGGGGACACCGAAGAGGCCAA |
| | | | GAAACAGATCAACGATTACGTGGAGAAGGGTACTCAAGGGAAAATTGTGGATTTGGTCAAGGAGCTTGACAGAGA |
| | | | CACAGTTTTTGCTCTGGTGAATTACATCTTCTTTAAAGGCAAATGGGAGAGACCCTTTGAAGTCAAGGACACCGAGG |
| | | | AAGAGGACTTCCACGTGGACCAGGTGACCACCGTGAAGGTGCCTATGATGAAGCGTTTAGGCATGTTTAACATCCA |
| | | | GCACTGTAAGAAGCTGTCCAGCTGGGTGCTGCTGATGAAATACCTGGGCAATGCCACCGCCATCTTCTTCCTGCCTG |
| | | | ATGAGGGGAAACTACAGCACCTGGAAAATGAACTCACCCACGATATCATCACCAAGTTCCTGGAAAATGAAGACAG |
| | | | AAGGTCTGCCAGCTTACATTTACCCAAACTGTCCATTACTGGAACCTATGATCTGAAGAGCGTCCTGGGTCAACTGG |
| | | | GCATCACTAAGGTCTTCAGCAATGGGGCTGACCTCTCCGGGGTCACAGAGGAGGCACCCCTGAAGCTCTCCAAGGC |
| | | | CGTGCATAAGGCTGTGCTGACCATCGACGAGAAAGGGACTGAAGCTGCTGGGGCCATGTTTTTAGAGGCCATACCC |
| | | | ATGTCTATCCCCCCCGAGGTCAAGTTCAACAAACCCTTTGTCTTCTTAATGATTGAACAAAATACCAAGTCTCCCCTCT |
| | | | TCATGGGAAAAGTGGTGAATCCCACCCAAAAAtaa |
| 7 | Full | SEQ ID NO: | TTGGCCACTCCCTCTCTGCGCGCTCGCTCGCTCACTGAGGCCGGGCGACCAAAGGTCGCCCGACGCCCGGGCTTTGC |
| | Sequence | 770 | CCGGGCGGCCTCAGTGAGCGAGCGAGCGCGCAGAGAGGGAGTGGCCAACTCCATCACTAGGGGTTCCTAGATCTA |
| | | | CTAGTtaggtcagtgaagagaagaacaaaaagcagcatattacagttagttgtcttcatcaatctttaaatatgttgtgtggtttttctctccctgtttcca |
| | | | cagttGAGGACCCCCAGGGAGATGCAGCCCAGAAGACAGACACCAGCCACCATGACCAGGACCACCCCACCTTCAAC |
| | | | AAGATCACCCCCAACCTGGCAGAGTTTGCCTTCAGCCTGTACAGGCAGCTGGCCCACCAGAGCAACAGCACCAACAT |
| | | | CTTCTTCAGCCCAGTGAGCATAGCCACAGCCTTTGCCATGCTGAGCCTGGGCACCAAGGCAGACACCCATGATGAGA |
| | | | TCCTGGAGGGCCTGAACTTCAACCTGACAGAGATCCCAGAGGCCCAGATCCATGAGGGCTTCCAGGAGCTGCTGAG |
| | | | GACCCTGAACCAGCCAGACAGCCAGCTGCAGCTGACCACAGGCAATGGCCTGTTCCTGTCTGAGGGCCTGAAGCTG |
| | | | GTGGACAAGTTCCTGGAGGATGTGAAGAAGCTGTACCACTCTGAGGCCTTCACAGTGAACTTTGGAGACACAGAGG |
| | | | AGGCCAAGAAGCAGATCAATGACTATGTGGAGAAGGGCACCCAGGGCAAGATAGTGGACCTGGTGAAGGAGCTG |
| | | | GACAGGGACACAGTGTTTGCCCTGGTGAACTACATCTTCTTCAAGGGCAAGTGGGAGAGGCCCTTTGAGGTGAAGG |
| | | | ACACAGAGGAGGAGGACTTCCATGTGGACCAGGTGACCACAGTGAAGGTGCCCATGATGAAGAGGCTGGGCATGT |
| | | | TCAATATCCAGCACTGCAAGAAGCTGAGCAGCTGGGTGCTGCTGATGAAGTACCTGGGCAATGCCACAGCCATCTTC |
| | | | TTCCTGCCAGATGAGGGCAAGCTGCAGCACCTGGAGAATGAGCTGACCCATGACATCATCACCAAGTTCCTGGAGA |
| | | | ATGAGGACAGGAGGTCTGCCAGCCTGCACCTGCCCAAGCTGAGCATCACAGGCACCTATGACCTGAAGTCTGTGCT |
| | | | GGGCCAGCTGGGCATCACCAAGGTGTTCAGCAATGGAGCAGACCTGTCTGGAGTGACAGAGGAGGCCCCCCTGAA |
| | | | GCTGAGCAAGGCAGTGCACAAGGCAGTGCTGACCATAGATGAGAAGGGCACAGAGGCAGCAGGAGCCATGTTCCT |
| | | | GGAGGCCATCCCCATGAGCATCCCCCCAGAGGTGAAGTTCAACAAGCCTTTTGTGTTCCTGATGATAGAGCAGAACA |
| | | | CCAAGAGCCCCCTGTTCATGGGCAAGGTGGTGAACCCCACCCAGAAGTAACAGACATGATAAGATACATTGATGAG |
| | | | TTTGGACAAACCACAACTAGAATGCAGTGAAAAAAATGCTTTATTTGTGAAATTTGTGATGCTATTGCTTTATTTGTA |
| | | | ACCATTATAAGCTGCAATAAACAAGTTAACAACAACAATTGCATTCATTTTATGTTTCAGGTTCAGGGGGAGGTGTG |
| | | | GGAGGTTTTTTggggataccccctagagccccagctggttcttttctcctcagaagCCATAGAGCCCATCTCATCCCCAGCATGCCTGC |
| | | | TATTGTCTTCCCAATCCTCCCCCTTGCTGTCCTGCCCCACCCCACCCCCCAGAATAGAATGACACCTACTCAGACAATT |
| | | | CTATGCAATTTCCTCATTTTATTAGGAAAGGACAGTGGGAGTGGCACCTTCCAGGGTCAAGGAAGGCATGGGGGAG |
| | | | GGGCAAACAACAGATGGCTGGCAACTAGAAGGCACAGTCTaggttaTTTTTGGGTGGGATTCACCACTTTTCCCATGA |
| | | | AGAGGGGTGATTTAGTGTTCTGCTCTATCATGAGAAATACAAAAGGTTTGTTGAACTTGACCTCTGGGGGGATAGA |
| | | | CATGGGTATGGCCTCTAAAAACATGGCCCCAGCAGCCTCTGTGCCCTTCTCATCTATGGTCAGCACAGCCTTATGCAC |
| | | | TGCCTTGGAGAGCTTCAGGGGTGCCTCCTCTGTGACCCCAGAGAGGTCAGCCCCATTGCTGAAGACCTTAGTGATGC |
| | | | CCAGTTGACCCAGGACAGACTTCAGATCATAGGTTCCAGTAATGGACAGTTTGGGTAAATGTAAGCTGGCAGACCTT |
| | | | CTGTCTTCATTTTCCAGGAACTTGGTGATGATATCATGGGTGAGTTCATTTTCCAGGTGCTGTAGTTTCCCCTCATCAG |
| | | | GCAGGAAGAAGATGGCTGTGGCATTGCCCAGGTATTTCATCAGCAGCACCCAGCTGGACAGCTTCTTACAGTGCTG |
| | | | GATATTGAACATACCAAGCCTTTTCATCATAGGCACCTTCACTGTGGTCACCTGGTCCACATGGAAGTCCTCTTCCTCT |
| | | | GTGTCCTTGACTTCAAAGGGTCTCTCCCATTTGCCTTTAAAGAAGATGTAATTCACCAGAGCAAAAACTGTGTCTCTG |
| | | | TCAAGCTCCTTGACCAAATCCACAATTTTCCCTTGAGTACCCTTCTCCACATAATCATTGATCTGTTTCTTGGCCTCTTC |
| | | | TGTGTCCCCAAAGTTGACAGTGAAGGCTTCTGAGTGGTACAACTTTTTAACATCCTCCAAAAACTTATCCACTAGCTT |
| | | | CAGGCCCTCAGAGAGGAACAGGCCATTGCCTGTGGTCAGCTGGAGCTGGCTGTCTGGCTGGTTGAGGGTTCTGAG |
| | | | GAGTTCCTGGAAGCCTTCATGGATCTGAGCCTCTGGAATCTCTGTGAGGTTGAAATTCAGGCCCTCCAGGATTTCAT |
| | | | CATGAGTGTCAGCCTTGGTCCCCAGGGAGAGCATTGCAAAGGCTGTAGCTATGCTCACTGGGGAGAAGAAGATATT |
| | | | GGTGCTGTTGGACTGGTGTGCCAGCTGTCTGTATAGGCTGAAGGCAAACTCAGCCAGGTTGGGGGTGATCTTGTTG |
| | | | AAGGTTGGGTGATCCTGATCATGGTGGGATGTATCTGTCTTCTGGGCAGCATCTCCCTGGGGATCCTCaactgtggaaa |
| | | | cagggagagaaaaaccacacaacatatttaaagattgatgaagacaactaactgtaatatgctgctttttgttcttctcttcactgacctaAGAGATCT |
| | | | AGGAACCCCTAGTGATGGAGTTGGCCACTCCCTCTCTGCGCGCTCGCTCGCTCACTGAGGCCGCCCGGGCAAAGCCC |
| | | | GGGCGTCGGGCGACCTTTGGTCGCCCGGCCTCAGTGAGCGAGCGAGCGCGCAGAGAGGGAGTGGCCAAacgcgtggt |
| | | | gtaatcatggtcatagctgtttcctgtgtgaaattgttatccgctcacaattccacacaacatacgagccggaagcataaagtgtaaagcctggggtgcct |
| | | | aatgagtgagctaactcacattaattgcgttgcgctcactgcccgctttccagtcgggaaacctgtcgtgccagctgcattaatgaatcggccaacgcgcg |
| | | | gggagaggcggtttgcgtattgggcgctcttccgcttcctcgctcactgactcgctgcgctcggtcgttcggctgcggcgagcggtatcagctcactcaaa |
| | | | ggcggtaatacggttatccacagaatcaggggataacgcaggaaagaacatgtgagcaaaaggccagcaaaaggccaggaaccgtaaaaaggccgc |
| | | | gttgctggcgtttttccataggctccgcccccctgacgagcatcacaaaaatcgacgctcaagtcagaggtggcgaaacccgacaggactataaagata |
| | | | ccaggcgtttccccctggaagctccctcgtgcgctctcctgttccgaccctgccgcttaccggatacctgtccgcctttctcccttcgggaagcgtggcgctt |
| | | | tctcatagctcacgctgtaggtatctcagttcggtgtaggtcgttcgctccaagctgggctgtgtgcacgaaccccccgttcagcccgaccgctgcgcctta |
| | | | tccggtaactatcgtcttgagtccaacccggtaagacacgacttatcgccactggcagcagccactggtaacaggattagcagagcgaggtatgtaggc |
| | | | ggtgctacagagttcttgaagtggtggcctaactacggctacactagaagaacagtatttggtatctgcgctctgctgaagccagttaccttcggaaaaa |
| | | | gagttggtagctcttgatccggcaaacaaaccaccgctggtagcggtggtttttttgtttgcaagcagcagattacgcgcagaaaaaaaggatctcaaga |
| | | | agatcctttgatcttttctacggggtctgacgctcagtggaacgaaaactcacgttaagggattttggtcatgagattatcaaaaaggatcttcacctagat |
| | | | ccttttaaattaaaaatgaagttttaaatcaagcccaatctgaataatgttacaaccaattaaccaattctgattagaaaaactcatcgagcatcaaatga |
| | | | aactgcaatttattcatatcaggattatcaataccatatttttgaaaaagccgtttctgtaatgaaggagaaaactcaccgaggcagttccataggatggc |
| | | | aagatcctggtatcggtctgcgattccgactcgtccaacatcaatacaacctattaatttcccctcgtcaaaaataaggttatcaagtgagaaatcaccat |
| | | | gagtgacgactgaatccggtgagaatggcaaaagtttatgcatttctttccagacttgttcaacaggccagccattacgctcgtcatcaaaatcactcgca |
| | | | tcaaccaaaccgttattcattcgtgattgcgcctgagcgagacgaaatacgcgatcgctgttaaaaggacaattacaaacaggaatcgaatgcaaccgg |
| | | | cgcaggaacactgccagcgcatcaacaatattttcacctgaatcaggatattcttctaatacctggaatgctgtttttccggggatcgcagtggtgagtaa |
| | | | ccatgcatcatcaggagtacggataaaatgcttgatggtcggaagaggcataaattccgtcagccagtttagtctgaccatctcatctgtaacatcattgg |
| | | | caacgctacctttgccatgtttcagaaacaactctggcgcatcgggcttcccatacaagcgatagattgtcgcacctgattgcccgacattatcgcgagcc |
| | | | catttatacccatataaatcagcatccatgttggaatttaatcgcggcctcgacgtttcccgttgaatatggctcataacaccccttgtattactgtttatgta |
| | | | agcagacagttttattgttcatgatgatatatttttatcttgtgcaatgtaacatcagagattttgagacacgggccagagctgcatcgcgcgtttcggtgat |
| | | | gacggtgaaaacctctgacacatgcagctcccggagacggtcacagcttgtctgtaagcggatgccgggagcagacaagcccgtcagggcgcgtcagc |
| | | | gggtgttggcgggtgtcggggctggcttaactatgcggcatcagagcagattgtactgagagtgcaccatatgcggtgtgaaataccgcacagatgcgt |
| | | | aaggagaaaataccgcatcaggcgccattcgccattcaggctgcgcaactgttgggaagggcgatcggtgcgggcctcttcgctattacgccagctggc |
| | | | gaaagggggatgtgctgcaaggcgattaagttgggtaacgccagggttttcccagtcacgacgttgtaaaacgacggccagagaattc |
| | SERPINA1 | A1AT w/o | GAGGACCCCCAGGGAGATGCAGCCCAGAAGACAGACACCAGCCACCATGACCAGGACCACCCCACCTTCAACAAGA |
| | copy 1 | SP | TCACCCCCAACCTGGCAGAGTTTGCCTTCAGCCTGTACAGGCAGCTGGCCCACCAGAGCAACAGCACCAACATCTTC |
| | | (alternate | TTCAGCCCAGTGAGCATAGCCACAGCCTTTGCCATGCTGAGCCTGGGCACCAAGGCAGACACCCATGATGAGATCCT |
| | | codon usage | GGAGGGCCTGAACTTCAACCTGACAGAGATCCCAGAGGCCCAGATCCATGAGGGCTTCCAGGAGCTGCTGAGGAC |
| | | 1) CpG | CCTGAACCAGCCAGACAGCCAGCTGCAGCTGACCACAGGCAATGGCCTGTTCCTGTCTGAGGGCCTGAAGCTGGTG |
| | | depleted | GACAAGTTCCTGGAGGATGTGAAGAAGCTGTACCACTCTGAGGCCTTCACAGTGAACTTTGGAGACACAGAGGAGG |
| | | (SEQ ID NO: | CCAAGAAGCAGATCAATGACTATGTGGAGAAGGGCACCCAGGGCAAGATAGTGGACCTGGTGAAGGAGCTGGAC |
| | | 771) | AGGGACACAGTGTTTGCCCTGGTGAACTACATCTTCTTCAAGGGCAAGTGGGAGAGGCCCTTTGAGGTGAAGGACA |
| | | | CAGAGGAGGAGGACTTCCATGTGGACCAGGTGACCACAGTGAAGGTGCCCATGATGAAGAGGCTGGGCATGTTCA |
| | | | ATATCCAGCACTGCAAGAAGCTGAGCAGCTGGGTGCTGCTGATGAAGTACCTGGGCAATGCCACAGCCATCTTCTTC |
| | | | CTGCCAGATGAGGGCAAGCTGCAGCACCTGGAGAATGAGCTGACCCATGACATCATCACCAAGTTCCTGGAGAATG |
| | | | AGGACAGGAGGTCTGCCAGCCTGCACCTGCCCAAGCTGAGCATCACAGGCACCTATGACCTGAAGTCTGTGCTGGG |
| | | | CCAGCTGGGCATCACCAAGGTGTTCAGCAATGGAGCAGACCTGTCTGGAGTGACAGAGGAGGCCCCCCTGAAGCT |
| | | | GAGCAAGGCAGTGCACAAGGCAGTGCTGACCATAGATGAGAAGGGCACAGAGGCAGCAGGAGCCATGTTCCTGG |
| | | | AGGCCATCCCCATGAGCATCCCCCCAGAGGTGAAGTTCAACAAGCCTTTTGTGTTCCTGATGATAGAGCAGAACACC |
| | | | AAGAGCCCCCTGTTCATGGGCAAGGTGGTGAACCCCACCCAGAAGTAA |
| | SERPINA1 | A1AT w/o | GAGGATCCCCAGGGAGATGCTGCCCAGAAGACAGATACATCCCACCATGATCAGGATCACCCAACCTTCAACAAGA |
| | copy 2 (rev | SP CpG | TCACCCCCAACCTGGCTGAGTTTGCCTTCAGCCTATACAGACAGCTGGCACACCAGTCCAACAGCACCAATATCTTCT |
| | comp) | depleted | TCTCCCCAGTGAGCATAGCTACAGCCTTTGCAATGCTCTCCCTGGGGACCAAGGCTGACACTCATGATGAAATCCTG |
| | | (SEQ ID NO: | GAGGGCCTGAATTTCAACCTCACAGAGATTCCAGAGGCTCAGATCCATGAAGGCTTCCAGGAACTCCTCAGAACCCT |
| | | 772) | CAACCAGCCAGACAGCCAGCTCCAGCTGACCACAGGCAATGGCCTGTTCCTCTCTGAGGGCCTGAAGCTAGTGGAT |
| | | | AAGTTTTTGGAGGATGTTAAAAAGTTGTACCACTCAGAAGCCTTCACTGTCAACTTTGGGGACACAGAAGAGGCCAA |
| | | | GAAACAGATCAATGATTATGTGGAGAAGGGTACTCAAGGGAAAATTGTGGATTTGGTCAAGGAGCTTGACAGAGA |
| | | | CACAGTTTTTGCTCTGGTGAATTACATCTTCTTTAAAGGCAAATGGGAGAGACCCTTTGAAGTCAAGGACACAGAGG |
| | | | AAGAGGACTTCCATGTGGACCAGGTGACCACAGTGAAGGTGCCTATGATGAAAAGGCTTGGTATGTTCAATATCCA |
| | | | GCACTGTAAGAAGCTGTCCAGCTGGGTGCTGCTGATGAAATACCTGGGCAATGCCACAGCCATCTTCTTCCTGCCTG |
| | | | ATGAGGGGAAACTACAGCACCTGGAAAATGAACTCACCCATGATATCATCACCAAGTTCCTGGAAAATGAAGACAG |
| | | | AAGGTCTGCCAGCTTACATTTACCCAAACTGTCCATTACTGGAACCTATGATCTGAAGTCTGTCCTGGGTCAACTGGG |
| | | | CATCACTAAGGTCTTCAGCAATGGGGCTGACCTCTCTGGGGTCACAGAGGAGGCACCCCTGAAGCTCTCCAAGGCA |
| | | | GTGCATAAGGCTGTGCTGACCATAGATGAGAAGGGCACAGAGGCTGCTGGGGCCATGTTTTTAGAGGCCATACCCA |
| | | | TGTCTATCCCCCCAGAGGTCAAGTTCAACAAACCTTTTGTATTTCTCATGATAGAGCAGAACACTAAATCACCCCTCTT |
| | | | CATGGGAAAAGTGGTGAATCCCACCCAAAAAtaa |
| 8 | Full | (SEQ ID NO: | tgtaacatcagagattttgagacacgggccagagctgcatcgcgcgtttcggtgatgacggtgaaaacctctgacacatgcagctcccggagacggtca |
| | Sequence | 780) | cagcttgtctgtaagcggatgccgggagcagacaagcccgtcagggcgcgtcagcgggtgttggcgggtgtcggggctggcttaactatgcggcatcag |
| | | | agcagattgtactgagagtgcaccatatgcggtgtgaaataccgcacagatgcgtaaggagaaaataccgcatcaggcgccattcgccattcaggctgc |
| | | | gcaactgttgggaagggcgatcggtgcgggcctcttcgctattacgccagctggcgaaagggggatgtgctgcaaggcgattaagttgggtaacgccag |
| | | | ggttttcccagtcacgacgttgtaaaacgacggccagagaattcTTGGCCACTCCCTCTCTGCGCGCTCGCTCGCTCACTGAGGCCG |
| | | | GGCGACCAAAGGTCGCCCGACGCCCGGGCTTTGCCCGGGCGGCCTCAGTGAGCGAGCGAGCGCGCAGAGAGGGA |
| | | | GTGGCCAACTCCATCACTAGGGGTTCCTAGATCTACTAGTtaggtcagtgaagagaagaacaaaaagcagcatattacagttagttgt |
| | | | cttcatcaatctttaaatatgttgtgtggtttttctctccctgtttccacagttGAGGACCCCCAGGGAGATGCTGCCCAGAAGACAGACA |
| | | | CATCTCACCATGACCAGGACCACCCCACCTTCAACAAGATCACTCCCAATCTTGCAGAGTTTGCATTCTCTCTCTACAG |
| | | | ACAGCTTGCACACCAGAGCAACTCTACTAACATCTTCTTCTCTCCAGTCAGCATAGCAACAGCATTTGCAATGCTCAG |
| | | | CCTTGGCACAAAGGCAGACACACATGATGAGATCCTTGAGGGCCTCAACTTCAATCTCACAGAGATCCCAGAAGCCC |
| | | | AGATCCATGAGGGCTTCCAGGAGCTGCTGAGAACACTCAACCAGCCTGACTCTCAGCTCCAGCTCACAACAGGCAAT |
| | | | GGGCTCTTCCTCTCTGAGGGCCTCAAGCTTGTAGACAAGTTCCTGGAGGATGTCAAGAAGCTCTACCACTCTGAAGC |
| | | | CTTCACAGTCAACTTTGGAGACACAGAGGAAGCCAAGAAGCAGATCAATGACTATGTAGAGAAGGGGACTCAGGG |
| | | | CAAGATAGTAGACCTTGTCAAGGAGCTGGACAGAGACACAGTCTTTGCACTGGTCAACTACATCTTCTTCAAGGGGA |
| | | | AGTGGGAGAGACCCTTTGAAGTCAAGGACACAGAGGAGGAGGACTTCCATGTAGACCAGGTGACAACAGTCAAGG |
| | | | TTCCCATGATGAAGAGACTTGGCATGTTCAATATCCAGCACTGCAAGAAGCTCAGCTCTTGGGTCCTCCTCATGAAGT |
| | | | ACCTTGGCAATGCAACAGCAATCTTCTTCCTTCCTGATGAGGGCAAGCTCCAGCACCTTGAGAATGAGCTGACACAT |
| | | | GACATCATCACAAAGTTCCTGGAGAATGAGGACAGAAGGTCTGCATCTCTCCACCTTCCAAAGCTCAGCATCACAGG |
| | | | CACCTATGACCTCAAGTCTGTCCTTGGCCAGCTTGGCATCACAAAGGTCTTCTCTAATGGTGCAGACCTCTCTGGAGT |
| | | | CACAGAGGAAGCCCCCCTCAAGCTCAGCAAGGCTGTGCACAAGGCTGTGCTCACAATAGATGAGAAGGGGACAGA |
| | | | GGCTGCAGGTGCCATGTTCCTGGAAGCCATCCCCATGAGCATCCCACCAGAAGTCAAGTTCAACAAGCCTTTTGTCTT |
| | | | CCTGATGATAGAGCAGAACACAAAGTCTCCCCTCTTCATGGGCAAGGTAGTCAACCCCACTCAAAAGTAACAGACAT |
| | | | GATAAGATACATTGATGAGTTTGGACAAACCACAACTAGAATGCAGTGAAAAAAATGCTTTATTTGTGAAATTTGTG |
| | | | ATGCTATTGCTTTATTTGTAACCATTATAAGCTGCAATAAACAAGTTAACAACAACAATTGCATTCATTTTATGTTTCA |
| | | | GGTTCAGGGGGAGGTGTGGGAGGTTTTTTggggataccccctagagccccagctggttcttttctcctcagaagCCATAGAGCCCAT |
| | | | CTCATCCCCAGCATGCCTGCTATTGTCTTCCCAATCCTCCCCCTTGCTGTCCTGCCCCACCCCACCCCCCAGAATAGAA |
| | | | TGACACCTACTCAGACAATTCTATGCAATTTCCTCATTTTATTAGGAAAGGACAGTGGGAGTGGCACCTTCCAGGGTC |
| | | | AAGGAAGGCATGGGGGAGGGGCAAACAACAGATGGCTGGCAACTAGAAGGCACAGTCTaggTTACTTCTGGGTGG |
| | | | GGTTCACCACCTTGCCCATGAACAGGGGGCTCTTGGTGTTCTGCTCTATCATCAGGAACACAAAAGGCTTGTTGAAC |
| | | | TTCACCTCTGGGGGGATGCTCATGGGGATGGCCTCCAGGAACATGGCTCCTGCTGCCTCTGTGCCCTTCTCATCTATG |
| | | | GTCAGCACTGCCTTGTGCACTGCCTTGCTCAGCTTCAGGGGGGCCTCCTCTGTCACTCCAGACAGGTCTGCTCCATTG |
| | | | CTGAACACCTTGGTGATGCCCAGCTGGCCCAGCACAGACTTCAGGTCATAGGTGCCTGTGATGCTCAGCTTGGGCA |
| | | | GGTGCAGGCTGGCAGACCTCCTGTCCTCATTCTCCAGGAACTTGGTGATGATGTCATGGGTCAGCTCATTCTCCAGG |
| | | | TGCTGCAGCTTGCCCTCATCTGGCAGGAAGAAGATGGCTGTGGCATTGCCCAGGTACTTCATCAGCAGCACCCAGCT |
| | | | GCTCAGCTTCTTGCAGTGCTGGATATTGAACATGCCCAGCCTCTTCATCATGGGCACCTTCACTGTGGTCACCTGGTC |
| | | | CACATGGAAGTCCTCCTCCTCTGTGTCCTTCACCTCAAAGGGCCTCTCCCACTTGCCCTTGAAGAAGATGTAGTTCAC |
| | | | CAGGGCAAACACTGTGTCCCTGTCCAGCTCCTTCACCAGGTCCACTATCTTGCCCTGGGTGCCCTTCTCCACATAGTC |
| | | | ATTGATCTGCTTCTTGGCCTCCTCTGTGTCTCCAAAGTTCACTGTGAAGGCCTCAGAGTGGTACAGCTTCTTCACATCC |
| | | | TCCAGGAACTTGTCCACCAGCTTCAGGCCCTCAGACAGGAACAGGCCATTGCCTGTGGTCAGCTGCAGCTGGCTGTC |
| | | | TGGCTGGTTCAGGGTCCTCAGCAGCTCCTGGAAGCCCTCATGGATCTGGGCCTCTGGGATCTCTGTCAGGTTGAAGT |
| | | | TCAGGCCCTCCAGGATCTCATCATGGGTGTCTGCCTTGGTGCCCAGGCTCAGCATGGCAAAGGCTGTGGCTATGCTC |
| | | | ACTGGGCTGAAGAAGATGTTGGTGCTGTTGCTCTGGTGGGCCAGCTGCCTGTACAGGCTGAAGGCAAACTCTGCCA |
| | | | GGTTGGGGGTGATCTTGTTGAAGGTGGGGTGGTCCTGGTCATGGTGGCTGGTGTCTGTCTTCTGGGCTGCATCTCC |
| | | | CTGGGGGTCCTCaactgtggaaacagggagagaaaaaccacacaacatatttaaagattgatgaagacaactaactgtaatatgctgctttttgtt |
| | | | cttctcttcactgacctaACTAGTAGATCTAGGAACCCCTAGTGATGGAGTTGGCCACTCCCTCTCTGCGCGCTCGCTCGCTC |
| | | | ACTGAGGCCGCCCGGGCAAAGCCCGGGCGTCGGGCGACCTTTGGTCGCCCGGCCTCAGTGAGCGAGCGAGCGCGC |
| | | | AGAGAGGGAGTGGCCAAacgcgtggtgtaatcatggtcatagctgtttcctgtgtgaaattgttatccgctcacaattccacacaacatacgagc |
| | | | cggaagcataaagtgtaaagcctggggtgcctaatgagtgagctaactcacattaattgcgttgcgctcactgcccgctttccagtcgggaaacctgtcgt |
| | | | gccagctgcattaatgaatcggccaacgcgcggggagaggcggtttgcgtattgggcgctcttccgcttcctcgctcactgactcgctgcgctcggtcgttc |
| | | | ggctgcggcgagcggtatcagctcactcaaaggcggtaatacggttatccacagaatcaggggataacgcaggaaagaacatgtgagcaaaaggcca |
| | | | gcaaaaggccaggaaccgtaaaaaggccgcgttgctggcgtttttccataggctccgcccccctgacgagcatcacaaaaatcgacgctcaagtcaga |
| | | | ggtggcgaaacccgacaggactataaagataccaggcgtttccccctggaagctccctcgtgcgctctcctgttccgaccctgccgcttaccggatacctg |
| | | | tccgcctttctcccttcgggaagcgtggcgctttctcatagctcacgctgtaggtatctcagttcggtgtaggtcgttcgctccaagctgggctgtgtgcacg |
| | | | aaccccccgttcagcccgaccgctgcgccttatccggtaactatcgtcttgagtccaacccggtaagacacgacttatcgccactggcagcagccactggt |
| | | | aacaggattagcagagcgaggtatgtaggcggtgctacagagttcttgaagtggtggcctaactacggctacactagaagaacagtatttggtatctgc |
| | | | gctctgctgaagccagttaccttcggaaaaagagttggtagctcttgatccggcaaacaaaccaccgctggtagcggtggtttttttgtttgcaagcagca |
| | | | gattacgcgcagaaaaaaaggatctcaagaagatcctttgatcttttctacggggtctgacgctcagtggaacgaaaactcacgttaagggattttggtc |
| | | | atgagattatcaaaaaggatcttcacctagatccttttaaattaaaaatgaagttttaaatcaagcccaatctgaataatgttacaaccaattaaccaatt |
| | | | ctgattagaaaaactcatcgagcatcaaatgaaactgcaatttattcatatcaggattatcaataccatatttttgaaaaagccgtttctgtaatgaagga |
| | | | gaaaactcaccgaggcagttccataggatggcaagatcctggtatcggtctgcgattccgactcgtccaacatcaatacaacctattaatttcccctcgtc |
| | | | aaaaataaggttatcaagtgagaaatcaccatgagtgacgactgaatccggtgagaatggcaaaagtttatgcatttctttccagacttgttcaacaggc |
| | | | cagccattacgctcgtcatcaaaatcactcgcatcaaccaaaccgttattcattcgtgattgcgcctgagcgagacgaaatacgcgatcgctgttaaaag |
| | | | gacaattacaaacaggaatcgaatgcaaccggcgcaggaacactgccagcgcatcaacaatattttcacctgaatcaggatattcttctaatacctgga |
| | | | atgctgtttttccggggatcgcagtggtgagtaaccatgcatcatcaggagtacggataaaatgcttgatggtcggaagaggcataaattccgtcagcca |
| | | | gtttagtctgaccatctcatctgtaacatcattggcaacgctacctttgccatgtttcagaaacaactctggcgcatcgggcttcccatacaagcgatagat |
| | | | tgtcgcacctgattgcccgacattatcgcgagcccatttatacccatataaatcagcatccatgttggaatttaatcgcggcctcgacgtttcccgttgaat |
| | | | atggctcataacaccccttgtattactgtttatgtaagcagacagttttattgttcatgatgatatatttttatcttgtgcaa |
| | SERPINA1 | A1AT w/o | GAGGACCCCCAGGGAGATGCTGCCCAGAAGACAGACACATCTCACCATGACCAGGACCACCCCACCTTCAACAAGA |
| | copy 1 | SP | TCACTCCCAATCTTGCAGAGTTTGCATTCTCTCTCTACAGACAGCTTGCACACCAGAGCAACTCTACTAACATCTTCTT |
| | | (alternate | CTCTCCAGTCAGCATAGCAACAGCATTTGCAATGCTCAGCCTTGGCACAAAGGCAGACACACATGATGAGATCCTTG |
| | | codon usage | AGGGCCTCAACTTCAATCTCACAGAGATCCCAGAAGCCCAGATCCATGAGGGCTTCCAGGAGCTGCTGAGAACACT |
| | | 2) CpG | CAACCAGCCTGACTCTCAGCTCCAGCTCACAACAGGCAATGGGCTCTTCCTCTCTGAGGGCCTCAAGCTTGTAGACA |
| | | depleted | AGTTCCTGGAGGATGTCAAGAAGCTCTACCACTCTGAAGCCTTCACAGTCAACTTTGGAGACACAGAGGAAGCCAA |
| | | (SEQ ID NO: | GAAGCAGATCAATGACTATGTAGAGAAGGGGACTCAGGGCAAGATAGTAGACCTTGTCAAGGAGCTGGACAGAGA |
| | | 781) | CACAGTCTTTGCACTGGTCAACTACATCTTCTTCAAGGGGAAGTGGGAGAGACCCTTTGAAGTCAAGGACACAGAG |
| | | | GAGGAGGACTTCCATGTAGACCAGGTGACAACAGTCAAGGTTCCCATGATGAAGAGACTTGGCATGTTCAATATCC |
| | | | AGCACTGCAAGAAGCTCAGCTCTTGGGTCCTCCTCATGAAGTACCTTGGCAATGCAACAGCAATCTTCTTCCTTCCTG |
| | | | ATGAGGGCAAGCTCCAGCACCTTGAGAATGAGCTGACACATGACATCATCACAAAGTTCCTGGAGAATGAGGACAG |
| | | | AAGGTCTGCATCTCTCCACCTTCCAAAGCTCAGCATCACAGGCACCTATGACCTCAAGTCTGTCCTTGGCCAGCTTGG |
| | | | CATCACAAAGGTCTTCTCTAATGGTGCAGACCTCTCTGGAGTCACAGAGGAAGCCCCCCTCAAGCTCAGCAAGGCTG |
| | | | TGCACAAGGCTGTGCTCACAATAGATGAGAAGGGGACAGAGGCTGCAGGTGCCATGTTCCTGGAAGCCATCCCCAT |
| | | | GAGCATCCCACCAGAAGTCAAGTTCAACAAGCCTTTTGTCTTCCTGATGATAGAGCAGAACACAAAGTCTCCCCTCTT |
| | | | CATGGGCAAGGTAGTCAACCCCACTCAAAAG |
| | SERPINA1 | A1AT w/o | GAGGACCCCCAGGGAGATGCAGCCCAGAAGACAGACACCAGCCACCATGACCAGGACCACCCCACCTTCAACAAGA |
| | copy 2 (rev | SP CpG | TCACCCCCAACCTGGCAGAGTTTGCCTTCAGCCTGTACAGGCAGCTGGCCCACCAGAGCAACAGCACCAACATCTTC |
| | comp) | depleted | TTCAGCCCAGTGAGCATAGCCACAGCCTTTGCCATGCTGAGCCTGGGCACCAAGGCAGACACCCATGATGAGATCCT |
| | | (SEQ ID NO: | GGAGGGCCTGAACTTCAACCTGACAGAGATCCCAGAGGCCCAGATCCATGAGGGCTTCCAGGAGCTGCTGAGGAC |
| | | 782) | CCTGAACCAGCCAGACAGCCAGCTGCAGCTGACCACAGGCAATGGCCTGTTCCTGTCTGAGGGCCTGAAGCTGGTG |
| | | | GACAAGTTCCTGGAGGATGTGAAGAAGCTGTACCACTCTGAGGCCTTCACAGTGAACTTTGGAGACACAGAGGAGG |
| | | | CCAAGAAGCAGATCAATGACTATGTGGAGAAGGGCACCCAGGGCAAGATAGTGGACCTGGTGAAGGAGCTGGAC |
| | | | AGGGACACAGTGTTTGCCCTGGTGAACTACATCTTCTTCAAGGGCAAGTGGGAGAGGCCCTTTGAGGTGAAGGACA |
| | | | CAGAGGAGGAGGACTTCCATGTGGACCAGGTGACCACAGTGAAGGTGCCCATGATGAAGAGGCTGGGCATGTTCA |
| | | | ATATCCAGCACTGCAAGAAGCTGAGCAGCTGGGTGCTGCTGATGAAGTACCTGGGCAATGCCACAGCCATCTTCTTC |
| | | | CTGCCAGATGAGGGCAAGCTGCAGCACCTGGAGAATGAGCTGACCCATGACATCATCACCAAGTTCCTGGAGAATG |
| | | | AGGACAGGAGGTCTGCCAGCCTGCACCTGCCCAAGCTGAGCATCACAGGCACCTATGACCTGAAGTCTGTGCTGGG |
| | | | CCAGCTGGGCATCACCAAGGTGTTCAGCAATGGAGCAGACCTGTCTGGAGTGACAGAGGAGGCCCCCCTGAAGCT |
| | | | GAGCAAGGCAGTGCACAAGGCAGTGCTGACCATAGATGAGAAGGGCACAGAGGCAGCAGGAGCCATGTTCCTGG |
| | | | AGGCCATCCCCATGAGCATCCCCCCAGAGGTGAAGTTCAACAAGCCTTTTGTGTTCCTGATGATAGAGCAGAACACC |
| | | | AAGAGCCCCCTGTTCATGGGCAAGGTGGTGAACCCCACCCAGAAGTAA |
| 2 | Full | (SEQ ID NO: | TTGGCCACTCCCTCTCTGCGCGCTCGCTCGCTCACTGAGGCCGGGCGACCAAAGGTCGCCCGACGCCCGGGCTTTGC |
| | Sequence | 720) | CCGGGCGGCCTCAGTGAGCGAGCGAGCGCGCAGAGAGGGAGTGGCCAACTCCATCACTAGGGGTTCCTAGATCTA |
| | | | CTAGTtaggtcagtgaagagaagaacaaaaagcagcatattacagttagttgtcttcatcaatctttaaatatgttgtgtggtttttctctccctgtttcca |
| | | | cagttGAGGACCCCCAGGGCGACGCCGCCCAGAAGACCGACACCAGCCACCACGACCAGGACCACCCCACCTTCAAC |
| | | | AAGATCACCCCCAACCTGGCCGAGTTCGCCTTCAGCCTGTACAGGCAGCTGGCCCACCAGAGCAACAGCACCAACAT |
| | | | CTTCTTCAGCCCCGTGAGCATCGCCACCGCCTTCGCCATGCTGAGCCTGGGCACCAAGGCCGACACCCACGACGAGA |
| | | | TCCTGGAGGGCCTGAACTTCAACCTGACCGAGATCCCCGAGGCCCAGATCCACGAGGGCTTCCAGGAGCTGCTGAG |
| | | | GACCCTGAACCAGCCCGACAGCCAGCTGCAGCTGACCACCGGCAACGGCCTGTTCCTGAGCGAGGGCCTGAAGCTG |
| | | | GTGGACAAGTTCCTGGAGGACGTGAAGAAGCTGTACCACAGCGAGGCCTTCACCGTGAACTTCGGCGACACCGAG |
| | | | GAGGCCAAGAAGCAGATCAACGACTACGTGGAGAAGGGCACCCAGGGCAAGATCGTGGACCTGGTGAAGGAGCT |
| | | | GGACAGGGACACCGTGTTCGCCCTGGTGAACTACATCTTCTTCAAGGGCAAGTGGGAGAGGCCCTTCGAGGTGAAG |
| | | | GACACCGAGGAGGAGGACTTCCACGTGGACCAGGTGACCACCGTGAAGGTGCCCATGATGAAGAGGCTGGGCATG |
| | | | TTCAACATCCAGCACTGCAAGAAGCTGAGCAGCTGGGTGCTGCTGATGAAGTACCTGGGCAACGCCACCGCCATCTT |
| | | | CTTCCTGCCCGACGAGGGCAAGCTGCAGCACCTGGAGAACGAGCTGACCCACGACATCATCACCAAGTTCCTGGAG |
| | | | AACGAGGACAGGAGGAGCGCCAGCCTGCACCTGCCCAAGCTGAGCATCACCGGCACCTACGACCTGAAGAGCGTG |
| | | | CTGGGCCAGCTGGGCATCACCAAGGTGTTCAGCAACGGCGCCGACCTGAGCGGCGTGACCGAGGAGGCCCCCCTG |
| | | | AAGCTGAGCAAGGCCGTGCACAAGGCCGTGCTGACCATCGACGAGAAGGGCACCGAGGCCGCCGGCGCCATGTTC |
| | | | CTGGAGGCCATCCCCATGAGCATCCCCCCCGAGGTGAAGTTCAACAAGCCTTTCGTGTTCCTGATGATCGAGCAGAA |
| | | | CACCAAGAGCCCCCTGTTCATGGGCAAGGTGGTGAACCCCACCCAGAAGTAACAGACATGATAAGATACATTGATG |
| | | | AGTTTGGACAAACCACAACTAGAATGCAGTGAAAAAAATGCTTTATTTGTGAAATTTGTGATGCTATTGCTTTATTTG |
| | | | TAACCATTATAAGCTGCAATAAACAAGTTAACAACAACAATTGCATTCATTTTATGTTTCAGGTTCAGGGGGAGGTGT |
| | | | GGGAGGTTTTTTggggataccccctagagccccagctggttctttccgcctcagaagCCATAGAGCCCACCGCATCCCCAGCATGCCT |
| | | | GCTATTGTCTTCCCAATCCTCCCCCTTGCTGTCCTGCCCCACCCCACCCCCCAGAATAGAATGACACCTACTCAGACAA |
| | | | TGCGATGCAATTTCCTCATTTTATTAGGAAAGGACAGTGGGAGTGGCACCTTCCAGGGTCAAGGAAGGCACGGGGG |
| | | | AGGGGCAAACAACAGATGGCTGGCAACTAGAAGGCACAGTCGaggttaTTTTTGGGGGGATTCACCACTTTTCCCAT |
| | | | GAAGAGGGGTGATTTAGTGTTCTGCTCGATCATGAGAAATACAAAAGGTTTGTTGAACTTGACCTCGGGGGGGATA |
| | | | GACATGGGTATGGCCTCTAAAAACATGGCCCCAGCAGCCTCGGTGCCCTTCTCGTCGATGGTCAGCACAGCCTTATG |
| | | | CACGGCCTTGGAGAGCTTCAGGGGTGCCTCCTCTGTGACCCCGGAGAGGTCAGCCCCATTGCTGAAGACCTTAGTG |
| | | | ATGCCCAGTTGACCCAGGACGCTCTTCAGATCATAGGTTCCAGTAATGGACAGTTTGGGTAAATGTAAGCTGGCAGA |
| | | | CCTTCTGTCTTCATTTTCCAGGAACTTGGTGATGATATCGTGGGTGAGTTCATTTTCCAGGTGCTGTAGTTTCCCCTCA |
| | | | TCAGGCAGGAAGAAGATGGCGGTGGCATTGCCCAGGTATTTCATCAGCAGCACCCAGCTGGACAGCTTCTTACAGT |
| | | | GCTGGATATTGAACATACCAAGCCTTTTCATCATAGGCACCTTCACGGTGGTCACCTGGTCCACGTGGAAGTCCTCTT |
| | | | CCTCGGTGTCCTTGACTTCAAAGGGTCTCTCCCATTTGCCTTTAAAGAAGATGTAATTCACCAGAGCAAAAACTGTGT |
| | | | CTCTGTCAAGCTCCTTGACCAAATCCACAATTTTCCCTTGAGTACCCTTCTCCACGTAATCGTTGATCTGTTTCTTGGCC |
| | | | TCTTCGGTGTCCCCGAAGTTGACAGTGAAGGCTTCTGAGTGGTACAACTTTTTAACATCCTCCAAAAACTTATCCACT |
| | | | AGCTTCAGGCCCTCGCTGAGGAACAGGCCATTGCCGGTGGTCAGCTGGAGCTGGCTGTCTGGCTGGTTGAGGGTAC |
| | | | GGAGGAGTTCCTGGAAGCCTTCATGGATCTGAGCCTCCGGAATCTCCGTGAGGTTGAAATTCAGGCCCTCCAGGATT |
| | | | TCATCGTGAGTGTCAGCCTTGGTCCCCAGGGAGAGCATTGCAAAGGCTGTAGCGATGCTCACTGGGGAGAAGAAG |
| | | | ATATTGGTGCTGTTGGACTGGTGTGCCAGCTGGCGGTATAGGCTGAAGGCGAACTCAGCCAGGTTGGGGGTGATCT |
| | | | TGTTGAAGGTTGGGTGATCCTGATCATGGTGGGATGTATCTGTCTTCTGGGCAGCATCTCCCTGGGGATCCTCaactgt |
| | | | ggaaacagggagagaaaaaccacacaacatatttaaagattgatgaagacaactaactgtaatatgctgctttttgttcttctcttcactgacctaACTA |
| | | | GTAGATCTAGGAACCCCTAGTGATGGAGTTGGCCACTCCCTCTCTGCGCGCTCGCTCGCTCACTGAGGCCGCCCGGG |
| | | | CAAAGCCCGGGCGTCGGGCGACCTTTGGTCGCCCGGCCTCAGTGAGCGAGCGAGCGCGCAGAGAGGGAGTGGCC |
| | | | AAacgcgtggtgtaatcatggtcatagctgtttcctgtgtgaaattgttatccgctcacaattccacacaacatacgagccggaagcataaagtgtaaag |
| | | | cctggggtgcctaatgagtgagctaactcacattaattgcgttgcgctcactgcccgctttccagtcgggaaacctgtcgtgccagctgcattaatgaatcg |
| | | | gccaacgcgcggggagaggcggtttgcgtattgggcgctcttccgcttcctcgctcactgactcgctgcgctcggtcgttcggctgcggcgagcggtatca |
| | | | gctcactcaaaggcggtaatacggttatccacagaatcaggggataacgcaggaaagaacatgtgagcaaaaggccagcaaaaggccaggaaccgt |
| | | | aaaaaggccgcgttgctggcgtttttccataggctccgcccccctgacgagcatcacaaaaatcgacgctcaagtcagaggtggcgaaacccgacagg |
| | | | actataaagataccaggcgtttccccctggaagctccctcgtgcgctctcctgttccgaccctgccgcttaccggatacctgtccgcctttctcccttcggga |
| | | | agcgtggcgctttctcatagctcacgctgtaggtatctcagttcggtgtaggtcgttcgctccaagctgggctgtgtgcacgaaccccccgttcagcccgac |
| | | | cgctgcgccttatccggtaactatcgtcttgagtccaacccggtaagacacgacttatcgccactggcagcagccactggtaacaggattagcagagcga |
| | | | ggtatgtaggcggtgctacagagttcttgaagtggtggcctaactacggctacactagaagaacagtatttggtatctgcgctctgctgaagccagttacc |
| | | | ttcggaaaaagagttggtagctcttgatccggcaaacaaaccaccgctggtagcggtggtttttttgtttgcaagcagcagattacgcgcagaaaaaaag |
| | | | gatctcaagaagatcctttgatcttttctacggggtctgacgctcagtggaacgaaaactcacgttaagggattttggtcatgagattatcaaaaaggatc |
| | | | ttcacctagatccttttaaattaaaaatgaagttttaaatcaagcccaatctgaataatgttacaaccaattaaccaattctgattagaaaaactcatcga |
| | | | gcatcaaatgaaactgcaatttattcatatcaggattatcaataccatatttttgaaaaagccgtttctgtaatgaaggagaaaactcaccgaggcagttc |
| | | | cataggatggcaagatcctggtatcggtctgcgattccgactcgtccaacatcaatacaacctattaatttcccctcgtcaaaaataaggttatcaagtga |
| | | | gaaatcaccatgagtgacgactgaatccggtgagaatggcaaaagtttatgcatttctttccagacttgttcaacaggccagccattacgctcgtcatcaa |
| | | | aatcactcgcatcaaccaaaccgttattcattcgtgattgcgcctgagcgagacgaaatacgcgatcgctgttaaaaggacaattacaaacaggaatcg |
| | | | aatgcaaccggcgcaggaacactgccagcgcatcaacaatattttcacctgaatcaggatattcttctaatacctggaatgctgtttttccggggatcgca |
| | | | gtggtgagtaaccatgcatcatcaggagtacggataaaatgcttgatggtcggaagaggcataaattccgtcagccagtttagtctgaccatctcatctgt |
| | | | aacatcattggcaacgctacctttgccatgtttcagaaacaactctggcgcatcgggcttcccatacaagcgatagattgtcgcacctgattgcccgacat |
| | | | tatcgcgagcccatttatacccatataaatcagcatccatgttggaatttaatcgcggcctcgacgtttcccgttgaatatggctcataacaccccttgtatt |
| | | | actgtttatgtaagcagacagttttattgttcatgatgatatatttttatcttgtgcaatgtaacatcagagattttgagacacgggccagagctgcatcgcg |
| | | | cgtttcggtgatgacggtgaaaacctctgacacatgcagctcccggagacggtcacagcttgtctgtaagcggatgccgggagcagacaagcccgtcag |
| | | | ggcgcgtcagcgggtgttggcgggtgtcggggctggcttaactatgcggcatcagagcagattgtactgagagtgcaccatatgcggtgtgaaataccg |
| | | | cacagatgcgtaaggagaaaataccgcatcaggcgccattcgccattcaggctgcgcaactgttgggaagggcgatcggtgcgggcctcttcgctatta |
| | | | cgccagctggcgaaagggggatgtgctgcaaggcgattaagttgggtaacgccagggttttcccagtcacgacgttgtaaaacgacggccagagaattc |
| | SERPINA1 | A1AT w/o | GAGGACCCCCAGGGCGACGCCGCCCAGAAGACCGACACCAGCCACCACGACCAGGACCACCCCACCTTCAACAAGA |
| | copy 1 | SP | TCACCCCCAACCTGGCCGAGTTCGCCTTCAGCCTGTACAGGCAGCTGGCCCACCAGAGCAACAGCACCAACATCTTC |
| | | (alternate | TTCAGCCCCGTGAGCATCGCCACCGCCTTCGCCATGCTGAGCCTGGGCACCAAGGCCGACACCCACGACGAGATCCT |
| | | codon usage | GGAGGGCCTGAACTTCAACCTGACCGAGATCCCCGAGGCCCAGATCCACGAGGGCTTCCAGGAGCTGCTGAGGACC |
| | | 1) | CTGAACCAGCCCGACAGCCAGCTGCAGCTGACCACCGGCAACGGCCTGTTCCTGAGCGAGGGCCTGAAGCTGGTGG |
| | | (SEQ ID NO: | ACAAGTTCCTGGAGGACGTGAAGAAGCTGTACCACAGCGAGGCCTTCACCGTGAACTTCGGCGACACCGAGGAGG |
| | | 721) | CCAAGAAGCAGATCAACGACTACGTGGAGAAGGGCACCCAGGGCAAGATCGTGGACCTGGTGAAGGAGCTGGAC |
| | | | AGGGACACCGTGTTCGCCCTGGTGAACTACATCTTCTTCAAGGGCAAGTGGGAGAGGCCCTTCGAGGTGAAGGACA |
| | | | CCGAGGAGGAGGACTTCCACGTGGACCAGGTGACCACCGTGAAGGTGCCCATGATGAAGAGGCTGGGCATGTTCA |
| | | | ACATCCAGCACTGCAAGAAGCTGAGCAGCTGGGTGCTGCTGATGAAGTACCTGGGCAACGCCACCGCCATCTTCTTC |
| | | | CTGCCCGACGAGGGCAAGCTGCAGCACCTGGAGAACGAGCTGACCCACGACATCATCACCAAGTTCCTGGAGAACG |
| | | | AGGACAGGAGGAGCGCCAGCCTGCACCTGCCCAAGCTGAGCATCACCGGCACCTACGACCTGAAGAGCGTGCTGG |
| | | | GCCAGCTGGGCATCACCAAGGTGTTCAGCAACGGCGCCGACCTGAGCGGCGTGACCGAGGAGGCCCCCCTGAAGC |
| | | | TGAGCAAGGCCGTGCACAAGGCCGTGCTGACCATCGACGAGAAGGGCACCGAGGCCGCCGGCGCCATGTTCCTGG |
| | | | AGGCCATCCCCATGAGCATCCCCCCCGAGGTGAAGTTCAACAAGCCTTTCGTGTTCCTGATGATCGAGCAGAACACC |
| | | | AAGAGCCCCCTGTTCATGGGCAAGGTGGTGAACCCCACCCAGAAGTAA |
| | SERPINA1 | A1AT w/o | GAGGATCCCCAGGGAGATGCTGCCCAGAAGACAGATACATCCCACCATGATCAGGATCACCCAACCTTCAACAAGA |
| | copy 2 (rev | SP | TCACCCCCAACCTGGCTGAGTTCGCCTTCAGCCTATACCGCCAGCTGGCACACCAGTCCAACAGCACCAATATCTTCT |
| | comp) | (SEQ ID NO: | TCTCCCCAGTGAGCATCGCTACAGCCTTTGCAATGCTCTCCCTGGGGACCAAGGCTGACACTCACGATGAAATCCTG |
| | | 722) | GAGGGCCTGAATTTCAACCTCACGGAGATTCCGGAGGCTCAGATCCATGAAGGCTTCCAGGAACTCCTCCGTACCCT |
| | | | CAACCAGCCAGACAGCCAGCTCCAGCTGACCACCGGCAATGGCCTGTTCCTCAGCGAGGGCCTGAAGCTAGTGGAT |
| | | | AAGTTTTTGGAGGATGTTAAAAAGTTGTACCACTCAGAAGCCTTCACTGTCAACTTCGGGGACACCGAAGAGGCCAA |
| | | | GAAACAGATCAACGATTACGTGGAGAAGGGTACTCAAGGGAAAATTGTGGATTTGGTCAAGGAGCTTGACAGAGA |
| | | | CACAGTTTTTGCTCTGGTGAATTACATCTTCTTTAAAGGCAAATGGGAGAGACCCTTTGAAGTCAAGGACACCGAGG |
| | | | AAGAGGACTTCCACGTGGACCAGGTGACCACCGTGAAGGTGCCTATGATGAAAAGGCTTGGTATGTTCAATATCCA |
| | | | GCACTGTAAGAAGCTGTCCAGCTGGGTGCTGCTGATGAAATACCTGGGCAATGCCACCGCCATCTTCTTCCTGCCTG |
| | | | ATGAGGGGAAACTACAGCACCTGGAAAATGAACTCACCCACGATATCATCACCAAGTTCCTGGAAAATGAAGACAG |
| | | | AAGGTCTGCCAGCTTACATTTACCCAAACTGTCCATTACTGGAACCTATGATCTGAAGAGCGTCCTGGGTCAACTGG |
| | | | GCATCACTAAGGTCTTCAGCAATGGGGCTGACCTCTCCGGGGTCACAGAGGAGGCACCCCTGAAGCTCTCCAAGGC |
| | | | CGTGCATAAGGCTGTGCTGACCATCGACGAGAAGGGCACCGAGGCTGCTGGGGCCATGTTTTTAGAGGCCATACCC |
| | | | ATGTCTATCCCCCCCGAGGTCAAGTTCAACAAACCTTTTGTATTTCTCATGATCGAGCAGAACACTAAATCACCCCTCT |
| | | | TCATGGGAAAAGTGGTGAATCCCACCCAAAAAtaa |
| 3 | Full | (SEQ ID NO: | TTGGCCACTCCCTCTCTGCGCGCTCGCTCGCTCACTGAGGCCGGGCGACCAAAGGTCGCCCGACGCCCGGGCTTTGC |
| | Sequence | 730) | CCGGGCGGCCTCAGTGAGCGAGCGAGCGCGCAGAGAGGGAGTGGCCAACTCCATCACTAGGGGTTCCTAGATCTA |
| | | | CTAGTATAACTTCGTATAGCATACATTATACGAAGTTATATGTATGCtaggtcagtgaagagaagaacaaaaagcagcatattaca |
| | | | gttagttgtcttcatcaatctttaaatatgttgtgtggtttttctctccctgtttccacagttGAGGACCCCCAGGGCGACGCCGCCCAGAAGA |
| | | | CCGACACCAGCCACCACGACCAGGACCACCCCACCTTCAACAAGATCACCCCCAACCTGGCCGAGTTCGCCTTCAGC |
| | | | CTGTACAGGCAGCTGGCCCACCAGAGCAACAGCACCAACATCTTCTTCAGCCCCGTGAGCATCGCCACCGCCTTCGC |
| | | | CATGCTGAGCCTGGGCACCAAGGCCGACACCCACGACGAGATCCTGGAGGGCCTGAACTTCAACCTGACCGAGATC |
| | | | CCCGAGGCCCAGATCCACGAGGGCTTCCAGGAGCTGCTGAGGACCCTGAACCAGCCCGACAGCCAGCTGCAGCTGA |
| | | | CCACCGGCAACGGCCTGTTCCTGAGCGAGGGCCTGAAGCTGGTGGACAAGTTCCTGGAGGACGTGAAGAAGCTGT |
| | | | ACCACAGCGAGGCCTTCACCGTGAACTTCGGCGACACCGAGGAGGCCAAGAAGCAGATCAACGACTACGTGGAGA |
| | | | AGGGCACCCAGGGCAAGATCGTGGACCTGGTGAAGGAGCTGGACAGGGACACCGTGTTCGCCCTGGTGAACTACA |
| | | | TCTTCTTCAAGGGCAAGTGGGAGAGGCCCTTCGAGGTGAAGGACACCGAGGAGGAGGACTTCCACGTGGACCAGG |
| | | | TGACCACCGTGAAGGTGCCCATGATGAAGAGGCTGGGCATGTTCAACATCCAGCACTGCAAGAAGCTGAGCAGCTG |
| | | | GGTGCTGCTGATGAAGTACCTGGGCAACGCCACCGCCATCTTCTTCCTGCCCGACGAGGGCAAGCTGCAGCACCTG |
| | | | GAGAACGAGCTGACCCACGACATCATCACCAAGTTCCTGGAGAACGAGGACAGGAGGAGCGCCAGCCTGCACCTG |
| | | | CCCAAGCTGAGCATCACCGGCACCTACGACCTGAAGAGCGTGCTGGGCCAGCTGGGCATCACCAAGGTGTTCAGCA |
| | | | ACGGCGCCGACCTGAGCGGCGTGACCGAGGAGGCCCCCCTGAAGCTGAGCAAGGCCGTGCACAAGGCCGTGCTGA |
| | | | CCATCGACGAGAAGGGCACCGAGGCCGCCGGCGCCATGTTCCTGGAGGCCATCCCCATGAGCATCCCCCCCGAGGT |
| | | | GAAGTTCAACAAGCCTTTCGTGTTCCTGATGATCGAGCAGAACACCAAGAGCCCCCTGTTCATGGGCAAGGTGGTG |
| | | | AACCCCACCCAGAAGTAACAGACATGATAAGATACATTGATGAGTTTGGACAAACCACAACTAGAATGCAGTGAAA |
| | | | AAAATGCTTTATTTGTGAAATTTGTGATGCTATTGCTTTATTTGTAACCATTATAAGCTGCAATAAACAAGTTAACAAC |
| | | | AACAATTGCATTCATTTTATGTTTCAGGTTCAGGGGGAGGTGTGGGAGGTTTTTTggggataccccctagagccccagctggtt |
| | | | ctttccgcctcagaagCCATAGAGCCCACCGCATCCCCAGCATGCCTGCTATTGTCTTCCCAATCCTCCCCCTTGCTGTCCTG |
| | | | CCCCACCCCACCCCCCAGAATAGAATGACACCTACTCAGACAATGCGATGCAATTTCCTCATTTTATTAGGAAAGGAC |
| | | | AGTGGGAGTGGCACCTTCCAGGGTCAAGGAAGGCACGGGGGAGGGGCAAACAACAGATGGCTGGCAACTAGAAG |
| | | | GCACAGTCGaggttaTTTTTGGGTGGGATTCACCACTTTTCCCATGAAGAGGGGTGATTTAGTGTTCTGCTCGATCATG |
| | | | AGAAATACAAAAGGTTTGTTGAACTTGACCTCGGGGGGGATAGACATGGGTATGGCCTCTAAAAACATGGCCCCAG |
| | | | CAGCCTCGGTGCCCTTCTCGTCGATGGTCAGCACAGCCTTATGCACGGCCTTGGAGAGCTTCAGGGGTGCCTCCTCT |
| | | | GTGACCCCGGAGAGGTCAGCCCCATTGCTGAAGACCTTAGTGATGCCCAGTTGACCCAGGACGCTCTTCAGATCATA |
| | | | GGTTCCAGTAATGGACAGTTTGGGTAAATGTAAGCTGGCAGACCTTCTGTCTTCATTTTCCAGGAACTTGGTGATGA |
| | | | TATCGTGGGTGAGTTCATTTTCCAGGTGCTGTAGTTTCCCCTCATCAGGCAGGAAGAAGATGGCGGTGGCATTGCCC |
| | | | AGGTATTTCATCAGCAGCACCCAGCTGGACAGCTTCTTACAGTGCTGGATATTGAACATACCAAGCCTTTTCATCATA |
| | | | GGCACCTTCACGGTGGTCACCTGGTCCACGTGGAAGTCCTCTTCCTCGGTGTCCTTGACTTCAAAGGGTCTCTCCCAT |
| | | | TTGCCTTTAAAGAAGATGTAATTCACCAGAGCAAAAACTGTGTCTCTGTCAAGCTCCTTGACCAAATCCACAATTTTC |
| | | | CCTTGAGTACCCTTCTCCACGTAATCGTTGATCTGTTTCTTGGCCTCTTCGGTGTCCCCGAAGTTGACAGTGAAGGCTT |
| | | | CTGAGTGGTACAACTTTTTAACATCCTCCAAAAACTTATCCACTAGCTTCAGGCCCTCGCTGAGGAACAGGCCATTGC |
| | | | CGGTGGTCAGCTGGAGCTGGCTGTCTGGCTGGTTGAGGGTACGGAGGAGTTCCTGGAAGCCTTCATGGATCTGAGC |
| | | | CTCCGGAATCTCCGTGAGGTTGAAATTCAGGCCCTCCAGGATTTCATCGTGAGTGTCAGCCTTGGTCCCCAGGGAGA |
| | | | GCATTGCAAAGGCTGTAGCGATGCTCACTGGGGAGAAGAAGATATTGGTGCTGTTGGACTGGTGTGCCAGCTGGC |
| | | | GGTATAGGCTGAAGGCGAACTCAGCCAGGTTGGGGGTGATCTTGTTGAAGGTTGGGTGATCCTGATCATGGTGGG |
| | | | ATGTATCTGTCTTCTGGGCAGCATCTCCCTGGGGATCCTCaactgtggaaacagggagagaaaaaccacacaacatatttaaagatt |
| | | | gatgaagacaactaactgtaatatgctgctttttgttcttctcttcactgacctaATGTATGCATAACTTCGTATAGCATACATTATACGAA |
| | | | GTTATACTAGTAGATCTAGGAACCCCTAGTGATGGAGTTGGCCACTCCCTCTCTGCGCGCTCGCTCGCTCACTGAGG |
| | | | CCGCCCGGGCAAAGCCCGGGCGTCGGGCGACCTTTGGTCGCCCGGCCTCAGTGAGCGAGCGAGCGCGCAGAGAGG |
| | | | GAGTGGCCAAacgcgtggtgtaatcatggtcatagctgtttcctgtgtgaaattgttatccgctcacaattccacacaacatacgagccggaagcata |
| | | | aagtgtaaagcctggggtgcctaatgagtgagctaactcacattaattgcgttgcgctcactgcccgctttccagtcgggaaacctgtcgtgccagctgca |
| | | | ttaatgaatcggccaacgcgcggggagaggcggtttgcgtattgggcgctcttccgcttcctcgctcactgactcgctgcgctcggtcgttcggctgcggc |
| | | | gagcggtatcagctcactcaaaggcggtaatacggttatccacagaatcaggggataacgcaggaaagaacatgtgagcaaaaggccagcaaaagg |
| | | | ccaggaaccgtaaaaaggccgcgttgctggcgtttttccataggctccgcccccctgacgagcatcacaaaaatcgacgctcaagtcagaggtggcgaa |
| | | | acccgacaggactataaagataccaggcgtttccccctggaagctccctcgtgcgctctcctgttccgaccctgccgcttaccggatacctgtccgcctttc |
| | | | tcccttcgggaagcgtggcgctttctcatagctcacgctgtaggtatctcagttcggtgtaggtcgttcgctccaagctgggctgtgtgcacgaaccccccg |
| | | | ttcagcccgaccgctgcgccttatccggtaactatcgtcttgagtccaacccggtaagacacgacttatcgccactggcagcagccactggtaacaggatt |
| | | | agcagagcgaggtatgtaggcggtgctacagagttcttgaagtggtggcctaactacggctacactagaagaacagtatttggtatctgcgctctgctga |
| | | | agccagttaccttcggaaaaagagttggtagctcttgatccggcaaacaaaccaccgctggtagcggtggtttttttgtttgcaagcagcagattacgcgc |
| | | | agaaaaaaaggatctcaagaagatcctttgatcttttctacggggtctgacgctcagtggaacgaaaactcacgttaagggattttggtcatgagattatc |
| | | | aaaaaggatcttcacctagatccttttaaattaaaaatgaagttttaaatcaagcccaatctgaataatgttacaaccaattaaccaattctgattagaaa |
| | | | aactcatcgagcatcaaatgaaactgcaatttattcatatcaggattatcaataccatatttttgaaaaagccgtttctgtaatgaaggagaaaactcacc |
| | | | gaggcagttccataggatggcaagatcctggtatcggtctgcgattccgactcgtccaacatcaatacaacctattaatttcccctcgtcaaaaataaggt |
| | | | tatcaagtgagaaatcaccatgagtgacgactgaatccggtgagaatggcaaaagtttatgcatttctttccagacttgttcaacaggccagccattacg |
| | | | ctcgtcatcaaaatcactcgcatcaaccaaaccgttattcattcgtgattgcgcctgagcgagacgaaatacgcgatcgctgttaaaaggacaattacaa |
| | | | acaggaatcgaatgcaaccggcgcaggaacactgccagcgcatcaacaatattttcacctgaatcaggatattcttctaatacctggaatgctgtttttcc |
| | | | ggggatcgcagtggtgagtaaccatgcatcatcaggagtacggataaaatgcttgatggtcggaagaggcataaattccgtcagccagtttagtctgac |
| | | | catctcatctgtaacatcattggcaacgctacctttgccatgtttcagaaacaactctggcgcatcgggcttcccatacaagcgatagattgtcgcacctga |
| | | | ttgcccgacattatcgcgagcccatttatacccatataaatcagcatccatgttggaatttaatcgcggcctcgacgtttcccgttgaatatggctcataac |
| | | | accccttgtattactgtttatgtaagcagacagttttattgttcatgatgatatatttttatcttgtgcaatgtaacatcagagattttgagacacgggccaga |
| | | | gctgcatcgcgcgtttcggtgatgacggtgaaaacctctgacacatgcagctcccggagacggtcacagcttgtctgtaagcggatgccgggagcagac |
| | | | aagcccgtcagggcgcgtcagcgggtgttggcgggtgtcggggctggcttaactatgcggcatcagagcagattgtactgagagtgcaccatatgcggt |
| | | | gtgaaataccgcacagatgcgtaaggagaaaataccgcatcaggcgccattcgccattcaggctgcgcaactgttgggaagggcgatcggtgcgggcc |
| | | | tcttcgctattacgccagctggcgaaagggggatgtgctgcaaggcgattaagttgggtaacgccagggttttcccagtcacgacgttgtaaaacgacgg |
| | | | ccagagaattc |
| | SERPINA1 | A1AT w/o | GAGGACCCCCAGGGCGACGCCGCCCAGAAGACCGACACCAGCCACCACGACCAGGACCACCCCACCTTCAACAAGA |
| | copy 1 | SP | TCACCCCCAACCTGGCCGAGTTCGCCTTCAGCCTGTACAGGCAGCTGGCCCACCAGAGCAACAGCACCAACATCTTC |
| | | (alternate | TTCAGCCCCGTGAGCATCGCCACCGCCTTCGCCATGCTGAGCCTGGGCACCAAGGCCGACACCCACGACGAGATCCT |
| | | codon usage | GGAGGGCCTGAACTTCAACCTGACCGAGATCCCCGAGGCCCAGATCCACGAGGGCTTCCAGGAGCTGCTGAGGACC |
| | | 1) | CTGAACCAGCCCGACAGCCAGCTGCAGCTGACCACCGGCAACGGCCTGTTCCTGAGCGAGGGCCTGAAGCTGGTGG |
| | | (SEQ ID NO: | ACAAGTTCCTGGAGGACGTGAAGAAGCTGTACCACAGCGAGGCCTTCACCGTGAACTTCGGCGACACCGAGGAGG |
| | | 731) | CCAAGAAGCAGATCAACGACTACGTGGAGAAGGGCACCCAGGGCAAGATCGTGGACCTGGTGAAGGAGCTGGAC |
| | | | AGGGACACCGTGTTCGCCCTGGTGAACTACATCTTCTTCAAGGGCAAGTGGGAGAGGCCCTTCGAGGTGAAGGACA |
| | | | CCGAGGAGGAGGACTTCCACGTGGACCAGGTGACCACCGTGAAGGTGCCCATGATGAAGAGGCTGGGCATGTTCA |
| | | | ACATCCAGCACTGCAAGAAGCTGAGCAGCTGGGTGCTGCTGATGAAGTACCTGGGCAACGCCACCGCCATCTTCTTC |
| | | | CTGCCCGACGAGGGCAAGCTGCAGCACCTGGAGAACGAGCTGACCCACGACATCATCACCAAGTTCCTGGAGAACG |
| | | | AGGACAGGAGGAGCGCCAGCCTGCACCTGCCCAAGCTGAGCATCACCGGCACCTACGACCTGAAGAGCGTGCTGG |
| | | | GCCAGCTGGGCATCACCAAGGTGTTCAGCAACGGCGCCGACCTGAGCGGCGTGACCGAGGAGGCCCCCCTGAAGC |
| | | | TGAGCAAGGCCGTGCACAAGGCCGTGCTGACCATCGACGAGAAGGGCACCGAGGCCGCCGGCGCCATGTTCCTGG |
| | | | AGGCCATCCCCATGAGCATCCCCCCCGAGGTGAAGTTCAACAAGCCTTTCGTGTTCCTGATGATCGAGCAGAACACC |
| | | | AAGAGCCCCCTGTTCATGGGCAAGGTGGTGAACCCCACCCAGAAGTAA |
| | SERPINA1 | A1AT w/o | GAGGATCCCCAGGGAGATGCTGCCCAGAAGACAGATACATCCCACCATGATCAGGATCACCCAACCTTCAACAAGA |
| | copy 2 (rev | SP | TCACCCCCAACCTGGCTGAGTTCGCCTTCAGCCTATACCGCCAGCTGGCACACCAGTCCAACAGCACCAATATCTTCT |
| | comp) | (SEQ ID NO: | TCTCCCCAGTGAGCATCGCTACAGCCTTTGCAATGCTCTCCCTGGGGACCAAGGCTGACACTCACGATGAAATCCTG |
| | | 732) | GAGGGCCTGAATTTCAACCTCACGGAGATTCCGGAGGCTCAGATCCATGAAGGCTTCCAGGAACTCCTCCGTACCCT |
| | | | CAACCAGCCAGACAGCCAGCTCCAGCTGACCACCGGCAATGGCCTGTTCCTCAGCGAGGGCCTGAAGCTAGTGGAT |
| | | | AAGTTTTTGGAGGATGTTAAAAAGTTGTACCACTCAGAAGCCTTCACTGTCAACTTCGGGGACACCGAAGAGGCCAA |
| | | | GAAACAGATCAACGATTACGTGGAGAAGGGTACTCAAGGGAAAATTGTGGATTTGGTCAAGGAGCTTGACAGAGA |
| | | | CACAGTTTTTGCTCTGGTGAATTACATCTTCTTTAAAGGCAAATGGGAGAGACCCTTTGAAGTCAAGGACACCGAGG |
| | | | AAGAGGACTTCCACGTGGACCAGGTGACCACCGTGAAGGTGCCTATGATGAAAAGGCTTGGTATGTTCAATATCCA |
| | | | GCACTGTAAGAAGCTGTCCAGCTGGGTGCTGCTGATGAAATACCTGGGCAATGCCACCGCCATCTTCTTCCTGCCTG |
| | | | ATGAGGGGAAACTACAGCACCTGGAAAATGAACTCACCCACGATATCATCACCAAGTTCCTGGAAAATGAAGACAG |
| | | | AAGGTCTGCCAGCTTACATTTACCCAAACTGTCCATTACTGGAACCTATGATCTGAAGAGCGTCCTGGGTCAACTGG |
| | | | GCATCACTAAGGTCTTCAGCAATGGGGCTGACCTCTCCGGGGTCACAGAGGAGGCACCCCTGAAGCTCTCCAAGGC |
| | | | CGTGCATAAGGCTGTGCTGACCATCGACGAGAAGGGCACCGAGGCTGCTGGGGCCATGTTTTTAGAGGCCATACCC |
| | | | ATGTCTATCCCCCCCGAGGTCAAGTTCAACAAACCTTTTGTATTTCTCATGATCGAGCAGAACACTAAATCACCCCTCT |
| | | | TCATGGGAAAAGTGGTGAATCCCACCCAAAAAtaa |
| 4 | Full | (SEQ ID NO: | ctcaaaggcggtaatacggttatccacagaatcaggggataacgcaggaaagaacatgtgagcaaaaggccagcaaaaggccaggaaccgtaaaaa |
| | Sequence | 740) | ggccgcgttgctggcgtttttccataggctccgcccccctgacgagcatcacaaaaatcgacgctcaagtcagaggtggcgaaacccgacaggactata |
| | | | aagataccaggcgtttccccctggaagctccctcgtgcgctctcctgttccgaccctgccgcttaccggatacctgtccgcctttctcccttcgggaagcgtg |
| | | | gcgctttctcatagctcacgctgtaggtatctcagttcggtgtaggtcgttcgctccaagctgggctgtgtgcacgaaccccccgttcagcccgaccgctgc |
| | | | gccttatccggtaactatcgtcttgagtccaacccggtaagacacgacttatcgccactggcagcagccactggtaacaggattagcagagcgaggtatg |
| | | | taggcggtgctacagagttcttgaagtggtggcctaactacggctacactagaagaacagtatttggtatctgcgctctgctgaagccagttaccttcgga |
| | | | aaaagagttggtagctcttgatccggcaaacaaaccaccgctggtagcggtggtttttttgtttgcaagcagcagattacgcgcagaaaaaaaggatctc |
| | | | aagaagatcctttgatcttttctacggggtctgacgctcagtggaacgaaaactcacgttaagggattttggtcatgagattatcaaaaaggatcttcacc |
| | | | tagatccttttaaattaaaaatgaagttttaaatcaagcccaatctgaataatgttacaaccaattaaccaattctgattagaaaaactcatcgagcatca |
| | | | aatgaaactgcaatttattcatatcaggattatcaataccatatttttgaaaaagccgtttctgtaatgaaggagaaaactcaccgaggcagttccatagg |
| | | | atggcaagatcctggtatcggtctgcgattccgactcgtccaacatcaatacaacctattaatttcccctcgtcaaaaataaggttatcaagtgagaaatc |
| | | | accatgagtgacgactgaatccggtgagaatggcaaaagtttatgcatttctttccagacttgttcaacaggccagccattacgctcgtcatcaaaatcac |
| | | | tcgcatcaaccaaaccgttattcattcgtgattgcgcctgagcgagacgaaatacgcgatcgctgttaaaaggacaattacaaacaggaatcgaatgca |
| | | | accggcgcaggaacactgccagcgcatcaacaatattttcacctgaatcaggatattcttctaatacctggaatgctgtttttccggggatcgcagtggtg |
| | | | agtaaccatgcatcatcaggagtacggataaaatgcttgatggtcggaagaggcataaattccgtcagccagtttagtctgaccatctcatctgtaacat |
| | | | cattggcaacgctacctttgccatgtttcagaaacaactctggcgcatcgggcttcccatacaagcgatagattgtcgcacctgattgcccgacattatcgc |
| | | | gagcccatttatacccatataaatcagcatccatgttggaatttaatcgcggcctcgacgtttcccgttgaatatggctcataacaccccttgtattactgtt |
| | | | tatgtaagcagacagttttattgttcatgatgatatatttttatcttgtgcaatgtaacatcagagattttgagacacgggccagagctgcatcgcgcgtttc |
| | | | ggtgatgacggtgaaaacctctgacacatgcagctcccggagacggtcacagcttgtctgtaagcggatgccgggagcagacaagcccgtcagggcgc |
| | | | gtcagcgggtgttggcgggtgtcggggctggcttaactatgcggcatcagagcagattgtactgagagtgcaccatatgcggtgtgaaataccgcacag |
| | | | atgcgtaaggagaaaataccgcatcaggcgccattcgccattcaggctgcgcaactgttgggaagggcgatcggtgcgggcctcttcgctattacgcca |
| | | | gctggcgaaagggggatgtgctgcaaggcgattaagttgggtaacgccagggttttcccagtcacgacgttgtaaaacgacggccagagaattcTTGG |
| | | | CCACTCCCTCTCTGCGCGCTCGCTCGCTCACTGAGGCCGGGCGACCAAAGGTCGCCCGACGCCCGGGCTTTGCCCGG |
| | | | GCGGCCTCAGTGAGCGAGCGAGCGCGCAGAGAGGGAGTGGCCAACTCCATCACTAGGGGTTCCTAGATCTACTAG |
| | | | Ttaggtcagtgaagagaagaacaaaaagcagcatattacagttagttgtcttcatcaatctttaaatatgttgtgtggtttttctctccctgtttccacagtt |
| | | | GAGGACCCCCAGGGCGACGCTGCCCAGAAGACGGACACGTCGCACCACGACCAGGACCACCCCACCTTCAACAAGA |
| | | | TCACTCCCAATCTCGCGGAGTTCGCGTTCTCGCTCTACCGCCAGCTCGCGCACCAGAGCAACTCGACTAACATCTTCT |
| | | | TCTCGCCCGTCAGCATCGCGACGGCGTTCGCGATGCTCAGCCTCGGCACGAAGGCGGACACGCACGACGAGATCCT |
| | | | CGAGGGCCTCAACTTCAATCTCACAGAGATCCCAGAAGCCCAGATCCACGAGGGCTTCCAGGAGCTGCTGCGGACG |
| | | | CTCAACCAGCCTGACTCGCAGCTCCAGCTCACGACGGGCAATGGGCTCTTCCTCAGCGAGGGCCTCAAGCTCGTCGA |
| | | | CAAGTTCCTGGAGGACGTCAAGAAGCTCTACCACTCGGAAGCCTTCACGGTCAACTTCGGCGACACAGAGGAAGCC |
| | | | AAGAAGCAGATCAACGACTACGTCGAGAAGGGGACTCAGGGCAAGATCGTCGACCTCGTCAAGGAGCTGGACCGA |
| | | | GACACGGTCTTCGCACTGGTCAACTACATCTTCTTCAAGGGGAAGTGGGAGCGCCCCTTCGAAGTCAAGGACACAG |
| | | | AGGAGGAGGACTTCCACGTCGACCAGGTGACGACGGTCAAGGTTCCCATGATGAAGCGCCTCGGCATGTTCAACAT |
| | | | CCAGCACTGCAAGAAGCTCAGCTCGTGGGTCCTCCTCATGAAGTACCTCGGCAACGCGACGGCGATCTTCTTCCTTC |
| | | | CTGACGAGGGCAAGCTCCAGCACCTCGAGAACGAGCTGACGCACGACATCATCACGAAGTTCCTGGAGAACGAGG |
| | | | ACCGCCGATCGGCGTCGCTCCACCTTCCAAAGCTCAGCATCACGGGCACCTACGACCTCAAGTCGGTCCTCGGCCAG |
| | | | CTCGGCATCACGAAGGTCTTCTCGAATGGTGCCGACCTCAGCGGCGTCACAGAGGAAGCCCCCCTCAAGCTCAGCA |
| | | | AGGCTGTGCACAAGGCTGTGCTCACGATCGACGAGAAGGGGACAGAGGCTGCCGGTGCCATGTTCCTGGAAGCCA |
| | | | TCCCCATGAGCATCCCACCAGAAGTCAAGTTCAACAAGCCTTTCGTCTTCCTGATGATAGAGCAGAACACGAAGTCG |
| | | | CCCCTCTTCATGGGCAAGGTCGTCAACCCCACTCAAAAGTAACAGACATGATAAGATACATTGATGAGTTTGGACAA |
| | | | ACCACAACTAGAATGCAGTGAAAAAAATGCTTTATTTGTGAAATTTGTGATGCTATTGCTTTATTTGTAACCATTATA |
| | | | AGCTGCAATAAACAAGTTAACAACAACAATTGCATTCATTTTATGTTTCAGGTTCAGGGGGAGGTGTGGGAGGTTTT |
| | | | TTggggataccccctagagccccagctggttctttccgcctcagaagCCATAGAGCCCACCGCATCCCCAGCATGCCTGCTATTGTCT |
| | | | TCCCAATCCTCCCCCTTGCTGTCCTGCCCCACCCCACCCCCCAGAATAGAATGACACCTACTCAGACAATGCGATGCA |
| | | | ATTTCCTCATTTTATTAGGAAAGGACAGTGGGAGTGGCACCTTCCAGGGTCAAGGAAGGCACGGGGGAGGGGCAA |
| | | | ACAACAGATGGCTGGCAACTAGAAGGCACAGTCGaggTTACTTCTGGGTGGGGTTCACCACCTTGCCCATGAACAGG |
| | | | GGGCTCTTGGTGTTCTGCTCGATCATCAGGAACACGAAAGGCTTGTTGAACTTCACCTCGGGGGGGATGCTCATGG |
| | | | GGATGGCCTCCAGGAACATGGCGCCGGCGGCCTCGGTGCCCTTCTCGTCGATGGTCAGCACGGCCTTGTGCACGGC |
| | | | CTTGCTCAGCTTCAGGGGGGCCTCCTCGGTCACGCCGCTCAGGTCGGCGCCGTTGCTGAACACCTTGGTGATGCCCA |
| | | | GCTGGCCCAGCACGCTCTTCAGGTCGTAGGTGCCGGTGATGCTCAGCTTGGGCAGGTGCAGGCTGGCGCTCCTCCT |
| | | | GTCCTCGTTCTCCAGGAACTTGGTGATGATGTCGTGGGTCAGCTCGTTCTCCAGGTGCTGCAGCTTGCCCTCGTCGG |
| | | | GCAGGAAGAAGATGGCGGTGGCGTTGCCCAGGTACTTCATCAGCAGCACCCAGCTGCTCAGCTTCTTGCAGTGCTG |
| | | | GATATTGAACATGCCCAGCCTCTTCATCATGGGCACCTTCACGGTGGTCACCTGGTCCACGTGGAAGTCCTCCTCCTC |
| | | | GGTGTCCTTCACCTCGAAGGGCCTCTCCCACTTGCCCTTGAAGAAGATGTAGTTCACCAGGGCGAACACGGTGTCCC |
| | | | TGTCCAGCTCCTTCACCAGGTCCACGATCTTGCCCTGGGTGCCCTTCTCCACGTAGTCGTTGATCTGCTTCTTGGCCTC |
| | | | CTCGGTGTCGCCGAAGTTCACGGTGAAGGCCTCGCTGTGGTACAGCTTCTTCACGTCCTCCAGGAACTTGTCCACCA |
| | | | GCTTCAGGCCCTCGCTCAGGAACAGGCCGTTGCCGGTGGTCAGCTGCAGCTGGCTGTCGGGCTGGTTCAGGGTCCT |
| | | | CAGCAGCTCCTGGAAGCCCTCGTGGATCTGGGCCTCGGGGATCTCGGTCAGGTTGAAGTTCAGGCCCTCCAGGATC |
| | | | TCGTCGTGGGTGTCGGCCTTGGTGCCCAGGCTCAGCATGGCGAAGGCGGTGGCGATGCTCACGGGGCTGAAGAAG |
| | | | ATGTTGGTGCTGTTGCTCTGGTGGGCCAGCTGCCTGTACAGGCTGAAGGCGAACTCGGCCAGGTTGGGGGTGATCT |
| | | | TGTTGAAGGTGGGGTGGTCCTGGTCGTGGTGGCTGGTGTCGGTCTTCTGGGCGGCGTCGCCCTGGGGGTCCTCaact |
| | | | gtggaaacagggagagaaaaaccacacaacatatttaaagattgatgaagacaactaactgtaatatgctgctttttgttcttctcttcactgacctaAC |
| | | | TAGTAGATCTAGGAACCCCTAGTGATGGAGTTGGCCACTCCCTCTCTGCGCGCTCGCTCGCTCACTGAGGCCGCCCG |
| | | | GGCAAAGCCCGGGCGTCGGGCGACCTTTGGTCGCCCGGCCTCAGTGAGCGAGCGAGCGCGCAGAGAGGGAGTGG |
| | | | CCAAacgcgtggtgtaatcatggtcatagctgtttcctgtgtgaaattgttatccgctcacaattccacacaacatacgagccggaagcataaagtgtaa |
| | | | agcctggggtgcctaatgagtgagctaactcacattaattgcgttgcgctcactgcccgctttccagtcgggaaacctgtcgtgccagctgcattaatgaa |
| | | | tcggccaacgcgcggggagaggcggtttgcgtattgggcgctcttccgcttcctcgctcactgactcgctgcgctcggtcgttcggctgcggcgagcggta |
| | | | tcagctca |
| | SERPINA1 | A1AT w/o | GAGGACCCCCAGGGCGACGCTGCCCAGAAGACGGACACGTCGCACCACGACCAGGACCACCCCACCTTCAACAAGA |
| | copy 1 | SP | TCACTCCCAATCTCGCGGAGTTCGCGTTCTCGCTCTACCGCCAGCTCGCGCACCAGAGCAACTCGACTAACATCTTCT |
| | | (alternate | TCTCGCCCGTCAGCATCGCGACGGCGTTCGCGATGCTCAGCCTCGGCACGAAGGCGGACACGCACGACGAGATCCT |
| | | codon usage | CGAGGGCCTCAACTTCAATCTCACAGAGATCCCAGAAGCCCAGATCCACGAGGGCTTCCAGGAGCTGCTGCGGACG |
| | | 2) | CTCAACCAGCCTGACTCGCAGCTCCAGCTCACGACGGGCAATGGGCTCTTCCTCAGCGAGGGCCTCAAGCTCGTCGA |
| | | (SEQ ID NO: | CAAGTTCCTGGAGGACGTCAAGAAGCTCTACCACTCGGAAGCCTTCACGGTCAACTTCGGCGACACAGAGGAAGCC |
| | | 741) | AAGAAGCAGATCAACGACTACGTCGAGAAGGGGACTCAGGGCAAGATCGTCGACCTCGTCAAGGAGCTGGACCGA |
| | | | GACACGGTCTTCGCACTGGTCAACTACATCTTCTTCAAGGGGAAGTGGGAGCGCCCCTTCGAAGTCAAGGACACAG |
| | | | AGGAGGAGGACTTCCACGTCGACCAGGTGACGACGGTCAAGGTTCCCATGATGAAGCGCCTCGGCATGTTCAACAT |
| | | | CCAGCACTGCAAGAAGCTCAGCTCGTGGGTCCTCCTCATGAAGTACCTCGGCAACGCGACGGCGATCTTCTTCCTTC |
| | | | CTGACGAGGGCAAGCTCCAGCACCTCGAGAACGAGCTGACGCACGACATCATCACGAAGTTCCTGGAGAACGAGG |
| | | | ACCGCCGATCGGCGTCGCTCCACCTTCCAAAGCTCAGCATCACGGGCACCTACGACCTCAAGTCGGTCCTCGGCCAG |
| | | | CTCGGCATCACGAAGGTCTTCTCGAATGGTGCCGACCTCAGCGGCGTCACAGAGGAAGCCCCCCTCAAGCTCAGCA |
| | | | AGGCTGTGCACAAGGCTGTGCTCACGATCGACGAGAAGGGGACAGAGGCTGCCGGTGCCATGTTCCTGGAAGCCA |
| | | | TCCCCATGAGCATCCCACCAGAAGTCAAGTTCAACAAGCCTTTCGTCTTCCTGATGATAGAGCAGAACACGAAGTCG |
| | | | CCCCTCTTCATGGGCAAGGTCGTCAACCCCACTCAAAAG |
| | SERPINA1 | A1AT w/o | GAGGACCCCCAGGGCGACGCCGCCCAGAAGACCGACACCAGCCACCACGACCAGGACCACCCCACCTTCAACAAGA |
| | copy 2 (rev | SP | TCACCCCCAACCTGGCCGAGTTCGCCTTCAGCCTGTACAGGCAGCTGGCCCACCAGAGCAACAGCACCAACATCTTC |
| | comp) | (alternate | TTCAGCCCCGTGAGCATCGCCACCGCCTTCGCCATGCTGAGCCTGGGCACCAAGGCCGACACCCACGACGAGATCCT |
| | | codon usage | GGAGGGCCTGAACTTCAACCTGACCGAGATCCCCGAGGCCCAGATCCACGAGGGCTTCCAGGAGCTGCTGAGGACC |
| | | 1) | CTGAACCAGCCCGACAGCCAGCTGCAGCTGACCACCGGCAACGGCCTGTTCCTGAGCGAGGGCCTGAAGCTGGTGG |
| | | (SEQ ID NO: | ACAAGTTCCTGGAGGACGTGAAGAAGCTGTACCACAGCGAGGCCTTCACCGTGAACTTCGGCGACACCGAGGAGG |
| | | 742) | CCAAGAAGCAGATCAACGACTACGTGGAGAAGGGCACCCAGGGCAAGATCGTGGACCTGGTGAAGGAGCTGGAC |
| | | | AGGGACACCGTGTTCGCCCTGGTGAACTACATCTTCTTCAAGGGCAAGTGGGAGAGGCCCTTCGAGGTGAAGGACA |
| | | | CCGAGGAGGAGGACTTCCACGTGGACCAGGTGACCACCGTGAAGGTGCCCATGATGAAGAGGCTGGGCATGTTCA |
| | | | ATATCCAGCACTGCAAGAAGCTGAGCAGCTGGGTGCTGCTGATGAAGTACCTGGGCAACGCCACCGCCATCTTCTTC |
| | | | CTGCCCGACGAGGGCAAGCTGCAGCACCTGGAGAACGAGCTGACCCACGACATCATCACCAAGTTCCTGGAGAACG |
| | | | AGGACAGGAGGAGCGCCAGCCTGCACCTGCCCAAGCTGAGCATCACCGGCACCTACGACCTGAAGAGCGTGCTGG |
| | | | GCCAGCTGGGCATCACCAAGGTGTTCAGCAACGGCGCCGACCTGAGCGGCGTGACCGAGGAGGCCCCCCTGAAGC |
| | | | TGAGCAAGGCCGTGCACAAGGCCGTGCTGACCATCGACGAGAAGGGCACCGAGGCCGCCGGCGCCATGTTCCTGG |
| | | | AGGCCATCCCCATGAGCATCCCCCCCGAGGTGAAGTTCAACAAGCCTTTCGTGTTCCTGATGATCGAGCAGAACACC |
| | | | AAGAGCCCCCTGTTCATGGGCAAGGTGGTGAACCCCACCCAGAAGTAA |
| 5 | Full | (SEQ ID NO: | TTGGCCACTCCCTCTCTGCGCGCTCGCTCGCTCACTGAGGCCGGGCGACCAAAGGTCGCCCGACGCCCGGGCTTTGC |
| | Sequence | 750) | CCGGGCGGCCTCAGTGAGCGAGCGAGCGCGCAGAGAGGGAGTGGCCAACTCCATCACTAGGGGTTCCTAGATCTA |
| | | | CTAGTATAACTTCGTATAGCATACATTATACGAAGTTATATGTATGCtaggtcagtgaagagaagaacaaaaagcagcatattaca |
| | | | gttagttgtcttcatcaatctttaaatatgttgtgtggtttttctctccctgtttccacagttGAGGACCCCCAGGGCGACGCTGCCCAGAAGA |
| | | | CGGACACGTCGCACCACGACCAGGACCACCCCACCTTCAACAAGATCACTCCCAATCTCGCGGAGTTCGCGTTCTCG |
| | | | CTCTACCGCCAGCTCGCGCACCAGAGCAACTCGACTAACATCTTCTTCTCGCCCGTCAGCATCGCGACGGCGTTCGCG |
| | | | ATGCTCAGCCTCGGCACGAAGGCGGACACGCACGACGAGATCCTCGAGGGCCTCAACTTCAATCTCACAGAGATCC |
| | | | CAGAAGCCCAGATCCACGAGGGCTTCCAGGAGCTGCTGCGGACGCTCAACCAGCCTGACTCGCAGCTCCAGCTCAC |
| | | | GACGGGCAATGGGCTCTTCCTCAGCGAGGGCCTCAAGCTCGTCGACAAGTTCCTGGAGGACGTCAAGAAGCTCTAC |
| | | | CACTCGGAAGCCTTCACGGTCAACTTCGGCGACACAGAGGAAGCCAAGAAGCAGATCAACGACTACGTCGAGAAG |
| | | | GGGACTCAGGGCAAGATCGTCGACCTCGTCAAGGAGCTGGACCGAGACACGGTCTTCGCACTGGTCAACTACATCT |
| | | | TCTTCAAGGGGAAGTGGGAGCGCCCCTTCGAAGTCAAGGACACAGAGGAGGAGGACTTCCACGTCGACCAGGTGA |
| | | | CGACGGTCAAGGTTCCCATGATGAAGCGCCTCGGCATGTTCAACATCCAGCACTGCAAGAAGCTCAGCTCGTGGGT |
| | | | CCTCCTCATGAAGTACCTCGGCAACGCGACGGCGATCTTCTTCCTTCCTGACGAGGGCAAGCTCCAGCACCTCGAGA |
| | | | ACGAGCTGACGCACGACATCATCACGAAGTTCCTGGAGAACGAGGACCGCCGATCGGCGTCGCTCCACCTTCCAAA |
| | | | GCTCAGCATCACGGGCACCTACGACCTCAAGTCGGTCCTCGGCCAGCTCGGCATCACGAAGGTCTTCTCGAATGGTG |
| | | | CCGACCTCAGCGGCGTCACAGAGGAAGCCCCCCTCAAGCTCAGCAAGGCTGTGCACAAGGCTGTGCTCACGATCGA |
| | | | CGAGAAGGGGACAGAGGCTGCCGGTGCCATGTTCCTGGAAGCCATCCCCATGAGCATCCCACCAGAAGTCAAGTTC |
| | | | AACAAGCCTTTCGTCTTCCTGATGATAGAGCAGAACACGAAGTCGCCCCTCTTCATGGGCAAGGTCGTCAACCCCAC |
| | | | TCAAAAGTAACAGACATGATAAGATACATTGATGAGTTTGGACAAACCACAACTAGAATGCAGTGAAAAAAATGCT |
| | | | TTATTTGTGAAATTTGTGATGCTATTGCTTTATTTGTAACCATTATAAGCTGCAATAAACAAGTTAACAACAACAATTG |
| | | | CATTCATTTTATGTTTCAGGTTCAGGGGGAGGTGTGGGAGGTTTTTTggggataccccctagagccccagctggttctttccgcctc |
| | | | agaagCCATAGAGCCCACCGCATCCCCAGCATGCCTGCTATTGTCTTCCCAATCCTCCCCCTTGCTGTCCTGCCCCACCC |
| | | | CACCCCCCAGAATAGAATGACACCTACTCAGACAATGCGATGCAATTTCCTCATTTTATTAGGAAAGGACAGTGGGA |
| | | | GTGGCACCTTCCAGGGTCAAGGAAGGCACGGGGGAGGGGCAAACAACAGATGGCTGGCAACTAGAAGGCACAGT |
| | | | CGaggTTACTTCTGGGTGGGGTTCACCACCTTGCCCATGAACAGGGGGCTCTTGGTGTTCTGCTCGATCATCAGGAAC |
| | | | ACGAAAGGCTTGTTGAACTTCACCTCGGGGGGGATGCTCATGGGGATGGCCTCCAGGAACATGGCGCCGGCGGCC |
| | | | TCGGTGCCCTTCTCGTCGATGGTCAGCACGGCCTTGTGCACGGCCTTGCTCAGCTTCAGGGGGGCCTCCTCGGTCAC |
| | | | GCCGCTCAGGTCGGCGCCGTTGCTGAACACCTTGGTGATGCCCAGCTGGCCCAGCACGCTCTTCAGGTCGTAGGTG |
| | | | CCGGTGATGCTCAGCTTGGGCAGGTGCAGGCTGGCGCTCCTCCTGTCCTCGTTCTCCAGGAACTTGGTGATGATGTC |
| | | | GTGGGTCAGCTCGTTCTCCAGGTGCTGCAGCTTGCCCTCGTCGGGCAGGAAGAAGATGGCGGTGGCGTTGCCCAGG |
| | | | TACTTCATCAGCAGCACCCAGCTGCTCAGCTTCTTGCAGTGCTGGATATTGAACATGCCCAGCCTCTTCATCATGGGC |
| | | | ACCTTCACGGTGGTCACCTGGTCCACGTGGAAGTCCTCCTCCTCGGTGTCCTTCACCTCGAAGGGCCTCTCCCACTTG |
| | | | CCCTTGAAGAAGATGTAGTTCACCAGGGCGAACACGGTGTCCCTGTCCAGCTCCTTCACCAGGTCCACGATCTTGCC |
| | | | CTGGGTGCCCTTCTCCACGTAGTCGTTGATCTGCTTCTTGGCCTCCTCGGTGTCGCCGAAGTTCACGGTGAAGGCCTC |
| | | | GCTGTGGTACAGCTTCTTCACGTCCTCCAGGAACTTGTCCACCAGCTTCAGGCCCTCGCTCAGGAACAGGCCGTTGC |
| | | | CGGTGGTCAGCTGCAGCTGGCTGTCGGGCTGGTTCAGGGTCCTCAGCAGCTCCTGGAAGCCCTCGTGGATCTGGGC |
| | | | CTCGGGGATCTCGGTCAGGTTGAAGTTCAGGCCCTCCAGGATCTCGTCGTGGGTGTCGGCCTTGGTGCCCAGGCTC |
| | | | AGCATGGCGAAGGCGGTGGCGATGCTCACGGGGCTGAAGAAGATGTTGGTGCTGTTGCTCTGGTGGGCCAGCTGC |
| | | | CTGTACAGGCTGAAGGCGAACTCGGCCAGGTTGGGGGTGATCTTGTTGAAGGTGGGGTGGTCCTGGTCGTGGTGG |
| | | | CTGGTGTCGGTCTTCTGGGCGGCGTCGCCCTGGGGGTCCTCaactgtggaaacagggagagaaaaaccacacaacatatttaaag |
| | | | attgatgaagacaactaactgtaatatgctgctttttgttcttctcttcactgacctaATGTATGCATAACTTCGTATAGCATACATTATACG |
| | | | AAGTTATACTAGTAGATCTAGGAACCCCTAGTGATGGAGTTGGCCACTCCCTCTCTGCGCGCTCGCTCGCTCACTGA |
| | | | GGCCGCCCGGGCAAAGCCCGGGCGTCGGGCGACCTTTGGTCGCCCGGCCTCAGTGAGCGAGCGAGCGCGCAGAGA |
| | | | GGGAGTGGCCAAacgcgtggtgtaatcatggtcatagctgtttcctgtgtgaaattgttatccgctcacaattccacacaacatacgagccggaagc |
| | | | ataaagtgtaaagcctggggtgcctaatgagtgagctaactcacattaattgcgttgcgctcactgcccgctttccagtcgggaaacctgtcgtgccagct |
| | | | gcattaatgaatcggccaacgcgcggggagaggcggtttgcgtattgggcgctcttccgcttcctcgctcactgactcgctgcgctcggtcgttcggctgc |
| | | | ggcgagcggtatcagctcactcaaaggcggtaatacggttatccacagaatcaggggataacgcaggaaagaacatgtgagcaaaaggccagcaaa |
| | | | aggccaggaaccgtaaaaaggccgcgttgctggcgtttttccataggctccgcccccctgacgagcatcacaaaaatcgacgctcaagtcagaggtggc |
| | | | gaaacccgacaggactataaagataccaggcgtttccccctggaagctccctcgtgcgctctcctgttccgaccctgccgcttaccggatacctgtccgcc |
| | | | tttctcccttcgggaagcgtggcgctttctcatagctcacgctgtaggtatctcagttcggtgtaggtcgttcgctccaagctgggctgtgtgcacgaacccc |
| | | | ccgttcagcccgaccgctgcgccttatccggtaactatcgtcttgagtccaacccggtaagacacgacttatcgccactggcagcagccactggtaacag |
| | | | gattagcagagcgaggtatgtaggcggtgctacagagttcttgaagtggtggcctaactacggctacactagaagaacagtatttggtatctgcgctctg |
| | | | ctgaagccagttaccttcggaaaaagagttggtagctcttgatccggcaaacaaaccaccgctggtagcggtggtttttttgtttgcaagcagcagattac |
| | | | gcgcagaaaaaaaggatctcaagaagatcctttgatcttttctacggggtctgacgctcagtggaacgaaaactcacgttaagggattttggtcatgaga |
| | | | ttatcaaaaaggatcttcacctagatccttttaaattaaaaatgaagttttaaatcaagcccaatctgaataatgttacaaccaattaaccaattctgatta |
| | | | gaaaaactcatcgagcatcaaatgaaactgcaatttattcatatcaggattatcaataccatatttttgaaaaagccgtttctgtaatgaaggagaaaact |
| | | | caccgaggcagttccataggatggcaagatcctggtatcggtctgcgattccgactcgtccaacatcaatacaacctattaatttcccctcgtcaaaaata |
| | | | aggttatcaagtgagaaatcaccatgagtgacgactgaatccggtgagaatggcaaaagtttatgcatttctttccagacttgttcaacaggccagccatt |
| | | | acgctcgtcatcaaaatcactcgcatcaaccaaaccgttattcattcgtgattgcgcctgagcgagacgaaatacgcgatcgctgttaaaaggacaatta |
| | | | caaacaggaatcgaatgcaaccggcgcaggaacactgccagcgcatcaacaatattttcacctgaatcaggatattcttctaatacctggaatgctgttt |
| | | | ttccggggatcgcagtggtgagtaaccatgcatcatcaggagtacggataaaatgcttgatggtcggaagaggcataaattccgtcagccagtttagtct |
| | | | gaccatctcatctgtaacatcattggcaacgctacctttgccatgtttcagaaacaactctggcgcatcgggcttcccatacaagcgatagattgtcgcac |
| | | | ctgattgcccgacattatcgcgagcccatttatacccatataaatcagcatccatgttggaatttaatcgcggcctcgacgtttcccgttgaatatggctcat |
| | | | aacaccccttgtattactgtttatgtaagcagacagttttattgttcatgatgatatatttttatcttgtgcaatgtaacatcagagattttgagacacgggcc |
| | | | agagctgcatcgcgcgtttcggtgatgacggtgaaaacctctgacacatgcagctcccggagacggtcacagcttgtctgtaagcggatgccgggagca |
| | | | gacaagcccgtcagggcgcgtcagcgggtgttggcgggtgtcggggctggcttaactatgcggcatcagagcagattgtactgagagtgcaccatatgc |
| | | | ggtgtgaaataccgcacagatgcgtaaggagaaaataccgcatcaggcgccattcgccattcaggctgcgcaactgttgggaagggcgatcggtgcgg |
| | | | gcctcttcgctattacgccagctggcgaaagggggatgtgctgcaaggcgattaagttgggtaacgccagggttttcccagtcacgacgttgtaaaacga |
| | | | cggccagagaattc |
| | SERPINA1 | AAT w/o SP | GAGGACCCCCAGGGCGACGCTGCCCAGAAGACGGACACGTCGCACCACGACCAGGACCACCCCACCTTCAACAAGA |
| | copy 1 | (alternate | TCACTCCCAATCTCGCGGAGTTCGCGTTCTCGCTCTACCGCCAGCTCGCGCACCAGAGCAACTCGACTAACATCTTCT |
| | | codon usage | TCTCGCCCGTCAGCATCGCGACGGCGTTCGCGATGCTCAGCCTCGGCACGAAGGCGGACACGCACGACGAGATCCT |
| | | 2) | CGAGGGCCTCAACTTCAATCTCACAGAGATCCCAGAAGCCCAGATCCACGAGGGCTTCCAGGAGCTGCTGCGGACG |
| | | (SEQ ID NO: | CTCAACCAGCCTGACTCGCAGCTCCAGCTCACGACGGGCAATGGGCTCTTCCTCAGCGAGGGCCTCAAGCTCGTCGA |
| | | 751) | CAAGTTCCTGGAGGACGTCAAGAAGCTCTACCACTCGGAAGCCTTCACGGTCAACTTCGGCGACACAGAGGAAGCC |
| | | | AAGAAGCAGATCAACGACTACGTCGAGAAGGGGACTCAGGGCAAGATCGTCGACCTCGTCAAGGAGCTGGACCGA |
| | | | GACACGGTCTTCGCACTGGTCAACTACATCTTCTTCAAGGGGAAGTGGGAGCGCCCCTTCGAAGTCAAGGACACAG |
| | | | AGGAGGAGGACTTCCACGTCGACCAGGTGACGACGGTCAAGGTTCCCATGATGAAGCGCCTCGGCATGTTCAACAT |
| | | | CCAGCACTGCAAGAAGCTCAGCTCGTGGGTCCTCCTCATGAAGTACCTCGGCAACGCGACGGCGATCTTCTTCCTTC |
| | | | CTGACGAGGGCAAGCTCCAGCACCTCGAGAACGAGCTGACGCACGACATCATCACGAAGTTCCTGGAGAACGAGG |
| | | | ACCGCCGATCGGCGTCGCTCCACCTTCCAAAGCTCAGCATCACGGGCACCTACGACCTCAAGTCGGTCCTCGGCCAG |
| | | | CTCGGCATCACGAAGGTCTTCTCGAATGGTGCCGACCTCAGCGGCGTCACAGAGGAAGCCCCCCTCAAGCTCAGCA |
| | | | AGGCTGTGCACAAGGCTGTGCTCACGATCGACGAGAAGGGGACAGAGGCTGCCGGTGCCATGTTCCTGGAAGCCA |
| | | | TCCCCATGAGCATCCCACCAGAAGTCAAGTTCAACAAGCCTTTCGTCTTCCTGATGATAGAGCAGAACACGAAGTCG |
| | | | CCCCTCTTCATGGGCAAGGTCGTCAACCCCACTCAAAAG |
| | SERPINA1 | A1AT w/o | GAGGACCCCCAGGGCGACGCCGCCCAGAAGACCGACACCAGCCACCACGACCAGGACCACCCCACCTTCAACAAGA |
| | copy 2 (rev | SP | TCACCCCCAACCTGGCCGAGTTCGCCTTCAGCCTGTACAGGCAGCTGGCCCACCAGAGCAACAGCACCAACATCTTC |
| | comp) | (alternate | TTCAGCCCCGTGAGCATCGCCACCGCCTTCGCCATGCTGAGCCTGGGCACCAAGGCCGACACCCACGACGAGATCCT |
| | | codon usage | GGAGGGCCTGAACTTCAACCTGACCGAGATCCCCGAGGCCCAGATCCACGAGGGCTTCCAGGAGCTGCTGAGGACC |
| | | 1) | CTGAACCAGCCCGACAGCCAGCTGCAGCTGACCACCGGCAACGGCCTGTTCCTGAGCGAGGGCCTGAAGCTGGTGG |
| | | (SEQ ID NO: | ACAAGTTCCTGGAGGACGTGAAGAAGCTGTACCACAGCGAGGCCTTCACCGTGAACTTCGGCGACACCGAGGAGG |
| | | 752) | CCAAGAAGCAGATCAACGACTACGTGGAGAAGGGCACCCAGGGCAAGATCGTGGACCTGGTGAAGGAGCTGGAC |
| | | | AGGGACACCGTGTTCGCCCTGGTGAACTACATCTTCTTCAAGGGCAAGTGGGAGAGGCCCTTCGAGGTGAAGGACA |
| | | | CCGAGGAGGAGGACTTCCACGTGGACCAGGTGACCACCGTGAAGGTGCCCATGATGAAGAGGCTGGGCATGTTCA |
| | | | ATATCCAGCACTGCAAGAAGCTGAGCAGCTGGGTGCTGCTGATGAAGTACCTGGGCAACGCCACCGCCATCTTCTTC |
| | | | CTGCCCGACGAGGGCAAGCTGCAGCACCTGGAGAACGAGCTGACCCACGACATCATCACCAAGTTCCTGGAGAACG |
| | | | AGGACAGGAGGAGCGCCAGCCTGCACCTGCCCAAGCTGAGCATCACCGGCACCTACGACCTGAAGAGCGTGCTGG |
| | | | GCCAGCTGGGCATCACCAAGGTGTTCAGCAACGGCGCCGACCTGAGCGGCGTGACCGAGGAGGCCCCCCTGAAGC |
| | | | TGAGCAAGGCCGTGCACAAGGCCGTGCTGACCATCGACGAGAAGGGCACCGAGGCCGCCGGCGCCATGTTCCTGG |
| | | | AGGCCATCCCCATGAGCATCCCCCCCGAGGTGAAGTTCAACAAGCCTTTCGTGTTCCTGATGATCGAGCAGAACACC |
| | | | AAGAGCCCCCTGTTCATGGGCAAGGTGGTGAACCCCACCCAGAAGTAA |
| 6 | Full | (SEQ ID NO: | TTGGCCACTCCCTCTCTGCGCGCTCGCTCGCTCACTGAGGCCGGGCGACCAAAGGTCGCCCGACGCCCGGGCTTTGC |
| | Sequence | 760) | CCGGGCGGCCTCAGTGAGCGAGCGAGCGCGCAGAGAGGGAGTGGCCAACTCCATCACTAGGGGTTCCTAGATCTA |
| | | | CTAGTtaggtcagtgaagagaagaacaaaaagcagcatattacagttagttgtcttcatcaatctttaaatatgttgtgtggtttttctctccctgtttcca |
| | | | cagttGAGGACCCCCAGGGAGATGCAGCCCAGAAGACAGACACCAGCCACCATGACCAGGACCACCCCACCTTCAAC |
| | | | AAGATCACCCCCAACCTGGCAGAGTTTGCCTTCAGCCTGTACAGGCAGCTGGCCCACCAGAGCAACAGCACCAACAT |
| | | | CTTCTTCAGCCCAGTGAGCATAGCCACAGCCTTTGCCATGCTGAGCCTGGGCACCAAGGCAGACACCCATGATGAGA |
| | | | TCCTGGAGGGCCTGAACTTCAACCTGACAGAGATCCCAGAGGCCCAGATCCATGAGGGCTTCCAGGAGCTGCTGAG |
| | | | GACCCTGAACCAGCCAGACAGCCAGCTGCAGCTGACCACAGGCAATGGCCTGTTCCTGTCTGAGGGCCTGAAGCTG |
| | | | GTGGACAAGTTCCTGGAGGATGTGAAGAAGCTGTACCACTCTGAGGCCTTCACAGTGAACTTTGGAGACACAGAGG |
| | | | AGGCCAAGAAGCAGATCAATGACTATGTGGAGAAGGGCACCCAGGGCAAGATAGTGGACCTGGTGAAGGAGCTG |
| | | | GACAGGGACACAGTGTTTGCCCTGGTGAACTACATCTTCTTCAAGGGCAAGTGGGAGAGGCCCTTTGAGGTGAAGG |
| | | | ACACAGAGGAGGAGGACTTCCATGTGGACCAGGTGACCACAGTGAAGGTGCCCATGATGAAGAGGCTGGGCATGT |
| | | | TCAACATCCAGCACTGCAAGAAGCTGAGCAGCTGGGTGCTGCTGATGAAGTACCTGGGCAATGCCACAGCCATCTT |
| | | | CTTCCTGCCAGATGAGGGCAAGCTGCAGCACCTGGAGAATGAGCTGACCCATGACATCATCACCAAGTTCCTGGAG |
| | | | AATGAGGACAGGAGGTCTGCCAGCCTGCACCTGCCCAAGCTGAGCATCACAGGCACCTATGACCTGAAGTCTGTGC |
| | | | TGGGCCAGCTGGGCATCACCAAGGTGTTCAGCAATGGAGCAGACCTGTCTGGAGTGACAGAGGAGGCCCCCCTGA |
| | | | AGCTGAGCAAGGCAGTGCACAAGGCAGTGCTGACCATAGATGAGAAGGGCACAGAGGCAGCAGGAGCCATGTTCC |
| | | | TGGAGGCCATCCCCATGAGCATCCCCCCAGAGGTGAAGTTCAACAAGCCCTTTGTGTTCCTGATGATAGAGCAGAAC |
| | | | ACCAAGAGCCCCCTGTTCATGGGCAAGGTGGTGAACCCCACCCAGAAGTAACAGACATGATAAGATACATTGATGA |
| | | | GTTTGGACAAACCACAACTAGAATGCAGTGAAAAAAATGCTTTATTTGTGAAATTTGTGATGCTATTGCTTTATTTGT |
| | | | AACCATTATAAGCTGCAATAAACAAGTTAACAACAACAATTGCATTCATTTTATGTTTCAGGTTCAGGGGGAGGTGT |
| | | | GGGAGGTTTTTTggggataccccctagagccccagctggttcttttctcctcagaagCCATAGAGCCCATCTCATCCCCAGCATGCCT |
| | | | GCTATTGTCTTCCCAATCCTCCCCCTTGCTGTCCTGCCCCACCCCACCCCCCAGAATAGAATGACACCTACTCAGACAA |
| | | | TTCTATGCAATTTCCTCATTTTATTAGGAAAGGACAGTGGGAGTGGCACCTTCCAGGGTCAAGGAAGGCATGGGGG |
| | | | AGGGGCAAACAACAGATGGCTGGCAACTAGAAGGCACAGTCTaggttaTTTTTGGGTGGGATTCACCACTTTTCCCAT |
| | | | GAAGAGGGGAGACTTGGTATTTTGTTCAATCATTAAGAAGACAAAGGGTTTGTTGAACTTGACCTCTGGGGGGATA |
| | | | GACATGGGTATGGCCTCTAAAAACATGGCCCCAGCAGCTTCAGTCCCTTTCTCATCTATGGTCAGCACAGCCTTATGC |
| | | | ACTGCCTTGGAGAGCTTCAGGGGTGCCTCCTCTGTGACCCCAGAGAGGTCAGCCCCATTGCTGAAGACCTTAGTGAT |
| | | | GCCCAGTTGACCCAGGACAGACTTCAGATCATAGGTTCCAGTAATGGACAGTTTGGGTAAATGTAAGCTGGCAGAC |
| | | | CTTCTGTCTTCATTTTCCAGGAACTTGGTGATGATATCATGGGTGAGTTCATTTTCCAGGTGCTGTAGTTTCCCCTCAT |
| | | | CAGGCAGGAAGAAGATGGCTGTGGCATTGCCCAGGTATTTCATCAGCAGCACCCAGCTGGACAGCTTCTTACAGTG |
| | | | CTGGATGTTAAACATGCCTAATCTCTTCATCATAGGCACCTTCACTGTGGTCACCTGGTCCACATGGAAGTCCTCTTCC |
| | | | TCTGTGTCCTTGACTTCAAAGGGTCTCTCCCATTTGCCTTTAAAGAAGATGTAATTCACCAGAGCAAAAACTGTGTCT |
| | | | CTGTCAAGCTCCTTGACCAAATCCACAATTTTCCCTTGAGTACCCTTCTCCACATAATCATTGATCTGTTTCTTGGCCTC |
| | | | TTCTGTGTCCCCAAAGTTGACAGTGAAGGCTTCTGAGTGGTACAACTTTTTAACATCCTCCAAAAACTTATCCACTAG |
| | | | CTTCAGGCCCTCAGAGAGGAACAGGCCATTGCCTGTGGTCAGCTGGAGCTGGCTGTCTGGCTGGTTGAGGGTTCTG |
| | | | AGGAGTTCCTGGAAGCCTTCATGGATCTGAGCCTCTGGAATCTCTGTGAGGTTGAAATTCAGGCCCTCCAGGATTTC |
| | | | ATCATGAGTGTCAGCCTTGGTCCCCAGGGAGAGCATTGCAAAGGCTGTAGCTATGCTCACTGGGGAGAAGAAGATA |
| | | | TTGGTGCTGTTGGACTGGTGTGCCAGCTGTCTGTATAGGCTGAAGGCAAACTCAGCCAGGTTGGGGGTGATCTTGT |
| | | | TGAAGGTTGGGTGATCCTGATCATGGTGGGATGTATCTGTCTTCTGGGCAGCATCTCCCTGGGGATCCTCaactgtgga |
| | | | aacagggagagaaaaaccacacaacatatttaaagattgatgaagacaactaactgtaatatgctgctttttgttcttctcttcactgacctaACTAGT |
| | | | AGATCTAGGAACCCCTAGTGATGGAGTTGGCCACTCCCTCTCTGCGCGCTCGCTCGCTCACTGAGGCCGCCCGGGCA |
| | | | AAGCCCGGGCGTCGGGCGACCTTTGGTCGCCCGGCCTCAGTGAGCGAGCGAGCGCGCAGAGAGGGAGTGGCCAAa |
| | | | cgcgtggtgtaatcatggtcatagctgtttcctgtgtgaaattgttatccgctcacaattccacacaacatacgagccggaagcataaagtgtaaagcctg |
| | | | gggtgcctaatgagtgagctaactcacattaattgcgttgcgctcactgcccgctttccagtcgggaaacctgtcgtgccagctgcattaatgaatcggcc |
| | | | aacgcgcggggagaggcggtttgcgtattgggcgctcttccgcttcctcgctcactgactcgctgcgctcggtcgttcggctgcggcgagcggtatcagct |
| | | | cactcaaaggcggtaatacggttatccacagaatcaggggataacgcaggaaagaacatgtgagcaaaaggccagcaaaaggccaggaaccgtaaa |
| | | | aaggccgcgttgctggcgtttttccataggctccgcccccctgacgagcatcacaaaaatcgacgctcaagtcagaggtggcgaaacccgacaggacta |
| | | | taaagataccaggcgtttccccctggaagctccctcgtgcgctctcctgttccgaccctgccgcttaccggatacctgtccgcctttctcccttcgggaagcg |
| | | | tggcgctttctcatagctcacgctgtaggtatctcagttcggtgtaggtcgttcgctccaagctgggctgtgtgcacgaaccccccgttcagcccgaccgct |
| | | | gcgccttatccggtaactatcgtcttgagtccaacccggtaagacacgacttatcgccactggcagcagccactggtaacaggattagcagagcgaggt |
| | | | atgtaggcggtgctacagagttcttgaagtggtggcctaactacggctacactagaagaacagtatttggtatctgcgctctgctgaagccagttaccttc |
| | | | ggaaaaagagttggtagctcttgatccggcaaacaaaccaccgctggtagcggtggtttttttgtttgcaagcagcagattacgcgcagaaaaaaagga |
| | | | tctcaagaagatcctttgatcttttctacggggtctgacgctcagtggaacgaaaactcacgttaagggattttggtcatgagattatcaaaaaggatcttc |
| | | | acctagatccttttaaattaaaaatgaagttttaaatcaagcccaatctgaataatgttacaaccaattaaccaattctgattagaaaaactcatcgagca |
| | | | tcaaatgaaactgcaatttattcatatcaggattatcaataccatatttttgaaaaagccgtttctgtaatgaaggagaaaactcaccgaggcagttccat |
| | | | aggatggcaagatcctggtatcggtctgcgattccgactcgtccaacatcaatacaacctattaatttcccctcgtcaaaaataaggttatcaagtgagaa |
| | | | atcaccatgagtgacgactgaatccggtgagaatggcaaaagtttatgcatttctttccagacttgttcaacaggccagccattacgctcgtcatcaaaat |
| | | | cactcgcatcaaccaaaccgttattcattcgtgattgcgcctgagcgagacgaaatacgcgatcgctgttaaaaggacaattacaaacaggaatcgaat |
| | | | gcaaccggcgcaggaacactgccagcgcatcaacaatattttcacctgaatcaggatattcttctaatacctggaatgctgtttttccggggatcgcagtg |
| | | | gtgagtaaccatgcatcatcaggagtacggataaaatgcttgatggtcggaagaggcataaattccgtcagccagtttagtctgaccatctcatctgtaa |
| | | | catcattggcaacgctacctttgccatgtttcagaaacaactctggcgcatcgggcttcccatacaagcgatagattgtcgcacctgattgcccgacattat |
| | | | cgcgagcccatttatacccatataaatcagcatccatgttggaatttaatcgcggcctcgacgtttcccgttgaatatggctcataacaccccttgtattact |
| | | | gtttatgtaagcagacagttttattgttcatgatgatatatttttatcttgtgcaatgtaacatcagagattttgagacacgggccagagctgcatcgcgcgt |
| | | | ttcggtgatgacggtgaaaacctctgacacatgcagctcccggagacggtcacagcttgtctgtaagcggatgccgggagcagacaagcccgtcagggc |
| | | | gcgtcagcgggtgttggcgggtgtcggggctggcttaactatgcggcatcagagcagattgtactgagagtgcaccatatgcggtgtgaaataccgcac |
| | | | agatgcgtaaggagaaaataccgcatcaggcgccattcgccattcaggctgcgcaactgttgggaagggcgatcggtgcgggcctcttcgctattacgc |
| | | | cagctggcgaaagggggatgtgctgcaaggcgattaagttgggtaacgccagggttttcccagtcacgacgttgtaaaacgacggccagagaattc |
| | SERPINA1 | A1AT w/o | GAGGACCCCCAGGGAGATGCAGCCCAGAAGACAGACACCAGCCACCATGACCAGGACCACCCCACCTTCAACAAGA |
| | copy 1 | SP | TCACCCCCAACCTGGCAGAGTTTGCCTTCAGCCTGTACAGGCAGCTGGCCCACCAGAGCAACAGCACCAACATCTTC |
| | | (alternate | TTCAGCCCAGTGAGCATAGCCACAGCCTTTGCCATGCTGAGCCTGGGCACCAAGGCAGACACCCATGATGAGATCCT |
| | | codon usage | GGAGGGCCTGAACTTCAACCTGACAGAGATCCCAGAGGCCCAGATCCATGAGGGCTTCCAGGAGCTGCTGAGGAC |
| | | 1) CpG | CCTGAACCAGCCAGACAGCCAGCTGCAGCTGACCACAGGCAATGGCCTGTTCCTGTCTGAGGGCCTGAAGCTGGTG |
| | | depleted | GACAAGTTCCTGGAGGATGTGAAGAAGCTGTACCACTCTGAGGCCTTCACAGTGAACTTTGGAGACACAGAGGAGG |
| | | (SEQ ID NO: | CCAAGAAGCAGATCAATGACTATGTGGAGAAGGGCACCCAGGGCAAGATAGTGGACCTGGTGAAGGAGCTGGAC |
| | | 761) | AGGGACACAGTGTTTGCCCTGGTGAACTACATCTTCTTCAAGGGCAAGTGGGAGAGGCCCTTTGAGGTGAAGGACA |
| | | | CAGAGGAGGAGGACTTCCATGTGGACCAGGTGACCACAGTGAAGGTGCCCATGATGAAGAGGCTGGGCATGTTCA |
| | | | ACATCCAGCACTGCAAGAAGCTGAGCAGCTGGGTGCTGCTGATGAAGTACCTGGGCAATGCCACAGCCATCTTCTTC |
| | | | CTGCCAGATGAGGGCAAGCTGCAGCACCTGGAGAATGAGCTGACCCATGACATCATCACCAAGTTCCTGGAGAATG |
| | | | AGGACAGGAGGTCTGCCAGCCTGCACCTGCCCAAGCTGAGCATCACAGGCACCTATGACCTGAAGTCTGTGCTGGG |
| | | | CCAGCTGGGCATCACCAAGGTGTTCAGCAATGGAGCAGACCTGTCTGGAGTGACAGAGGAGGCCCCCCTGAAGCT |
| | | | GAGCAAGGCAGTGCACAAGGCAGTGCTGACCATAGATGAGAAGGGCACAGAGGCAGCAGGAGCCATGTTCCTGG |
| | | | AGGCCATCCCCATGAGCATCCCCCCAGAGGTGAAGTTCAACAAGCCCTTTGTGTTCCTGATGATAGAGCAGAACACC |
| | | | AAGAGCCCCCTGTTCATGGGCAAGGTGGTGAACCCCACCCAGAAGTAA |
| | SERPINA1 | A1AT w/o | GAGGATCCCCAGGGAGATGCTGCCCAGAAGACAGATACATCCCACCATGATCAGGATCACCCAACCTTCAACAAGA |
| | copy 2 (rev | SP CpG | TCACCCCCAACCTGGCTGAGTTTGCCTTCAGCCTATACAGACAGCTGGCACACCAGTCCAACAGCACCAATATCTTCT |
| | comp) | depleted | TCTCCCCAGTGAGCATAGCTACAGCCTTTGCAATGCTCTCCCTGGGGACCAAGGCTGACACTCATGATGAAATCCTG |
| | | (SEQ ID NO: | GAGGGCCTGAATTTCAACCTCACAGAGATTCCAGAGGCTCAGATCCATGAAGGCTTCCAGGAACTCCTCAGAACCCT |
| | | 762) | CAACCAGCCAGACAGCCAGCTCCAGCTGACCACAGGCAATGGCCTGTTCCTCTCTGAGGGCCTGAAGCTAGTGGAT |
| | | | AAGTTTTTGGAGGATGTTAAAAAGTTGTACCACTCAGAAGCCTTCACTGTCAACTTTGGGGACACAGAAGAGGCCAA |
| | | | GAAACAGATCAATGATTATGTGGAGAAGGGTACTCAAGGGAAAATTGTGGATTTGGTCAAGGAGCTTGACAGAGA |
| | | | CACAGTTTTTGCTCTGGTGAATTACATCTTCTTTAAAGGCAAATGGGAGAGACCCTTTGAAGTCAAGGACACAGAGG |
| | | | AAGAGGACTTCCATGTGGACCAGGTGACCACAGTGAAGGTGCCTATGATGAAGAGATTAGGCATGTTTAACATCCA |
| | | | GCACTGTAAGAAGCTGTCCAGCTGGGTGCTGCTGATGAAATACCTGGGCAATGCCACAGCCATCTTCTTCCTGCCTG |
| | | | ATGAGGGGAAACTACAGCACCTGGAAAATGAACTCACCCATGATATCATCACCAAGTTCCTGGAAAATGAAGACAG |
| | | | AAGGTCTGCCAGCTTACATTTACCCAAACTGTCCATTACTGGAACCTATGATCTGAAGTCTGTCCTGGGTCAACTGGG |
| | | | CATCACTAAGGTCTTCAGCAATGGGGCTGACCTCTCTGGGGTCACAGAGGAGGCACCCCTGAAGCTCTCCAAGGCA |
| | | | GTGCATAAGGCTGTGCTGACCATAGATGAGAAAGGGACTGAAGCTGCTGGGGCCATGTTTTTAGAGGCCATACCCA |
| | | | TGTCTATCCCCCCAGAGGTCAAGTTCAACAAACCCTTTGTCTTCTTAATGATTGAACAAAATACCAAGTCTCCCCTCTT |
| | | | CATGGGAAAAGTGGTGAATCCCACCCAAAAAtaa |
| 9 | Full | (SEQ ID NO: | TTGGCCACTCCCTCTCTGCGCGCTCGCTCGCTCACTGAGGCCGGGCGACCAAAGGTCGCCCGACGCCCGGGCTTTGC |
| | Sequence | 790) | CCGGGCGGCCTCAGTGAGCGAGCGAGCGCGCAGAGAGGGAGTGGCCAACTCCATCACTAGGGGTTCCTAGATCTA |
| | | | CTAGTATAACTTCGTATAGCATACATTATACGAAGTTATATGTATGCtaggtcagtgaagagaagaacaaaaagcagcatattaca |
| | | | gttagttgtcttcatcaatctttaaatatgttgtgtggtttttctctccctgtttccacagttGAGGACCCCCAGGGAGATGCTGCCCAGAAGA |
| | | | CAGACACATCTCACCATGACCAGGACCACCCCACCTTCAACAAGATCACTCCCAATCTTGCAGAGTTTGCATTCTCTCT |
| | | | CTACAGACAGCTTGCACACCAGAGCAACTCTACTAACATCTTCTTCTCTCCAGTCAGCATAGCAACAGCATTTGCAAT |
| | | | GCTCAGCCTTGGCACAAAGGCAGACACACATGATGAGATCCTTGAGGGCCTCAACTTCAATCTCACAGAGATCCCAG |
| | | | AAGCCCAGATCCATGAGGGCTTCCAGGAGCTGCTGAGAACACTCAACCAGCCTGACTCTCAGCTCCAGCTCACAACA |
| | | | GGCAATGGGCTCTTCCTCTCTGAGGGCCTCAAGCTTGTAGACAAGTTCCTGGAGGATGTCAAGAAGCTCTACCACTC |
| | | | TGAAGCCTTCACAGTCAACTTTGGAGACACAGAGGAAGCCAAGAAGCAGATCAATGACTATGTAGAGAAGGGGAC |
| | | | TCAGGGCAAGATAGTAGACCTTGTCAAGGAGCTGGACAGAGACACAGTCTTTGCACTGGTCAACTACATCTTCTTCA |
| | | | AGGGGAAGTGGGAGAGACCCTTTGAAGTCAAGGACACAGAGGAGGAGGACTTCCATGTAGACCAGGTGACAACA |
| | | | GTCAAGGTTCCCATGATGAAGAGACTTGGCATGTTCAATATCCAGCACTGCAAGAAGCTCAGCTCTTGGGTCCTCCT |
| | | | CATGAAGTACCTTGGCAATGCAACAGCAATCTTCTTCCTTCCTGATGAGGGCAAGCTCCAGCACCTTGAGAATGAGC |
| | | | TGACACATGACATCATCACAAAGTTCCTGGAGAATGAGGACAGAAGGTCTGCATCTCTCCACCTTCCAAAGCTCAGC |
| | | | ATCACAGGCACCTATGACCTCAAGTCTGTCCTTGGCCAGCTTGGCATCACAAAGGTCTTCTCTAATGGTGCAGACCTC |
| | | | TCTGGAGTCACAGAGGAAGCCCCCCTCAAGCTCAGCAAGGCTGTGCACAAGGCTGTGCTCACAATAGATGAGAAGG |
| | | | GGACAGAGGCTGCAGGTGCCATGTTCCTGGAAGCCATCCCCATGAGCATCCCACCAGAAGTCAAGTTCAACAAGCC |
| | | | TTTTGTCTTCCTGATGATAGAGCAGAACACAAAGTCTCCCCTCTTCATGGGCAAGGTAGTCAACCCCACTCAAAAGTA |
| | | | ACAGACATGATAAGATACATTGATGAGTTTGGACAAACCACAACTAGAATGCAGTGAAAAAAATGCTTTATTTGTGA |
| | | | AATTTGTGATGCTATTGCTTTATTTGTAACCATTATAAGCTGCAATAAACAAGTTAACAACAACAATTGCATTCATTTT |
| | | | ATGTTTCAGGTTCAGGGGGAGGTGTGGGAGGTTTTTTggggataccccctagagccccagctggttcttttctcctcagaagCCATA |
| | | | GAGCCCATCTCATCCCCAGCATGCCTGCTATTGTCTTCCCAATCCTCCCCCTTGCTGTCCTGCCCCACCCCACCCCCCA |
| | | | GAATAGAATGACACCTACTCAGACAATTCTATGCAATTTCCTCATTTTATTAGGAAAGGACAGTGGGAGTGGCACCT |
| | | | TCCAGGGTCAAGGAAGGCATGGGGGAGGGGCAAACAACAGATGGCTGGCAACTAGAAGGCACAGTCTaggTTACT |
| | | | TCTGGGTGGGGTTCACCACCTTGCCCATGAACAGGGGGCTCTTGGTGTTCTGCTCTATCATCAGGAACACAAAAGGC |
| | | | TTGTTGAACTTCACCTCTGGGGGGATGCTCATGGGGATGGCCTCCAGGAACATGGCTCCTGCTGCCTCTGTGCCCTT |
| | | | CTCATCTATGGTCAGCACTGCCTTGTGCACTGCCTTGCTCAGCTTCAGGGGGGCCTCCTCTGTCACTCCAGACAGGTC |
| | | | TGCTCCATTGCTGAACACCTTGGTGATGCCCAGCTGGCCCAGCACAGACTTCAGGTCATAGGTGCCTGTGATGCTCA |
| | | | GCTTGGGCAGGTGCAGGCTGGCAGACCTCCTGTCCTCATTCTCCAGGAACTTGGTGATGATGTCATGGGTCAGCTCA |
| | | | TTCTCCAGGTGCTGCAGCTTGCCCTCATCTGGCAGGAAGAAGATGGCTGTGGCATTGCCCAGGTACTTCATCAGCAG |
| | | | CACCCAGCTGCTCAGCTTCTTGCAGTGCTGGATATTGAACATGCCCAGCCTCTTCATCATGGGCACCTTCACTGTGGT |
| | | | CACCTGGTCCACATGGAAGTCCTCCTCCTCTGTGTCCTTCACCTCAAAGGGCCTCTCCCACTTGCCCTTGAAGAAGAT |
| | | | GTAGTTCACCAGGGCAAACACTGTGTCCCTGTCCAGCTCCTTCACCAGGTCCACTATCTTGCCCTGGGTGCCCTTCTC |
| | | | CACATAGTCATTGATCTGCTTCTTGGCCTCCTCTGTGTCTCCAAAGTTCACTGTGAAGGCCTCAGAGTGGTACAGCTT |
| | | | CTTCACATCCTCCAGGAACTTGTCCACCAGCTTCAGGCCCTCAGACAGGAACAGGCCATTGCCTGTGGTCAGCTGCA |
| | | | GCTGGCTGTCTGGCTGGTTCAGGGTCCTCAGCAGCTCCTGGAAGCCCTCATGGATCTGGGCCTCTGGGATCTCTGTC |
| | | | AGGTTGAAGTTCAGGCCCTCCAGGATCTCATCATGGGTGTCTGCCTTGGTGCCCAGGCTCAGCATGGCAAAGGCTGT |
| | | | GGCTATGCTCACTGGGCTGAAGAAGATGTTGGTGCTGTTGCTCTGGTGGGCCAGCTGCCTGTACAGGCTGAAGGCA |
| | | | AACTCTGCCAGGTTGGGGGTGATCTTGTTGAAGGTGGGGTGGTCCTGGTCATGGTGGCTGGTGTCTGTCTTCTGGG |
| | | | CTGCATCTCCCTGGGGGTCCTCaactgtggaaacagggagagaaaaaccacacaacatatttaaagattgatgaagacaactaactgtaata |
| | | | tgctgctttttgttcttctcttcactgacctaATGTATGCATAACTTCGTATAGCATACATTATACGAAGTTATACTAGTAGATCTA |
| | | | GGAACCCCTAGTGATGGAGTTGGCCACTCCCTCTCTGCGCGCTCGCTCGCTCACTGAGGCCGCCCGGGCAAAGCCC |
| | | | GGGCGTCGGGCGACCTTTGGTCGCCCGGCCTCAGTGAGCGAGCGAGCGCGCAGAGAGGGAGTGGCCAAacgcgtggt |
| | | | gtaatcatggtcatagctgtttcctgtgtgaaattgttatccgctcacaattccacacaacatacgagccggaagcataaagtgtaaagcctggggtgcct |
| | | | aatgagtgagctaactcacattaattgcgttgcgctcactgcccgctttccagtcgggaaacctgtcgtgccagctgcattaatgaatcggccaacgcgcg |
| | | | gggagaggcggtttgcgtattgggcgctcttccgcttcctcgctcactgactcgctgcgctcggtcgttcggctgcggcgagcggtatcagctcactcaaa |
| | | | ggcggtaatacggttatccacagaatcaggggataacgcaggaaagaacatgtgagcaaaaggccagcaaaaggccaggaaccgtaaaaaggccgc |
| | | | gttgctggcgtttttccataggctccgcccccctgacgagcatcacaaaaatcgacgctcaagtcagaggtggcgaaacccgacaggactataaagata |
| | | | ccaggcgtttccccctggaagctccctcgtgcgctctcctgttccgaccctgccgcttaccggatacctgtccgcctttctcccttcgggaagcgtggcgctt |
| | | | tctcatagctcacgctgtaggtatctcagttcggtgtaggtcgttcgctccaagctgggctgtgtgcacgaaccccccgttcagcccgaccgctgcgcctta |
| | | | tccggtaactatcgtcttgagtccaacccggtaagacacgacttatcgccactggcagcagccactggtaacaggattagcagagcgaggtatgtaggc |
| | | | ggtgctacagagttcttgaagtggtggcctaactacggctacactagaagaacagtatttggtatctgcgctctgctgaagccagttaccttcggaaaaa |
| | | | gagttggtagctcttgatccggcaaacaaaccaccgctggtagcggtggtttttttgtttgcaagcagcagattacgcgcagaaaaaaaggatctcaaga |
| | | | agatcctttgatcttttctacggggtctgacgctcagtggaacgaaaactcacgttaagggattttggtcatgagattatcaaaaaggatcttcacctagat |
| | | | ccttttaaattaaaaatgaagttttaaatcaagcccaatctgaataatgttacaaccaattaaccaattctgattagaaaaactcatcgagcatcaaatga |
| | | | aactgcaatttattcatatcaggattatcaataccatatttttgaaaaagccgtttctgtaatgaaggagaaaactcaccgaggcagttccataggatggc |
| | | | aagatcctggtatcggtctgcgattccgactcgtccaacatcaatacaacctattaatttcccctcgtcaaaaataaggttatcaagtgagaaatcaccat |
| | | | gagtgacgactgaatccggtgagaatggcaaaagtttatgcatttctttccagacttgttcaacaggccagccattacgctcgtcatcaaaatcactcgca |
| | | | tcaaccaaaccgttattcattcgtgattgcgcctgagcgagacgaaatacgcgatcgctgttaaaaggacaattacaaacaggaatcgaatgcaaccgg |
| | | | cgcaggaacactgccagcgcatcaacaatattttcacctgaatcaggatattcttctaatacctggaatgctgtttttccggggatcgcagtggtgagtaa |
| | | | ccatgcatcatcaggagtacggataaaatgcttgatggtcggaagaggcataaattccgtcagccagtttagtctgaccatctcatctgtaacatcattgg |
| | | | caacgctacctttgccatgtttcagaaacaactctggcgcatcgggcttcccatacaagcgatagattgtcgcacctgattgcccgacattatcgcgagcc |
| | | | catttatacccatataaatcagcatccatgttggaatttaatcgcggcctcgacgtttcccgttgaatatggctcataacaccccttgtattactgtttatgta |
| | | | agcagacagttttattgttcatgatgatatatttttatcttgtgcaatgtaacatcagagattttgagacacgggccagagctgcatcgcgcgtttcggtgat |
| | | | gacggtgaaaacctctgacacatgcagctcccggagacggtcacagcttgtctgtaagcggatgccgggagcagacaagcccgtcagggcgcgtcagc |
| | | | gggtgttggcgggtgtcggggctggcttaactatgcggcatcagagcagattgtactgagagtgcaccatatgcggtgtgaaataccgcacagatgcgt |
| | | | aaggagaaaataccgcatcaggcgccattcgccattcaggctgcgcaactgttgggaagggcgatcggtgcgggcctcttcgctattacgccagctggc |
| | | | gaaagggggatgtgctgcaaggcgattaagttgggtaacgccagggttttcccagtcacgacgttgtaaaacgacggccagagaattc |
| | SERPINA1 | A1AT w/o | GAGGACCCCCAGGGAGATGCTGCCCAGAAGACAGACACATCTCACCATGACCAGGACCACCCCACCTTCAACAAGA |
| | copy 1 | SP | TCACTCCCAATCTTGCAGAGTTTGCATTCTCTCTCTACAGACAGCTTGCACACCAGAGCAACTCTACTAACATCTTCTT |
| | | (alternate | CTCTCCAGTCAGCATAGCAACAGCATTTGCAATGCTCAGCCTTGGCACAAAGGCAGACACACATGATGAGATCCTTG |
| | | codon usage | AGGGCCTCAACTTCAATCTCACAGAGATCCCAGAAGCCCAGATCCATGAGGGCTTCCAGGAGCTGCTGAGAACACT |
| | | 2) CpG | CAACCAGCCTGACTCTCAGCTCCAGCTCACAACAGGCAATGGGCTCTTCCTCTCTGAGGGCCTCAAGCTTGTAGACA |
| | | depleted | AGTTCCTGGAGGATGTCAAGAAGCTCTACCACTCTGAAGCCTTCACAGTCAACTTTGGAGACACAGAGGAAGCCAA |
| | | (SEQ ID NO: | GAAGCAGATCAATGACTATGTAGAGAAGGGGACTCAGGGCAAGATAGTAGACCTTGTCAAGGAGCTGGACAGAGA |
| | | 791) | CACAGTCTTTGCACTGGTCAACTACATCTTCTTCAAGGGGAAGTGGGAGAGACCCTTTGAAGTCAAGGACACAGAG |
| | | | GAGGAGGACTTCCATGTAGACCAGGTGACAACAGTCAAGGTTCCCATGATGAAGAGACTTGGCATGTTCAATATCC |
| | | | AGCACTGCAAGAAGCTCAGCTCTTGGGTCCTCCTCATGAAGTACCTTGGCAATGCAACAGCAATCTTCTTCCTTCCTG |
| | | | ATGAGGGCAAGCTCCAGCACCTTGAGAATGAGCTGACACATGACATCATCACAAAGTTCCTGGAGAATGAGGACAG |
| | | | AAGGTCTGCATCTCTCCACCTTCCAAAGCTCAGCATCACAGGCACCTATGACCTCAAGTCTGTCCTTGGCCAGCTTGG |
| | | | CATCACAAAGGTCTTCTCTAATGGTGCAGACCTCTCTGGAGTCACAGAGGAAGCCCCCCTCAAGCTCAGCAAGGCTG |
| | | | TGCACAAGGCTGTGCTCACAATAGATGAGAAGGGGACAGAGGCTGCAGGTGCCATGTTCCTGGAAGCCATCCCCAT |
| | | | GAGCATCCCACCAGAAGTCAAGTTCAACAAGCCTTTTGTCTTCCTGATGATAGAGCAGAACACAAAGTCTCCCCTCTT |
| | | | CATGGGCAAGGTAGTCAACCCCACTCAAAAG |
| | SERPINA1 | A1AT w/o | GAGGACCCCCAGGGAGATGCAGCCCAGAAGACAGACACCAGCCACCATGACCAGGACCACCCCACCTTCAACAAGA |
| | copy 2 (rev | SP | TCACCCCCAACCTGGCAGAGTTTGCCTTCAGCCTGTACAGGCAGCTGGCCCACCAGAGCAACAGCACCAACATCTTC |
| | comp) | (alternate | TTCAGCCCAGTGAGCATAGCCACAGCCTTTGCCATGCTGAGCCTGGGCACCAAGGCAGACACCCATGATGAGATCCT |
| | | codon usage | GGAGGGCCTGAACTTCAACCTGACAGAGATCCCAGAGGCCCAGATCCATGAGGGCTTCCAGGAGCTGCTGAGGAC |
| | | 1) CpG | CCTGAACCAGCCAGACAGCCAGCTGCAGCTGACCACAGGCAATGGCCTGTTCCTGTCTGAGGGCCTGAAGCTGGTG |
| | | depleted | GACAAGTTCCTGGAGGATGTGAAGAAGCTGTACCACTCTGAGGCCTTCACAGTGAACTTTGGAGACACAGAGGAGG |
| | | (SEQ ID NO: | CCAAGAAGCAGATCAATGACTATGTGGAGAAGGGCACCCAGGGCAAGATAGTGGACCTGGTGAAGGAGCTGGAC |
| | | 792) | AGGGACACAGTGTTTGCCCTGGTGAACTACATCTTCTTCAAGGGCAAGTGGGAGAGGCCCTTTGAGGTGAAGGACA |
| | | | CAGAGGAGGAGGACTTCCATGTGGACCAGGTGACCACAGTGAAGGTGCCCATGATGAAGAGGCTGGGCATGTTCA |
| | | | ATATCCAGCACTGCAAGAAGCTGAGCAGCTGGGTGCTGCTGATGAAGTACCTGGGCAATGCCACAGCCATCTTCTTC |
| | | | CTGCCAGATGAGGGCAAGCTGCAGCACCTGGAGAATGAGCTGACCCATGACATCATCACCAAGTTCCTGGAGAATG |
| | | | AGGACAGGAGGTCTGCCAGCCTGCACCTGCCCAAGCTGAGCATCACAGGCACCTATGACCTGAAGTCTGTGCTGGG |
| | | | CCAGCTGGGCATCACCAAGGTGTTCAGCAATGGAGCAGACCTGTCTGGAGTGACAGAGGAGGCCCCCCTGAAGCT |
| | | | GAGCAAGGCAGTGCACAAGGCAGTGCTGACCATAGATGAGAAGGGCACAGAGGCAGCAGGAGCCATGTTCCTGG |
| | | | AGGCCATCCCCATGAGCATCCCCCCAGAGGTGAAGTTCAACAAGCCTTTTGTGTTCCTGATGATAGAGCAGAACACC |
| | | | AAGAGCCCCCTGTTCATGGGCAAGGTGGTGAACCCCACCCAGAAGTAA |
| 10 | Full | (SEQ ID NO: | TTGGCCACTCCCTCTCTGCGCGCTCGCTCGCTCACTGAGGCCGGGCGACCAAAGGTCGCCCGACGCCCGGGCTTTGC |
| | Sequence | 795 | CCGGGCGGCCTCAGTGAGCGAGCGAGCGCGCAGAGAGGGAGTGGCCAACTCCATCACTAGGGGTTCCTAGATCTA |
| | | | CTAGTATAACTTCGTATAGCATACATTATACGAAGTTATATGTATGCtaggtcagtgaagagaagaacaaaaagcagcatattaca |
| | | | gttagttgtcttcatcaatctttaaatatgttgtgtggtttttctctccctgtttccacagttGAGGACCCCCAGGGAGATGCAGCCCAGAAGA |
| | | | CAGACACCAGCCACCATGACCAGGACCACCCCACCTTCAACAAGATCACCCCCAACCTGGCAGAGTTTGCCTTCAGC |
| | | | CTGTACAGGCAGCTGGCCCACCAGAGCAACAGCACCAACATCTTCTTCAGCCCAGTGAGCATAGCCACAGCCTTTGC |
| | | | CATGCTGAGCCTGGGCACCAAGGCAGACACCCATGATGAGATCCTGGAGGGCCTGAACTTCAACCTGACAGAGATC |
| | | | CCAGAGGCCCAGATCCATGAGGGCTTCCAGGAGCTGCTGAGGACCCTGAACCAGCCAGACAGCCAGCTGCAGCTG |
| | | | ACCACAGGCAATGGCCTGTTCCTGTCTGAGGGCCTGAAGCTGGTGGACAAGTTCCTGGAGGATGTGAAGAAGCTGT |
| | | | ACCACTCTGAGGCCTTCACAGTGAACTTTGGAGACACAGAGGAGGCCAAGAAGCAGATCAATGACTATGTGGAGAA |
| | | | GGGCACCCAGGGCAAGATAGTGGACCTGGTGAAGGAGCTGGACAGGGACACAGTGTTTGCCCTGGTGAACTACAT |
| | | | CTTCTTCAAGGGCAAGTGGGAGAGGCCCTTTGAGGTGAAGGACACAGAGGAGGAGGACTTCCATGTGGACCAGGT |
| | | | GACCACAGTGAAGGTGCCCATGATGAAGAGGCTGGGCATGTTCAATATCCAGCACTGCAAGAAGCTGAGCAGCTG |
| | | | GGTGCTGCTGATGAAGTACCTGGGCAATGCCACAGCCATCTTCTTCCTGCCAGATGAGGGCAAGCTGCAGCACCTG |
| | | | GAGAATGAGCTGACCCATGACATCATCACCAAGTTCCTGGAGAATGAGGACAGGAGGTCTGCCAGCCTGCACCTGC |
| | | | CCAAGCTGAGCATCACAGGCACCTATGACCTGAAGTCTGTGCTGGGCCAGCTGGGCATCACCAAGGTGTTCAGCAA |
| | | | TGGAGCAGACCTGTCTGGAGTGACAGAGGAGGCCCCCCTGAAGCTGAGCAAGGCAGTGCACAAGGCAGTGCTGAC |
| | | | CATAGATGAGAAGGGCACAGAGGCAGCAGGAGCCATGTTCCTGGAGGCCATCCCCATGAGCATCCCCCCAGAGGT |
| | | | GAAGTTCAACAAGCCTTTTGTGTTCCTGATGATAGAGCAGAACACCAAGAGCCCCCTGTTCATGGGCAAGGTGGTG |
| | | | AACCCCACCCAGAAGTAACAGACATGATAAGATACATTGATGAGTTTGGACAAACCACAACTAGAATGCAGTGAAA |
| | | | AAAATGCTTTATTTGTGAAATTTGTGATGCTATTGCTTTATTTGTAACCATTATAAGCTGCAATAAACAAGTTAACAAC |
| | | | AACAATTGCATTCATTTTATGTTTCAGGTTCAGGGGGAGGTGTGGGAGGTTTTTTggggataccccctagagccccagctggtt |
| | | | cttttctcctcagaagCCATAGAGCCCATCTCATCCCCAGCATGCCTGCTATTGTCTTCCCAATCCTCCCCCTTGCTGTCCTG |
| | | | CCCCACCCCACCCCCCAGAATAGAATGACACCTACTCAGACAATTCTATGCAATTTCCTCATTTTATTAGGAAAGGAC |
| | | | AGTGGGAGTGGCACCTTCCAGGGTCAAGGAAGGCATGGGGGAGGGGCAAACAACAGATGGCTGGCAACTAGAAG |
| | | | GCACAGTCTaggttaTTTTTGGGTGGGATTCACCACTTTTCCCATGAAGAGGGGTGATTTAGTGTTCTGCTCTATCATG |
| | | | AGAAATACAAAAGGTTTGTTGAACTTGACCTCTGGGGGGATAGACATGGGTATGGCCTCTAAAAACATGGCCCCAG |
| | | | CAGCCTCTGTGCCCTTCTCATCTATGGTCAGCACAGCCTTATGCACTGCCTTGGAGAGCTTCAGGGGTGCCTCCTCTG |
| | | | TGACCCCAGAGAGGTCAGCCCCATTGCTGAAGACCTTAGTGATGCCCAGTTGACCCAGGACAGACTTCAGATCATAG |
| | | | GTTCCAGTAATGGACAGTTTGGGTAAATGTAAGCTGGCAGACCTTCTGTCTTCATTTTCCAGGAACTTGGTGATGAT |
| | | | ATCATGGGTGAGTTCATTTTCCAGGTGCTGTAGTTTCCCCTCATCAGGCAGGAAGAAGATGGCTGTGGCATTGCCCA |
| | | | GGTATTTCATCAGCAGCACCCAGCTGGACAGCTTCTTACAGTGCTGGATATTGAACATACCAAGCCTTTTCATCATAG |
| | | | GCACCTTCACTGTGGTCACCTGGTCCACATGGAAGTCCTCTTCCTCTGTGTCCTTGACTTCAAAGGGTCTCTCCCATTT |
| | | | GCCTTTAAAGAAGATGTAATTCACCAGAGCAAAAACTGTGTCTCTGTCAAGCTCCTTGACCAAATCCACAATTTTCCC |
| | | | TTGAGTACCCTTCTCCACATAATCATTGATCTGTTTCTTGGCCTCTTCTGTGTCCCCAAAGTTGACAGTGAAGGCTTCT |
| | | | GAGTGGTACAACTTTTTAACATCCTCCAAAAACTTATCCACTAGCTTCAGGCCCTCAGAGAGGAACAGGCCATTGCCT |
| | | | GTGGTCAGCTGGAGCTGGCTGTCTGGCTGGTTGAGGGTTCTGAGGAGTTCCTGGAAGCCTTCATGGATCTGAGCCT |
| | | | CTGGAATCTCTGTGAGGTTGAAATTCAGGCCCTCCAGGATTTCATCATGAGTGTCAGCCTTGGTCCCCAGGGAGAGC |
| | | | ATTGCAAAGGCTGTAGCTATGCTCACTGGGGAGAAGAAGATATTGGTGCTGTTGGACTGGTGTGCCAGCTGTCTGT |
| | | | ATAGGCTGAAGGCAAACTCAGCCAGGTTGGGGGTGATCTTGTTGAAGGTTGGGTGATCCTGATCATGGTGGGATGT |
| | | | ATCTGTCTTCTGGGCAGCATCTCCCTGGGGATCCTCaactgtggaaacagggagagaaaaaccacacaacatatttaaagattgatga |
| | | | agacaactaactgtaatatgctgctttttgttcttctcttcactgacctaATGTATGCATAACTTCGTATAGCATACATTATACGAAGTTA |
| | | | TACTAGTAGATCTAGGAACCCCTAGTGATGGAGTTGGCCACTCCCTCTCTGCGCGCTCGCTCGCTCACTGAGGCCGC |
| | | | CCGGGCAAAGCCCGGGCGTCGGGCGACCTTTGGTCGCCCGGCCTCAGTGAGCGAGCGAGCGCGCAGAGAGGGAG |
| | | | TGGCCAAacgcgtggtgtaatcatggtcatagctgtttcctgtgtgaaattgttatccgctcacaattccacacaacatacgagccggaagcataaagt |
| | | | gtaaagcctggggtgcctaatgagtgagctaactcacattaattgcgttgcgctcactgcccgctttccagtcgggaaacctgtcgtgccagctgcattaa |
| | | | tgaatcggccaacgcgcggggagaggcggtttgcgtattgggcgctcttccgcttcctcgctcactgactcgctgcgctcggtcgttcggctgcggcgagc |
| | | | ggtatcagctcactcaaaggcggtaatacggttatccacagaatcaggggataacgcaggaaagaacatgtgagcaaaaggccagcaaaaggccag |
| | | | gaaccgtaaaaaggccgcgttgctggcgtttttccataggctccgcccccctgacgagcatcacaaaaatcgacgctcaagtcagaggtggcgaaaccc |
| | | | gacaggactataaagataccaggcgtttccccctggaagctccctcgtgcgctctcctgttccgaccctgccgcttaccggatacctgtccgcctttctccct |
| | | | tcgggaagcgtggcgctttctcatagctcacgctgtaggtatctcagttcggtgtaggtcgttcgctccaagctgggctgtgtgcacgaaccccccgttcag |
| | | | cccgaccgctgcgccttatccggtaactatcgtcttgagtccaacccggtaagacacgacttatcgccactggcagcagccactggtaacaggattagca |
| | | | gagcgaggtatgtaggcggtgctacagagttcttgaagtggtggcctaactacggctacactagaagaacagtatttggtatctgcgctctgctgaagcc |
| | | | agttaccttcggaaaaagagttggtagctcttgatccggcaaacaaaccaccgctggtagcggtggtttttttgtttgcaagcagcagattacgcgcaga |
| | | | aaaaaaggatctcaagaagatcctttgatcttttctacggggtctgacgctcagtggaacgaaaactcacgttaagggattttggtcatgagattatcaa |
| | | | aaaggatcttcacctagatccttttaaattaaaaatgaagttttaaatcaagcccaatctgaataatgttacaaccaattaaccaattctgattagaaaaa |
| | | | ctcatcgagcatcaaatgaaactgcaatttattcatatcaggattatcaataccatatttttgaaaaagccgtttctgtaatgaaggagaaaactcaccga |
| | | | ggcagttccataggatggcaagatcctggtatcggtctgcgattccgactcgtccaacatcaatacaacctattaatttcccctcgtcaaaaataaggtta |
| | | | tcaagtgagaaatcaccatgagtgacgactgaatccggtgagaatggcaaaagtttatgcatttctttccagacttgttcaacaggccagccattacgctc |
| | | | gtcatcaaaatcactcgcatcaaccaaaccgttattcattcgtgattgcgcctgagcgagacgaaatacgcgatcgctgttaaaaggacaattacaaaca |
| | | | ggaatcgaatgcaaccggcgcaggaacactgccagcgcatcaacaatattttcacctgaatcaggatattcttctaatacctggaatgctgtttttccggg |
| | | | gatcgcagtggtgagtaaccatgcatcatcaggagtacggataaaatgcttgatggtcggaagaggcataaattccgtcagccagtttagtctgaccatc |
| | | | tcatctgtaacatcattggcaacgctacctttgccatgtttcagaaacaactctggcgcatcgggcttcccatacaagcgatagattgtcgcacctgattgc |
| | | | ccgacattatcgcgagcccatttatacccatataaatcagcatccatgttggaatttaatcgcggcctcgacgtttcccgttgaatatggctcataacaccc |
| | | | cttgtattactgtttatgtaagcagacagttttattgttcatgatgatatatttttatcttgtgcaatgtaacatcagagattttgagacacgggccagagctg |
| | | | catcgcgcgtttcggtgatgacggtgaaaacctctgacacatgcagctcccggagacggtcacagcttgtctgtaagcggatgccgggagcagacaagc |
| | | | ccgtcagggcgcgtcagcgggtgttggcgggtgtcggggctggcttaactatgcggcatcagagcagattgtactgagagtgcaccatatgcggtgtga |
| | | | aataccgcacagatgcgtaaggagaaaataccgcatcaggcgccattcgccattcaggctgcgcaactgttgggaagggcgatcggtgcgggcctcttc |
| | | | gctattacgccagctggcgaaagggggatgtgctgcaaggcgattaagttgggtaacgccagggttttcccagtcacgacgttgtaaaacgacggccag |
| | | | agaattc |
| | SERPINA1 | A1AT w/o | GAGGACCCCCAGGGAGATGCAGCCCAGAAGACAGACACCAGCCACCATGACCAGGACCACCCCACCTTCAACAAGA |
| | copy 1 | SP | TCACCCCCAACCTGGCAGAGTTTGCCTTCAGCCTGTACAGGCAGCTGGCCCACCAGAGCAACAGCACCAACATCTTC |
| | | (alternate | TTCAGCCCAGTGAGCATAGCCACAGCCTTTGCCATGCTGAGCCTGGGCACCAAGGCAGACACCCATGATGAGATCCT |
| | | codon usage | GGAGGGCCTGAACTTCAACCTGACAGAGATCCCAGAGGCCCAGATCCATGAGGGCTTCCAGGAGCTGCTGAGGAC |
| | | 1) CpG | CCTGAACCAGCCAGACAGCCAGCTGCAGCTGACCACAGGCAATGGCCTGTTCCTGTCTGAGGGCCTGAAGCTGGTG |
| | | depleted | GACAAGTTCCTGGAGGATGTGAAGAAGCTGTACCACTCTGAGGCCTTCACAGTGAACTTTGGAGACACAGAGGAGG |
| | | (SEQ ID NO: | CCAAGAAGCAGATCAATGACTATGTGGAGAAGGGCACCCAGGGCAAGATAGTGGACCTGGTGAAGGAGCTGGAC |
| | | 796) | AGGGACACAGTGTTTGCCCTGGTGAACTACATCTTCTTCAAGGGCAAGTGGGAGAGGCCCTTTGAGGTGAAGGACA |
| | | | CAGAGGAGGAGGACTTCCATGTGGACCAGGTGACCACAGTGAAGGTGCCCATGATGAAGAGGCTGGGCATGTTCA |
| | | | ATATCCAGCACTGCAAGAAGCTGAGCAGCTGGGTGCTGCTGATGAAGTACCTGGGCAATGCCACAGCCATCTTCTTC |
| | | | CTGCCAGATGAGGGCAAGCTGCAGCACCTGGAGAATGAGCTGACCCATGACATCATCACCAAGTTCCTGGAGAATG |
| | | | AGGACAGGAGGTCTGCCAGCCTGCACCTGCCCAAGCTGAGCATCACAGGCACCTATGACCTGAAGTCTGTGCTGGG |
| | | | CCAGCTGGGCATCACCAAGGTGTTCAGCAATGGAGCAGACCTGTCTGGAGTGACAGAGGAGGCCCCCCTGAAGCT |
| | | | GAGCAAGGCAGTGCACAAGGCAGTGCTGACCATAGATGAGAAGGGCACAGAGGCAGCAGGAGCCATGTTCCTGG |
| | | | AGGCCATCCCCATGAGCATCCCCCCAGAGGTGAAGTTCAACAAGCCTTTTGTGTTCCTGATGATAGAGCAGAACACC |
| | | | AAGAGCCCCCTGTTCATGGGCAAGGTGGTGAACCCCACCCAGAAGTAA |
| | SERPINA1 | A1AT w/o | GAGGATCCCCAGGGAGATGCTGCCCAGAAGACAGATACATCCCACCATGATCAGGATCACCCAACCTTCAACAAGA |
| | copy 2 (rev | SP CpG | TCACCCCCAACCTGGCTGAGTTTGCCTTCAGCCTATACAGACAGCTGGCACACCAGTCCAACAGCACCAATATCTTCT |
| | comp) | depleted | TCTCCCCAGTGAGCATAGCTACAGCCTTTGCAATGCTCTCCCTGGGGACCAAGGCTGACACTCATGATGAAATCCTG |
| | | (SEQ ID NO: | GAGGGCCTGAATTTCAACCTCACAGAGATTCCAGAGGCTCAGATCCATGAAGGCTTCCAGGAACTCCTCAGAACCCT |
| | | 797) | CAACCAGCCAGACAGCCAGCTCCAGCTGACCACAGGCAATGGCCTGTTCCTCTCTGAGGGCCTGAAGCTAGTGGAT |
| | | | AAGTTTTTGGAGGATGTTAAAAAGTTGTACCACTCAGAAGCCTTCACTGTCAACTTTGGGGACACAGAAGAGGCCAA |
| | | | GAAACAGATCAATGATTATGTGGAGAAGGGTACTCAAGGGAAAATTGTGGATTTGGTCAAGGAGCTTGACAGAGA |
| | | | CACAGTTTTTGCTCTGGTGAATTACATCTTCTTTAAAGGCAAATGGGAGAGACCCTTTGAAGTCAAGGACACAGAGG |
| | | | AAGAGGACTTCCATGTGGACCAGGTGACCACAGTGAAGGTGCCTATGATGAAAAGGCTTGGTATGTTCAATATCCA |
| | | | GCACTGTAAGAAGCTGTCCAGCTGGGTGCTGCTGATGAAATACCTGGGCAATGCCACAGCCATCTTCTTCCTGCCTG |
| | | | ATGAGGGGAAACTACAGCACCTGGAAAATGAACTCACCCATGATATCATCACCAAGTTCCTGGAAAATGAAGACAG |
| | | | AAGGTCTGCCAGCTTACATTTACCCAAACTGTCCATTACTGGAACCTATGATCTGAAGTCTGTCCTGGGTCAACTGGG |
| | | | CATCACTAAGGTCTTCAGCAATGGGGCTGACCTCTCTGGGGTCACAGAGGAGGCACCCCTGAAGCTCTCCAAGGCA |
| | | | GTGCATAAGGCTGTGCTGACCATAGATGAGAAGGGCACAGAGGCTGCTGGGGCCATGTTTTTAGAGGCCATACCCA |
| | | | TGTCTATCCCCCCAGAGGTCAAGTTCAACAAACCTTTTGTATTTCTCATGATAGAGCAGAACACTAAATCACCCCTCTT |
| | | | CATGGGAAAAGTGGTGAATCCCACCCAAAAAtaa |
| 11 | Full | (SEQ ID NO: | tgtaacatcagagattttgagacacgggccagagctgcatcgcgcgtttcggtgatgacggtgaaaacctctgacacatgcagctcccggagacggtca |
| | Sequence | 1564) | cagcttgtctgtaagcggatgccgggagcagacaagcccgtcagggcgcgtcagcgggtgttggcgggtgtcggggctggcttaactatgcggcatcag |
| | | | agcagattgtactgagagtgcaccatatgcggtgtgaaataccgcacagatgcgtaaggagaaaataccgcatcaggcgccattcgccattcaggctgc |
| | | | gcaactgttgggaagggcgatcggtgcgggcctcttcgctattacgccagctggcgaaagggggatgtgctgcaaggcgattaagttgggtaacgccag |
| | | | ggttttcccagtcacgacgttgtaaaacgacggccagagaattcTTGGCCACTCCCTCTCTGCGCGCTCGCTCGCTCACTGAGGCCG |
| | | | GGCGACCAAAGGTCGCCCGACGCCCGGGCTTTGCCCGGGCGGCCTCAGTGAGCGAGCGAGCGCGCAGAGAGGGA |
| | | | GTGGCCAACTCCATCACTAGGGGTTCCTAGATCTACTAGTTGCATAATCTAAGTCAAATGGAAAGAAATATAAAAAG |
| | | | TAACATTATTACTTCTTGTTTTCTTCAGTATTTAACAATCCttttttttCTTCCCTTGCCCAGttGAGGACCCCCAGGGAGAT |
| | | | GCTGCCCAGAAGACAGACACATCTCACCATGACCAGGACCACCCCACCTTCAACAAGATCACTCCCAATCTTGCAGA |
| | | | GTTTGCATTCTCTCTCTACAGACAGCTTGCACACCAGAGCAACTCTACTAACATCTTCTTCTCTCCAGTCAGCATAGCA |
| | | | ACAGCATTTGCAATGCTCAGCCTTGGCACAAAGGCAGACACACATGATGAGATCCTTGAGGGCCTCAACTTCAATCT |
| | | | CACAGAGATCCCAGAAGCCCAGATCCATGAGGGCTTCCAGGAGCTGCTGAGAACACTCAACCAGCCTGACTCTCAG |
| | | | CTCCAGCTCACAACAGGCAATGGGCTCTTCCTCTCTGAGGGCCTCAAGCTTGTAGACAAGTTCCTGGAGGATGTCAA |
| | | | GAAGCTCTACCACTCTGAAGCCTTCACAGTCAACTTTGGAGACACAGAGGAAGCCAAGAAGCAGATCAATGACTAT |
| | | | GTAGAGAAGGGGACTCAGGGCAAGATAGTAGACCTTGTCAAGGAGCTGGACAGAGACACAGTCTTTGCACTGGTC |
| | | | AACTACATCTTCTTCAAGGGGAAGTGGGAGAGACCCTTTGAAGTCAAGGACACAGAGGAGGAGGACTTCCATGTAG |
| | | | ACCAGGTGACAACAGTCAAGGTTCCCATGATGAAGAGACTTGGCATGTTCAATATCCAGCACTGCAAGAAGCTCAG |
| | | | CTCTTGGGTCCTCCTCATGAAGTACCTTGGCAATGCAACAGCAATCTTCTTCCTTCCTGATGAGGGCAAGCTCCAGCA |
| | | | CCTTGAGAATGAGCTGACACATGACATCATCACAAAGTTCCTGGAGAATGAGGACAGAAGGTCTGCATCTCTCCACC |
| | | | TTCCAAAGCTCAGCATCACAGGCACCTATGACCTCAAGTCTGTCCTTGGCCAGCTTGGCATCACAAAGGTCTTCTCTA |
| | | | ATGGTGCAGACCTCTCTGGAGTCACAGAGGAAGCCCCCCTCAAGCTCAGCAAGGCTGTGCACAAGGCTGTGCTCAC |
| | | | AATAGATGAGAAGGGGACAGAGGCTGCAGGTGCCATGTTCCTGGAAGCCATCCCCATGAGCATCCCACCAGAAGTC |
| | | | AAGTTCAACAAGCCTTTTGTCTTCCTGATGATAGAGCAGAACACAAAGTCTCCCCTCTTCATGGGCAAGGTAGTCAAC |
| | | | CCCACTCAAAAGTAACAGACATGATAAGATACATTGATGAGTTTGGACAAACCACAACTAGAATGCAGTGAAAAAA |
| | | | ATGCTTTATTTGTGAAATTTGTGATGCTATTGCTTTATTTGTAACCATTATAAGCTGCAATAAACAAGTTAACAACAAC |
| | | | AATTGCATTCATTTTATGTTTCAGGTTCAGGGGGAGGTGTGGGAGGTTTTTTggggataccccctagagccccagctggttctttt |
| | | | ctcctcagaagCCATAGAGCCCATCTCATCCCCAGCATGCCTGCTATTGTCTTCCCAATCCTCCCCCTTGCTGTCCTGCCCC |
| | | | ACCCCACCCCCCAGAATAGAATGACACCTACTCAGACAATTCTATGCAATTTCCTCATTTTATTAGGAAAGGACAGTG |
| | | | GGAGTGGCACCTTCCAGGGTCAAGGAAGGCATGGGGGAGGGGCAAACAACAGATGGCTGGCAACTAGAAGGCAC |
| | | | AGTCTaggTTACTTCTGGGTGGGGTTCACCACCTTGCCCATGAACAGGGGGCTCTTGGTGTTCTGCTCTATCATCAGG |
| | | | AACACAAAAGGCTTGTTGAACTTCACCTCTGGGGGGATGCTCATGGGGATGGCCTCCAGGAACATGGCTCCTGCTG |
| | | | CCTCTGTGCCCTTCTCATCTATGGTCAGCACTGCCTTGTGCACTGCCTTGCTCAGCTTCAGGGGGGCCTCCTCTGTCAC |
| | | | TCCAGACAGGTCTGCTCCATTGCTGAACACCTTGGTGATGCCCAGCTGGCCCAGCACAGACTTCAGGTCATAGGTGC |
| | | | CTGTGATGCTCAGCTTGGGCAGGTGCAGGCTGGCAGACCTCCTGTCCTCATTCTCCAGGAACTTGGTGATGATGTCA |
| | | | TGGGTCAGCTCATTCTCCAGGTGCTGCAGCTTGCCCTCATCTGGCAGGAAGAAGATGGCTGTGGCATTGCCCAGGTA |
| | | | CTTCATCAGCAGCACCCAGCTGCTCAGCTTCTTGCAGTGCTGGATATTGAACATGCCCAGCCTCTTCATCATGGGCAC |
| | | | CTTCACTGTGGTCACCTGGTCCACATGGAAGTCCTCCTCCTCTGTGTCCTTCACCTCAAAGGGCCTCTCCCACTTGCCC |
| | | | TTGAAGAAGATGTAGTTCACCAGGGCAAACACTGTGTCCCTGTCCAGCTCCTTCACCAGGTCCACTATCTTGCCCTGG |
| | | | GTGCCCTTCTCCACATAGTCATTGATCTGCTTCTTGGCCTCCTCTGTGTCTCCAAAGTTCACTGTGAAGGCCTCAGAGT |
| | | | GGTACAGCTTCTTCACATCCTCCAGGAACTTGTCCACCAGCTTCAGGCCCTCAGACAGGAACAGGCCATTGCCTGTG |
| | | | GTCAGCTGCAGCTGGCTGTCTGGCTGGTTCAGGGTCCTCAGCAGCTCCTGGAAGCCCTCATGGATCTGGGCCTCTGG |
| | | | GATCTCTGTCAGGTTGAAGTTCAGGCCCTCCAGGATCTCATCATGGGTGTCTGCCTTGGTGCCCAGGCTCAGCATGG |
| | | | CAAAGGCTGTGGCTATGCTCACTGGGCTGAAGAAGATGTTGGTGCTGTTGCTCTGGTGGGCCAGCTGCCTGTACAG |
| | | | GCTGAAGGCAAACTCTGCCAGGTTGGGGGTGATCTTGTTGAAGGTGGGGTGGTCCTGGTCATGGTGGCTGGTGTCT |
| | | | GTCTTCTGGGCTGCATCTCCCTGGGGGTCCTCaaCTGGGCAAGGGAAGaaaaaaaaGGATTGTTAAATACTGAAGAAA |
| | | | ACAAGAAGTAATAATGTTACTTTTTATATTTCTTTCCATTTGACTTAGATTATGCAACTAGTAGATCTAGGAACCCCTA |
| | | | GTGATGGAGTTGGCCACTCCCTCTCTGCGCGCTCGCTCGCTCACTGAGGCCGCCCGGGCAAAGCCCGGGCGTCGGG |
| | | | CGACCTTTGGTCGCCCGGCCTCAGTGAGCGAGCGAGCGCGCAGAGAGGGAGTGGCCAAacgcgtggtgtaatcatggtcat |
| | | | agctgtttcctgtgtgaaattgttatccgctcacaattccacacaacatacgagccggaagcataaagtgtaaagcctggggtgcctaatgagtgagcta |
| | | | actcacattaattgcgttgcgctcactgcccgctttccagtcgggaaacctgtcgtgccagctgcattaatgaatcggccaacgcgcggggagaggcggtt |
| | | | tgcgtattgggcgctcttccgcttcctcgctcactgactcgctgcgctcggtcgttcggctgcggcgagcggtatcagctcactcaaaggcggtaatacggt |
| | | | tatccacagaatcaggggataacgcaggaaagaacatgtgagcaaaaggccagcaaaaggccaggaaccgtaaaaaggccgcgttgctggcgttttt |
| | | | ccataggctccgcccccctgacgagcatcacaaaaatcgacgctcaagtcagaggtggcgaaacccgacaggactataaagataccaggcgtttcccc |
| | | | ctggaagctccctcgtgcgctctcctgttccgaccctgccgcttaccggatacctgtccgcctttctcccttcgggaagcgtggcgctttctcatagctcacg |
| | | | ctgtaggtatctcagttcggtgtaggtcgttcgctccaagctgggctgtgtgcacgaaccccccgttcagcccgaccgctgcgccttatccggtaactatcg |
| | | | tcttgagtccaacccggtaagacacgacttatcgccactggcagcagccactggtaacaggattagcagagcgaggtatgtaggcggtgctacagagtt |
| | | | cttgaagtggtggcctaactacggctacactagaagaacagtatttggtatctgcgctctgctgaagccagttaccttcggaaaaagagttggtagctctt |
| | | | gatccggcaaacaaaccaccgctggtagcggtggtttttttgtttgcaagcagcagattacgcgcagaaaaaaaggatctcaagaagatcctttgatctt |
| | | | ttctacggggtctgacgctcagtggaacgaaaactcacgttaagggattttggtcatgagattatcaaaaaggatcttcacctagatccttttaaattaaa |
| | | | aatgaagttttaaatcaagcccaatctgaataatgttacaaccaattaaccaattctgattagaaaaactcatcgagcatcaaatgaaactgcaatttatt |
| | | | catatcaggattatcaataccatatttttgaaaaagccgtttctgtaatgaaggagaaaactcaccgaggcagttccataggatggcaagatcctggtat |
| | | | cggtctgcgattccgactcgtccaacatcaatacaacctattaatttcccctcgtcaaaaataaggttatcaagtgagaaatcaccatgagtgacgactga |
| | | | atccggtgagaatggcaaaagtttatgcatttctttccagacttgttcaacaggccagccattacgctcgtcatcaaaatcactcgcatcaaccaaaccgt |
| | | | tattcattcgtgattgcgcctgagcgagacgaaatacgcgatcgctgttaaaaggacaattacaaacaggaatcgaatgcaaccggcgcaggaacact |
| | | | gccagcgcatcaacaatattttcacctgaatcaggatattcttctaatacctggaatgctgtttttccggggatcgcagtggtgagtaaccatgcatcatca |
| | | | ggagtacggataaaatgcttgatggtcggaagaggcataaattccgtcagccagtttagtctgaccatctcatctgtaacatcattggcaacgctaccttt |
| | | | gccatgtttcagaaacaactctggcgcatcgggcttcccatacaagcgatagattgtcgcacctgattgcccgacattatcgcgagcccatttatacccat |
| | | | ataaatcagcatccatgttggaatttaatcgcggcctcgacgtttcccgttgaatatggctcataacaccccttgtattactgtttatgtaagcagacagttt |
| | | | tattgttcatgatgatatatttttatcttgtgcaa |
| | SERPINA1 | A1AT w/o | GAGGACCCCCAGGGAGATGCTGCCCAGAAGACAGACACATCTCACCATGACCAGGACCACCCCACCTTCAACAAGA |
| | copy 1 | SP | TCACTCCCAATCTTGCAGAGTTTGCATTCTCTCTCTACAGACAGCTTGCACACCAGAGCAACTCTACTAACATCTTCTT |
| | | (alternate | CTCTCCAGTCAGCATAGCAACAGCATTTGCAATGCTCAGCCTTGGCACAAAGGCAGACACACATGATGAGATCCTTG |
| | | codon usage | AGGGCCTCAACTTCAATCTCACAGAGATCCCAGAAGCCCAGATCCATGAGGGCTTCCAGGAGCTGCTGAGAACACT |
| | | 2) CpG | CAACCAGCCTGACTCTCAGCTCCAGCTCACAACAGGCAATGGGCTCTTCCTCTCTGAGGGCCTCAAGCTTGTAGACA |
| | | depleted | AGTTCCTGGAGGATGTCAAGAAGCTCTACCACTCTGAAGCCTTCACAGTCAACTTTGGAGACACAGAGGAAGCCAA |
| | | SEQ ID NO: | GAAGCAGATCAATGACTATGTAGAGAAGGGGACTCAGGGCAAGATAGTAGACCTTGTCAAGGAGCTGGACAGAGA |
| | | 781 | CACAGTCTTTGCACTGGTCAACTACATCTTCTTCAAGGGGAAGTGGGAGAGACCCTTTGAAGTCAAGGACACAGAG |
| | | | GAGGAGGACTTCCATGTAGACCAGGTGACAACAGTCAAGGTTCCCATGATGAAGAGACTTGGCATGTTCAATATCC |
| | | | AGCACTGCAAGAAGCTCAGCTCTTGGGTCCTCCTCATGAAGTACCTTGGCAATGCAACAGCAATCTTCTTCCTTCCTG |
| | | | ATGAGGGCAAGCTCCAGCACCTTGAGAATGAGCTGACACATGACATCATCACAAAGTTCCTGGAGAATGAGGACAG |
| | | | AAGGTCTGCATCTCTCCACCTTCCAAAGCTCAGCATCACAGGCACCTATGACCTCAAGTCTGTCCTTGGCCAGCTTGG |
| | | | CATCACAAAGGTCTTCTCTAATGGTGCAGACCTCTCTGGAGTCACAGAGGAAGCCCCCCTCAAGCTCAGCAAGGCTG |
| | | | TGCACAAGGCTGTGCTCACAATAGATGAGAAGGGGACAGAGGCTGCAGGTGCCATGTTCCTGGAAGCCATCCCCAT |
| | | | GAGCATCCCACCAGAAGTCAAGTTCAACAAGCCTTTTGTCTTCCTGATGATAGAGCAGAACACAAAGTCTCCCCTCTT |
| | | | CATGGGCAAGGTAGTCAACCCCACTCAAAAG |
| | SERPINA1 | A1AT w/o | GAGGACCCCCAGGGAGATGCAGCCCAGAAGACAGACACCAGCCACCATGACCAGGACCACCCCACCTTCAACAAGA |
| | copy 2 (rev | SP CpG | TCACCCCCAACCTGGCAGAGTTTGCCTTCAGCCTGTACAGGCAGCTGGCCCACCAGAGCAACAGCACCAACATCTTC |
| | comp) | depleted | TTCAGCCCAGTGAGCATAGCCACAGCCTTTGCCATGCTGAGCCTGGGCACCAAGGCAGACACCCATGATGAGATCCT |
| | | SEQ ID NO: | GGAGGGCCTGAACTTCAACCTGACAGAGATCCCAGAGGCCCAGATCCATGAGGGCTTCCAGGAGCTGCTGAGGAC |
| | | 782 | CCTGAACCAGCCAGACAGCCAGCTGCAGCTGACCACAGGCAATGGCCTGTTCCTGTCTGAGGGCCTGAAGCTGGTG |
| | | | GACAAGTTCCTGGAGGATGTGAAGAAGCTGTACCACTCTGAGGCCTTCACAGTGAACTTTGGAGACACAGAGGAGG |
| | | | CCAAGAAGCAGATCAATGACTATGTGGAGAAGGGCACCCAGGGCAAGATAGTGGACCTGGTGAAGGAGCTGGAC |
| | | | AGGGACACAGTGTTTGCCCTGGTGAACTACATCTTCTTCAAGGGCAAGTGGGAGAGGCCCTTTGAGGTGAAGGACA |
| | | | CAGAGGAGGAGGACTTCCATGTGGACCAGGTGACCACAGTGAAGGTGCCCATGATGAAGAGGCTGGGCATGTTCA |
| | | | ATATCCAGCACTGCAAGAAGCTGAGCAGCTGGGTGCTGCTGATGAAGTACCTGGGCAATGCCACAGCCATCTTCTTC |
| | | | CTGCCAGATGAGGGCAAGCTGCAGCACCTGGAGAATGAGCTGACCCATGACATCATCACCAAGTTCCTGGAGAATG |
| | | | AGGACAGGAGGTCTGCCAGCCTGCACCTGCCCAAGCTGAGCATCACAGGCACCTATGACCTGAAGTCTGTGCTGGG |
| | | | CCAGCTGGGCATCACCAAGGTGTTCAGCAATGGAGCAGACCTGTCTGGAGTGACAGAGGAGGCCCCCCTGAAGCT |
| | | | GAGCAAGGCAGTGCACAAGGCAGTGCTGACCATAGATGAGAAGGGCACAGAGGCAGCAGGAGCCATGTTCCTGG |
| | | | AGGCCATCCCCATGAGCATCCCCCCAGAGGTGAAGTTCAACAAGCCTTTTGTGTTCCTGATGATAGAGCAGAACACC |
| | | | AAGAGCCCCCTGTTCATGGGCAAGGTGGTGAACCCCACCCAGAAGTAA |
| 20 | A1AT w/ SP | 1380 | ATGCCGTCTTCTGTCTCGTGGGGCATCCTCCTGCTGGCAGGCCTGTGCTGCCTGGTCCCTGTCTCCCTGGCTGAGGAT |
| | | | CCCCAGGGAGATGCTGCCCAGAAGACAGATACATCCCACCATGATCAGGATCACCCAACCTTCAACAAGATCACCCC |
| | | | CAACCTGGCTGAGTTCGCCTTCAGCCTATACCGCCAGCTGGCACACCAGTCCAACAGCACCAATATCTTCTTCTCCCC |
| | | | AGTGAGCATCGCTACAGCCTTTGCAATGCTCTCCCTGGGGACCAAGGCTGACACTCACGATGAAATCCTGGAGGGC |
| | | | CTGAATTTCAACCTCACGGAGATTCCGGAGGCTCAGATCCATGAAGGCTTCCAGGAACTCCTCCGTACCCTCAACCA |
| | | | GCCAGACAGCCAGCTCCAGCTGACCACCGGCAATGGCCTGTTCCTCAGCGAGGGCCTGAAGCTAGTGGATAAGTTT |
| | | | TTGGAGGATGTTAAAAAGTTGTACCACTCAGAAGCCTTCACTGTCAACTTCGGGGACACCGAAGAGGCCAAGAAAC |
| | | | AGATCAACGATTACGTGGAGAAGGGTACTCAAGGGAAAATTGTGGATTTGGTCAAGGAGCTTGACAGAGACACAG |
| | | | TTTTTGCTCTGGTGAATTACATCTTCTTTAAAGGCAAATGGGAGAGACCCTTTGAAGTCAAGGACACCGAGGAAGAG |
| | | | GACTTCCACGTGGACCAGGTGACCACCGTGAAGGTGCCTATGATGAAGCGTTTAGGCATGTTTAACATCCAGCACTG |
| | | | TAAGAAGCTGTCCAGCTGGGTGCTGCTGATGAAATACCTGGGCAATGCCACCGCCATCTTCTTCCTGCCTGATGAGG |
| | | | GGAAACTACAGCACCTGGAAAATGAACTCACCCACGATATCATCACCAAGTTCCTGGAAAATGAAGACAGAAGGTC |
| | | | TGCCAGCTTACATTTACCCAAACTGTCCATTACTGGAACCTATGATCTGAAGAGCGTCCTGGGTCAACTGGGCATCAC |
| | | | TAAGGTCTTCAGCAATGGGGCTGACCTCTCCGGGGTCACAGAGGAGGCACCCCTGAAGCTCTCCAAGGCCGTGCAT |
| | | | AAGGCTGTGCTGACCATCGACGAGAAAGGGACTGAAGCTGCTGGGGCCATGTTTTTAGAGGCCATACCCATGTCTA |
| | | | TCCCCCCCGAGGTCAAGTTCAACAAACCCTTTGTCTTCTTAATGATTGAACAAAATACCAAGTCTCCCCTCTTCATGGG |
| | | | AAAAGTGGTGAATCCCACCCAAAAATAA |
| 21 | A1AT w/o | 1382 | ATGAAGTGGGTAACCTTTATTTCCCTTCTTTTTCTCTTTAGCTCGGCTTATTCCAGGGGTGTGTTTCGTCGAGATGCAC |
| | SP | | ttGAGGATCCCCAGGGAGATGCTGCCCAGAAGACAGATACATCCCACCATGATCAGGATCACCCAACCTTCAACAAG |
| | | | ATCACCCCCAACCTGGCTGAGTTCGCCTTCAGCCTATACCGCCAGCTGGCACACCAGTCCAACAGCACCAATATCTTC |
| | | | TTCTCCCCAGTGAGCATCGCTACAGCCTTTGCAATGCTCTCCCTGGGGACCAAGGCTGACACTCACGATGAAATCCTG |
| | | | GAGGGCCTGAATTTCAACCTCACGGAGATTCCGGAGGCTCAGATCCATGAAGGCTTCCAGGAACTCCTCCGTACCCT |
| | | | CAACCAGCCAGACAGCCAGCTCCAGCTGACCACCGGCAATGGCCTGTTCCTCAGCGAGGGCCTGAAGCTAGTGGAT |
| | | | AAGTTTTTGGAGGATGTTAAAAAGTTGTACCACTCAGAAGCCTTCACTGTCAACTTCGGGGACACCGAAGAGGCCAA |
| | | | GAAACAGATCAACGATTACGTGGAGAAGGGTACTCAAGGGAAAATTGTGGATTTGGTCAAGGAGCTTGACAGAGA |
| | | | CACAGTTTTTGCTCTGGTGAATTACATCTTCTTTAAAGGCAAATGGGAGAGACCCTTTGAAGTCAAGGACACCGAGG |
| | | | AAGAGGACTTCCACGTGGACCAGGTGACCACCGTGAAGGTGCCTATGATGAAGCGTTTAGGCATGTTTAACATCCA |
| | | | GCACTGTAAGAAGCTGTCCAGCTGGGTGCTGCTGATGAAATACCTGGGCAATGCCACCGCCATCTTCTTCCTGCCTG |
| | | | ATGAGGGGAAACTACAGCACCTGGAAAATGAACTCACCCACGATATCATCACCAAGTTCCTGGAAAATGAAGACAG |
| | | | AAGGTCTGCCAGCTTACATTTACCCAAACTGTCCATTACTGGAACCTATGATCTGAAGAGCGTCCTGGGTCAACTGG |
| | | | GCATCACTAAGGTCTTCAGCAATGGGGCTGACCTCTCCGGGGTCACAGAGGAGGCACCCCTGAAGCTCTCCAAGGC |
| | | | CGTGCATAAGGCTGTGCTGACCATCGACGAGAAAGGGACTGAAGCTGCTGGGGCCATGTTTTTAGAGGCCATACCC |
| | | | ATGTCTATCCCCCCCGAGGTCAAGTTCAACAAACCCTTTGTCTTCTTAATGATTGAACAAAATACCAAGTCTCCCCTCT |
| | | | TCATGGGAAAAGTGGTGAATCCCACCCAAAAAtaa |
| 22 | A1AT w/o | 1384 | ATGAAGTGGGTAACCTTTATTTCCCTTCTTTTTCTCTTTAGCTCGGCTTATTCCAGGGGTGTGTTTCGTCGAGATGCAC |
| | SP CpG | | ttGAGGACCCCCAGGGAGATGCAGCCCAGAAGACAGACACCAGCCACCATGACCAGGACCACCCCACCTTCAACAA |
| | depleted | | GATCACCCCCAACCTGGCAGAGTTTGCCTTCAGCCTGTACAGGCAGCTGGCCCACCAGAGCAACAGCACCAACATCT |
| | | | TCTTCAGCCCAGTGAGCATAGCCACAGCCTTTGCCATGCTGAGCCTGGGCACCAAGGCAGACACCCATGATGAGATC |
| | | | CTGGAGGGCCTGAACTTCAACCTGACAGAGATCCCAGAGGCCCAGATCCATGAGGGCTTCCAGGAGCTGCTGAGGA |
| | | | CCCTGAACCAGCCAGACAGCCAGCTGCAGCTGACCACAGGCAATGGCCTGTTCCTGTCTGAGGGCCTGAAGCTGGT |
| | | | GGACAAGTTCCTGGAGGATGTGAAGAAGCTGTACCACTCTGAGGCCTTCACAGTGAACTTTGGAGACACAGAGGAG |
| | | | GCCAAGAAGCAGATCAATGACTATGTGGAGAAGGGCACCCAGGGCAAGATAGTGGACCTGGTGAAGGAGCTGGA |
| | | | CAGGGACACAGTGTTTGCCCTGGTGAACTACATCTTCTTCAAGGGCAAGTGGGAGAGGCCCTTTGAGGTGAAGGAC |
| | | | ACAGAGGAGGAGGACTTCCATGTGGACCAGGTGACCACAGTGAAGGTGCCCATGATGAAGAGGCTGGGCATGTTC |
| | | | AATATCCAGCACTGCAAGAAGCTGAGCAGCTGGGTGCTGCTGATGAAGTACCTGGGCAATGCCACAGCCATCTTCTT |
| | | | CCTGCCAGATGAGGGCAAGCTGCAGCACCTGGAGAATGAGCTGACCCATGACATCATCACCAAGTTCCTGGAGAAT |
| | | | GAGGACAGGAGGTCTGCCAGCCTGCACCTGCCCAAGCTGAGCATCACAGGCACCTATGACCTGAAGTCTGTGCTGG |
| | | | GCCAGCTGGGCATCACCAAGGTGTTCAGCAATGGAGCAGACCTGTCTGGAGTGACAGAGGAGGCCCCCCTGAAGC |
| | | | TGAGCAAGGCAGTGCACAAGGCAGTGCTGACCATAGATGAGAAGGGCACAGAGGCAGCAGGAGCCATGTTCCTGG |
| | | | AGGCCATCCCCATGAGCATCCCCCCAGAGGTGAAGTTCAACAAGCCTTTTGTGTTCCTGATGATAGAGCAGAACACC |
| | | | AAGAGCCCCCTGTTCATGGGCAAGGTGGTGAACCCCACCCAGAAGTAA |
| 23 | A1AT w/o | 1386 | ATGAAGTGGGTAACCTTTATTTCCCTTCTTTTTCTCTTTAGCTCGGCTTATTCCAGGGGTGTGTTTCGTCGAGATGCAC |
| | SP (altern- | | ttGAGGATCCCCAGGGAGATGCTGCCCAGAAGACAGATACATCCCACCATGATCAGGATCACCCAACCTTCAACAAG |
| | ative | | ATCACCCCCAACCTGGCTGAGTTTGCCTTCAGCCTATACAGACAGCTGGCACACCAGTCCAACAGCACCAATATCTTC |
| | codon usage | | TTCTCCCCAGTGAGCATAGCTACAGCCTTTGCAATGCTCTCCCTGGGGACCAAGGCTGACACTCATGATGAAATCCTG |
| | 1) CpG | | GAGGGCCTGAATTTCAACCTCACAGAGATTCCAGAGGCTCAGATCCATGAAGGCTTCCAGGAACTCCTCAGAACCCT |
| | depleted | | CAACCAGCCAGACAGCCAGCTCCAGCTGACCACAGGCAATGGCCTGTTCCTCTCTGAGGGCCTGAAGCTAGTGGAT |
| | | | AAGTTTTTGGAGGATGTTAAAAAGTTGTACCACTCAGAAGCCTTCACTGTCAACTTTGGGGACACAGAAGAGGCCAA |
| | | | GAAACAGATCAATGATTATGTGGAGAAGGGTACTCAAGGGAAAATTGTGGATTTGGTCAAGGAGCTTGACAGAGA |
| | | | CACAGTTTTTGCTCTGGTGAATTACATCTTCTTTAAAGGCAAATGGGAGAGACCCTTTGAAGTCAAGGACACAGAGG |
| | | | AAGAGGACTTCCATGTGGACCAGGTGACCACAGTGAAGGTGCCTATGATGAAAAGGCTTGGTATGTTCAATATCCA |
| | | | GCACTGTAAGAAGCTGTCCAGCTGGGTGCTGCTGATGAAATACCTGGGCAATGCCACAGCCATCTTCTTCCTGCCTG |
| | | | ATGAGGGGAAACTACAGCACCTGGAAAATGAACTCACCCATGATATCATCACCAAGTTCCTGGAAAATGAAGACAG |
| | | | AAGGTCTGCCAGCTTACATTTACCCAAACTGTCCATTACTGGAACCTATGATCTGAAGTCTGTCCTGGGTCAACTGGG |
| | | | CATCACTAAGGTCTTCAGCAATGGGGCTGACCTCTCTGGGGTCACAGAGGAGGCACCCCTGAAGCTCTCCAAGGCA |
| | | | GTGCATAAGGCTGTGCTGACCATAGATGAGAAGGGCACAGAGGCTGCTGGGGCCATGTTTTTAGAGGCCATACCCA |
| | | | TGTCTATCCCCCCAGAGGTCAAGTTCAACAAACCTTTTGTATTTCTCATGATAGAGCAGAACACTAAATCACCCCTCTT |
| | | | CATGGGAAAAGTGGTGAATCCCACCCAAAAAtaa |
| 24 | A1AT w/o | 1388 | ATGAAGTGGGTAACCTTTATTTCCCTTCTTTTTCTCTTTAGCTCGGCTTATTCCAGGGGTGTGTTTCGTCGAGATGCAC |
| | SP altern- | | ttGAGGACCCCCAGGGAGATGCTGCCCAGAAGACAGACACATCTCACCATGACCAGGACCACCCCACCTTCAACAAG |
| | (ative | | ATCACTCCCAATCTTGCAGAGTTTGCATTCTCTCTCTACAGACAGCTTGCACACCAGAGCAACTCTACTAACATCTTCT |
| | codon usage | | TCTCTCCAGTCAGCATAGCAACAGCATTTGCAATGCTCAGCCTTGGCACAAAGGCAGACACACATGATGAGATCCTT |
| | 2) CpG | | GAGGGCCTCAACTTCAATCTCACAGAGATCCCAGAAGCCCAGATCCATGAGGGCTTCCAGGAGCTGCTGAGAACAC |
| | depleted | | TCAACCAGCCTGACTCTCAGCTCCAGCTCACAACAGGCAATGGGCTCTTCCTCTCTGAGGGCCTCAAGCTTGTAGACA |
| | | | AGTTCCTGGAGGATGTCAAGAAGCTCTACCACTCTGAAGCCTTCACAGTCAACTTTGGAGACACAGAGGAAGCCAA |
| | | | GAAGCAGATCAATGACTATGTAGAGAAGGGGACTCAGGGCAAGATAGTAGACCTTGTCAAGGAGCTGGACAGAGA |
| | | | CACAGTCTTTGCACTGGTCAACTACATCTTCTTCAAGGGGAAGTGGGAGAGACCCTTTGAAGTCAAGGACACAGAG |
| | | | GAGGAGGACTTCCATGTAGACCAGGTGACAACAGTCAAGGTTCCCATGATGAAGAGACTTGGCATGTTCAATATCC |
| | | | AGCACTGCAAGAAGCTCAGCTCTTGGGTCCTCCTCATGAAGTACCTTGGCAATGCAACAGCAATCTTCTTCCTTCCTG |
| | | | ATGAGGGCAAGCTCCAGCACCTTGAGAATGAGCTGACACATGACATCATCACAAAGTTCCTGGAGAATGAGGACAG |
| | | | AAGGTCTGCATCTCTCCACCTTCCAAAGCTCAGCATCACAGGCACCTATGACCTCAAGTCTGTCCTTGGCCAGCTTGG |
| | | | CATCACAAAGGTCTTCTCTAATGGTGCAGACCTCTCTGGAGTCACAGAGGAAGCCCCCCTCAAGCTCAGCAAGGCTG |
| | | | TGCACAAGGCTGTGCTCACAATAGATGAGAAGGGGACAGAGGCTGCAGGTGCCATGTTCCTGGAAGCCATCCCCAT |
| | | | GAGCATCCCACCAGAAGTCAAGTTCAACAAGCCTTTTGTCTTCCTGATGATAGAGCAGAACACAAAGTCTCCCCTCTT |
| | | | CATGGGCAAGGTAGTCAACCCCACTCAAAAG |
| Construct | A1AT w/o | 1390 | ATGAAGTGGGTAACCTTTATTTCCCTTCTTTTTCTCTTTAGCTCGGCTTATTCCAGGGG |
| 23 design | SP altern- | | TGTGTTTCGTCGAGATGCACttGAGGACCCCCAGGGAGATGCAGCCCAGAAGACAGAC |
| | (ative | | ACCAGCCACCATGACCAGGACCACCCCACCTTCAACAAGATCACCCCCAACCTGGCA |
| | codon usage | | GAGTTTGCCTTCAGCCTGTACAGGCAGCTGGCCCACCAGAGCAACAGCACCAACATC |
| | 1) CpG | | TTCTTCAGCCCAGTGAGCATAGCCACAGCCTTTGCCATGCTGAGCCTGGGCACCAAGG |
| | depleted | | CAGACACCCATGATGAGATCCTGGAGGGCCTGAACTTCAACCTGACAGAGATCCCAG |
| | | | AGGCCCAGATCCATGAGGGCTTCCAGGAGCTGCTGAGGACCCTGAACCAGCCAGACA |
| | | | GCCAGCTGCAGCTGACCACAGGCAATGGCCTGTTCCTGTCTGAGGGCCTGAAGCTGG |
| | | | TGGACAAGTTCCTGGAGGATGTGAAGAAGCTGTACCACTCTGAGGCCTTCACAGTGA |
| | | | ACTTTGGAGACACAGAGGAGGCCAAGAAGCAGATCAATGACTATGTGGAGAAGGGC |
| | | | ACCCAGGGCAAGATAGTGGACCTGGTGAAGGAGCTGGACAGGGACACAGTGTTTGCC |
| | | | CTGGTGAACTACATCTTCTTCAAGGGCAAGTGGGAGAGGCCCTTTGAGGTGAAGGAC |
| | | | ACAGAGGAGGAGGACTTCCATGTGGACCAGGTGACCACAGTGAAGGTGCCCATGATG |
| | | | AAGAGGCTGGGCATGTTCAATATCCAGCACTGCAAGAAGCTGAGCAGCTGGGTGCTG |
| | | | CTGATGAAGTACCTGGGCAATGCCACAGCCATCTTCTTCCTGCCAGATGAGGGCAAG |
| | | | CTGCAGCACCTGGAGAATGAGCTGACCCATGACATCATCACCAAGTTCCTGGAGAAT |
| | | | GAGGACAGGAGGTCTGCCAGCCTGCACCTGCCCAAGCTGAGCATCACAGGCACCTAT |
| | | | GACCTGAAGTCTGTGCTGGGCCAGCTGGGCATCACCAAGGTGTTCAGCAATGGAGCA |
| | | | GACCTGTCTGGAGTGACAGAGGAGGCCCCCCTGAAGCTGAGCAAGGCAGTGCACAAG |
| | | | GCAGTGCTGACCATAGATGAGAAGGGCACAGAGGCAGCAGGAGCCATGTTCCTGGA |
| | | | GGCCATCCCCATGAGCATCCCCCCAGAGGTGAAGTTCAACAAGCCTTTTGTGTTCCTG |
| | | | ATGATAGAGCAGAACACCAAGAGCCCCCTGTTCATGGGCAAGGTGGTGAACCCCACC |
| | | | CAGAAGTAA |

Universal to templates provided in SEQ ID NOs: 770, 710, 720, 730, 740, 750, 760, 780, 790, 795, and 1564 are the following sequences:

Splice acceptor Fwd:
(SEQ ID NO: 1301)
taggtcagtgaagagaagaacaaaaagcagcatattacagttagttg
tcttcatcaatctttaaatatgttgtgtggtttttctctccctgttt
ccacag
Splice acceptor Rev:
(SEQ ID NO: 1302)
ctgtggaaacagggagagaaaaaccacacaacatatttaaagattga
tgaagacaactaactgtaatatgctgctttttgttcttctcttcact
gaccta
Splice acceptor Fwd for SEQ ID NO: 1564
(SEQ ID NO: 1554)
TGCATAATCTAAGTCAAATGGAAAGAAATATAAAAAGTAACATTATT
ACTTCTTGTTTTCTTCAGTATTTAACAATCCttttttttCTTCCCTT
GCCCAG
Splice acceptor Rev for SEQ ID NO: 1564
(SEQ ID NO: 1555)
CTGGGCAAGGGAAGaaaaaaaaGGATTGTTAAATACTGAAGAAAACA
AGAAGTAATAATGTTACTTTTTATATTTCTTTCCATTTGACTTAGAT
TATGCA
Universal to all templates are the following
sequences Terminator fwd:
(SEQ ID NO: 1304)
CAGACATGATAAGATACATTGATGAGTTTGGACAAACCACAACTAGA
ATGCAGTGAAAAAAATGCTTTATTTGTGAAATTTGTGATGCTATTGC
TTTATTTGTAACCATTATAAGCTGCAATAAACAAGTTAACAACAACA
ATTGCATTCATTTTATGTTTCAGGTTCAGGGGGAGGTGTGGGAGGTT
TTTT
Terminator Rev:
(SEQ ID NO: 1305)
ggggataccccctagagccccagctggttcttttctcctcagaagCC
ATAGAGCCCATCTCATCCCCAGCATGCCTGCTATTGTCTTCCCAATC
CTCCCCCTTGCTGTCCTGCCCCACCCCACCCCCCAGAATAGAATGAC
ACCTACTCAGACAATTCTATGCAATTTCCTCATTTTATTAGGAAAGG
ACAGTGGGAGTGGCACCTTCCAGGGTCAAGGAAGGCATGGGGGAGGG
GCAAACAACAGATGGCTGGCAACTAGAAGGCACAGTCTagg

TABLE 9B

| SEQ | | |
|---|---|---|
| ID | | |
| NO | Name | Sequence |
| 1400 | wt | GAGGACCCCCAGGGCGACGCCGCCCAGAAGACCGACACCAGCCACC |
| | SERPINA1 | ACGACCAGGACCACCCCACCTTCAACAAGATCACCCCCAACCTGGCC |
| | from | GAGTTCGCCTTCAGCCTGTACAGGCAGCTGGCCCACCAGAGCAACAG |
| | Construct 1 | CACCAACATCTTCTTCAGCCCCGTGAGCATCGCCACCGCCTTCGCCAT |
| | | GCTGAGCCTGGGCACCAAGGCCGACACCCACGACGAGATCCTGGAGG |
| | | GCCTGAACTTCAACCTGACCGAGATCCCCGAGGCCCAGATCCACGAG |
| | | GGCTTCCAGGAGCTGCTGAGGACCCTGAACCAGCCCGACAGCCAGCT |
| | | GCAGCTGACCACCGGCAACGGCCTGTTCCTGAGCGAGGGCCTGAAGC |
| | | TGGTGGACAAGTTCCTGGAGGACGTGAAGAAGCTGTACCACAGCGAG |
| | | GCCTTCACCGTGAACTTCGGCGACACCGAGGAGGCCAAGAAGCAGAT |
| | | CAACGACTACGTGGAGAAGGGCACCCAGGGCAAGATCGTGGACCTG |
| | | GTGAAGGAGCTGGACAGGGACACCGTGTTCGCCCTGGTGAACTACAT |
| | | CTTCTTCAAGGGCAAGTGGGAGAGGCCCTTCGAGGTGAAGGACACCG |
| | | AGGAGGAGGACTTCCACGTGGACCAGGTGACCACCGTGAAGGTGCCC |
| | | ATGATGAAGAGGCTGGGCATGTTCAACATCCAGCACTGCAAGAAGCT |
| | | GAGCAGCTGGGTGCTGCTGATGAAGTACCTGGGCAACGCCACCGCCA |
| | | TCTTCTTCCTGCCCGACGAGGGCAAGCTGCAGCACCTGGAGAACGAG |
| | | CTGACCCACGACATCATCACCAAGTTCCTGGAGAACGAGGACAGGAG |
| | | GAGCGCCAGCCTGCACCTGCCCAAGCTGAGCATCACCGGCACCTACG |
| | | ACCTGAAGAGCGTGCTGGGCCAGCTGGGCATCACCAAGGTGTTCAGC |
| | | AACGGCGCCGACCTGAGCGGCGTGACCGAGGAGGCCCCCCTGAAGCT |
| | | GAGCAAGGCCGTGCACAAGGCCGTGCTGACCATCGACGAGAAGGGC |
| | | ACCGAGGCCGCCGGCGCCATGTTCCTGGAGGCCATCCCCATGAGCAT |
| | | CCCCCCCGAGGTGAAGTTCAACAAGCCTTTCGTGTTCCTGATGATCGA |
| | | GCAGAACACCAAGAGCCCCCTGTTCATGGGCAAGGTGGTGAACCCCA |
| | | CCCAGAAGTAA |
| 1401 | wt | ttaTTTTTGGGTGGGATTCACCACTTTTCCCATGAAGAGGGGTGATTTAG |
| | SERPINA1- | TGTTCTGCTCGATCATGAGAAATACAAAAGGTTTGTTGAACTTGACCT |
| | alternative | CGGGGGGGATAGACATGGGTATGGCCTCTAAAAACATGGCCCCAGCA |
| | codon usage | GCCTCGGTGCCCTTCTCGTCGATGGTCAGCACAGCCTTATGCACGGCC |
| | 1-from | TTGGAGAGCTTCAGGGGTGCCTCCTCTGTGACCCCGGAGAGGTCAGC |
| | Construct 1 | CCCATTGCTGAAGACCTTAGTGATGCCCAGTTGACCCAGGACGCTCTT |
| | | CAGATCATAGGTTCCAGTAATGGACAGTTTGGGTAAATGTAAGCTGG |
| | | CAGACCTTCTGTCTTCATTTTCCAGGAACTTGGTGATGATATCGTGGG |
| | | TGAGTTCATTTTCCAGGTGCTGTAGTTTCCCCTCATCAGGCAGGAAGA |
| | | AGATGGCGGTGGCATTGCCCAGGTATTTCATCAGCAGCACCCAGCTG |
| | | GACAGCTTCTTACAGTGCTGGATATTGAACATACCAAGCCTTTTCATC |
| | | ATAGGCACCTTCACGGTGGTCACCTGGTCCACGTGGAAGTCCTCTTCC |
| | | TCGGTGTCCTTGACTTCAAAGGGTCTCTCCCATTTGCCTTTAAAGAAG |
| | | ATGTAATTCACCAGAGCAAAAACTGTGTCTCTGTCAAGCTCCTTGACC |
| | | AAATCCACAATTTTCCCTTGAGTACCCTTCTCCACGTAATCGTTGATCT |
| | | GTTTCTTGGCCTCTTCGGTGTCCCCGAAGTTGACAGTGAAGGCTTCTG |
| | | AGTGGTACAACTTTTTAACATCCTCCAAAAACTTATCCACTAGCTTCA |
| | | GGCCCTCGCTGAGGAACAGGCCATTGCCGGTGGTCAGCTGGAGCTGG |
| | | CTGTCTGGCTGGTTGAGGGTACGGAGGAGTTCCTGGAAGCCTTCATG |
| | | GATCTGAGCCTCCGGAATCTCCGTGAGGTTGAAATTCAGGCCCTCCAG |
| | | GATTTCATCGTGAGTGTCAGCCTTGGTCCCCAGGGAGAGCATTGCAA |
| | | AGGCTGTAGCGATGCTCACTGGGGAGAAGAAGATATTGGTGCTGTTG |
| | | GACTGGTGTGCCAGCTGGCGGTATAGGCTGAAGGCGAACTCAGCCAG |
| | | GTTGGGGGTGATCTTGTTGAAGGTTGGGTGATCCTGATCATGGTGGGA |
| | | TGTATCTGTCTTCTGGGCAGCATCTCCCTGGGGATCCTC |
| 1402 | wt | GAGGACCCCCAGGGAGATGCAGCCCAGAAGACAGACACCAGCCACC |
| | SERPINA1 | ATGACCAGGACCACCCCACCTTCAACAAGATCACCCCCAACCTGGCA |
| | with CpG | GAGTTTGCCTTCAGCCTGTACAGGCAGCTGGCCCACCAGAGCAACAG |
| | depletion | CACCAACATCTTCTTCAGCCCAGTGAGCATAGCCACAGCCTTTGCCAT |
| | from | GCTGAGCCTGGGCACCAAGGCAGACACCCATGATGAGATCCTGGAGG |
| | Construct 7 | GCCTGAACTTCAACCTGACAGAGATCCCAGAGGCCCAGATCCATGAG |
| | | GGCTTCCAGGAGCTGCTGAGGACCCTGAACCAGCCAGACAGCCAGCT |
| | | GCAGCTGACCACAGGCAATGGCCTGTTCCTGTCTGAGGGCCTGAAGC |
| | | TGGTGGACAAGTTCCTGGAGGATGTGAAGAAGCTGTACCACTCTGAG |
| | | GCCTTCACAGTGAACTTTGGAGACACAGAGGAGGCCAAGAAGCAGAT |
| | | CAATGACTATGTGGAGAAGGGCACCCAGGGCAAGATAGTGGACCTGG |
| | | TGAAGGAGCTGGACAGGGACACAGTGTTTGCCCTGGTGAACTACATC |
| | | TTCTTCAAGGGCAAGTGGGAGAGGCCCTTTGAGGTGAAGGACACAGA |
| | | GGAGGAGGACTTCCATGTGGACCAGGTGACCACAGTGAAGGTGCCCA |
| | | TGATGAAGAGGCTGGGCATGTTCAATATCCAGCACTGCAAGAAGCTG |
| | | AGCAGCTGGGTGCTGCTGATGAAGTACCTGGGCAATGCCACAGCCAT |
| | | CTTCTTCCTGCCAGATGAGGGCAAGCTGCAGCACCTGGAGAATGAGC |
| | | TGACCCATGACATCATCACCAAGTTCCTGGAGAATGAGGACAGGAGG |
| | | TCTGCCAGCCTGCACCTGCCCAAGCTGAGCATCACAGGCACCTATGA |
| | | CCTGAAGTCTGTGCTGGGCCAGCTGGGCATCACCAAGGTGTTCAGCA |
| | | ATGGAGCAGACCTGTCTGGAGTGACAGAGGAGGCCCCCCTGAAGCTG |
| | | AGCAAGGCAGTGCACAAGGCAGTGCTGACCATAGATGAGAAGGGCA |
| | | CAGAGGCAGCAGGAGCCATGTTCCTGGAGGCCATCCCCATGAGCATC |
| | | CCCCCAGAGGTGAAGTTCAACAAGCCTTTTGTGTTCCTGATGATAGAG |
| | | CAGAACACCAAGAGCCCCCTGTTCATGGGCAAGGTGGTGAACCCCAC |
| | | CCAGAAGTAA |
| 1403 | wt | ttaTTTTTGGGTGGGATTCACCACTTTTCCCATGAAGAGGGGTGATTTAG |
| | SERPINA1- | TGTTCTGCTCTATCATGAGAAATACAAAAGGTTTGTTGAACTTGACCT |
| | alternative | CTGGGGGGATAGACATGGGTATGGCCTCTAAAAACATGGCCCCAGCA |
| | codon usage | GCCTCTGTGCCCTTCTCATCTATGGTCAGCACAGCCTTATGCACTGCC |
| | 1-CpG | TTGGAGAGCTTCAGGGGTGCCTCCTCTGTGACCCCAGAGAGGTCAGC |
| | depletion | CCCATTGCTGAAGACCTTAGTGATGCCCAGTTGACCCAGGACAGACTT |
| | from | CAGATCATAGGTTCCAGTAATGGACAGTTTGGGTAAATGTAAGCTGG |
| | Construct 7/8 | CAGACCTTCTGTCTTCATTTTCCAGGAACTTGGTGATGATATCATGGG |
| | | TGAGTTCATTTTCCAGGTGCTGTAGTTTCCCCTCATCAGGCAGGAAGA |
| | | AGATGGCTGTGGCATTGCCCAGGTATTTCATCAGCAGCACCCAGCTG |
| | | GACAGCTTCTTACAGTGCTGGATATTGAACATACCAAGCCTTTTCATC |
| | | ATAGGCACCTTCACTGTGGTCACCTGGTCCACATGGAAGTCCTCTTCC |
| | | TCTGTGTCCTTGACTTCAAAGGGTCTCTCCCATTTGCCTTTAAAGAAG |
| | | ATGTAATTCACCAGAGCAAAAACTGTGTCTCTGTCAAGCTCCTTGACC |
| | | AAATCCACAATTTTCCCTTGAGTACCCTTCTCCACATAATCATTGATCT |
| | | GTTTCTTGGCCTCTTCTGTGTCCCCAAAGTTGACAGTGAAGGCTTCTG |
| | | AGTGGTACAACTTTTTAACATCCTCCAAAAACTTATCCACTAGCTTCA |
| | | GGCCCTCAGAGAGGAACAGGCCATTGCCTGTGGTCAGCTGGAGCTGG |
| | | CTGTCTGGCTGGTTGAGGGTTCTGAGGAGTTCCTGGAAGCCTTCATGG |
| | | ATCTGAGCCTCTGGAATCTCTGTGAGGTTGAAATTCAGGCCCTCCAGG |
| | | ATTTCATCATGAGTGTCAGCCTTGGTCCCCAGGGAGAGCATTGCAAA |
| | | GGCTGTAGCTATGCTCACTGGGGAGAAGAAGATATTGGTGCTGTTGG |
| | | ACTGGTGTGCCAGCTGTCTGTATAGGCTGAAGGCAAACTCAGCCAGG |
| | | TTGGGGGTGATCTTGTTGAAGGTTGGGTGATCCTGATCATGGTGGGAT |
| | | GTATCTGTCTTCTGGGCAGCATCTCCCTGGGGATCCTC |
| 1404 | wt | GAGGACCCCCAGGGAGATGCTGCCCAGAAGACAGACACATCTCACCA |
| | SERPINA1- | TGACCAGGACCACCCCACCTTCAACAAGATCACTCCCAATCTTGCAG |
| | alternative | AGTTTGCATTCTCTCTCTACAGACAGCTTGCACACCAGAGCAACTCTA |
| | codon usage | CTAACATCTTCTTCTCTCCAGTCAGCATAGCAACAGCATTTGCAATGC |
| | 2-CpG | TCAGCCTTGGCACAAAGGCAGACACACATGATGAGATCCTTGAGGGC |
| | depletion | CTCAACTTCAATCTCACAGAGATCCCAGAAGCCCAGATCCATGAGGG |
| | from | CTTCCAGGAGCTGCTGAGAACACTCAACCAGCCTGACTCTCAGCTCCA |
| | Construct 8 | GCTCACAACAGGCAATGGGCTCTTCCTCTCTGAGGGCCTCAAGCTTGT |
| | | AGACAAGTTCCTGGAGGATGTCAAGAAGCTCTACCACTCTGAAGCCT |
| | | TCACAGTCAACTTTGGAGACACAGAGGAAGCCAAGAAGCAGATCAAT |
| | | GACTATGTAGAGAAGGGGACTCAGGGCAAGATAGTAGACCTTGTCAA |
| | | GGAGCTGGACAGAGACACAGTCTTTGCACTGGTCAACTACATCTTCTT |
| | | CAAGGGGAAGTGGGAGAGACCCTTTGAAGTCAAGGACACAGAGGAG |
| | | GAGGACTTCCATGTAGACCAGGTGACAACAGTCAAGGTTCCCATGAT |
| | | GAAGAGACTTGGCATGTTCAATATCCAGCACTGCAAGAAGCTCAGCT |
| | | CTTGGGTCCTCCTCATGAAGTACCTTGGCAATGCAACAGCAATCTTCT |
| | | TCCTTCCTGATGAGGGCAAGCTCCAGCACCTTGAGAATGAGCTGACA |
| | | CATGACATCATCACAAAGTTCCTGGAGAATGAGGACAGAAGGTCTGC |
| | | ATCTCTCCACCTTCCAAAGCTCAGCATCACAGGCACCTATGACCTCAA |
| | | GTCTGTCCTTGGCCAGCTTGGCATCACAAAGGTCTTCTCTAATGGTGC |
| | | AGACCTCTCTGGAGTCACAGAGGAAGCCCCCCTCAAGCTCAGCAAGG |
| | | CTGTGCACAAGGCTGTGCTCACAATAGATGAGAAGGGGACAGAGGCT |
| | | GCAGGTGCCATGTTCCTGGAAGCCATCCCCATGAGCATCCCACCAGA |
| | | AGTCAAGTTCAACAAGCCTTTTGTCTTCCTGATGATAGAGCAGAACAC |
| | | AAAGTCTCCCCTCTTCATGGGCAAGGTAGTCAACCCCACTCAAAAG |
| 1405 | WT SERPINA1 | ATGCCGTCTTCTGTCTCGTGGGGCATCCTCCTGCTGGCAGGCCTGTGC |
| | ORF | TGCCTGGTCCCTGTCTCCCTGGCTGAGGATCCCCAGGGAGATGCTGCC |
| | | CAGAAGACAGATACATCCCACCATGATCAGGATCACCCAACCTTCAA |
| | | CAAGATCACCCCCAACCTGGCTGAGTTCGCCTTCAGCCTATACCGCCA |
| | | GCTGGCACACCAGTCCAACAGCACCAATATCTTCTTCTCCCCAGTGAG |
| | | CATCGCTACAGCCTTTGCAATGCTCTCCCTGGGGACCAAGGCTGACAC |
| | | TCACGATGAAATCCTGGAGGGCCTGAATTTCAACCTCACGGAGATTC |
| | | CGGAGGCTCAGATCCATGAAGGCTTCCAGGAACTCCTCCGTACCCTC |
| | | AACCAGCCAGACAGCCAGCTCCAGCTGACCACCGGCAATGGCCTGTT |
| | | CCTCAGCGAGGGCCTGAAGCTAGTGGATAAGTTTTTGGAGGATGTTA |
| | | AAAAGTTGTACCACTCAGAAGCCTTCACTGTCAACTTCGGGGACACC |
| | | GAAGAGGCCAAGAAACAGATCAACGATTACGTGGAGAAGGGTACTC |
| | | AAGGGAAAATTGTGGATTTGGTCAAGGAGCTTGACAGAGACACAGTT |
| | | TTTGCTCTGGTGAATTACATCTTCTTTAAAGGCAAATGGGAGAGACCC |
| | | TTTGAAGTCAAGGACACCGAGGAAGAGGACTTCCACGTGGACCAGGT |
| | | GACCACCGTGAAGGTGCCTATGATGAAGCGTTTAGGCATGTTTAACA |
| | | TCCAGCACTGTAAGAAGCTGTCCAGCTGGGTGCTGCTGATGAAATAC |
| | | CTGGGCAATGCCACCGCCATCTTCTTCCTGCCTGATGAGGGGAAACTA |
| | | CAGCACCTGGAAAATGAACTCACCCACGATATCATCACCAAGTTCCT |
| | | GGAAAATGAAGACAGAAGGTCTGCCAGCTTACATTTACCCAAACTGT |
| | | CCATTACTGGAACCTATGATCTGAAGAGCGTCCTGGGTCAACTGGGC |
| | | ATCACTAAGGTCTTCAGCAATGGGGCTGACCTCTCCGGGGTCACAGA |
| | | GGAGGCACCCCTGAAGCTCTCCAAGGCCGTGCATAAGGCTGTGCTGA |
| | | CCATCGACGAGAAAGGGACTGAAGCTGCTGGGGCCATGTTTTTAGAG |
| | | GCCATACCCATGTCTATCCCCCCCGAGGTCAAGTTCAACAAACCCTTT |
| | | GTCTTCTTAATGATTGAACAAAATACCAAGTCTCCCCTCTTCATGGGA |
| | | AAAGTGGTGAATCCCACCCAAAAATAA |
| 1406 | SERPINA1 WT | MPSSVSWGILLLAGLCCLVPVSLAEDPQGDAAQKTDTSHHDQDHPTFNK |
| | amino acid | ITPNLAEFAFSLYRQLAHQSNSTNIFFSPVSIATAFAMLSLGTKADTHDEIL |
| | sequence | EGLNFNLTEIPEAQIHEGFQELLRTLNQPDSQLQLTTGNGLFLSEGLKLVD |
| | | KFLEDVKKLYHSEAFTVNFGDTEEAKKQINDYVEKGTQGKIVDLVKELD |
| | | RDTVFALVNYIFFKGKWERPFEVKDTEEEDFHVDQVTTVKVPMMKRLG |
| | | MFNIQHCKKLSSWVLLMKYLGNATAIFFLPDEGKLQHLENELTHDIITKF |
| | | LENEDRRSASLHLPKLSITGTYDLKSVLGQLGITKVFSNGADLSGVTEEAP |
| | | LKLSKAVHKAVLTIDEKGTEAAGAMFLEAIPMSIPPEVKFNKPFVFLMIE |
| | | QNTKSPLFMGKVVNPTQK |
| 1407 | hSERPINA1 | MKWVTFISLLFLFSSAYSRGVFRRDALEDPQGDAAQKTDTSHHDQDHPT |
| | with hAlbumin | FNKITPNLAEFAFSLYRQLAHQSNSTNIFFSPVSIATAFAMLSLGTKADTH |
| | signal peptide | DEILEGLNFNLTEIPEAQIHEGFQELLRTLNQPDSQLQLTTGNGLFLSEGL |
| | encoded | KLVDKFLEDVKKLYHSEAFTVNFGDTEEAKKQINDYVEKGTQGKIVDLV |
| | insertion | KELDRDTVFALVNYIFFKGKWERPFEVKDTEEEDFHVDQVTTVKVPMM |
| | product | KRLGMFNIQHCKKLSSWVLLMKYLGNATAIFFLPDEGKLQHLENELTHD |
| | | IITKFLENEDRRSASLHLPKLSITGTYDLKSVLGQLGITKVFSNGADLSGV |
| | | TEEAPLKLSKAVHKAVLTIDEKGTEAAGAMFLEAIPMSIPPEVKFNKPFV |
| | | FLMIEQNTKSPLFMGKVVNPTQK |
| 1408 | hSERPINA1 | DALEDPQGDAAQKTDTSHHDQDHPTFNKITPNLAEFAFSLYRQLAHQSN |
| | with hAlbumin | STNIFFSPVSIATAFAMLSLGTKADTHDEILEGLNFNLTEIPEAQIHEGFQE |
| | signal peptide | LLRTLNQPDSQLQLTTGNGLFLSEGLKLVDKFLEDVKKLYHSEAFTVNF |
| | encoded | GDTEEAKKQINDYVEKGTQGKIVDLVKELDRDTVFALVNYIFFKGKWER |
| | insertion product | PFEVKDTEEEDFHVDQVTTVKVPMMKRLGMFNIQHCKKLSSWVLLMKY |
| | after signal | LGNATAIFFLPDEGKLQHLENELTHDIITKFLENEDRRSASLHLPKLSITGT |
| | peptide cleavage | YDLKSVLGQLGITKVFSNGADLSGVTEEAPLKLSKAVHKAVLTIDEKGTE |
| | | AAGAMFLEAIPMSIPPEVKFNKPFVFLMIEQNTKSPLFMGKVVNPTQK |
| 1409 | native | MPSSVSWGILLLAGLCCLVPVSLAEDPQGDAAQKTDTSHHDQDHPTFNK |
| | hSERPINA1 seq, | ITPNLAEFAFSLYRQLAHQSNSTNIFFSPVSIATAFAMLSLGTKADTHDEIL |
| | with SERPINA1 | EGLNFNLTEIPEAQIHEGFQELLRTLNQPDSQLQLTTGNGLFLSEGLKLVD |
| | signal peptide | KFLEDVKKLYHSEAFTVNFGDTEEAKKQINDYVEKGTQGKIVDLVKELD |
| | | RDTVFALVNYIFFKGKWERPFEVKDTEEEDFHVDQVTTVKVPMMKRLG |
| | | MFNIQHCKKLSSWVLLMKYLGNATAIFFLPDEGKLQHLENELTHDIITKF |
| | | LENEDRRSASLHLPKLSITGTYDLKSVLGQLGITKVFSNGADLSGVTEEAP |
| | | LKLSKAVHKAVLTIDEKGTEAAGAMFLEAIPMSIPPEVKFNKPFVFLMIE |
| | | QNTKSPLFMGKVVNPTQK |
| 1410 | native | EDPQGDAAQKTDTSHHDQDHPTFNKITPNLAEFAFSLYRQLAHQSNSTNI |
| | hSERPINA1 seq, | FFSPVSIATAFAMLSLGTKADTHDEILEGLNFNLTEIPEAQIHEGFQELLRT |
| | with SERPINA1 | LNQPDSQLQLTTGNGLFLSEGLKLVDKFLEDVKKLYHSEAFTVNFGDTE |
| | signal peptide | EAKKQINDYVEKGTQGKIVDLVKELDRDTVFALVNYIFFKGKWERPFEV |
| | after signal | KDTEEEDFHVDQVTTVKVPMMKRLGMFNIQHCKKLSSWVLLMKYLGN |
| | peptide cleavage | ATAIFFLPDEGKLQHLENELTHDIITKFLENEDRRSASLHLPKLSITGTYDL |
| | | KSVLGQLGITKVFSNGADLSGVTEEAPLKLSKAVHKAVLTIDEKGTEAA |
| | | GAMFLEAIPMSIPPEVKFNKPFVFLMIEQNTKSPLFMGKVVNPTQK |
| 857 | Recombinant | MDKKYSIGLDIGTNSVGWAVITDEYKVPSKKFKVLGNTDRHSIKKNLIG |
| | Cas9-NLS amino | ALLFDSGETAEATRLKRTARRRYTRRKNRICYLQEIFSNEMAKVDDSFFH |
| | acid sequence | RLEESFLVEEDKKHERHPIFGNIVDEVAYHEKYPTIYHLRKKLVDSTDKA |
| | | DLRLIYLALAHMIKFRGHFLIEGDLNPDNSDVDKLFQLVQTYNQLFEENP |
| | | INASGVDAKAILSARLSKSRRLENLIAQLPGEKKNGLFGNLIALSLGLTPN |
| | | FKSNFDLAEDAKLQLSKDTYDDDLDNLLAQIGDQYADLFLAAKNLSDAI |
| | | LLSDILRVNTEITKAPLSASMIKRYDEHHQDLTLLKALVRQQLPEKYKEIF |
| | | FDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVG |
| | | PLARGNSRFAWMTRKSEETITPWNFEEVVDKGASAQSFIERMTNFDKNL |
| | | PNEKVLPKHSLLYEYFTVYNELTKVKYVTEGMRKPAFLSGEQKKAIVDL |
| | | LFKTNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHDDSL |
| | | TFKEDIQKAQVSGQGDSLHEHIANLAGSPAIKKGILQTVKVVDELVKVM |
| | | GRHKPENIVIEMARENQTTQKGQKNSRERMKRIEEGIKELGSQILKEHPV |
| | | ENTQLQNEKLYLYYLQNGRDMYVDQELDINRLSDYDVDHIVPQSFLKD |
| | | DSIDNKVLTRSDKNRGKSDNVPSEEVVKKMKNYWRQLLNAKLITQRKF |
| | | DNLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVGT |
| | | ALIKKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQVNIV |
| | | KKTEVQTGGFSKESILPKRNSDKLIARKKDWDPKKYGGFDSPTVAYSVL |
| | | VVAKVEKGKSKKLKSVKELLGITIMERSSFEKNPIDFLEAKGYKEVKKDL |
| | | IIKLPKYSLFELENGRKRMLASAGELQKGNELALPSKYVNFLYLASHYEK |
| | | LKGSPEDNEQKQLFVEQHKHYLDEIIEQISEFSKRVILADANLDKVLSAY |
| | | NKHRDKPIREQAENIIHLFTLTNLGAPAAFKYFDTTIDRKRYTSTKEVLDA |
| | | TLIHQSITGLYETRIDLSQLGGDGGGSPKKKRKV |
| 858 | ORF encoding | ATGGACAAGAAGTACAGCATCGGACTGGACATCGGAACAAACAGCG |
| | Sp. Cas9 | TCGGATGGGCAGTCATCACAGACGAATACAAGGTCCCGAGCAAGAAG |
| | | TTCAAGGTCCTGGGAAACACAGACAGACACAGCATCAAGAAGAACCT |
| | | GATCGGAGCACTGCTGTTCGACAGCGGAGAAACAGCAGAAGCAACA |
| | | AGACTGAAGAGAACAGCAAGAAGAAGATACACAAGAAGAAAGAACA |
| | | GAATCTGCTACCTGCAGGAAATCTTCAGCAACGAAATGGCAAAGGTC |
| | | GACGACAGCTTCTTCCACAGACTGGAAGAAAGCTTCCTGGTCGAAGA |
| | | AGACAAGAAGCACGAAAGACACCCGATCTTCGGAAACATCGTCGACG |
| | | AAGTCGCATACCACGAAAAGTACCCGACAATCTACCACCTGAGAAAG |
| | | AAGCTGGTCGACAGCACAGACAAGGCAGACCTGAGACTGATCTACCT |
| | | GGCACTGGCACACATGATCAAGTTCAGAGGACACTTCCTGATCGAAG |
| | | GAGACCTGAACCCGGACAACAGCGACGTCGACAAGCTGTTCATCCAG |
| | | CTGGTCCAGACATACAACCAGCTGTTCGAAGAAAACCCGATCAACGC |
| | | AAGCGGAGTCGACGCAAAGGCAATCCTGAGCGCAAGACTGAGCAAG |
| | | AGCAGAAGACTGGAAAACCTGATCGCACAGCTGCCGGGAGAAAAGA |
| | | AGAACGGACTGTTCGGAAACCTGATCGCACTGAGCCTGGGACTGACA |
| | | CCGAACTTCAAGAGCAACTTCGACCTGGCAGAAGACGCAAAGCTGCA |
| | | GCTGAGCAAGGACACATACGACGACGACCTGGACAACCTGCTGGCAC |
| | | AGATCGGAGACCAGTACGCAGACCTGTTCCTGGCAGCAAAGAACCTG |
| | | AGCGACGCAATCCTGCTGAGCGACATCCTGAGAGTCAACACAGAAAT |
| | | CACAAAGGCACCGCTGAGCGCAAGCATGATCAAGAGATACGACGAA |
| | | CACCACCAGGACCTGACACTGCTGAAGGCACTGGTCAGACAGCAGCT |
| | | GCCGGAAAAGTACAAGGAAATCTTCTTCGACCAGAGCAAGAACGGAT |
| | | ACGCAGGATACATCGACGGAGGAGCAAGCCAGGAAGAATTCTACAA |
| | | GTTCATCAAGCCGATCCTGGAAAAGATGGACGGAACAGAAGAACTGC |
| | | TGGTCAAGCTGAACAGAGAAGACCTGCTGAGAAAGCAGAGAACATTC |
| | | GACAACGGAAGCATCCCGCACCAGATCCACCTGGGAGAACTGCACGC |
| | | AATCCTGAGAAGACAGGAAGACTTCTACCCGTTCCTGAAGGACAACA |
| | | GAGAAAAGATCGAAAAGATCCTGACATTCAGAATCCCGTACTACGTC |
| | | GGACCGCTGGCAAGAGGAAACAGCAGATTCGCATGGATGACAAGAA |
| | | AGAGCGAAGAAACAATCACACCGTGGAACTTCGAAGAAGTCGTCGAC |
| | | AAGGGAGCAAGCGCACAGAGCTTCATCGAAAGAATGACAAACTTCG |
| | | ACAAGAACCTGCCGAACGAAAAGGTCCTGCCGAAGCACAGCCTGCTG |
| | | TACGAATACTTCACAGTCTACAACGAACTGACAAAGGTCAAGTACGT |
| | | CACAGAAGGAATGAGAAAGCCGGCATTCCTGAGCGGAGAACAGAAG |
| | | AAGGCAATCGTCGACCTGCTGTTCAAGACAAACAGAAAGGTCACAGT |
| | | CAAGCAGCTGAAGGAAGACTACTTCAAGAAGATCGAATGCTTCGACA |
| | | GCGTCGAAATCAGCGGAGTCGAAGACAGATTCAACGCAAGCCTGGGA |
| | | ACATACCACGACCTGCTGAAGATCATCAAGGACAAGGACTTCCTGGA |
| | | CAACGAAGAAAACGAAGACATCCTGGAAGACATCGTCCTGACACTGA |
| | | CACTGTTCGAAGACAGAGAAATGATCGAAGAAAGACTGAAGACATA |
| | | CGCACACCTGTTCGACGACAAGGTCATGAAGCAGCTGAAGAGAAGAA |
| | | GATACACAGGATGGGGAAGACTGAGCAGAAAGCTGATCAACGGAAT |
| | | CAGAGACAAGCAGAGCGGAAAGACAATCCTGGACTTCCTGAAGAGC |
| | | GACGGATTCGCAAACAGAAACTTCATGCAGCTGATCCACGACGACAG |
| | | CCTGACATTCAAGGAAGACATCCAGAAGGCACAGGTCAGCGGACAG |
| | | GGAGACAGCCTGCACGAACACATCGCAAACCTGGCAGGAAGCCCGG |
| | | CAATCAAGAAGGGAATCCTGCAGACAGTCAAGGTCGTCGACGAACTG |
| | | GTCAAGGTCATGGGAAGACACAAGCCGGAAAACATCGTCATCGAAAT |
| | | GGCAAGAGAAAACCAGACAACACAGAAGGGACAGAAGAACAGCAG |
| | | AGAAAGAATGAAGAGAATCGAAGAAGGAATCAAGGAACTGGGAAGC |
| | | CAGATCCTGAAGGAACACCCGGTCGAAAACACACAGCTGCAGAACG |
| | | AAAAGCTGTACCTGTACTACCTGCAGAACGGAAGAGACATGTACGTC |
| | | GACCAGGAACTGGACATCAACAGACTGAGCGACTACGACGTCGACCA |
| | | CATCGTCCCGCAGAGCTTCCTGAAGGACGACAGCATCGACAACAAGG |
| | | TCCTGACAAGAAGCGACAAGAACAGAGGAAAGAGCGACAACGTCCC |
| | | GAGCGAAGAAGTCGTCAAGAAGATGAAGAACTACTGGAGACAGCTG |
| | | CTGAACGCAAAGCTGATCACACAGAGAAAGTTCGACAACCTGACAAA |
| | | GGCAGAGAGAGGAGGACTGAGCGAACTGGACAAGGCAGGATTCATC |
| | | AAGAGACAGCTGGTCGAAACAAGACAGATCACAAAGCACGTCGCAC |
| | | AGATCCTGGACAGCAGAATGAACACAAAGTACGACGAAAACGACAA |
| | | GCTGATCAGAGAAGTCAAGGTCATCACACTGAAGAGCAAGCTGGTCA |
| | | GCGACTTCAGAAAGGACTTCCAGTTCTACAAGGTCAGAGAAATCAAC |
| | | AACTACCACCACGCACACGACGCATACCTGAACGCAGTCGTCGGAAC |
| | | AGCACTGATCAAGAAGTACCCGAAGCTGGAAAGCGAATTCGTCTACG |
| | | GAGACTACAAGGTCTACGACGTCAGAAAGATGATCGCAAAGAGCGA |
| | | ACAGGAAATCGGAAAGGCAACAGCAAAGTACTTCTTCTACAGCAACA |
| | | TCATGAACTTCTTCAAGACAGAAATCACACTGGCAAACGGAGAAATC |
| | | AGAAAGAGACCGCTGATCGAAACAAACGGAGAAACAGGAGAAATCG |
| | | TCTGGGACAAGGGAAGAGACTTCGCAACAGTCAGAAAGGTCCTGAGC |
| | | ATGCCGCAGGTCAACATCGTCAAGAAGACAGAAGTCCAGACAGGAG |
| | | GATTCAGCAAGGAAAGCATCCTGCCGAAGAGAAACAGCGACAAGCT |
| | | GATCGCAAGAAAGAAGGACTGGGACCCGAAGAAGTACGGAGGATTC |
| | | GACAGCCCGACAGTCGCATACAGCGTCCTGGTCGTCGCAAAGGTCGA |
| | | AAAGGGAAAGAGCAAGAAGCTGAAGAGCGTCAAGGAACTGCTGGGA |
| | | ATCACAATCATGGAAAGAAGCAGCTTCGAAAAGAACCCGATCGACTT |
| | | CCTGGAAGCAAAGGGATACAAGGAAGTCAAGAAGGACCTGATCATC |
| | | AAGCTGCCGAAGTACAGCCTGTTCGAACTGGAAAACGGAAGAAAGA |
| | | GAATGCTGGCAAGCGCAGGAGAACTGCAGAAGGGAAACGAACTGGC |
| | | ACTGCCGAGCAAGTACGTCAACTTCCTGTACCTGGCAAGCCACTACG |
| | | AAAAGCTGAAGGGAAGCCCGGAAGACAACGAACAGAAGCAGCTGTT |
| | | CGTCGAACAGCACAAGCACTACCTGGACGAAATCATCGAACAGATCA |
| | | GCGAATTCAGCAAGAGAGTCATCCTGGCAGACGCAAACCTGGACAAG |
| | | GTCCTGAGCGCATACAACAAGCACAGAGACAAGCCGATCAGAGAAC |
| | | AGGCAGAAAACATCATCCACCTGTTCACACTGACAAACCTGGGAGCA |
| | | CCGGCAGCATTCAAGTACTTCGACACAACAATCGACAGAAAGAGATA |
| | | CACAAGCACAAAGGAAGTCCTGGACGCAACACTGATCCACCAGAGCA |
| | | TCACAGGACTGTACGAAACAAGAATCGACCTGAGCCAGCTGGGAGGA |
| | | GACGGAGGAGGAAGCCCGAAGAAGAAGAGAAAGGTCTAG |
| 859 | ORF encoding | ATGGACAAGAAGTACTCCATCGGCCTGGACATCGGCACCAACTCCGT |
| | Sp. Cas9 | GGGCTGGGCCGTGATCACCGACGAGTACAAGGTGCCCTCCAAGAAGT |
| | | TCAAGGTGCTGGGCAACACCGACCGGCACTCCATCAAGAAGAACCTG |
| | | ATCGGCGCCCTGCTGTTCGACTCCGGCGAGACCGCCGAGGCCACCCG |
| | | GCTGAAGCGGACCGCCCGGCGGCGGTACACCCGGCGGAAGAACCGG |
| | | ATCTGCTACCTGCAGGAGATCTTCTCCAACGAGATGGCCAAGGTGGA |
| | | CGACTCCTTCTTCCACCGGCTGGAGGAGTCCTTCCTGGTGGAGGAGGA |
| | | CAAGAAGCACGAGCGGCACCCCATCTTCGGCAACATCGTGGACGAGG |
| | | TGGCCTACCACGAGAAGTACCCCACCATCTACCACCTGCGGAAGAAG |
| | | CTGGTGGACTCCACCGACAAGGCCGACCTGCGGCTGATCTACCTGGC |
| | | CCTGGCCCACATGATCAAGTTCCGGGGCCACTTCCTGATCGAGGGCG |
| | | ACCTGAACCCCGACAACTCCGACGTGGACAAGCTGTTCATCCAGCTG |
| | | GTGCAGACCTACAACCAGCTGTTCGAGGAGAACCCCATCAACGCCTC |
| | | CGGCGTGGACGCCAAGGCCATCCTGTCCGCCCGGCTGTCCAAGTCCC |
| | | GGCGGCTGGAGAACCTGATCGCCCAGCTGCCCGGCGAGAAGAAGAA |
| | | CGGCCTGTTCGGCAACCTGATCGCCCTGTCCCTGGGCCTGACCCCCAA |
| | | CTTCAAGTCCAACTTCGACCTGGCCGAGGACGCCAAGCTGCAGCTGT |
| | | CCAAGGACACCTACGACGACGACCTGGACAACCTGCTGGCCCAGATC |
| | | GGCGACCAGTACGCCGACCTGTTCCTGGCCGCCAAGAACCTGTCCGA |
| | | CGCCATCCTGCTGTCCGACATCCTGCGGGTGAACACCGAGATCACCA |
| | | AGGCCCCCCTGTCCGCCTCCATGATCAAGCGGTACGACGAGCACCAC |
| | | CAGGACCTGACCCTGCTGAAGGCCCTGGTGCGGCAGCAGCTGCCCGA |
| | | GAAGTACAAGGAGATCTTCTTCGACCAGTCCAAGAACGGCTACGCCG |
| | | GCTACATCGACGGCGGCGCCTCCCAGGAGGAGTTCTACAAGTTCATC |
| | | AAGCCCATCCTGGAGAAGATGGACGGCACCGAGGAGCTGCTGGTGAA |
| | | GCTGAACCGGGAGGACCTGCTGCGGAAGCAGCGGACCTTCGACAACG |
| | | GCTCCATCCCCCACCAGATCCACCTGGGCGAGCTGCACGCCATCCTGC |
| | | GGCGGCAGGAGGACTTCTACCCCTTCCTGAAGGACAACCGGGAGAAG |
| | | ATCGAGAAGATCCTGACCTTCCGGATCCCCTACTACGTGGGCCCCCTG |
| | | GCCCGGGGCAACTCCCGGTTCGCCTGGATGACCCGGAAGTCCGAGGA |
| | | GACCATCACCCCCTGGAACTTCGAGGAGGTGGTGGACAAGGGCGCCT |
| | | CCGCCCAGTCCTTCATCGAGCGGATGACCAACTTCGACAAGAACCTG |
| | | CCCAACGAGAAGGTGCTGCCCAAGCACTCCCTGCTGTACGAGTACTT |
| | | CACCGTGTACAACGAGCTGACCAAGGTGAAGTACGTGACCGAGGGCA |
| | | TGCGGAAGCCCGCCTTCCTGTCCGGCGAGCAGAAGAAGGCCATCGTG |
| | | GACCTGCTGTTCAAGACCAACCGGAAGGTGACCGTGAAGCAGCTGAA |
| | | GGAGGACTACTTCAAGAAGATCGAGTGCTTCGACTCCGTGGAGATCT |
| | | CCGGCGTGGAGGACCGGTTCAACGCCTCCCTGGGCACCTACCACGAC |
| | | CTGCTGAAGATCATCAAGGACAAGGACTTCCTGGACAACGAGGAGAA |
| | | CGAGGACATCCTGGAGGACATCGTGCTGACCCTGACCCTGTTCGAGG |
| | | ACCGGGAGATGATCGAGGAGCGGCTGAAGACCTACGCCCACCTGTTC |
| | | GACGACAAGGTGATGAAGCAGCTGAAGCGGCGGCGGTACACCGGCT |
| | | GGGGCCGGCTGTCCCGGAAGCTGATCAACGGCATCCGGGACAAGCAG |
| | | TCCGGCAAGACCATCCTGGACTTCCTGAAGTCCGACGGCTTCGCCAAC |
| | | CGGAACTTCATGCAGCTGATCCACGACGACTCCCTGACCTTCAAGGA |
| | | GGACATCCAGAAGGCCCAGGTGTCCGGCCAGGGCGACTCCCTGCACG |
| | | AGCACATCGCCAACCTGGCCGGCTCCCCCGCCATCAAGAAGGGCATC |
| | | CTGCAGACCGTGAAGGTGGTGGACGAGCTGGTGAAGGTGATGGGCCG |
| | | GCACAAGCCCGAGAACATCGTGATCGAGATGGCCCGGGAGAACCAG |
| | | ACCACCCAGAAGGGCCAGAAGAACTCCCGGGAGCGGATGAAGCGGA |
| | | TCGAGGAGGGCATCAAGGAGCTGGGCTCCCAGATCCTGAAGGAGCAC |
| | | CCCGTGGAGAACACCCAGCTGCAGAACGAGAAGCTGTACCTGTACTA |
| | | CCTGCAGAACGGCCGGGACATGTACGTGGACCAGGAGCTGGACATCA |
| | | ACCGGCTGTCCGACTACGACGTGGACCACATCGTGCCCCAGTCCTTCC |
| | | TGAAGGACGACTCCATCGACAACAAGGTGCTGACCCGGTCCGACAAG |
| | | AACCGGGGCAAGTCCGACAACGTGCCCTCCGAGGAGGTGGTGAAGA |
| | | AGATGAAGAACTACTGGCGGCAGCTGCTGAACGCCAAGCTGATCACC |
| | | CAGCGGAAGTTCGACAACCTGACCAAGGCCGAGCGGGGCGGCCTGTC |
| | | CGAGCTGGACAAGGCCGGCTTCATCAAGCGGCAGCTGGTGGAGACCC |
| | | GGCAGATCACCAAGCACGTGGCCCAGATCCTGGACTCCCGGATGAAC |
| | | ACCAAGTACGACGAGAACGACAAGCTGATCCGGGAGGTGAAGGTGA |
| | | TCACCCTGAAGTCCAAGCTGGTGTCCGACTTCCGGAAGGACTTCCAGT |
| | | TCTACAAGGTGCGGGAGATCAACAACTACCACCACGCCCACGACGCC |
| | | TACCTGAACGCCGTGGTGGGCACCGCCCTGATCAAGAAGTACCCCAA |
| | | GCTGGAGTCCGAGTTCGTGTACGGCGACTACAAGGTGTACGACGTGC |
| | | GGAAGATGATCGCCAAGTCCGAGCAGGAGATCGGCAAGGCCACCGC |
| | | CAAGTACTTCTTCTACTCCAACATCATGAACTTCTTCAAGACCGAGAT |
| | | CACCCTGGCCAACGGCGAGATCCGGAAGCGGCCCCTGATCGAGACCA |
| | | ACGGCGAGACCGGCGAGATCGTGTGGGACAAGGGCCGGGACTTCGCC |
| | | ACCGTGCGGAAGGTGCTGTCCATGCCCCAGGTGAACATCGTGAAGAA |
| | | GACCGAGGTGCAGACCGGCGGCTTCTCCAAGGAGTCCATCCTGCCCA |
| | | AGCGGAACTCCGACAAGCTGATCGCCCGGAAGAAGGACTGGGACCCC |
| | | AAGAAGTACGGCGGCTTCGACTCCCCCACCGTGGCCTACTCCGTGCTG |
| | | GTGGTGGCCAAGGTGGAGAAGGGCAAGTCCAAGAAGCTGAAGTCCG |
| | | TGAAGGAGCTGCTGGGCATCACCATCATGGAGCGGTCCTCCTTCGAG |
| | | AAGAACCCCATCGACTTCCTGGAGGCCAAGGGCTACAAGGAGGTGAA |
| | | GAAGGACCTGATCATCAAGCTGCCCAAGTACTCCCTGTTCGAGCTGG |
| | | AGAACGGCCGGAAGCGGATGCTGGCCTCCGCCGGCGAGCTGCAGAA |
| | | GGGCAACGAGCTGGCCCTGCCCTCCAAGTACGTGAACTTCCTGTACCT |
| | | GGCCTCCCACTACGAGAAGCTGAAGGGCTCCCCCGAGGACAACGAGC |
| | | AGAAGCAGCTGTTCGTGGAGCAGCACAAGCACTACCTGGACGAGATC |
| | | ATCGAGCAGATCTCCGAGTTCTCCAAGCGGGTGATCCTGGCCGACGC |
| | | CAACCTGGACAAGGTGCTGTCCGCCTACAACAAGCACCGGGACAAGC |
| | | CCATCCGGGAGCAGGCCGAGAACATCATCCACCTGTTCACCCTGACC |
| | | AACCTGGGCGCCCCCGCCGCCTTCAAGTACTTCGACACCACCATCGA |
| | | CCGGAAGCGGTACACCTCCACCAAGGAGGTGCTGGACGCCACCCTGA |
| | | TCCACCAGTCCATCACCGGCCTGTACGAGACCCGGATCGACCTGTCCC |
| | | AGCTGGGCGGCGACGGCGGCGGCTCCCCCAAGAAGAAGCGGAAGGT |
| | | GTGA |
| 860 | ORF encoding | AUGGACAAGAAGUACAGCAUCGGCCUGGACAUCGGCACGAACAGCG |
| | Sp. Cas9 | UUGGCUGGGCUGUGAUCACGGACGAGUACAAGGUUCCCUCAAAGAA |
| | | GUUCAAGGUGCUGGGCAACACGGACCGGCACAGCAUCAAGAAGAAU |
| | | CUCAUCGGUGCACUGCUGUUCGACAGCGGUGAGACGGCCGAAGCCA |
| | | CGCGGCUGAAGCGGACGGCCCGCCGGCGGUACACGCGGCGGAAGAA |
| | | CCGGAUCUGCUACCUGCAGGAGAUCUUCAGCAACGAGAUGGCCAAG |
| | | GUGGACGACAGCUUCUUCCACCGGCUGGAGGAGAGCUUCCUGGUGG |
| | | AGGAGGACAAGAAGCACGAGCGGCACCCCAUCUUCGGCAACAUCGU |
| | | GGACGAAGUCGCCUACCACGAGAAGUACCCCACCAUCUACCACCUG |
| | | CGGAAGAAGCUGGUGGACUCGACUGACAAGGCCGACCUGCGGCUGA |
| | | UCUACCUGGCACUGGCCCACAUGAUAAAGUUCCGGGGCCACUUCCU |
| | | GAUCGAGGGCGACCUGAACCCUGACAACAGCGACGUGGACAAGCUG |
| | | UUCAUCCAGCUGGUGCAGACCUACAACCAGCUGUUCGAGGAGAACC |
| | | CCAUCAACGCCAGCGGCGUGGACGCCAAGGCCAUCCUCAGCGCCCG |
| | | CCUCAGCAAGAGCCGGCGGCUGGAGAAUCUCAUCGCCCAGCUUCCA |
| | | GGUGAGAAGAAGAAUGGGCUGUUCGGCAAUCUCAUCGCACUCAGCC |
| | | UGGGCCUGACUCCCAACUUCAAGAGCAACUUCGACCUGGCCGAGGA |
| | | CGCCAAGCUGCAGCUCAGCAAGGACACCUACGACGACGACCUGGAC |
| | | AAUCUCCUGGCCCAGAUCGGCGACCAGUACGCCGACCUGUUCCUGG |
| | | CUGCCAAGAAUCUCAGCGACGCCAUCCUGCUCAGCGACAUCCUGCG |
| | | GGUGAACACAGAGAUCACGAAGGCCCCCCUCAGCGCCAGCAUGAUA |
| | | AAGCGGUACGACGAGCACCACCAGGACCUGACGCUGCUGAAGGCAC |
| | | UGGUGCGGCAGCAGCUUCCAGAGAAGUACAAGGAGAUCUUCUUCGA |
| | | CCAGAGCAAGAAUGGGUACGCCGGGUACAUCGACGGUGGUGCCAGC |
| | | CAGGAGGAGUUCUACAAGUUCAUCAAGCCCAUCCUGGAGAAGAUGG |
| | | ACGGCACAGAGGAGCUGCUGGUGAAGCUGAACAGGGAGGACCUGCU |
| | | GCGGAAGCAGCGGACGUUCGACAAUGGGAGCAUCCCCCACCAGAUC |
| | | CACCUGGGUGAGCUGCACGCCAUCCUGCGGCGGCAGGAGGACUUCU |
| | | ACCCCUUCCUGAAGGACAACAGGGAGAAGAUCGAGAAGAUCCUGAC |
| | | GUUCCGGAUCCCCUACUACGUUGGCCCCCUGGCCCGCGGCAACAGC |
| | | CGGUUCGCCUGGAUGACGCGGAAGAGCGAGGAGACGAUCACUCCCU |
| | | GGAACUUCGAGGAAGUCGUGGACAAGGGUGCCAGCGCCCAGAGCUU |
| | | CAUCGAGCGGAUGACGAACUUCGACAAGAAUCUUCCAAACGAGAAG |
| | | GUGCUUCCAAAGCACAGCCUGCUGUACGAGUACUUCACGGUGUACA |
| | | ACGAGCUGACGAAGGUGAAGUACGUGACAGAGGGCAUGCGGAAGC |
| | | CCGCCUUCCUCAGCGGUGAGCAGAAGAAGGCCAUCGUGGACCUGCU |
| | | GUUCAAGACGAACCGGAAGGUGACGGUGAAGCAGCUGAAGGAGGA |
| | | CUACUUCAAGAAGAUCGAGUGCUUCGACAGCGUGGAGAUCAGCGGC |
| | | GUGGAGGACCGGUUCAACGCCAGCCUGGGCACCUACCACGACCUGC |
| | | UGAAGAUCAUCAAGGACAAGGACUUCCUGGACAACGAGGAGAACG |
| | | AGGACAUCCUGGAGGACAUCGUGCUGACGCUGACGCUGUUCGAGGA |
| | | CAGGGAGAUGAUAGAGGAGCGGCUGAAGACCUACGCCCACCUGUUC |
| | | GACGACAAGGUGAUGAAGCAGCUGAAGCGGCGGCGGUACACGGGCU |
| | | GGGGCCGGCUCAGCCGGAAGCUGAUCAAUGGGAUCCGAGACAAGCA |
| | | GAGCGGCAAGACGAUCCUGGACUUCCUGAAGAGCGACGGCUUCGCC |
| | | AACCGGAACUUCAUGCAGCUGAUCCACGACGACAGCCUGACGUUCA |
| | | AGGAGGACAUCCAGAAGGCCCAGGUCAGCGGCCAGGGCGACAGCCU |
| | | GCACGAGCACAUCGCCAAUCUCGCCGGGAGCCCCGCCAUCAAGAAG |
| | | GGGAUCCUGCAGACGGUGAAGGUGGUGGACGAGCUGGUGAAGGUG |
| | | AUGGGCCGGCACAAGCCAGAGAACAUCGUGAUCGAGAUGGCCAGGG |
| | | AGAACCAGACGACUCAAAAGGGGCAGAAGAACAGCAGGGAGCGGA |
| | | UGAAGCGGAUCGAGGAGGGCAUCAAGGAGCUGGGCAGCCAGAUCCU |
| | | GAAGGAGCACCCCGUGGAGAACACUCAACUGCAGAACGAGAAGCUG |
| | | UACCUGUACUACCUGCAGAAUGGGCGAGACAUGUACGUGGACCAGG |
| | | AGCUGGACAUCAACCGGCUCAGCGACUACGACGUGGACCACAUCGU |
| | | UCCCCAGAGCUUCCUGAAGGACGACAGCAUCGACAACAAGGUGCUG |
| | | ACGCGGAGCGACAAGAACCGGGGCAAGAGCGACAACGUUCCCUCAG |
| | | AGGAAGUCGUGAAGAAGAUGAAGAACUACUGGCGGCAGCUGCUGA |
| | | ACGCCAAGCUGAUCACUCAACGGAAGUUCGACAAUCUCACGAAGGC |
| | | CGAGCGGGGUGGCCUCAGCGAGCUGGACAAGGCCGGGUUCAUCAAG |
| | | CGGCAGCUGGUGGAGACGCGGCAGAUCACGAAGCACGUGGCCCAGA |
| | | UCCUGGACAGCCGGAUGAACACGAAGUACGACGAGAACGACAAGCU |
| | | GAUCAGGGAAGUCAAGGUGAUCACGCUGAAGAGCAAGCUGGUCAG |
| | | CGACUUCCGGAAGGACUUCCAGUUCUACAAGGUGAGGGAGAUCAAC |
| | | AACUACCACCACGCCCACGACGCCUACCUGAACGCUGUGGUUGGCA |
| | | CGGCACUGAUCAAGAAGUACCCCAAGCUGGAGAGCGAGUUCGUGUA |
| | | CGGCGACUACAAGGUGUACGACGUGCGGAAGAUGAUAGCCAAGAGC |
| | | GAGCAGGAGAUCGGCAAGGCCACGGCCAAGUACUUCUUCUACAGCA |
| | | ACAUCAUGAACUUCUUCAAGACAGAGAUCACGCUGGCCAAUGGUGA |
| | | GAUCCGGAAGCGGCCCCUGAUCGAGACGAAUGGUGAGACGGGUGAG |
| | | AUCGUGUGGGACAAGGGGCGAGACUUCGCCACGGUGCGGAAGGUGC |
| | | UCAGCAUGCCCCAGGUGAACAUCGUGAAGAAGACAGAAGUCCAGAC |
| | | GGGUGGCUUCAGCAAGGAGAGCAUCCUUCCAAAGCGGAACAGCGAC |
| | | AAGCUGAUCGCCCGCAAGAAGGACUGGGACCCCAAGAAGUACGGUG |
| | | GCUUCGACAGCCCCACCGUGGCCUACAGCGUGCUGGUGGUGGCCAA |
| | | GGUGGAGAAGGGGAAGAGCAAGAAGCUGAAGAGCGUGAAGGAGCU |
| | | GCUGGGCAUCACGAUCAUGGAGCGGAGCAGCUUCGAGAAGAACCCC |
| | | AUCGACUUCCUGGAAGCCAAGGGGUACAAGGAAGUCAAGAAGGACC |
| | | UGAUCAUCAAGCUUCCAAAGUACAGCCUGUUCGAGCUGGAGAAUGG |
| | | GCGGAAGCGGAUGCUGGCCAGCGCCGGUGAGCUGCAGAAGGGGAAC |
| | | GAGCUGGCACUUCCCUCAAAGUACGUGAACUUCCUGUACCUGGCCA |
| | | GCCACUACGAGAAGCUGAAGGGGAGCCCAGAGGACAACGAGCAGAA |
| | | GCAGCUGUUCGUGGAGCAGCACAAGCACUACCUGGACGAGAUCAUC |
| | | GAGCAGAUCAGCGAGUUCAGCAAGCGGGUGAUCCUGGCCGACGCCA |
| | | AUCUCGACAAGGUGCUCAGCGCCUACAACAAGCACCGAGACAAGCC |
| | | CAUCAGGGAGCAGGCCGAGAACAUCAUCCACCUGUUCACGCUGACG |
| | | AAUCUCGGUGCCCCCGCUGCCUUCAAGUACUUCGACACGACGAUCG |
| | | ACCGGAAGCGGUACACGUCGACUAAGGAAGUCCUGGACGCCACGCU |
| | | GAUCCACCAGAGCAUCACGGGCCUGUACGAGACGCGGAUCGACCUC |
| | | AGCCAGCUGGGUGGCGACGGUGGUGGCAGCCCCAAGAAGAAGCGGA |
| | | AGGUGUAG |
| 861 | ORF encoding | AUGGACAAGAAGUACAGCAUCGGCCUCGACAUCGGCACCAACAGCG |
| | Sp. Cas9 | UCGGCUGGGCCGUCAUCACCGACGAGUACAAGGUCCCCAGCAAGAA |
| | | GUUCAAGGUCCUCGGCAACACCGACCGCCACAGCAUCAAGAAGAAC |
| | | CUCAUCGGCGCCCUCCUCUUCGACAGCGGCGAGACCGCCGAGGCCA |
| | | CCCGCCUCAAGCGCACCGCCCGCCGCCGCUACACCCGCCGCAAGAAC |
| | | CGCAUCUGCUACCUCCAGGAGAUCUUCAGCAACGAGAUGGCCAAGG |
| | | UCGACGACAGCUUCUUCCACCGCCUCGAGGAGAGCUUCCUCGUCGA |
| | | GGAGGACAAGAAGCACGAGCGCCACCCCAUCUUCGGCAACAUCGUC |
| | | GACGAGGUCGCCUACCACGAGAAGUACCCCACCAUCUACCACCUCC |
| | | GCAAGAAGCUCGUCGACAGCACCGACAAGGCCGACCUCCGCCUCAU |
| | | CUACCUCGCCCUCGCCCACAUGAUCAAGUUCCGCGGCCACUUCCUC |
| | | AUCGAGGGCGACCUCAACCCCGACAACAGCGACGUCGACAAGCUCU |
| | | UCAUCCAGCUCGUCCAGACCUACAACCAGCUCUUCGAGGAGAACCC |
| | | CAUCAACGCCAGCGGCGUCGACGCCAAGGCCAUCCUCAGCGCCCGC |
| | | CUCAGCAAGAGCCGCCGCCUCGAGAACCUCAUCGCCCAGCUCCCCG |
| | | GCGAGAAGAAGAACGGCCUCUUCGGCAACCUCAUCGCCCUCAGCCU |
| | | CGGCCUCACCCCCAACUUCAAGAGCAACUUCGACCUCGCCGAGGAC |
| | | GCCAAGCUCCAGCUCAGCAAGGACACCUACGACGACGACCUCGACA |
| | | ACCUCCUCGCCCAGAUCGGCGACCAGUACGCCGACCUCUUCCUCGC |
| | | CGCCAAGAACCUCAGCGACGCCAUCCUCCUCAGCGACAUCCUCCGC |
| | | GUCAACACCGAGAUCACCAAGGCCCCCCUCAGCGCCAGCAUGAUCA |
| | | AGCGCUACGACGAGCACCACCAGGACCUCACCCUCCUCAAGGCCCU |
| | | CGUCCGCCAGCAGCUCCCCGAGAAGUACAAGGAGAUCUUCUUCGAC |
| | | CAGAGCAAGAACGGCUACGCCGGCUACAUCGACGGCGGCGCCAGCC |
| | | AGGAGGAGUUCUACAAGUUCAUCAAGCCCAUCCUCGAGAAGAUGGA |
| | | CGGCACCGAGGAGCUCCUCGUCAAGCUCAACCGCGAGGACCUCCUC |
| | | CGCAAGCAGCGCACCUUCGACAACGGCAGCAUCCCCCACCAGAUCC |
| | | ACCUCGGCGAGCUCCACGCCAUCCUCCGCCGCCAGGAGGACUUCUA |
| | | CCCCUUCCUCAAGGACAACCGCGAGAAGAUCGAGAAGAUCCUCACC |
| | | UUCCGCAUCCCCUACUACGUCGGCCCCCUCGCCCGCGGCAACAGCCG |
| | | CUUCGCCUGGAUGACCCGCAAGAGCGAGGAGACCAUCACCCCCUGG |
| | | AACUUCGAGGAGGUCGUCGACAAGGGCGCCAGCGCCCAGAGCUUCA |
| | | UCGAGCGCAUGACCAACUUCGACAAGAACCUCCCCAACGAGAAGGU |
| | | CCUCCCCAAGCACAGCCUCCUCUACGAGUACUUCACCGUCUACAAC |
| | | GAGCUCACCAAGGUCAAGUACGUCACCGAGGGCAUGCGCAAGCCCG |
| | | CCUUCCUCAGCGGCGAGCAGAAGAAGGCCAUCGUCGACCUCCUCUU |
| | | CAAGACCAACCGCAAGGUCACCGUCAAGCAGCUCAAGGAGGACUAC |
| | | UUCAAGAAGAUCGAGUGCUUCGACAGCGUCGAGAUCAGCGGCGUCG |
| | | AGGACCGCUUCAACGCCAGCCUCGGCACCUACCACGACCUCCUCAA |
| | | GAUCAUCAAGGACAAGGACUUCCUCGACAACGAGGAGAACGAGGAC |
| | | AUCCUCGAGGACAUCGUCCUCACCCUCACCCUCUUCGAGGACCGCG |
| | | AGAUGAUCGAGGAGCGCCUCAAGACCUACGCCCACCUCUUCGACGA |
| | | CAAGGUCAUGAAGCAGCUCAAGCGCCGCCGCUACACCGGCUGGGGC |
| | | CGCCUCAGCCGCAAGCUCAUCAACGGCAUCCGCGACAAGCAGAGCG |
| | | GCAAGACCAUCCUCGACUUCCUCAAGAGCGACGGCUUCGCCAACCG |
| | | CAACUUCAUGCAGCUCAUCCACGACGACAGCCUCACCUUCAAGGAG |
| | | GACAUCCAGAAGGCCCAGGUCAGCGGCCAGGGCGACAGCCUCCACG |
| | | AGCACAUCGCCAACCUCGCCGGCAGCCCCGCCAUCAAGAAGGGCAU |
| | | CCUCCAGACCGUCAAGGUCGUCGACGAGCUCGUCAAGGUCAUGGGC |
| | | CGCCACAAGCCCGAGAACAUCGUCAUCGAGAUGGCCCGCGAGAACC |
| | | AGACCACCCAGAAGGGCCAGAAGAACAGCCGCGAGCGCAUGAAGCG |
| | | CAUCGAGGAGGGCAUCAAGGAGCUCGGCAGCCAGAUCCUCAAGGAG |
| | | CACCCCGUCGAGAACACCCAGCUCCAGAACGAGAAGCUCUACCUCU |
| | | ACUACCUCCAGAACGGCCGCGACAUGUACGUCGACCAGGAGCUCGA |
| | | CAUCAACCGCCUCAGCGACUACGACGUCGACCACAUCGUCCCCCAG |
| | | AGCUUCCUCAAGGACGACAGCAUCGACAACAAGGUCCUCACCCGCA |
| | | GCGACAAGAACCGCGGCAAGAGCGACAACGUCCCCAGCGAGGAGGU |
| | | CGUCAAGAAGAUGAAGAACUACUGGCGCCAGCUCCUCAACGCCAAG |
| | | CUCAUCACCCAGCGCAAGUUCGACAACCUCACCAAGGCCGAGCGCG |
| | | GCGGCCUCAGCGAGCUCGACAAGGCCGGCUUCAUCAAGCGCCAGCU |
| | | CGUCGAGACCCGCCAGAUCACCAAGCACGUCGCCCAGAUCCUCGAC |
| | | AGCCGCAUGAACACCAAGUACGACGAGAACGACAAGCUCAUCCGCG |
| | | AGGUCAAGGUCAUCACCCUCAAGAGCAAGCUCGUCAGCGACUUCCG |
| | | CAAGGACUUCCAGUUCUACAAGGUCCGCGAGAUCAACAACUACCAC |
| | | CACGCCCACGACGCCUACCUCAACGCCGUCGUCGGCACCGCCCUCAU |
| | | CAAGAAGUACCCCAAGCUCGAGAGCGAGUUCGUCUACGGCGACUAC |
| | | AAGGUCUACGACGUCCGCAAGAUGAUCGCCAAGAGCGAGCAGGAGA |
| | | UCGGCAAGGCCACCGCCAAGUACUUCUUCUACAGCAACAUCAUGAA |
| | | CUUCUUCAAGACCGAGAUCACCCUCGCCAACGGCGAGAUCCGCAAG |
| | | CGCCCCCUCAUCGAGACCAACGGCGAGACCGGCGAGAUCGUCUGGG |
| | | ACAAGGGCCGCGACUUCGCCACCGUCCGCAAGGUCCUCAGCAUGCC |
| | | CCAGGUCAACAUCGUCAAGAAGACCGAGGUCCAGACCGGCGGCUUC |
| | | AGCAAGGAGAGCAUCCUCCCCAAGCGCAACAGCGACAAGCUCAUCG |
| | | CCCGCAAGAAGGACUGGGACCCCAAGAAGUACGGCGGCUUCGACAG |
| | | CCCCACCGUCGCCUACAGCGUCCUCGUCGUCGCCAAGGUCGAGAAG |
| | | GGCAAGAGCAAGAAGCUCAAGAGCGUCAAGGAGCUCCUCGGCAUCA |
| | | CCAUCAUGGAGCGCAGCAGCUUCGAGAAGAACCCCAUCGACUUCCU |
| | | CGAGGCCAAGGGCUACAAGGAGGUCAAGAAGGACCUCAUCAUCAAG |
| | | CUCCCCAAGUACAGCCUCUUCGAGCUCGAGAACGGCCGCAAGCGCA |
| | | UGCUCGCCAGCGCCGGCGAGCUCCAGAAGGGCAACGAGCUCGCCCU |
| | | CCCCAGCAAGUACGUCAACUUCCUCUACCUCGCCAGCCACUACGAG |
| | | AAGCUCAAGGGCAGCCCCGAGGACAACGAGCAGAAGCAGCUCUUCG |
| | | UCGAGCAGCACAAGCACUACCUCGACGAGAUCAUCGAGCAGAUCAG |
| | | CGAGUUCAGCAAGCGCGUCAUCCUCGCCGACGCCAACCUCGACAAG |
| | | GUCCUCAGCGCCUACAACAAGCACCGCGACAAGCCCAUCCGCGAGC |
| | | AGGCCGAGAACAUCAUCCACCUCUUCACCCUCACCAACCUCGGCGC |
| | | CCCCGCCGCCUUCAAGUACUUCGACACCACCAUCGACCGCAAGCGC |
| | | UACACCAGCACCAAGGAGGUCCUCGACGCCACCCUCAUCCACCAGA |
| | | GCAUCACCGGCCUCUACGAGACCCGCAUCGACCUCAGCCAGCUCGG |
| | | CGGCGACGGCGGCGGCAGCCCCAAGAAGAAGCGCAAGGUCUAG |
| 862 | Open reading | AUGGACAAGAAGUACUCCAUCGGCCUGGACAUCGGCACCAACUCCG |
| | frame for Cas9 | UGGGCUGGGCCGUGAUCACCGACGAGUACAAGGUGCCCUCCAAGAA |
| | with Hibit tag | GUUCAAGGUGCUGGGCAACACCGACCGGCACUCCAUCAAGAAGAAC |
| | | CUGAUCGGCGCCCUGCUGUUCGACUCCGGCGAGACCGCCGAGGCCA |
| | | CCCGGCUGAAGCGGACCGCCCGGCGGCGGUACACCCGGCGGAAGAA |
| | | CCGGAUCUGCUACCUGCAGGAGAUCUUCUCCAACGAGAUGGCCAAG |
| | | GUGGACGACUCCUUCUUCCACCGGCUGGAGGAGUCCUUCCUGGUGG |
| | | AGGAGGACAAGAAGCACGAGCGGCACCCCAUCUUCGGCAACAUCGU |
| | | GGACGAGGUGGCCUACCACGAGAAGUACCCCACCAUCUACCACCUG |
| | | CGGAAGAAGCUGGUGGACUCCACCGACAAGGCCGACCUGCGGCUGA |
| | | UCUACCUGGCCCUGGCCCACAUGAUCAAGUUCCGGGGCCACUUCCU |
| | | GAUCGAGGGCGACCUGAACCCCGACAACUCCGACGUGGACAAGCUG |
| | | UUCAUCCAGCUGGUGCAGACCUACAACCAGCUGUUCGAGGAGAACC |
| | | CCAUCAACGCCUCCGGCGUGGACGCCAAGGCCAUCCUGUCCGCCCG |
| | | GCUGUCCAAGUCCCGGCGGCUGGAGAACCUGAUCGCCCAGCUGCCC |
| | | GGCGAGAAGAAGAACGGCCUGUUCGGCAACCUGAUCGCCCUGUCCC |
| | | UGGGCCUGACCCCCAACUUCAAGUCCAACUUCGACCUGGCCGAGGA |
| | | CGCCAAGCUGCAGCUGUCCAAGGACACCUACGACGACGACCUGGAC |
| | | AACCUGCUGGCCCAGAUCGGCGACCAGUACGCCGACCUGUUCCUGG |
| | | CCGCCAAGAACCUGUCCGACGCCAUCCUGCUGUCCGACAUCCUGCG |
| | | GGUGAACACCGAGAUCACCAAGGCCCCCCUGUCCGCCUCCAUGAUC |
| | | AAGCGGUACGACGAGCACCACCAGGACCUGACCCUGCUGAAGGCCC |
| | | UGGUGCGGCAGCAGCUGCCCGAGAAGUACAAGGAGAUCUUCUUCGA |
| | | CCAGUCCAAGAACGGCUACGCCGGCUACAUCGACGGCGGCGCCUCC |
| | | CAGGAGGAGUUCUACAAGUUCAUCAAGCCCAUCCUGGAGAAGAUGG |
| | | ACGGCACCGAGGAGCUGCUGGUGAAGCUGAACCGGGAGGACCUGCU |
| | | GCGGAAGCAGCGGACCUUCGACAACGGCUCCAUCCCCCACCAGAUC |
| | | CACCUGGGCGAGCUGCACGCCAUCCUGCGGCGGCAGGAGGACUUCU |
| | | ACCCCUUCCUGAAGGACAACCGGGAGAAGAUCGAGAAGAUCCUGAC |
| | | CUUCCGGAUCCCCUACUACGUGGGCCCCCUGGCCCGGGGCAACUCC |
| | | CGGUUCGCCUGGAUGACCCGGAAGUCCGAGGAGACCAUCACCCCCU |
| | | GGAACUUCGAGGAGGUGGUGGACAAGGGCGCCUCCGCCCAGUCCUU |
| | | CAUCGAGCGGAUGACCAACUUCGACAAGAACCUGCCCAACGAGAAG |
| | | GUGCUGCCCAAGCACUCCCUGCUGUACGAGUACUUCACCGUGUACA |
| | | ACGAGCUGACCAAGGUGAAGUACGUGACCGAGGGCAUGCGGAAGCC |
| | | CGCCUUCCUGUCCGGCGAGCAGAAGAAGGCCAUCGUGGACCUGCUG |
| | | UUCAAGACCAACCGGAAGGUGACCGUGAAGCAGCUGAAGGAGGACU |
| | | ACUUCAAGAAGAUCGAGUGCUUCGACUCCGUGGAGAUCUCCGGCGU |
| | | GGAGGACCGGUUCAACGCCUCCCUGGGCACCUACCACGACCUGCUG |
| | | AAGAUCAUCAAGGACAAGGACUUCCUGGACAACGAGGAGAACGAG |
| | | GACAUCCUGGAGGACAUCGUGCUGACCCUGACCCUGUUCGAGGACC |
| | | GGGAGAUGAUCGAGGAGCGGCUGAAGACCUACGCCCACCUGUUCGA |
| | | CGACAAGGUGAUGAAGCAGCUGAAGCGGCGGCGGUACACCGGCUGG |
| | | GGCCGGCUGUCCCGGAAGCUGAUCAACGGCAUCCGGGACAAGCAGU |
| | | CCGGCAAGACCAUCCUGGACUUCCUGAAGUCCGACGGCUUCGCCAA |
| | | CCGGAACUUCAUGCAGCUGAUCCACGACGACUCCCUGACCUUCAAG |
| | | GAGGACAUCCAGAAGGCCCAGGUGUCCGGCCAGGGCGACUCCCUGC |
| | | ACGAGCACAUCGCCAACCUGGCCGGCUCCCCCGCCAUCAAGAAGGG |
| | | CAUCCUGCAGACCGUGAAGGUGGUGGACGAGCUGGUGAAGGUGAU |
| | | GGGCCGGCACAAGCCCGAGAACAUCGUGAUCGAGAUGGCCCGGGAG |
| | | AACCAGACCACCCAGAAGGGCCAGAAGAACUCCCGGGAGCGGAUGA |
| | | AGCGGAUCGAGGAGGGCAUCAAGGAGCUGGGCUCCCAGAUCCUGAA |
| | | GGAGCACCCCGUGGAGAACACCCAGCUGCAGAACGAGAAGCUGUAC |
| | | CUGUACUACCUGCAGAACGGCCGGGACAUGUACGUGGACCAGGAGC |
| | | UGGACAUCAACCGGCUGUCCGACUACGACGUGGACCACAUCGUGCC |
| | | CCAGUCCUUCCUGAAGGACGACUCCAUCGACAACAAGGUGCUGACC |
| | | CGGUCCGACAAGAACCGGGGCAAGUCCGACAACGUGCCCUCCGAGG |
| | | AGGUGGUGAAGAAGAUGAAGAACUACUGGCGGCAGCUGCUGAACG |
| | | CCAAGCUGAUCACCCAGCGGAAGUUCGACAACCUGACCAAGGCCGA |
| | | GCGGGGCGGCCUGUCCGAGCUGGACAAGGCCGGCUUCAUCAAGCGG |
| | | CAGCUGGUGGAGACCCGGCAGAUCACCAAGCACGUGGCCCAGAUCC |
| | | UGGACUCCCGGAUGAACACCAAGUACGACGAGAACGACAAGCUGAU |
| | | CCGGGAGGUGAAGGUGAUCACCCUGAAGUCCAAGCUGGUGUCCGAC |
| | | UUCCGGAAGGACUUCCAGUUCUACAAGGUGCGGGAGAUCAACAACU |
| | | ACCACCACGCCCACGACGCCUACCUGAACGCCGUGGUGGGCACCGC |
| | | CCUGAUCAAGAAGUACCCCAAGCUGGAGUCCGAGUUCGUGUACGGC |
| | | GACUACAAGGUGUACGACGUGCGGAAGAUGAUCGCCAAGUCCGAGC |
| | | AGGAGAUCGGCAAGGCCACCGCCAAGUACUUCUUCUACUCCAACAU |
| | | CAUGAACUUCUUCAAGACCGAGAUCACCCUGGCCAACGGCGAGAUC |
| | | CGGAAGCGGCCCCUGAUCGAGACCAACGGCGAGACCGGCGAGAUCG |
| | | UGUGGGACAAGGGCCGGGACUUCGCCACCGUGCGGAAGGUGCUGUC |
| | | CAUGCCCCAGGUGAACAUCGUGAAGAAGACCGAGGUGCAGACCGGC |
| | | GGCUUCUCCAAGGAGUCCAUCCUGCCCAAGCGGAACUCCGACAAGC |
| | | UGAUCGCCCGGAAGAAGGACUGGGACCCCAAGAAGUACGGCGGCUU |
| | | CGACUCCCCCACCGUGGCCUACUCCGUGCUGGUGGUGGCCAAGGUG |
| | | GAGAAGGGCAAGUCCAAGAAGCUGAAGUCCGUGAAGGAGCUGCUG |
| | | GGCAUCACCAUCAUGGAGCGGUCCUCCUUCGAGAAGAACCCCAUCG |
| | | ACUUCCUGGAGGCCAAGGGCUACAAGGAGGUGAAGAAGGACCUGA |
| | | UCAUCAAGCUGCCCAAGUACUCCCUGUUCGAGCUGGAGAACGGCCG |
| | | GAAGCGGAUGCUGGCCUCCGCCGGCGAGCUGCAGAAGGGCAACGAG |
| | | CUGGCCCUGCCCUCCAAGUACGUGAACUUCCUGUACCUGGCCUCCC |
| | | ACUACGAGAAGCUGAAGGGCUCCCCCGAGGACAACGAGCAGAAGCA |
| | | GCUGUUCGUGGAGCAGCACAAGCACUACCUGGACGAGAUCAUCGAG |
| | | CAGAUCUCCGAGUUCUCCAAGCGGGUGAUCCUGGCCGACGCCAACC |
| | | UGGACAAGGUGCUGUCCGCCUACAACAAGCACCGGGACAAGCCCAU |
| | | CCGGGAGCAGGCCGAGAACAUCAUCCACCUGUUCACCCUGACCAAC |
| | | CUGGGCGCCCCCGCCGCCUUCAAGUACUUCGACACCACCAUCGACC |
| | | GGAAGCGGUACACCUCCACCAAGGAGGUGCUGGACGCCACCCUGAU |
| | | CCACCAGUCCAUCACCGGCCUGUACGAGACCCGGAUCGACCUGUCC |
| | | CAGCUGGGCGGCGACGGCGGCGGCUCCCCCAAGAAGAAGCGGAAGG |
| | | UGUCCGAGUCCGCCACCCCCGAGUCCGUGUCCGGCUGGCGGCUGUU |
| | | CAAGAAGAUCUCCUGA |
| 863 | Amino acid | MDKKYSIGLDIGTNSVGWAVITDEYKVPSKKFKVLGNTDRHSIKKNLIG |
| | sequence for | ALLFDSGETAEATRLKRTARRRYTRRKNRICYLQEIFSNEMAKVDDSFFH |
| | Cas9 encoded by | RLEESFLVEEDKKHERHPIFGNIVDEVAYHEKYPTIYHLRKKLVDSTDKA |
| | SEQ ID Nos. | DLRLIYLALAHMIKFRGHFLIEGDLNPDNSDVDKLFIQLVQTYNQLFEEN |
| | 858-862 | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYKE |
| | | IFFDQSKNGYAGYIDGGASQEEFYKFIKPILEKMDGTEELLVKLNREDLL |
| | | RKQRTFDNGSIPHQIHLGELHAILRRQEDFYPFLKDNREKIEKILTFRIPYY |
| | | VGPLARGNSRFAWMTRKSEETITPWNFEEVVDKGASAQSFIERMTNFDK |
| | | NLPNEKVLPKHSLLYEYFTVYNELTKVKYVTEGMRKPAFLSGEQKKAIV |
| | | DLLFKTNRKVTVKQLKEDYFKKIECFDSVEISGVEDRFNASLGTYHDLLK |
| | | IIKDKDFLDNEENEDILEDIVLTLTLFEDREMIEERLKTYAHLFDDKVMKQ |
| | | LKRRRYTGWGRLSRKLINGIRDKQSGKTILDFLKSDGFANRNFMQLIHDD |
| | | SLTFKEDIQKAQVSGQGDSLHEHIANLAGSPAIKKGILQTVKVVDELVKV |
| | | MGRHKPENIVIEMARENQTTQKGQKNSRERMKRIEEGIKELGSQILKEHP |
| | | VENTQLQNEKLYLYYLQNGRDMYVDQELDINRLSDYDVDHIVPQSFLK |
| | | DDSIDNKVLTRSDKNRGKSDNVPSEEVVKKMKNYWRQLLNAKLITQRK |
| | | FDNLTKAERGGLSELDKAGFIKRQLVETRQITKHVAQILDSRMNTKYDE |
| | | NDKLIREVKVITLKSKLVSDFRKDFQFYKVREINNYHHAHDAYLNAVVG |
| | | TALIKKYPKLESEFVYGDYKVYDVRKMIAKSEQEIGKATAKYFFYSNIM |
| | | NFFKTEITLANGEIRKRPLIETNGETGEIVWDKGRDFATVRKVLSMPQVNI |
| | | VKKTEVQTGGFSKESILPKRNSDKLIARKKDWDPKKYGGFDSPTVAYSV |
| | | LVVAKVEKGKSKKLKSVKELLGITIMERSSFEKNPIDFLEAKGYKEVKKD |
| | | LIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKEVLD |
| | | ATLIHQSITGLYETRIDLSQLGGDGGGSPKKKRKV |
| 864 | Amino acid | MDKKYSIGLDIGTNSVGWAVITDEYKVPSKKFKVLGNTDRHSIKKNLIG |
| | sequence for | ALLFDSGETAEATRLKRTARRRYTRRKNRICYLQEIFSNEMAKVDDSFFH |
| | Cas9 with Hibit | RLEESFLVEEDKKHERHPIFGNIVDEVAYHEKYPTIYHLRKKLVDSTDKA |
| | tag | DLRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNLSD |
| | | AILLSDILRVNTEITKAPLSASMIKRYDEHHQDLTLLKALVRQQLPEKYKE |
| | | IFFDQSKNGYAGYIDGGASQEEFYKFIKPILEKMDGTEELLVKLNREDLL |
| | | RKQRTFDNGSIPHQIHLGELHAILRRQEDFYPFLKDNREKIEKILTFRIPYY |
| | | VGPLARGNSRFAWMTRKSEETITPWNFEEVVDKGASAQSFIERMTNFDK |
| | | NLPNEKVLPKHSLLYEYFTVYNELTKVKYVTEGMRKPAFLSGEQKKAIV |
| | | DLLFKTNRKVTVKQLKEDYFKKIECFDSVEISGVEDRFNASLGTYHDLLK |
| | | IIKDKDFLDNEENEDILEDIVLTLTLFEDREMIEERLKTYAHLFDDKVMKQ |
| | | LKRRRYTGWGRLSRKLINGIRDKQSGKTILDFLKSDGFANRNFMQLIHDD |
| | | SLTFKEDIQKAQVSGQGDSLHEHIANLAGSPAIKKGILQTVKVVDELVKV |
| | | MGRHKPENIVIEMARENQTTQKGQKNSRERMKRIEEGIKELGSQILKEHP |
| | | VENTQLQNEKLYLYYLQNGRDMYVDQELDINRLSDYDVDHIVPQSFLK |
| | | DDSIDNKVLTRSDKNRGKSDNVPSEEVVKKMKNYWRQLLNAKLITQRK |
| | | FDNLTKAERGGLSELDKAGFIKRQLVETRQITKHVAQILDSRMNTKYDE |
| | | NDKLIREVKVITLKSKLVSDFRKDFQFYKVREINNYHHAHDAYLNAVVG |
| | | TALIKKYPKLESEFVYGDYKVYDVRKMIAKSEQEIGKATAKYFFYSNIM |
| | | NFFKTEITLANGEIRKRPLIETNGETGEIVWDKGRDFATVRKVLSMPQVNI |
| | | VKKTEVQTGGFSKESILPKRNSDKLIARKKDWDPKKYGGFDSPTVAYSV |
| | | LVVAKVEKGKSKKLKSVKELLGITIMERSSFEKNPIDFLEAKGYKEVKKD |
| | | LIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKEVLD |
| | | ATLIHQSITGLYETRIDLSQLGGDGGGSPKKKRKVSESATPESVSGWRLF |
| | | KKIS |

In some embodiments, the insertion template comprises the SERPINA1 sequence of SEQ ID NO: 717 (Construct 7) or 719 (Construct 8). In some embodiments, the insertion template comprises a nucleic acid sequence having at least 95, 96, 97, 98, 99% identity to SEQ ID NO: 717 (Construct 7) or 719 (Construct 8). In some embodiments, the insertion template comprises non-wt codon usage at a region (or one or more regions) of the sequence corresponding to bases 409-431, 409-410, 412-431, 415-418, 506-528, 506-525, 519-522, 527-528, 538-560, 538-557, 551-554, 559-560, 957-977, 970-976, 1403-1436, 1403-1425, 1410-1436, 1418-1424, 1423-1435, or any combination thereof.

### EXAMPLES

The following examples are provided to illustrate certain disclosed embodiments and are not to be construed as limiting the scope of this disclosure in any way.

### Example 1. Materials and Methods

### Next-Generation Sequencing (“NGS”) and Analysis for On-Target Cleavage Efficiency

Genomic DNA was extracted using a commercial kit, e.g. Zymo Research DNA Extraction Kit (Catalog #D3012), according to manufacturer's protocol.

To quantitatively determine the efficiency of editing at the target location in the genome, deep sequencing was utilized to identify the presence of insertions and deletions introduced by gene editing. PCR primers were designed around the target site within the gene of interest (e.g., SERPINA1), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “indel percent”) as used in the examples is defined as the total number of sequence reads with insertions or deletions (“indels”) over the total number of sequence reads, including wild type.

### Preparation of Lipid Nanoparticles

The lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

The lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, 1,2-distearoyl-sn-glycero-3-phosphocholine (DSPC), and 1,2-dimyristoyl-rac-glycero-3-methylpolyoxyethylene glycol 2000 (PEG2k-DMG) in a 50:38:9:3 molar ratio, respectively. The lipid nucleic acid assemblies were formulated with a lipid amine to RNA phosphate (N:P) molar ratio of about 6, and a ratio of gRNA to mRNA of 1:2 by weight unless otherwise specified.

Lipid nanoparticles (LNPs) were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2.). The LNPs were held for 1 hour at room temperature (RT), and further diluted with water (approximately 1:1 v/v). LNPs were concentrated using tangential flow filtration on a flat sheet cartridge (Sartorius, 100 kD MWCO) and buffer exchanged into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation sequence was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine Rnase inhibitor (NEB); 0.004 U/μL Inorganic E. coli pyrophosphatase (NEB); and 1× reaction buffer. TURBO Dnase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a Rneasy Maxi kit (Qiagen) per the manufacturers' protocols. Alternatively, the mRNA was purified through a precipitation protocol, which in some cases was followed by HPLC-based purification. Briefly, after the Dnase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID NOs: 857-864 (see sequences in Table 9B). When SEQ ID NOs: 857-864 are referred to below with respect to RNAs, it is understood that Ts should be replaced with Us (which were N1-methyl pseudouridines as described above). Messenger RNAs used in the Examples include a 5′ cap and a 3′ poly-A tail, e.g., up to 100 nts, and are identified by the SEQ ID NOs: 858-862 in Table 9B. Guide RNAs are chemically synthesized by methods known in the art.

### Cloning and Plasmid Preparation

A bidirectional insertion construct flanked by AAV2 ITRs was synthesized and cloned into pUC57-Kan by a commercial vendor. The resulting construct (P00147) was used as the parental cloning vector for other vectors. The other insertion constructs (without ITRs) were also commercially synthesized and cloned into pUC57. Purified plasmid was digested with BglII restriction enzyme (New England BioLabs, cat #R0144S), and the insertion constructs were cloned into the parental vector. Plasmid was propagated in Stbl3™ Chemically Competent E. coli (Thermo Fisher, Cat #C737303).

### AAV Production

Triple transfection in HEK293 cells was used to package genomes with constructs of interest for AAV8 and AAV-DJ production and resulting vectors were purified from both lysed cells and culture media using routine methods, e.g., chromatography or iodixanol gradient ultracentrifugation (See, e.g., Lock et al., Hum Gene Ther. 2010 October; 21(10):1259-71). Isolated AAV was dialyzed in storage buffer (PBS with 0.001% Pluronic F68). AAV titer was determined by qPCR using primers/probe located within the ITR region.

### In Vivo Delivery of LNP and AAV

Mice at 6-8 weeks in age were dosed with both AAV and LNP, or vehicle (PBS+0.001% Pluronic for AAV vehicle, TSS for LNP vehicle) via the lateral tail vein. AAV were administered in a volume of 0.1 mL per animal with amounts (vector genomes/mouse, “vg/ms”) as described herein. LNPs were diluted in TSS and administered at amounts as indicated herein, at about 5 l/gram body weight. Volumes of LNP and AAV are mixed pre-dose and dosed simultaneously. At various times points post-treatment, serum was collected for certain analyses as described further below.

Human Alpha 1-Antitrypsin (hA1AT) ELISA Analysis

For in vivo studies, blood was collected, and the serum was isolated as indicated. The total human alpha 1-antitripsin levels were determined using an Alpha 1-Antitrypsin ELISA Kit (Human) (Aviva Biosystems, Cat #OKIA00048) according to manufacturer's protocol. Serum hA1AT levels were quantitated off a standard curve using 4 parameter logistic fit and expressed as g/mL of serum.

It is understood that guide sequences may or may not include the zeros before the guide number. That is G000400 is the same as G400, or with intermediate numbers of zeros prior to 400.

### Example 2—In Vivo Editing of hSERPINA1 PIZ Transgene

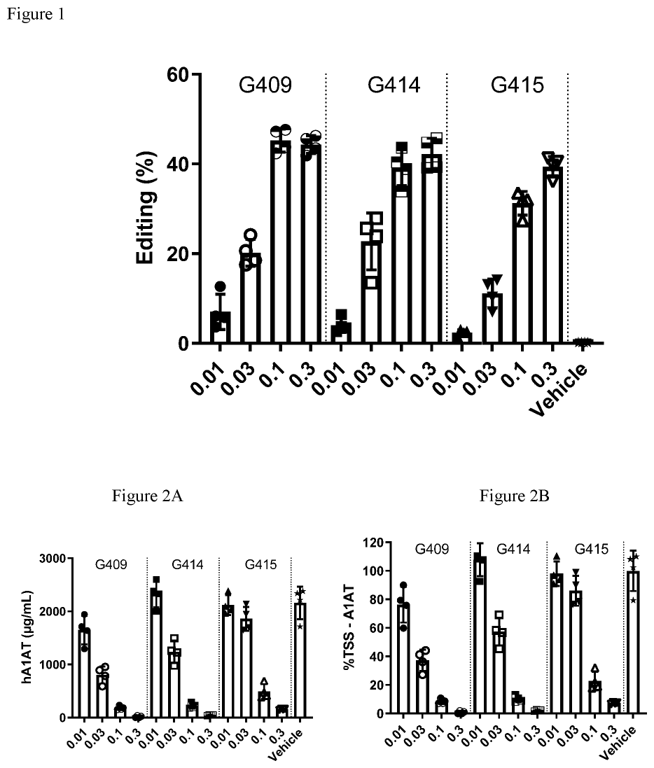

Three sgRNA were assessed for editing via indel formation and expression of Alpha-1-anti-trypsin (A1AT) protein from hSERPINA1 PIZ variant transgene. LNPs tested in this Example were prepared and delivered to mice as described in Example 1. The three sgRNAs specified in Table 8 were each assessed at four dose levels (0.3, 0.1. 0.03, and 0.01 mg/kg) in a dose response assay. Three weeks post dose, the animals were euthanized, liver tissue and blood were collected to assess liver editing and hA1AT expression levels in serum, respectively. Indel formation was determined by NGS as described in Example 1. Human A1AT levels in serum were determined by ELISA (Aviva Biosystems, Cat #OKIA00048) as described in Example 1. Editing results at the hSERPINA1 locus are shown in FIG. 1 and Table 10. Serum hA1AT levels are shown in FIG. 2A and Table 11. Relative expression of A1AT in serum was calculated as a percent in comparison to the TSS group and is shown in FIG. 2B and Table 11.

TABLE 10
Mean percent editing in mouse liver

| Treatment | | Dose | Mean | | |
|---|---|---|---|---|---|
| Group | Guide | (mpk) | % Indel | SD | Samples |
| Group 1 | G000409 | 0.01 | 7.0 | 3.9 | 4 |
| Group 2 | G000409 | 0.03 | 20.2 | 3.0 | 4 |
| Group 3 | G000409 | 0.1 | 45.3 | 2.6 | 4 |
| Group 4 | G000409 | 0.3 | 44.3 | 2.0 | 4 |
| Group 5 | G000414 | 0.01 | 4.1 | 1.6 | 4 |
| Group 6 | G000414 | 0.03 | 22.7 | 6.4 | 4 |
| Group 7 | G000414 | 0.1 | 39.2 | 4.0 | 4 |
| Group 8 | G000414 | 0.3 | 42.2 | 3.5 | 4 |
| Group 9 | G000415 | 0.01 | 2.4 | 0.6 | 4 |
| Group 10 | G000415 | 0.03 | 11.1 | 3.2 | 4 |
| Group 11 | G000415 | 0.1 | 31.2 | 2.6 | 4 |
| Group 12 | G000415 | 0.3 | 39.4 | 2.3 | 4 |
| Group 13 | TSS | — | 0.1 | 0.0 | 4 |

TABLE 11
hA1AT levels in serum

| | | | Mean | | % | |
|---|---|---|---|---|---|---|
| Treatment | | Dose | μg/mL | | A1AT | |
| Group | Guide | (mpk) | A1AT | SD | KD | Samples |
| Group 1 | G000409 | 0.01 | 1647.6 | 270.2 | 23.8 | 4 |
| Group 2 | G000409 | 0.03 | 804.4 | 159.8 | 62.8 | 4 |
| Group 3 | G000409 | 0.1 | 181.5 | 35.2 | 91.6 | 4 |
| Group 4 | G000409 | 0.3 | 14.9 | 18.2 | 99.3 | 4 |
| Group 5 | G000414 | 0.01 | 2328.8 | 247.7 | 0.0 | 4 |
| Group 6 | G000414 | 0.03 | 1239.7 | 210.7 | 42.6 | 4 |
| Group 7 | G000414 | 0.1 | 220.4 | 48.9 | 89.8 | 4 |
| Group 8 | G000414 | 0.3 | 47.1 | 7.8 | 97.8 | 4 |
| Group 9 | G000415 | 0.01 | 2118.0 | 186.3 | 2.0 | 4 |
| Group 10 | G000415 | 0.03 | 1858.9 | 225.3 | 14.0 | 4 |
| Group 11 | G000415 | 0.1 | 489.2 | 140.3 | 77.4 | 4 |
| Group 12 | G000415 | 0.3 | 156.1 | 12.6 | 92.8 | 4 |
| Group 13 | TSS | — | 2161.0 | 306.1 | — | 4 |

### Example 3. Off-Target Analysis of sgRNAs Targeted to Human SERPINA1

A biochemical assay (See, e.g., Cameron et al., Nature Methods. 6, 600-606; 2017) was used to discover potential off-target genomic sites cleaved by Cas9 targeting SERPINA1. Purified genomic DNA (gDNA) from cells were digested with in vitro assembled ribonucleoprotein (RNP) of Cas9 and sgRNA, to induce DNA cleavage at the on-target site and potential off-target sites with homology to the sgRNA spacer sequence. After gDNA digestion, the free gDNA fragment ends were ligated with adapters to facilitate edited fragment enrichment and NGS library construction. The NGS libraries were sequenced and through bioinformatic analysis, the reads were analyzed to determine the genomic coordinates of the free DNA ends. Locations in the human genome with an accumulation of reads were then annotated as potential off-target sites.

In known off-target detection assays, such as the biochemical assay used above, a large number of potential off-target sites are typically recovered, by design, so as to “cast a wide net” for potential sites that can be validated in other contexts, e.g., in a primary cell of interest. For example, the biochemical assay typically overrepresents the number of potential off-target sites as the assay utilizes purified high molecular weight genomic DNA free of the cell environment and is dependent on the dose of Cas9 ribonucleoprotein used. Accordingly, potential off-target sites identified by these assays were validated using targeted sequencing of the identified potential off-target sites.

In one approach to targeted sequencing, Cas9 and a sgRNA of interest (e.g., a sgRNA having potential off-target sites for evaluation) were introduced to PHH or PCH cells. The cells were then lysed and primers flanking the potential off-target site(s) were used to generate an amplicon for NGS analysis. Identification of indels at a certain level can be used to validate potential off-target site, whereas the lack of indels found at the potential off-target site can indicate a false positive in the off-target assay that was utilized.

Guides showing on target indel activity were tested for potential off-target genomic cleavage sites with this assay. Repair structures were manually inspected at loci with statistically relevant indel rates at the off-target cleavage sites to validate the repair structures.

No validated off-target editing activity was identified for any of guides G000409, G000414, and G000415.

### Example 4. In Vitro SERPINA1 Insertion Template Validation in Primary Mouse Hepatocytes

Primary Mouse Hepatocytes (PMH)(Gibco, Amarillo, Texas, Lot #MC837) were plated at 45,000 cells per well in 96-well Bio-Coat plates from Corning (Corning, NY, Cat #354407). Forty-eight hours after plating, LNP containing mouse albumin intron 1-targeting sgRNA with Cas9 mRNA (2:1 guide to mRNA ratio) were thawed on ice as well as AAV containing the listed insertion plasmids. LNP was diluted to 1 mg Cas9 mRNA/mL in 3% FBS William's E Media (ThermoFisher, Waltham, MA, Cat #A1217601) and 100 μL/well was administered to all experimental wells except those being “untreated” or receiving “AAV only”. The AAV preparations were diluted in 10 μL water/well to achieve a multiplicity of infection (MOI) of 5e5 for each well where AAV was administered. The cells were incubated at 37° C. for 96 hours.

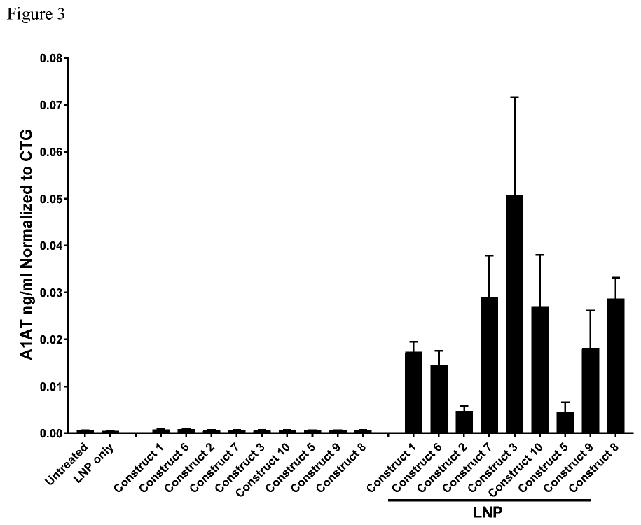

After 96 hours, media was removed, fresh media was added, and cells were incubated at 37° C. After an additional 96 hours, cells plates were removed from incubator and media was collected for hAAT quantification via ELISA (Aviva Biosystems, San Diego, CA, Cat #OKIA00048). The ELISA was carried out according to manufacturer protocol. Meanwhile, the remaining cells were utilized for CellTiter Glo 2.0 Cell Viability Assay (Promega, Madison, WI, Cat #G9241) to quantify relative cell number in each well. The A1AT ELISA results were normalized to Cell Titer Glo values to correct for cell number. Results are shown in FIG. 3.

### Example 5 In Vivo Insertion of hSERPINA1 into mAlbumin Locus with Mice Expressing hSERPINA1 PIZ Transene

In vivo insertion of hSERPINA1 into mAlbumin locus was assessed in male NSG-PIZ mice expressing the hSERPINA1 PIZ variant transgene and in male wildtype NSG mice to evaluate durability of protein expression out to 6 months post insertion. NSG-PiZ mice are transgenic mice harboring multiple copies of the human SERPINA1 PiZ variant (Glu342Lys) on the immunodeficient NOD scid gamma (NSG) background. Both NSG-PiZ and wild type NSG mice are from Jackson Laboratory. The ssAAV and LNPs tested in this Example were prepared and delivered to mice as described in Example 1 to male NSG mice (Groups 1-3) and NSG-PIZ male mice (Group 4-6).

Mice were dosed with 1 mg/kg (with respect to total RNA cargo content) LNP carrying Cas9 mRNA and sgRNA G000666 (targeting mouse albumin) prepared as described above. Groups 2 and 5 were dosed additionally with ssAAV derived from Construct Nanoluc (nanoluc) at 5e11 vg/mouse. Groups 3 and 6 were dosed additionally with ssAAV derived from Construct 1 A1AT Template at 5e11 vg/mouse (Table 12). Human A1AT levels in the serum were determined by ELISA (Aviva Biosystems, Cat #OKIA00048) at one, two, and three weeks after dosing then monthly thereafter up to 6 months post-dose. This kit is specific for human A1AT and detects both PiZ variant and wild-type A1AT produced by the inserted template. Six months post-dose, the animals were euthanized, blood was collected, and serum was prepared to assess hA1AT serum levels. Serum was sent to IDEXX Laboratories for liver enzyme quantitation.

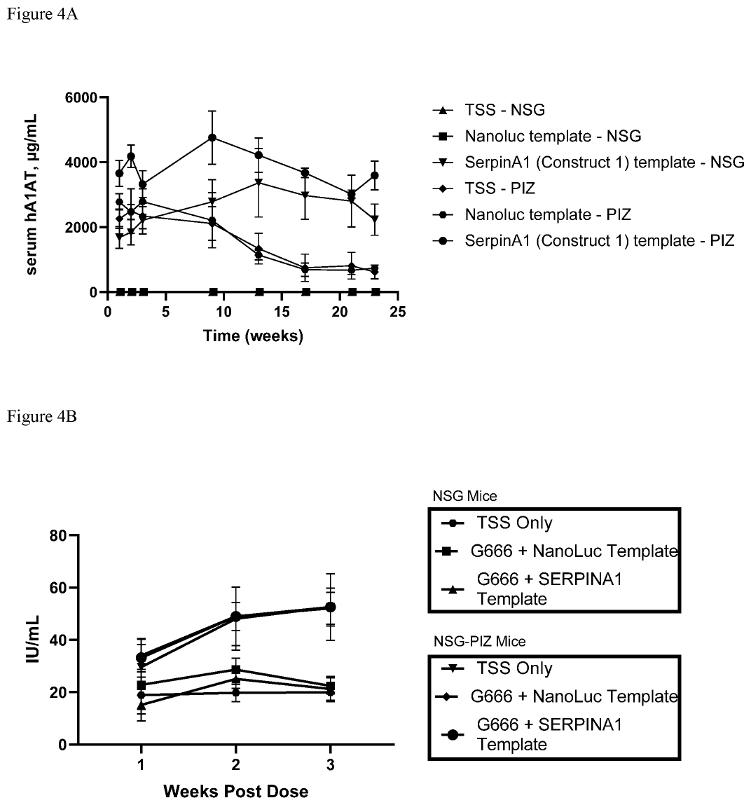

FIG. 4A and Table 13 shows hA1AT protein levels in serum at various time points as measured by ELISA. FIG. 4B shows serum ALT activity and Table 14 shows serum ALT and AST activity.

TABLE 12

| Treatment Group | Strain | AAV | Guide |
|---|---|---|---|
| Group 1 | NSG | Vehicle | Vehicle |
| Group 2 | NSG | Construct Nanoluc | G000666 |
| Group 3 | NSG | Construct 1 | G000666 |
| Group 4 | NGS-PiZ | Vehicle | Vehicle |
| Group 5 | NGS-PiZ | Construct Nanoluc | G000666 |
| Group 6 | NGS-PiZ | Construct 1 | G000666 |

TABLE 13
hA1AT levels in serum as measured by ELISA

| Treatment | Data | Week | Week | Week | Week | Week | Week | Week | Week |
|---|---|---|---|---|---|---|---|---|---|
| Group | Type | 1 | 2 | 3 | 9 | 13 | 17 | 21 | 23 |
| Group 1 | Mean (μg/ml) | 0 | 0 | 0 | 0 | 0 | 0 | 0 | 0 |
| | SD | 0 | 0 | 0 | 0 | 0 | 0 | 0 | 0 |
| | Samples (n) | 5 | 5 | 5 | 5 | 5 | 5 | 5 | 5 |
| Group 2 | Mean (μg/ml) | 0 | 0 | 0 | 0 | 0 | 0 | 0 | 0 |
| | SD | 0 | 0 | 0 | 0 | 0 | 0 | 0 | 0 |
| | Samples (n) | 5 | 5 | 5 | 5 | 5 | 5 | 5 | 5 |
| Group 3 | Mean (μg/ml) | 1585.6 | 1807.4 | 2214.1 | 2783.5 | 3368.7 | 2973.3 | 2803.9 | 2233.0 |
| | SD | 323.4 | 272.0 | 421.4 | 674.6 | 1054.1 | 732.1 | 800.5 | 479.5 |
| | Samples (n) | 5 | 5 | 5 | 5 | 5 | 5 | 5 | 5 |
| Group 4 | Mean (μg/ml) | 1999.3 | 1860.2 | 2343.9 | 2112.5 | 1336.7 | 748.9 | 813.9 | 617.2 |
| | SD | 226.8 | 399.4 | 398.4 | 519.6 | 472.0 | 420.9 | 412.4 | 209.6 |
| | Samples (n) | 5 | 5 | 5 | 5 | 5 | 5 | 5 | 5 |
| Group 5 | Mean (μg/ml) | 2180.7 | 2021.7 | 2789.8 | 2214.6 | 1142.8 | 692.6 | 674.7 | 739.5 |
| | SD | 179.7 | 218.6 | 392.4 | 850.5 | 149.8 | 206.8 | 132.4 | 82.6 |
| | Samples (n) | 5 | 5 | 5 | 5 | 5 | 5 | 5 | 5 |
| Group 6 | Mean (μg/ml) | 2771.6 | 2995.5 | 3321.0 | 4755.7 | 4217.0 | 3670.4 | 3017.7 | 3590.3 |
| | SD | 382.3 | 342.9 | 414.5 | 823.3 | 531.7 | 149.1 | 126.1 | 443.4 |
| | Samples (n) | 5 | 5 | 5 | 5 | 5 | 5 | 4* | 4* |

*one mouse was found moribund and euthanized before week 21

TABLE 14
Liver enzyme serum levels (AST and ALT)

| | | | Mean | AST | Mean | ALT |
|---|---|---|---|---|---|---|
| Group | Strain | AAV | AST | SD | ALT | SD |
| 1 | NSG | Vehicle | 83.6 | 47.5 | 46.6 | 34.1 |
| 2 | NSG | Nanoluc | 107.0 | 87.1 | 61.0 | 80.0 |
| 3 | NSG | Construct 1 | 130.6 | 102.0 | 44.4 | 47.2 |
| 4 | NSG-PiZ | Vehicle | 100.8 | 14.4 | 35.0 | 11.0 |
| 5 | NSG-PiZ | Nanoluc | 158.4 | 90.1 | 38.4 | 7.3 |
| 6 | NSG-PiZ | Construct 1 | 225.2 | 61.9 | 52.5 | 12.9 |

### Example 6—In Vivo Insertion of hSERPINA1 into the mAlbumin Locus: AAV Template Screen

Insertion of hSERPINA1 into male C57BL mouse albumin locus using seven bidirectional ssAAV constructs was tested. The ssAAV and LNPs tested in this Example were prepared and delivered to mice as described in Example 1.

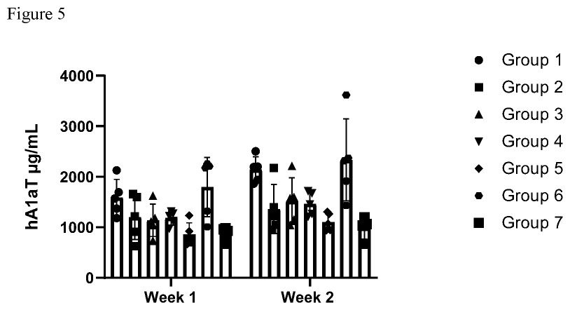

Mice at 6-8 weeks of age were dosed with 1 mg/kg (with respect to total RNA cargo content) LNP carrying Cas9 mRNA and sgRNA G000666 (targeting mouse albumin). The seven ssAAV were assessed at a dose of 5e11 vg/ms (Table 15). Blood was collected at weeks one, two, and three weeks post-dose. Four weeks post dose, the animals were euthanized, liver tissue and blood were collected to assess liver editing and hA1AT expression levels in serum, respectively. Indel formation was determined by NGS. and sera was prepared to measure human alpha1 antitrypsin (hA1AT) serum expression by ELISA (Aviva Biosystems, Cat #OKIA00048). Serum hA1AT levels are shown in FIG. 5 and Table 16 at one, two, three, and four weeks post dose.

TABLE 15

| Treatment | Guide | AAV Construct | AAV dose |
|---|---|---|---|
| Group | (1 mpk) | ID | (vg/ms) |
| 1 | G000666 | Construct 1 | 5e11 |
| 2 | G000666 | Construct 2 | 5e11 |
| 3 | G000666 | Construct 7 | 5e11 |
| 4 | G000666 | Construct 3 | 5e11 |
| 5 | G000666 | Construct 10 | 5e11 |
| 6 | G000666 | Construct 5 | 5e11 |
| 7 | G000666 | Construct 9 | 5e11 |

TABLE 16

| Treatment | | Data | | | | |
|---|---|---|---|---|---|---|
| Group | AAV ID | Type | Week 1 | Week 2 | Week 3 | Week 4 |
| Group 1 | Construct 1 | Mean | 1589.5 | 2142.0 | 2233.5 | 1607.6 |
| | | (μg/ml) | | | | |
| | | SD | 359.0 | 252.4 | 637.4 | 312.4 |
| | | Samples | 5 | 5 | 5 | 5 |
| | | (n) | | | | |
| Group 2 | Construct 2 | Mean | 1202.0 | 1360.4 | 2128.4 | 2494.3 |
| | | (μg/ml) | | | | |
| | | SD | 442.2 | 486.4 | 991.6 | 10.4 |
| | | Samples | 5 | 5 | 5 | 2** |
| | | (n) | | | | |
| Group 3 | Construct 7 | Mean | 1140.0 | 1518.1 | 2285.1 | 1578.2 |
| | | (μg/ml) | | | | |
| | | SD | 320.8 | 463.9 | 686.4 | 531.2 |
| | | Samples | 5 | 5 | 5 | 5 |
| | | (n) | | | | |
| Group 4 | Construct 3 | Mean | 1181.6 | 1463.3 | 2344.5 | 1520.8 |
| | | (μg/ml) | | | | |
| | | SD | 136.5 | 231.4 | 339.5 | 352.5 |
| | | Samples | 5 | 5 | 5 | 5 |
| | | (n) | | | | |
| Group 5 | Construct 10 | Mean | 859.7 | 1104.9 | 1771.1 | 1078.6 |
| | | (μg/ml) | | | | |
| | | SD | 228.4 | 173.3 | 208.6 | 189.3 |
| | | Samples | 5 | 5 | 5 | 5 |
| | | (n) | | | | |
| Group 6 | Construct 5 | Mean | 1795.6 | 2332.1 | 3115.9 | 2291.5 |
| | | (μg/ml) | | | | |
| | | SD | 585.3 | 811.4 | 1084.3 | 639.1 |
| | | Samples | 5 | 5 | 5 | 5 |
| | | (n) | | | | |
| Group 7 | Construct 9 | Mean | 851.6 | 990.6 | 1508.9 | 1082.4 |
| | | (μg/ml) | | | | |
| | | SD | 145.5 | 483.5 | 341.3 | 507.5 |
| | | Samples | 5 | 5 | 4 | 4 |
| | | (n) | | | | |

**The day before week 4 takedown, 3 mice were found dead and 2 moribund. Blood was collected from 2 moribund animals and assayed per protocol.

### Example 7—In Vivo Insertion of hSERPINA1 into the mAlbumin Locus: Dose Response

Insertion of hSERPINA1 into male C57BL mouse albumin locus using three bidirectional ssAAV constructs was tested in a dose response assay. The ssAAV and LNPs tested in this Example were prepared and delivered to mice as described in Example 1.

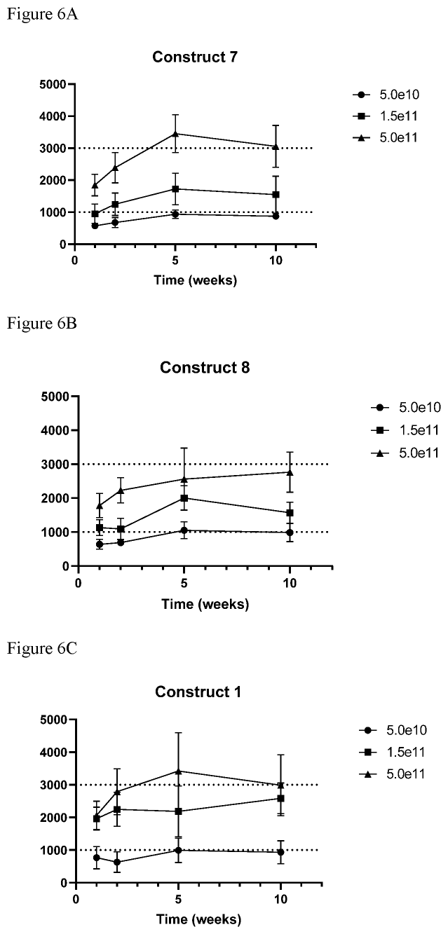

Mice at 6-8 weeks of age were dosed with 1 mg/kg (with respect to total RNA cargo content) LNP carrying Cas9 mRNA and sgRNA G000666 (targeting mouse albumin). The three ssAAV derived from P00450 were assessed at three doses: 5e10, 1e11, and 5e11 vg/ms (Table 17). Blood was collected at weeks one, two, five, ten, and fourteen weeks post-dose and sera was prepared to measure human alpha1 antitrypsin (hA1AT) serum expression by ELISA (Aviva Biosystems, Cat #OKIA00048). Serum hA1AT levels are shown in FIGS. 6A-6C and Table 18 at one, two, five, ten, and fourteen (in Table 18) weeks post dose.

TABLE 17

| Treatment | Guide | AAV Construct | AAV dose |
|---|---|---|---|
| Group | (1 mpk) | ID | (vg/ms) |
| 1 | G000666 | Construct 7 | 5e10 |
| 2 | G000666 | Construct 7 | 1e11 |
| 3 | G000666 | Construct 7 | 5e11 |
| 4 | G000666 | Construct 8 | 5e10 |
| 5 | G000666 | Construct 8 | 1e11 |
| 6 | G000666 | Construct 8 | 5e11 |
| 7 | G000666 | Construct 1 | 5e10 |
| 8 | G000666 | Construct 1 | 1e11 |
| 9 | G000666 | Construct 1 | 5e11 |

TABLE 18

| Treatment | AAV ID | Data | | | | | |
|---|---|---|---|---|---|---|---|
| Group | vg/ms | Type | Week 1 | Week 2 | Week 5 | Week 10 | Week 14 |
| Group 1 | Construct 7 | Mean (μg/ml) | 572.0 | 676.7 | 934.5 | 872.6 | 1264.9 |
| | 5e10 | SD | 81.1 | 152.6 | 134.6 | 96.2 | 201.6 |
| | | Samples (n) | 5 | 5 | 4* | 4* | 4* |
| Group 2 | Construct 7 | Mean (μg/ml) | 952.2 | 1249.0 | 1728.3 | 1547.5 | 2027.5 |
| | 1e11 | SD | 299.7 | 353.0 | 493.8 | 577.1 | 583.5 |
| | | Samples (n) | 5 | 5 | 5 | 5 | 5 |
| Group 3 | Construct 7 | Mean (μg/ml) | 1848.1 | 2391.3 | 3453.1 | 3056.7 | 4836.0 |
| | 5e11 | SD | 337.9 | 476.5 | 592.5 | 653.7 | 994.1 |
| | | Samples (n) | 5 | 5 | 5 | 5 | 5 |
| Group 4 | Construct 8 | Mean (μg/ml) | 637.9 | 689.8 | 1052.3 | 983.8 | 1329.5 |
| | 5e10 | SD | 146.6 | 92.8 | 244.4 | 268.0 | 311.0 |
| | | Samples (n) | 5 | 5 | 5 | 5 | 5 |
| Group 5 | Construct 8 | Mean (μg/ml) | 1132.4 | 1092.4 | 2001.4 | 1568.5 | 1921.9 |
| | 1e11 | SD | 229.2 | 315.1 | 361.2 | 312.4 | 488.3 |
| | | Samples (n) | 5 | 5 | 4* | 4* | 4* |
| Group 6 | Construct 8 | Mean (μg/ml) | 1779.5 | 2225.6 | 2561.0 | 2766.5 | 3194.2 |
| | 5e11 | SD | 357.7 | 372.2 | 911.6 | 592.2 | 1196.3 |
| | | Samples (n) | 5 | 5 | 5 | 5 | 5 |
| Group 7 | Construct 1 | Mean (μg/ml) | 769.9 | 632.3 | 995.6 | 936.3 | 1449.3 |
| | 5e10 | SD | 344.6 | 313.8 | 377.8 | 350.8 | 409.0 |
| | | Samples (n) | 5 | 5 | 5 | 5 | 5 |
| Group 8 | Construct 1 | Mean (μg/ml) | 1964.3 | 2248.7 | 2187.2 | 2584.2 | 3459.8 |
| | 1e11 | SD | 351.4 | 521.3 | 779.6 | 473.2 | 593.7 |
| | | Samples (n) | 5 | 5 | 5 | 5 | 5 |
| Group 9 | Construct 1 | Mean (μg/ml) | 2063.0 | 2789.0 | 3421.7 | 2988.5 | 4409.3 |
| | 5e11 | SD | 434.0 | 703.7 | 1176.6 | 936.2 | 1657.4 |
| | | Samples (n) | 5 | 5 | 5 | 5 | 5 |

*mice died during bleeding in restraint device.

### Example 8—Susceptibility of SERPINA1 Open Reading Frames to Sequence Specific Nucleic Acid A2Ents

Lentiviral plasmid constructs were individually designed with single copies of the SERPINA1 open reading frames, each corresponding to the various gene of interest (GOI) sequences from insertion constructs Construct 1, Construct 7, and Construct 8. The lentiviral vectors contain EF1a promoters to drive GOI expression, and puromycin resistance for selection.

The designs were based on the insertion constructs shown in Table 19:

TABLE 19

| | | Component of |
|---|---|---|
| Lentivirus | | insertion |
| construct | Description | constructs |
| Construct 20 | SERPINA1 w/native signal sequence | None |
| Construct 21 | SERPINA1, no signal sequence | Construct 1 |
| Construct 22 | SERPINA1, no signal sequence, CpG | Construct 7 |
| | depleted | |
| Construct 23 | SERPINA1, no signal sequence, CpG | Construct 7, |
| | depleted, alternative codon usage 1 | Construct 8 |
| Construct 24 | SERPINA1, no signal sequence, CpG | Construct 8 |
| | depleted, alternative codon usage 2 | |

Upon sequencing, the lentiviral constructs, changes from the designed constructs were identified in Construct 23. Specifically, rather than having three mismatches from the targeting sequence of G000409, there was only one mismatch. The changes from the designs did not result in a change in the encoded amino acid sequence. The alignment of the targeting sequence of G000409, the wild type sequence of SERPINA1, the Construct 20, and Construct 7/8 is shown, with the differences from the G000409 targeting site underlined:

| | |
|---|---|
| G000409 | ACTCACGATGAAATCCTGGA (SEQ ID NO: 1567) |
| Con 20 | ACTCATGATGAAATCCTGGA (SEQ ID NO: 1568) |
| Con 7/8 | ACCCATGATGAGATCCTGGA (SEQ ID NO: 1569) |
| | ** ** ***** ******** |

Sequence specific nucleic acid agents shown in Table 20 were tested in the experiment:

TABLE 20
Nucleic Acid Agents

| | Target sequence | |
|---|---|---|
| Name | SEQ ID NO: 703. | SEQ ID NO: |
| siRNA2 | 1405-1425 | 980 (sense) 982 (antisense) |
| siRNA3 | 957-977 | 981 (sense) 984 (antisense) |
| G000409 | 506-525 | 1129 |
| G000414 | 538-557 | 1130 |
| G000415 | 413-431 | 1131 |

Hepa1.6 mouse hepatoma cells (ATCC, Manassas, VA, Cat #CRL-1380) were plated at 250,000 cells/well in 6-well dishes (Thermo Fisher, Waltham, MA, Cat #140675) with DMEM media (Millipore Sigma, Burlington, MA, Cat #D5796) and 10% Fetal Bovine Serum and incubated at 37° C. After 24 hrs, lentivirus was administered to the cells at an MOI of 6 (assuming a doubling of cells after 24 hr to total cell number in each well equaling 500,000 cells) to enable integration and expression of the lentiviral gene constructs.

After 24 hrs, transduced and control cells were treated with LNP containing shRNA (final concentration 10 nM shRNA per well) or sgRNA/Cas9 mRNA (1:2 ratio, at 3 μg total RNA/well) targeting wild-type SERPINA1 and returned to 37° C. incubation.

Forty-eight hours after treatment with the LNP, RNA was harvested using Qiagen RNAeasy Mini Kit (Hilden, Germany, Cat #74104) and converted to cDNA using High-Capacity RNA-to-cDNA Kit (Thermo Fisher, Waltham, MA, Cat #4388950), both per manufacturer's protocols.

Droplet digital PCR (ddPCR) primer-probe sets were designed to detect the transcripts resulting from expression of each lentiviral construct (Bio-Rad, Hercules, CA, Cat #10031277). A control primer-probe set to detect mouse beta-actin expression was also ordered from Bio-Rad (Cat #10031256). The cDNA samples were analyzed with the appropriate primer-probe sets via ddPCR according to manufacturer protocols.

For experiments involving cDNA quantification, 1:10,000 dilutions of cDNA (generated in 20 μL reaction with 1 μg RNA input) were performed in water. Bio-Rad ddPCR Supermix for Probes (No dUTP, Cat #1863024) was thawed on ice. 20 μL reactions were generated for each sample (10 μL Supermix+7 μL water+1 μL 10,000× diluted cDNA+1 μL SERPINA1 probeset+1 μL control gene probeset) and arrayed in 96-well plates (Bio-Rad Cat #12001925).

Droplets were generated using a Bio-Rad Automated Droplet Generator (Cat #1864101) per manufacturer protocols. Droplets generated with this machine were then thermocycled with the following manufacturer conditions, using an Applied Biosystems VeritiPro Thermal Cycler (Cat #A48141) (Table 21).

TABLE 21
Thermocyclin conditions

| | | | |
|---|---|---|---|
| Cycling | Temperature | | Number |
| Step | ° C. | Time | of Cycles |

| [symbol] ONE [symbol] | | | 2 [symbol] | 3 | min | 1 |
|---|---|---|---|---|---|---|
| [symbol] | | | [symbol] | 10 | min | 1 |
| [symbol] | | | [symbol] | [symbol] | | 40 |
| [symbol] | | | [symbol] | 1 [symbol] | | 40 |
| [symbol] | | | [symbol] | 10 | min | 1 |
| | [symbol] | [symbol] | 4 | [symbol] | | 1 |
| | [symbol] | [symbol] | 2 [symbol] | 1 | min | 1 |

[symbol]

[symbol]

indicates data missing or illegible when filed

After thermocycling, ddPCR samples were loaded onto the Bio-Rad QX200 Droplet Reader (Cat #184003) and samples were analyzed as gene expression “GEX” assay. The reader generated results for each sample, providing concentration (copies/μL) of each target, SERPINA1 and control gene).

Concentration of SERPINA1 transcript for each sample was determined and normalized to the concentration of mouse beta-actin to correct for cell-number variation. Normalized values were then compared to non-treated control samples to determine relative reduction of transcript after shRNA or CRISPR-KO treatment, with a value of 1 being indicative of 100% reduction of SERPINA1 mRNA level and 0 being indicative of no reduction of SERPINA1 mRNA level. Table 22 shows percent reduction of hSERPINA1 transcript compared to non-targeting control. Each sample was treated first with lentiviral vector (indicated by row in table) and then with LNP containing shRNA or CRISPR sgRNA (indicated by column in table).

TABLE 22
Percent reduction of hSERPINA1 transcript compared to non-targeting control.

| Primary | Secondary Treatment | | | | | |
|---|---|---|---|---|---|---|
| Treatment | Non- | | | | | |
| Lentiviral | targeting | | | | | |
| Construct | LNP | siRNA2 | siRNA3 | G000409 | G000414 | G000415 |
| Construct 20 | 0 | 0.87 | 0.83 | 0.72 | 0.72 | 0.55 |
| Construct 21 | 0 | 0.69 | 0.62 | 0.69 | 0.30 | −0.10 |
| Construct 22 | 0 | 0.10 | −0.18 | 0.38 | 0.07 | −0.29 |
| Construct 23 | 0 | 0.14 | −0.53 | 0.41 | −0.04 | −0.61 |
| Construct 24 | 0 | 0.03 | −0.02 | 0.00 | −0.30 | −0.05 |

### Example 9—In Vivo Insertion of hSERPINA1 into the Cynomolgus Albumin Locus Followed by In Vivo Knockdown of cSERPINA1 Transgene

AAV Preparation for Delivery hSERPINA1

Triple transfection of suspension Viral Production cells (Thermo Fisher, Cat #A35347) was used to package genomes with genes of interest (GOI) for AAV8 using routine methods production. Three days post transfection, AAV vectors were harvested from cell culture via cell lysis including Benzonase treatment to digest plasmid, host cell, and any other free DNA and RNA. Harvest material were then clarified by depth filtration to remove any cell debris and large molecules followed by a tangential flow filtration for removal of small molecules, buffer exchange, and volume reduction. AAV vectors were subsequently purified through an affinity chromatography, and full AAV particles (assessed by the ratio of genome titer to capsid titer) were enriched by an anion-exchange chromatography. At last, purified AAV vectors were buffer exchanged and concentrated into the final formulation buffer (PBS with 0.001% Pluronic F68, pH7.4) using centrifugation filter units. A panel of 12 tests was provided for each batch of production including a ddPCR using primers/probe located within the ITR region for genome titer determination.

Cynomolgus and Human Alpha 1-Antitrypsin (hA1AT) LC-MS/MS Analysis from Cynomolgus Serum

For in vivo studies, blood was collected, and the serum was isolated as indicated. The total cA1AT and hA1AT levels were determined using liquid chromatography-tandem mass spectrometry (LC-MS/MS). Purified lyophilized native hA1AT derived from human plasma was obtained from Athens Research & Technology. Purified lyophilized native cA1AT derived from cynomolgus serum was made internally. Lyophilized cA1AT and hA1AT were dissolved in fetal calf serum at the appropriate concentration for standards and quality controls. Serum samples were diluted 10-fold into fetal calf serum. 5 μL of 1900 ng/mL stable labeled internal standards were added to 5 μL of the fetal calf serum diluted samples, standards, and quality controls. Samples were then denatured with 25 μL trifluoroethanol, diluted with 25 μL 50 mM ammonium bicarbonate immediately before 5 μL of 200 mM DTT was added and incubated for 30 min at 55° C. The reduced samples were treated with 10 μL of 200 mM iodacetamide and incubated for one hour at room temperature in the dark with shaking. The samples were diluted with 400 μL of 50 mM ammonium bicarbonate:Methol (65:35) and treated with 20 μL of 1 μg/L trypsin, and incubated overnight at 37° C. Digestion was terminated with 10 μL of formic acid.

Identification of Wild-Type cA1AT and hA1AT Peptides

The pure A1AT digest was analyzed by LC-MS/MS and signature peptides that contained the wild-type alleles were identified. Specifically, the wild-type cA1AT was detected using heavy labeled specific peptide (SANLHLPR; SEQ ID NO: 1559), and the wild-type hA1AT was detected using a different heavy labeled wild-type specific peptide (SASLHLPK; SEQ ID NO: 1560). The combined wild-type cA1AT and hA1AT concentration was detected using a third heavy labeled peptide (AVLTIDEK; SEQ ID NO: 1561). Each of these peptides were synthesized by incorporation of a single 13C615N-leucine at the position noted by bold underline.

Determining Levels of Serum cA1AT and hA1AT Using Mass Spectrometry Serum was digested according to the methods described above. After digestion, the digested serum was loaded onto the column and analyzed by LC-MS/MS as described below. Identification of wild-type cA1AT and hA1AT levels were obtained by comparison to calibration curves.

### LC-MS/MS Conditions

LC-MS/MS analysis was performed with a 2.1×50 mm C8 column. Mobile phase A consisted of 0.10% formic acid in water and mobile phase B consisted of 0.10% formic acid in acetonitrile. A needle wash consisted of 0.1% Formic Acid, 1% dimethylsulfoxide in Methanol: Water (35:65). Analysis of the A1AT digest was performed on a mass spectrometer with the following parameters: (a) Ion Source: Turbo Spray IonDrive; (b) Curtain Gas: 35.0; (c) Collision Gas: Medium; (d) IonSpray Voltage: 5500; (e) Temperature: 500° C.; (f) Ion Source Gas 1: 50; and (g) Ion Source Gas 2: 50.

In Vivo Insertion of hSERPINA1 into the Cynomolgus Albumin Locus Followed by In Vivo Knockdown of cSERPINA1 Transgene

A human SERPINA1 bidirectional construct (Construct 1) in an AAV8 expression vector (AAV8-SERPINA1) combination with a formulated sgRNA cross-reactive with the human and cynomolgus albumin genes (G009860) was evaluated for human SERPINA1 gene insertion in male cynomolgus monkeys. The target site of the human albumin sgRNA is conserved in cynomolgus monkeys, allowing for the human SERPINA1 transgene to be inserted into the cynomolgus monkey albumin locus. Following insertion of the human SERPINA1 gene, a guide specific to cynomolgus SERPINA1 (G014418) was evaluated for cynomolgus (c)SERPINA1 gene knockout was assessed by detection of serum cynomolgus (c)A1AT as a marker of gene editing. The guides used are shown in the table below.

TABLE 23
sgRNAs

| | Target | Unmodified | Modified |
|---|---|---|---|
| sgRNA | sequence | guide | guide |
| G009860 | UAAAGCAUAG | UAAAGCAUAGUGCA | mU*mA*mA*AG |
| (human/ | UGCAAUGGAU | AUGGAUGUUUUAGA | CAUAGUGCAAU |
| cyno) | (SEQ ID | GCUAGAAAUAGCAA | GGAUGUUUUAG |
| | NO: 8) | GUUAAAAUAAGGCU | AmGmCmUmAmG |
| | | AGUCCGUUAUCAAC | mAmAmAmUmAm |
| | | UUGAAAAAGUGGCA | GmCAAGUUAAA |
| | | CCGAGUCGGUGCUU | AUAAGGCUAGU |
| | | UU | CCGUUAUCAmA |
| | | (SEQ ID | mCmUmUmGmAm |
| | | NO: 1500 | AmAmAmAmGmU |
| | | | mGmGmCmAmCm |
| | | | CmGmAmGmUmC |
| | | | mGmGmUmGmCm |
| | | | U*mU*mU*mU |
| | | | (SEQ ID |
| | | | NO: 72) |
| G014418 | AGACCUUAGU | AGACCUUAGUGAUA | mA*mG*mA*CC |
| (cyno | GAUACCCAGG | CCCAGGGUUUUAGA | UUAGUGAUACC |
| specific) | (SEQ ID | GCUAGAAAUAGCAA | CAGGGUUUUAG |
| | NO: 1502) | GUUAAAAUAAGGCU | AmGmCmUmAmG |
| | | AGUCCGUUAUCAAC | mAmAmAmUmAm |
| | | UUGAAAAAGUGGCA | GmCAAGUUAAA |
| | | CCGAGUCGGUGCUU | AUAAGGCUAGU |
| | | UU | CCGUUAUCAmA |
| | | (SEQ ID | mCmUmUmGmAm |
| | | NO: 1504) | AmAmAmAmGmU |
| | | | mGmGmCmAmCm |
| | | | CmGmAmGmUmC |
| | | | mGmGmUmGmCm |
| | | | U*mU*mU*mU |
| | | | (SEQ ID |
| | | | NO: 1506) |

Monkeys (n=3) were dosed intravenously with a bolus dose of AAV8-SERPINA1 (1.5E13 vg/kg) followed by a 30-minute IV infusion of G009860 formulated in an LNP with Cas9 mRNA as provided above (3.0 mg/kg) on study day 1. On study day 245, monkeys were dosed a 30-min IV infusion of the cynomolgus specific SERPINA1 guide G014418 formulated in an LNP with Cas9 mRNA as provided above (3.0 mg/kg). On study day 1 a vehicle control group (n=3) was dosed with a bolus dose of AAV buffer followed by a 30-minute infusion of LNP buffer. On study day 245, the vehicle control group was dosed with a 30-minute infusion of LNP buffer. All monkeys were pre-treated with a bolus dose of 2 mg/kg dexamethasone 1 hour prior to the AAV bolus on study day 1, and 1-hour prior to LNP infusion on study day 245. The AAV and LNPs tested in this study were prepared as described in the materials and methods. Serum cA1AT/hA1AT levels and gene editing were measured as described in the materials and methods.

All animals were prescreened for single-nucleotide variants in the sgRNA target sequence and for pre-existing anti-AAV8 neutralizing antibodies. Pharmacokinetic evaluation of AAV and LNP components in plasma were within historical ranges for all treated animals indicating successful dosing of all products. Clinical pathology (clinical chemistry, hematology, coagulation) and cytokine monitoring did not yield any unusual findings with any parameter elevations returning to baseline within one week.

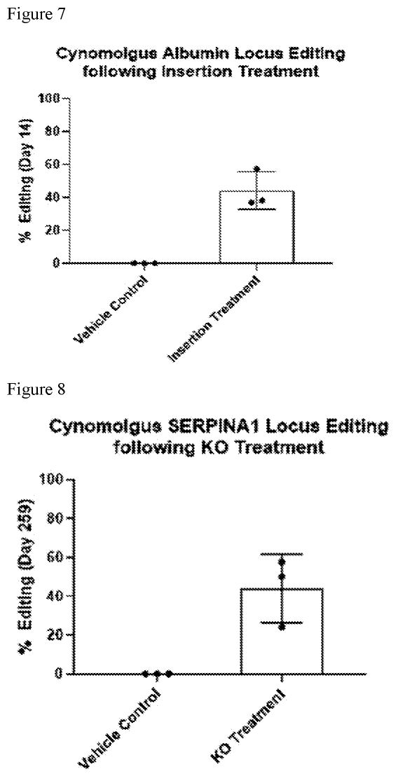

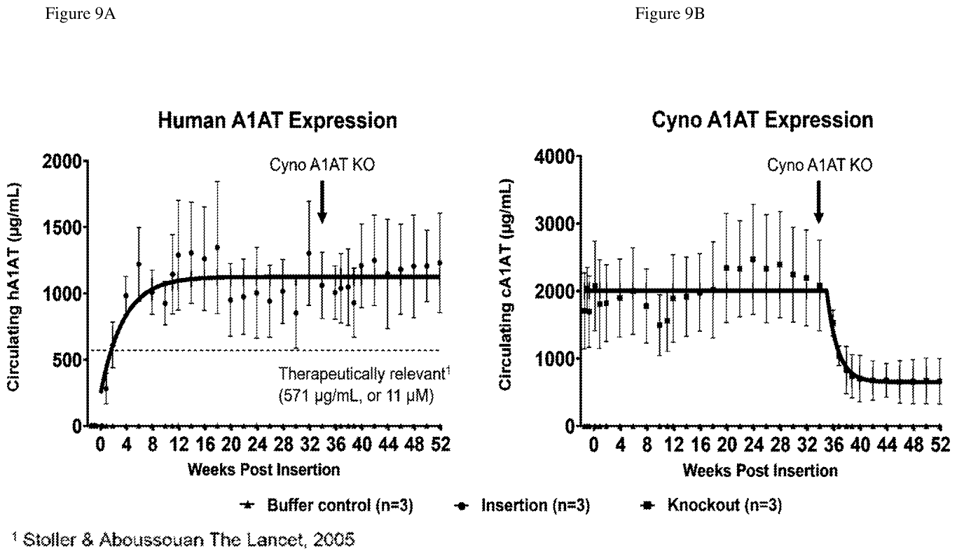

Animals treated with AAV8-SERPINA1 and formulated G009860 expressed increased level of serum hA1AT (Table 24 and FIGS. 9A and 9B) while no hA1AT expression was observed in the buffer control group. Animals treated with the formulated G009860 had an average % Indel of 44.2 while none was observed for the buffer control group (Table 25 and FIG. 7). hA1AT levels reached maximal plateau at week 4 and were maintained through week 52 at an average steady-state level of 1126 μg/mL, as modeled with nonlinear fitting one-phase association. No change in human hA1AT was observed following knockout treatment with formulated G014418 on day 259 (Table 27 and FIG. 8).

Following cA1AT knockout treatment on day 245, animals treated with formulated G014418 expressed decreased level of serum cA1AT while no change in expression was observed in the buffer control group (Table 26 and FIGS. 9A and 9B). Animals treated with formulated G014418 had an average % Indel of 44.0 while none was observed for the buffer control group (Table 27 and FIG. 8). cA1AT levels were maintained at 2005 μg/mL prior to knockout treatment, after which maximal cA1AT reduction was observed in 4 weeks and maintained through week 52 at an average steady-state level of 652 μg/mL, as modeled with nonlinear fitting plateau followed by one phase decay. No change in hA1AT was observed following cA1AT knockout treatment.

TABLE 24
hA1AT levels in serum

| | hA1AT Serum Concentration (μg/mL) in NHP | |
|---|---|---|
| Study | measured by SASLHLPK (SEQ ID NO: 1560) | |
| Day | Vehicle Control | Insertion Treatment |

| Label | 1001 | 1002 | 1003 | 2001 | 2002 | 3003 |
|---|---|---|---|---|---|---|
| D-10 | BQL | BQL | BQL | BQL | BQL | BQL |
| D-7 | BQL | BQL | BQL | BQL | BQL | BQL |
| D-5 | BQL | BQL | BQL | BQL | BQL | BQL |
| D1 | BQL | BQL | BQL | BQL | BQL | BQL |
| D7 | BQL | BQL | BQL | 384 | 158 | 305 |
| D14 | BQL | BQL | BQL | 635 | 429 | 772 |
| D28 | BQL | BQL | BQL | 1030 | 819 | 1100 |
| D42 | BQL | BQL | BQL | 1270 | 922 | 1470 |
| D56 | BQL | BQL | BQL | 1120 | 816 | 1090 |
| D70 | BQL | BQL | BQL | 1110 | 867 | 800 |
| D78 | BQL | BQL | BQL | 1260 | 804 | 1370 |
| D84 | BQL | BQL | BQL | 1345 | 849 | 1670 |
| D98 | BQL | BQL | BQL | 1285 | 935 | 1700 |
| D112 | BQL | BQL | BQL | 1290 | 858 | 1640 |
| D126 | BQL | BQL | BQL | 1345 | 848 | 1845 |
| D140 | BQL | BQL | BQL | 922 | 692 | 1240 |
| D154 | BQL | BQL | BQL | 973 | 691 | 1260 |
| D168 | BQL | BQL | BQL | 981 | 674 | 1360 |
| D182 | BQL | BQL | BQL | 1040 | 634 | 1150 |
| D196 | BQL | BQL | BQL | 1030 | 767 | 1250 |
| D210 | BQL | BQL | BQL | 911 | 564 | 1090 |
| D224 | NR | NR | BQL | 1350 | 889 | 1670 |
| D238 | BQL | BQL | BQL | 1140 | 780 | 1260 |
| D252 | BQL | BQL | BQL | 1080 | 779 | 1160 |
| D258 | BQL | BQL | BQL | 1160 | 738 | 1220 |
| D266 | BQL | BQL | BQL | 1060 | 752 | 1330 |
| D272 | BQL | BQL | BQL | 1110 | 632 | 1050 |
| D280 | BQL | BQL | BQL | 1300 | 857 | 1470 |
| D294 | BQL | BQL | BQL | 1390 | 860 | 1500 |
| D308 | BQL | BQL | BQL | 1230 | 699 | 1510 |
| D322 | BQL | BQL | BQL | 1300 | 800 | 1450 |
| D336 | BQL | BQL | BQL | 1280 | 785 | 1550 |
| D350 | BQL | BQL | BQL | 1420 | 906 | 1300 |
| D364 | BQL | BQL | BQL | 1310 | 821 | 1560 |

BQL: Below Quantitation Limit, NR: Not reported due to analytical issue.

TABLE 25
Editing at Cynomolgus Albumin Locus from Day 14 Liver Biopsy

| | Mean | | |
|---|---|---|---|
| Condition | % Indel | SD | Samples |
| Vehicle Control | <1 | | 3 |
| Insertion Treatment | 44.2 | 11.5 | 3 |

TABLE 26
cA1AT levels in serum

| | cA1AT Serum Concentration (μg/mL) in NHP | |
|---|---|---|
| Study | measured by SANLHLPR (SEQ ID NO: 1559) | |
| Day | Vehicle Control | Insertion Treatment |

| Label | 1001 | 1002 | 1003 | 2001 | 2002 | 3003 |
|---|---|---|---|---|---|---|
| D-10 | 2050 | 2100 | 2370 | 1870 | 1080 | 2170 |
| D-7 | 2140 | 2020 | 2460 | 1810 | NR | 2260 |
| D-5 | 2320 | 2190 | 2400 | 1880 | 1100 | 2110 |
| D1 | 2710 | 2620 | 2890 | 2430 | 1310 | 2490 |
| D7 | 2540 | 2100 | 2290 | 2120 | 1050 | 2250 |
| D14 | 2530 | 2350 | 2490 | 1900 | 1220 | 2350 |
| D28 | 2120 | 2100 | 2200 | 2200 | 1230 | 2260 |
| D42 | 2290 | 2180 | 2800 | 2320 | 1260 | 2420 |
| D56 | 1910 | 2060 | 2370 | 2280 | 1190 | 1870 |
| D70 | 1790 | 1900 | 1900 | 1380 | 1110 | 1990 |
| D78 | 1820 | 1710 | 1710 | 1510 | 1130 | 2040 |
| D84 | 2175 | 2220 | 2260 | 2095 | 1165 | 2415 |
| D98 | 2130 | 1945 | 2085 | 2065 | 1270 | 2415 |
| D112 | 2225 | 2080 | 2385 | 2310 | 1320 | 2310 |
| D126 | 2430 | 2315 | 2340 | 2375 | 1195 | 2480 |
| D140 | 2890 | 2800 | 2740 | 2970 | 1430 | 2630 |
| D154 | 2940 | 2820 | 2770 | 2610 | 1520 | 2860 |
| D168 | 3000 | 2670 | 2930 | 2980 | 1530 | 2900 |
| D182 | 3110 | 2710 | 2930 | 2750 | 1410 | 2840 |
| D196 | 3330 | 2860 | 2970 | 2770 | 1490 | 2920 |
| D210 | 2890 | 2950 | 2980 | 2500 | 1450 | 2780 |
| D224 | NR | NR | 2790 | 2330 | 1430 | 2830 |
| D238 | 2450 | 2300 | 2710 | 2340 | 1320 | 2590 |
| D252 | 2450 | 2440 | 2940 | 1540 | 1330 | 1710 |
| D258 | 2350 | 2360 | 2650 | 878 | 1100 | 1150 |
| D266 | 2630 | 2420 | 2790 | 519 | 1210 | 762 |
| D272 | 2420 | 2030 | 2560 | 487 | 1100 | 631 |
| D280 | 2600 | 2470 | 2680 | 472 | 1100 | 536 |
| D294 | 2630 | 2430 | 2700 | 439 | 1000 | 588 |
| D308 | 2340 | 2430 | 2540 | 446 | 943 | 644 |
| D322 | 2520 | 2550 | 2620 | 411 | 1010 | 545 |
| D336 | 2390 | 2540 | 2630 | 410 | 1030 | 533 |
| D350 | 2690 | 2390 | 2640 | 428 | 1060 | 525 |
| D364 | 2610 | 2310 | 2490 | 428 | 1050 | 512 |

NR: Not reported due to analytical issue.

TABLE 27
Editing at Cynomolgus SERPINA1
Locus from day 259 Liver Biopsy

| | Mean | | |
|---|---|---|---|
| Condition | % Indel | SD | Samples |
| Vehicle Control | <1 | | 3 |
| Insertion Treatment | 44.0 | 17.7 | 3 |

### Example 10—In Vivo Insertion of hSERPINA1 into the Cynomolgus Albumin

AAVs with unique hSERPINA1 sequences (Construct 7 and Construct 8) in combination with the formulated albumin guide G009860 were evaluated for human SERPINA1 gene insertion in male cynomolgus monkeys as provided above.

Two groups of monkeys (n=4/group, 2 male and 2 female) were dosed intravenously with a bolus dose of AAV8 (1.5E13 vg/kg with either Construct 7 or Construct 8 hSERPINA1 sequences) followed by a 30-minute IV infusion of the formulated albumin guide G009860 (3.0 mg/kg). A vehicle control group (n=2, 1 male and 1 female) was dosed with a bolus dose of AAV buffer followed by a 30-minute infusion of LNP buffer. All monkeys were pre-treated with a bolus dose of 2 mg/kg dexamethasone 1 hour prior to the AAV bolus. The AAV and LNPs tested in this study were prepared as described in the materials and methods. Serum cA1AT/hA1AT levels and gene editing were measured as described in the materials and methods.

All animals were prescreened for single-nucleotide variants in the sgRNA target sequence and for pre-existing anti-AAV8 neutralizing antibodies. Pharmacokinetic evaluation of AAV and LNP components in plasma were within historical ranges for all treated animals except for the AAV component in animal 3502. Study documents for animal 3502 noted a mis-dose during AAV administration. Plasma exposures for AAV in animal 3502 were 10× lower than historical ranges indicating a dosing issue. Taking these considerations into account, animal 3502 was excluded from efficacy assessments. Clinical pathology (clinical chemistry, hematology, coagulation) and cytokine monitoring did not yield any usual findings with any parameter elevations returning to baseline within one week.

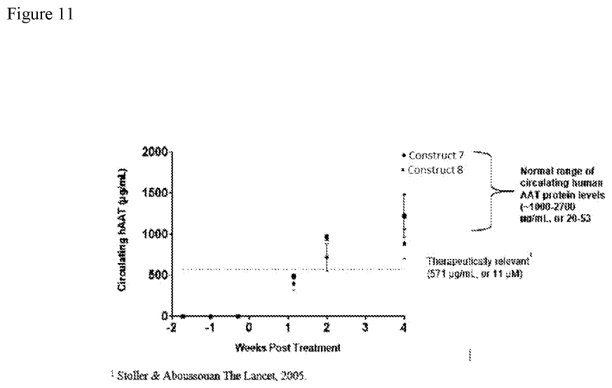

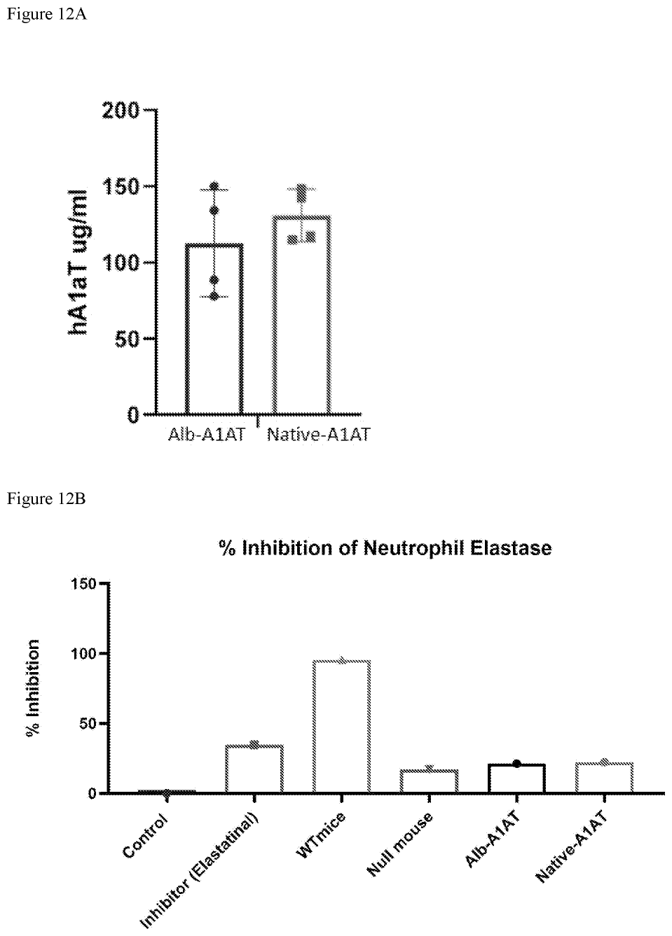

Animals treated with AAV containing Construct 7 or Construct 8 and the formulated albumin guide G009860 expressed increased levels of serum hA1AT while no expression was observed in the buffer control group (Table 28 and FIG. 11). Animals treated with the formulated albumin guide G009860 had an average % Indel of 37.6 in the Construct 7 group and 42.2 in the Construct 8 group. No indels were observed for the buffer control group (Table 29 and FIG. 10). hA1AT levels reached maximal plateau at week 4 with an average of 882 μg/mL in the Construct 7 group and an average of 1223 μg/mL in the Construct 8 group. cA1AT levels were unaffected by either insertion treatment (Table 30).

TABLE 28
hA1AT levels in serum

| | hA1AT Serum Concentration (μg/mL) in NHP | | | | | | | | | |
|---|---|---|---|---|---|---|---|---|---|---|
| | measured by SASLHLPK (SEQ ID NO: 1560) | | | | | | | | | |
| Study | Vehicle | | | | | | | | | |
| Day | Control | | Construct 7 | | | | Construct 8 | | | |
| Label | 1001 | 1002 | 2001 | 2002 | 2501 | 2502 | 3001 | 3002 | 3501 | 3502 |
| D-12 | BQL | BQL | BQL | BQL | BQL | BQL | BQL | BQL | BQL | Excl. |
| D-7 | BQL | BQL | BQL | BQL | BQL | BQL | BQL | BQL | BQL | Excl. |
| D-2 | BQL | BQL | BQL | BQL | BQL | BQL | BQL | BQL | BQL | Excl. |
| D8 | NR | NR | 437 | 459 | 290 | 389 | 458 | 486 | 514 | Excl. |
| D14 | BQL | BQL | 547 | 841 | 613 | 878 | 996 | 928 | 962 | Excl. |
| D28 | BQL | BQL | 648 | 937 | 863 | 1080 | 1520 | 1120 | 1030 | Excl. |

BQL: Below Quantitation Limit,
NR: Not reported due to analytical issue.,
Excl.: Values Excluded

TABLE 29
Editing at Cynomolgus Albumin Locus from day 14 Liver Biopsy

| | Mean | | |
|---|---|---|---|
| AAV | % Indel | SD | Samples |
| Vehicle Control | <1 | | 2 |
| Construct 7 | 37.6 | 6.3 | 4 |
| Construct 8 | 42.2 | 1.5 | 3 |

TABLE 30
cA1AT levels in serum

| | cA1AT Serum Concentration (μg/mL) in NHP | | | | | | | | | |
|---|---|---|---|---|---|---|---|---|---|---|
| | measured by SANLHLPR (SEQ ID NO: 1559) | | | | | | | | | |
| Study | Vehicle | | | | | | | | | |
| Day | Control | | Construct 7 | | | | Construct 8 | | | |
| Label | 1001 | 1002 | 2001 | 2002 | 2501 | 2502 | 3001 | 3002 | 3501 | 3502 |
| D-12 | 2240 | 2250 | 2090 | 3010 | 2220 | 2430 | 2590 | 2220 | 922 | Excl. |
| D-7 | 2430 | 2400 | 2150 | 2590 | 1540 | 2270 | 2860 | 2290 | 1030 | Excl. |
| D-2 | 2270 | 2600 | 2230 | 2600 | 2490 | 2700 | 2420 | 2190 | 1040 | Excl. |
| D8 | NR | NR | 2730 | 3240 | 2710 | 3050 | 2830 | 2690 | 1210 | Excl. |
| D14 | 2410 | 2710 | 2470 | 3220 | 2590 | 3140 | 2870 | 2330 | 1390 | Excl. |
| D28 | 2000 | 2790 | 2230 | 2800 | 2720 | 2780 | 2610 | 2030 | 1670 | Excl. |

NR: Not reported due to analytical issue.,
Excl: Values Excluded

### Example 11—Evaluation of Serum hA1AT for Neutrophil Elastase Inhibition

Neutrophil elastase inhibition activity of native human A1AT was compared to activity of hA1AT sequence that is expressed from the bidirectional construct in SerpinA1 null mice. The hA1AT protein expressed from the bidirectional construct after insertion into the albumin locus contains 3 amino acids at the N-terminus from human albumin insertion site that are not present in the native human A1AT protein.

mRNAs encoding native human A1AT (native-A1AT) or the human A1AT expressed from the bidirectional construct after insertion into the albumin locus (Alb-A1AT) were lipid formulated and delivered intravenously at a dose of 2 mg/kg to SerpinA1 null mice (Jackson Laboratories, n=4 per group). Six hours after administration, blood was collected and serum was prepared for quantification of human A1AT by ELISA (Aviva Biosystems, Cat #OKIA00048), and inhibition of neutrophil elastase as compared to control null mice not treated with mRNA encoding an A1AT, and wild type mice expressing endogenous A1AT.

Expression of A1AT from the expression constructs as determined by ELISA is shown in FIG. 12A and in Table 31.

TABLE 31
Expression of A1AT from in SerpinA1 null mice

| | |
|---|---|
| Alb-A1AT | Native-A1AT |

| Average | | | Average | | |
|---|---|---|---|---|---|
| hA1AT | SD hA1AT | | hA1AT | SD hA1AT | |
| (μg/mL) | (μg/mL) | N | (μg/mL) | (μg/mL) | N |
| 112.73 | 34.99 | 4 | 131.02 | 17.15 | 4 |

The commercially available Neutrophil Elastase Colorimetric Drug Discovery Kit (Cat #: BLM-AK947; Enzo Life Sciences Inc., Farmingdale, NY), was employed to determine the ability of serum A1AT to inhibit neutrophil elastase. Serum from in vivo studies was prepared to enable accurate evaluation of A1AT. Serum samples were diluted 3× in PBS and filtered through a 0.22 μm spin filter (Cat #UFC30GV; Sigma). Two-hundred microliters of Alpha 1 Select Resin (Cat #17547201; Cytiva, Marlborough, MA) was added into an empty column (Cat #731-1550; BioRad) and washed three times with 600 μL of PBS. 600 μL of the filtered A1AT-containing serum sample was introduced to the column and incubated with rotation for 40 minutes at room temperature. Columns were washed three times with PBS and A1AT protein was eluted by adding 500 μL of elution buffer (2M MgCl2, 20 mM Tris pH7.5).

Purified samples were then employed in the neutrophil elastase inhibition assay performed according to manufacturer's protocol. Briefly, kit components were thawed on ice and inhibitors and substrates were diluted to working stock concentrations. Neutrophil elastase enzyme and elastatinal inhibitor control were diluted in assay buffer and added to appropriate wells of a microplate. Purified serum samples were diluted at various concentrations. The plate was incubated for 30 minutes at 37° C. to allow inhibitor/enzyme interaction. Colorimetric substrate was then introduced, and the plates were read on a plate reader at A_{405 nm} at 1 minute time interval for 10 minutes. To determine percent inhibition of purified serum samples, the standard values were plotted as mOD versus time and the range of time points during which the reaction was linear were determined. The rection velocity (mOD/min) was determined and the slope of a line fit to the linear portion of the data plot was defined. The percent inhibition is shown in Table 32 and FIG. 12B

TABLE 32
Percent inhibition of Neutrophil Elastase in purified serum samples

| Sample | Average % Inhibition | SD % Inhibition | N |
|---|---|---|---|
| Alb-A1AT | 21.27 | 5.07 | 5 |
| native A1AT | 22.28 | 0.79 | 5 |
| WT Mice | 95.56 | 1.62 | 4 |
| Null Mice (Control) | 17.25 | 0 | 1 |
| 125 μg/mL inhibitor | 88.22 | 0 | 1 |
| (Elastatinal) (Control) | | | |

Alb-A1AT
(SEQ ID NO: 1562)
GGGAAGCUCAGAAUAAACGCUCAACUUUGGCCGGAUCUGGCGCGCCAC
CAUGAAGUGGGUAACCUUUAUUUCCCUUCUUUUUCUCUUUAGCUCGGC
UUAUUCCAGGGGUGUGUUUCGUCGAGAUGCACUUGAGGAUCCCCAGGG
AGAUGCUGCCCAGAAGACAGAUACAUCCCACCAUGAUCAGGAUCACCC
AACCUUCAACAAGAUCACCCCCAACCUGGCUGAGUUCGCCUUCAGCCU
AUACCGCCAGCUGGCACACCAGUCCAACAGCACCAAUAUCUUCUUCUC
CCCAGUGAGCAUCGCUACAGCCUUUGCAAUGCUCUCCCUGGGGACCAA
GGCUGACACUCACGAUGAAAUCCUGGAGGGCCUGAAUUUCAACCUCAC
GGAGAUUCCGGAGGCUCAGAUCCAUGAAGGCUUCCAGGAACUCCUCCG
UACCCUCAACCAGCCAGACAGCCAGCUCCAGCUGACCACCGGCAAUGG
CCUGUUCCUCAGCGAGGGCCUGAAGCUAGUGGAUAAGUUUUUGGAGGA
UGUUAAAAAGUUGUACCACUCAGAAGCCUUCACUGUCAACUUCGGGGA
CACCGAAGAGGCCAAGAAACAGAUCAACGAUUACGUGGAGAAGGGUAC
UCAAGGGAAAAUUGUGGAUUUGGUCAAGGAGCUUGACAGAGACACAGU
UUUUGCUCUGGUGAAUUACAUCUUCUUUAAAGGCAAAUGGGAGAGACC
CUUUGAAGUCAAGGACACCGAGGAAGAGGACUUCCACGUGGACCAGGU
GACCACCGUGAAGGUGCCUAUGAUGAAGCGUUUAGGCAUGUUUAACAU
CCAGCACUGUAAGAAGCUGUCCAGCUGGGUGCUGCUGAUGAAAUACCU
GGGCAAUGCCACCGCCAUCUUCUUCCUGCCUGAUGAGGGGAAACUACA
GCACCUGGAAAAUGAACUCACCCACGAUAUCAUCACCAAGUUCCUGGA
AAAUGAAGACAGAAGGUCUGCCAGCUUACAUUUACCCAAACUGUCCAU
UACUGGAACCUAUGAUCUGAAGAGCGUCCUGGGUCAACUGGGCAUCAC
UAAGGUCUUCAGCAAUGGGGCUGACCUCUCCGGGGUCACAGAGGAGGC
ACCCCUGAAGCUCUCCAAGGCCGUGCAUAAGGCUGUGCUGACCAUCGA
CGAGAAAGGGACUGAAGCUGCUGGGGCCAUGUUUUUAGAGGCCAUACC
CAUGUCUAUCCCCCCCGAGGUCAAGUUCAACAAACCCUUUGUCUUCUU
AAUGAUUGAACAAAAUACCAAGUCUCCCCUCUUCAUGGGAAAAGUGGU
GAAUCCCACCCAAAAAUAAUAGGCUAGCCACCAGCCUCAAGAACACCC
GAAUGGAGUCUCUAAGCUACAUAAUACCAACUUACACUUUACAAAAUG
UUGUCCCCCAAAAUGUAGCCAUUCGUAUCUGCUCCUAAUAAAAAGAAA
GUUUCUUCACAUUCUCUCGAGAAAAAAAAAAAAUGGAAAAAAAAAAAA
CGGAAAAAAAAAAAGGUAAAAAAAAAAAAUAUAAAAAAAAAAACAUAA
AAAAAAAAAACGAAAAAAAAAAAACGUAAAAAAAAAAAACUCAAAAAA
AAAAAGAUAAAAAAAAAAAACCUAAAAAAAAAAAAUGUAAAAAAAAAA
AAGGGAAAAAAAAAAACGCAAAAAAAAAAAACACAAAAAAAAAAAAUG
CAAAAAAAAAAAAUCGAAAAAAAAAAAAUCUAAAAAAAAAAAACGAAA
AAAAAAAAACCCAAAAAAAAAAAAGACAAAAAAAAAAAAUAGAAAAAA
AAAAAGUUAAAAAAAAAAAACUGAAAAAAAAAAAAUUUAAAAAAAAAA
AAUCUAG
Native A1AT
(SEQ ID NO: 1563)
GGGAAGCUCAGAAUAAACGCUCAACUUUGGCCGGAUCUGGCGCGCCAC
CAUGCCGUCUUCUGUCUCGUGGGGCAUCCUCCUGCUGGCAGGCCUGUG
CUGCCUGGUCCCUGUCUCCCUGGCUGAGGAUCCCCAGGGAGAUGCUGC
CCAGAAGACAGAUACAUCCCACCAUGAUCAGGAUCACCCAACCUUCAA
CAAGAUCACCCCCAACCUGGCUGAGUUCGCCUUCAGCCUAUACCGCCA
GCUGGCACACCAGUCCAACAGCACCAAUAUCUUCUUCUCCCCAGUGAG
CAUCGCUACAGCCUUUGCAAUGCUCUCCCUGGGGACCAAGGCUGACAC
UCACGAUGAAAUCCUGGAGGGCCUGAAUUUCAACCUCACGGAGAUUCC
GGAGGCUCAGAUCCAUGAAGGCUUCCAGGAACUCCUCCGUACCCUCAA
CCAGCCAGACAGCCAGCUCCAGCUGACCACCGGCAAUGGCCUGUUCCU
CAGCGAGGGCCUGAAGCUAGUGGAUAAGUUUUUGGAGGAUGUUAAAAA
GUUGUACCACUCAGAAGCCUUCACUGUCAACUUCGGGGACACCGAAGA
GGCCAAGAAACAGAUCAACGAUUACGUGGAGAAGGGUACUCAAGGGAA
AAUUGUGGAUUUGGUCAAGGAGCUUGACAGAGACACAGUUUUUGCUCU
GGUGAAUUACAUCUUCUUUAAAGGCAAAUGGGAGAGACCCUUUGAAGU
CAAGGACACCGAGGAAGAGGACUUCCACGUGGACCAGGUGACCACCGU
GAAGGUGCCUAUGAUGAAGCGUUUAGGCAUGUUUAACAUCCAGCACUG
UAAGAAGCUGUCCAGCUGGGUGCUGCUGAUGAAAUACCUGGGCAAUGC
CACCGCCAUCUUCUUCCUGCCUGAUGAGGGGAAACUACAGCACCUGGA
AAAUGAACUCACCCACGAUAUCAUCACCAAGUUCCUGGAAAAUGAAGA
CAGAAGGUCUGCCAGCUUACAUUUACCCAAACUGUCCAUUACUGGAAC
CUAUGAUCUGAAGAGCGUCCUGGGUCAACUGGGCAUCACUAAGGUCUU
CAGCAAUGGGGCUGACCUCUCCGGGGUCACAGAGGAGGCACCCCUGAA
GCUCUCCAAGGCCGUGCAUAAGGCUGUGCUGACCAUCGACGAGAAAGG
GACUGAAGCUGCUGGGGCCAUGUUUUUAGAGGCCAUACCCAUGUCUAU
CCCCCCCGAGGUCAAGUUCAACAAACCCUUUGUCUUCUUAAUGAUUGA
ACAAAAUACCAAGUCUCCCCUCUUCAUGGGAAAAGUGGUGAAUCCCAC
CCAAAAAUAAUAGGCUAGCCACCAGCCUCAAGAACACCCGAAUGGAGU
CUCUAAGCUACAUAAUACCAACUUACACUUUACAAAAUGUUGUCCCCC
AAAAUGUAGCCAUUCGUAUCUGCUCCUAAUAAAAAGAAAGUUUCUUCA
CAUUCUCUCGAGAAAAAAAAAAAAUGGAAAAAAAAAAAACGGAAAAAA
AAAAAGGUAAAAAAAAAAAAUAUAAAAAAAAAAACAUAAAAAAAAAAA
ACGAAAAAAAAAAAACGUAAAAAAAAAAAACUCAAAAAAAAAAAGAUA
AAAAAAAAAAACCUAAAAAAAAAAAAUGUAAAAAAAAAAAAGGGAAAA
AAAAAAACGCAAAAAAAAAAAACACAAAAAAAAAAAAUGCAAAAAAAA
AAAAUCGAAAAAAAAAAAAUCUAAAAAAAAAAAACGAAAAAAAAAAAA
CCCAAAAAAAAAAAAGACAAAAAAAAAAAAUAGAAAAAAAAAAAGUUA
AAAAAAAAAAACUGAAAAAAAAAAAAUUUAAAAAAAAAAAAUCUAG

### Example 12—Resistance of Template Insertion Sequences to Sequential siRNA Silencing and CRISPR Editing in SERPINA1 Null Mice

Nuclease resistance of insertion template sequences was tested in SERPINA1 null mice by inserting the template and following-on with siRNA treatment targeting wild type human SERPINA1. Construct 1 includes a wild type coding sequence and a codon optimized sequence for SERPINA1. The codon optimized sequence is not fully complementary to the antisense sequence of siRNA2 and siRNA3.

At Day 0, SERPINA1 null mice (n=9 male, 9 female) were dosed with 1 mg/kg (with respect to total RNA cargo content) LNP carrying Cas9 mRNA and sgRNA G000666 (targeting mouse albumin), and with ssAAV derived from Construct 1 A1AT Template at 1.5e11 vg/mouse. All reagents were prepared and dosed as described above. Blood was collected and serum prepared prior to treatment with an siRNA at Days 14 and 28. At Days 28, 29, and 30, mice (n=3 male and 3 female, per group) were treated with LNP formulated of siRNA2 or siRNA3 (0.3 mg/kg), or vehicle control. Blood was collected and serum prepared at Day 32.

Human A1AT levels in the serum were determined by ELISA (Aviva Biosystems, Cat #OKIA00048) according to manufacturer's protocol.

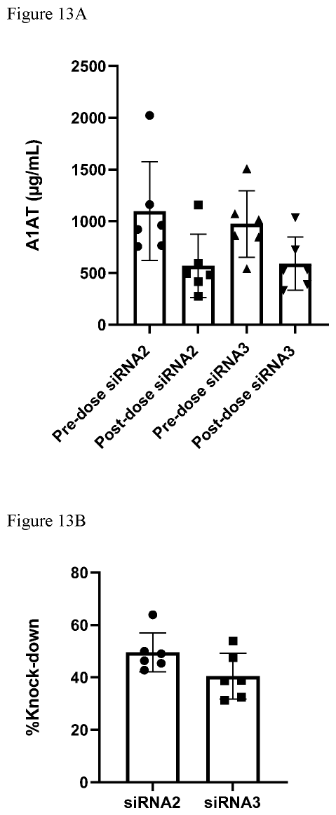

FIG. 13A and Table 33 shows hA1AT protein levels as measured by ELISA at Day 28 (pre-dose), and at Day 32 (post-dose). FIG. 13B and Table 34 show the percent knockdown of A1AT following dosing of either siRNA2 or siRNA3.

TABLE 33
hA1AT levels as measured by ELISA pre and post dose of siRNA

| | siRNA2 | | | siRNA3 | | |
|---|---|---|---|---|---|---|
| | Average | SD | | Average | SD | |
| | A1AT | A1AT | | A1AT | A1AT | |
| Day | (μg/mL) | (μg/mL) | N | (μg/mL) | (μg/mL) | N |
| Day 28 | 1098.09 | 476.74 | 6 | 973.73 | 319.92 | 6 |
| Day 32 | 569.32 | 306.84 | 6 | 590.08 | 257.15 | 6 |

TABLE 34
Percent knockdown following dose of siRNA2 and siRNA3

| | siRNA2 | | | siRNA3 | | |
|---|---|---|---|---|---|---|
| | Average | SD | | Average | SD | |
| | A1AT | A1AT | | A1AT | A1AT | |
| siRNA | (μg/mL) | (μg/mL) | N | (μg/mL) | (μg/mL) | N |
| Day 28 | 1098.09 | 476.74 | 6 | 973.73 | 319.92 | 6 |
| Day 32 | 569.32 | 306.84 | 6 | 590.08 | 257.15 | 6 |

### Example 13—SERPINA1 Insertion with a Bidirectional Constructs with Various Splice Acceptors

Construct 11 is a bidirectional construct with the SERPINA1 coding sequences of Construct 8 with human serum albumin splice acceptor sites. Insertion of hSERPINA1 into C57BL mouse albumin locus using bidirectional ssAAV Constructs 7 and 11 was tested. The ssAAV and LNPs tested in this Example were prepared and delivered to mice as described in Example 1.

Mice at 8-9 weeks of age were dosed with 1 mg/kg (with respect to total RNA cargo content) LNP carrying Cas9 mRNA and sgRNA G000666 (targeting mouse albumin). The ssAAV were assessed at the doses provided in Table 35.

TABLE 35
Dosing regimen for Constructs 7 and 11

| | LNP dose | AAV Dose | N |
|---|---|---|---|
| Vehicle | X | X | 4 |
| Construct 11 | 1 mpk | 2.5e13 vg/kg | 5 |
| Construct 11 | 1 mpk | 7.5e12 vg/kg | 5 |
| Construct 11 | 1 mpk | 2.5e12 vg/kg | 5 |
| Construct 7 | 1 mpk | 2.5e13 vg/kg | 5 |
| Construct 7 | 1 mpk | 7.5e12 vg/kg | 5 |
| Construct 7 | 1 mpk | 2.5e12 vg/kg | 5 |

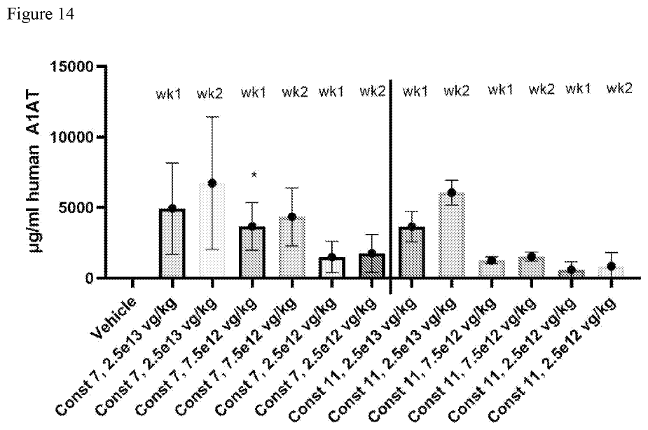

Blood was collected at weeks one and two post-dose. Four weeks post dose, the animals are euthanized, liver tissue and blood are collected to assess liver editing and hA1AT expression levels in serum, respectively. Indel formation is determined by NGS. Sera was prepared to measure human alpha1 antitrypsin (hA1AT) serum expression by ELISA (Aviva Biosystems, Cat #OKIA00048). Serum hA1AT levels are shown in FIG. 14 and Table 36 at one week and two weeks post dose.

TABLE 36
Serum A1AT levels after dosing with Constructs 7 and 11

| | | | | Average | |
|---|---|---|---|---|---|
| | | Average | | A1AT, | |
| | AAV | A1AT, week | SD A1AT | week 2 | SD A1AT |
| | Dose | 1 (μg/mL) | (μg/mL) | (μg/mL) | (μg/mL) |
| Vehicle | X | BLOD | | BLOD | |
| Construct 11 | 2.5e13 | 3646.10 | 1079.49 | 6066.59 | 882.25 |
| | vg/kg | | | | |
| Construct 11 | 7.5e12 | 1271.45 | 234.99 | 1522.53 | 320.70 |
| | vg/kg | | | | |
| Construct 11 | 2.5e12 | 596.52 | 561.83 | 843.55 | 969.81 |
| | vg/kg | | | | |
| Construct 7 | 2.5e13 | 4926.10 | 3244.26 | 6730.24 | 4690.71 |
| | vg/kg | | | | |
| Construct 7 | 7.5e12 | 3665.04 | 1690.07 | 4340.04 | 2048.45 |
| | vg/kg | | | | |
| Construct 7 | 2.5e12 | 1498.00 | 1113.63 | 1758.13 | 1339.48 |
| | vg/kg | | | | |

BLOD = below limit of detection

TABLE 37
Additional Sequences

| Construct | Sequence |
|---|---|
| Nanoluc | taggtcagtgaagagaagaacaaaaagcagcatattacagttagttgtcttcatcaa |
| | tctttaaatatgttgtgtggtttttctctccctgtttccacagtttttcttgatcat |
| | gaaaacgccaacaaaattctgaatcggccaaagaggtataattcaggtaaattggaa |
| | gagtttgttcaagggaaccttgagagagaatgtatggaagaaaagtgtagttttgaa |
| | gaagcaGTATTCACTTTGGAGGACTTTGTCGGTGACTGGAGGCAAACCGCTGGTTAT |
| | AATCTCGACCAaGTACTGGAACAGGGCGGGGTAAGTTCCCTCTTTCAGAATTTGGGT |
| | GTAAGCGTCACACCAATCCAGCGGATTGTGTTGTCTGGAGAGAACGGACTCAAAATT |
| | GACATCCATGTTATCATTCCATATGAAGGTCTCAGTGGAGACCAAATGGGGCAGATC |
| | GAGAAGATTTTCAAGGTAGTTTACCCAGTCGACGATCACCACTTCAAAGTCATtCTC |
| | CACTATGGCACACTTGTTATCGACGGAGTAACTCCTAATATGATTGATTACTTTGGT |
| | CGCCCGTATGAGGGCATCGCAGTGTTTGATGGCAAAAAGATCACCGTAACAGGAACG |
| | TTGTGGAATGGGAACAAGATAATCGACGAGAGATTGATAAATCCAGACGGGTCACTC |
| | CTGTTCAGGGTTACAATTAACGGCGTCACAGGATGGAGACTCTGTGAACGAATACTG |
| | GCCacaaatttttcactcctgaagcaggccggagacgtggaggaaaacccagggccc |
| | gtgAGCAAGGGCGAGGAGCTGTTCACCGGGGTGGTGCCCATCCTGGTCGAGCTGGAC |
| | GGCGACGTAAACGGCCACAAGTTCAGCGTGTCCGGCGAGGGCGAGGGCGATGCCACC |
| | TACGGCAAGCTGACCCTGAAGTTCATCTGCACCACCGGCAAGCTGCCCGTGCCCTGG |
| | CCCACCCTCGTGACCACCCTGACCTACGGCGTGCAGTGCTTCAGCCGCTACCCCGAC |
| | CACATGAAGCAGCACGACTTCTTCAAGTCCGCCATGCCCGAAGGCTACGTCCAGGAG |
| | CGCACCATCTTCTTCAAGGACGACGGCAACTACAAGACCCGCGCCGAGGTGAAGTTC |
| | GAGGGCGACACCCTGGTGAACCGCATCGAGCTGAAGGGCATCGACTTCAAGGAGGAC |
| | GGCAACATCCTGGGGCACAAGCTGGAGTACAACTACAACAGCCACAACGTCTATATC |
| | ATGGCCGACAAGCAGAAGAACGGCATCAAGGTGAACTTCAAGATCCGCCACAACATC |
| | GAGGACGGCAGCGTGCAGCTCGCCGACCACTACCAGCAGAACACCCCCATCGGCGAC |
| | GGCCCCGTGCTGCTGCCCGACAACCACTACCTGAGCACCCAGTCCGCCCTGAGCAAA |
| | GACCCCAACGAGAAGCGCGATCACATGGTCCTGCTGGAGTTCGTGACCGCCGCCGGG |
| | ATCACTCTCGGCATGGACGAGCTGTACAAGGGAGGAGGAAGCCCGAAGAAGAAGAGA |
| | AAGGTCTAAcctCGACTGTGCCTTCTAGTTGCCAGCCATCTGTTGTTTGCCCCTCCC |
| | CCGTGCCTTCCTTGACCCTGGAAGGTGCCACTCCCACTGTCCTTTCCTAATAAAATG |
| | AGGAAATTGCATCGCATTGTCTGAGTAGGTGTCATTCTATTCTGGGGGGTGGGGTGG |
| | GGCAGGACAGCAAGGGGGAGGATTGGGAAGACAATAGCAGGCATGCTGGGGATGCGG |
| | TGGGCTCTATGGcttctgaggcggaaagaaccagctggggctctagggggtatcccc |
| | AAAAAACCTCCCACACCTCCCCCTGAACCTGAAACATAAAATGAATGCAATTGTTGT |
| | TGTTAACTTGTTTATTGCAGCTTATAATGGTTACAAATAAAGCAATAGCATCACAAA |
| | TTTCACAAATAAAGCATTTTTTTCACTGCATTCTAGTTGTGGTTTGTCCAAACTCAT |
| | CAATGTATCTTATCATGTCTGTTACACCTTCCTCTTCTTCTTGGGGCTGCCGCCGCC |
| | CTTGTACAGCTCGTCCATGCCCAGGGTGATGCCGGCGGCGGTCACGAACTCCAGCAG |
| | CACCATGTGGTCCCTCTTCTCGTTGGGGTCCTTGCTCAGGGCGCTCTGGGTGCTCAG |
| | GTAGTGGTTGTCGGGCAGCAGCACGGGGCCGTCGCCGATGGGGGTGTTCTGCTGGTA |
| | GTGGTCGGCCAGCTGCACGCTGCCGTCCTCGATGTTGTGCCTGATCTTGAAGTTCAC |
| | CTTGATGCCGTTCTTCTGCTTGTCGGCCATGATGTACACGTTGTGGCTGTTGTAGTT |
| | GTACTCCAGCTTGTGGCCCAGGATGTTGCCGTCCTCCTTGAAGTCGATGCCCTTCAG |
| | CTCGATCCTGTTCACCAGGGTGTCGCCCTCGAACTTCACCTCGGCCCTGGTCTTGTA |
| | GTTGCCGTCGTCCTTGAAGAAGATGGTCCTCTCCTGCACGTAGCCCTCGGGCATGGC |
| | GCTCTTGAAGAAGTCGTGCTGCTTCATGTGGTCGGGGTACCTGCTGAAGCACTGCAC |
| | GCCGTAGGTCAGGGTGGTCACCAGGGTGGGCCAGGGCACGGGCAGCTTGCCGGTGGT |
| | GCAGATGAACTTCAGGGTCAGCTTGCCGTAGGTGGCGTCGCCCTCGCCCTCGCCGCT |
| | CACGCTGAACTTGTGGCCGTTCACGTCGCCGTCCAGCTCCACCAGGATGGGCACCAC |
| | GCCGGTGAACAGCTCCTCGCCCTTGCTCACGGGGCCGGGGTTCTCCTCCACGTCGCC |
| | GGCCTGCTTCAGCAGGCTGAAGTTGGTGGCCAGGATCCTCTCGCACAGCCTCCAGCC |
| | GGTCACGCCGTTGATGGTCACCCTGAACAGCAGGCTGCCGTCGGGGTTGATCAGCCT |
| | CTCGTCGATGATCTTGTTGCCGTTCCACAGGGTGCCGGTCACGGTGATCTTCTTGCC |
| | GTCGAACACGGCGATGCCCTCGTAGGGCCTGCCGAAGTAGTCGATCATGTTGGGGGT |
| | CACGCCGTCGATCACCAGGGTGCCGTAGTGCAGGATCACCTTGAAGTGGTGGTCGTC |
| | CACGGGGTACACCACCTTGAAAATCTTCTCGATCTGGCCCATCTGGTCGCCGCTCAG |
| | GCCCTCGTAGGGGATGATCACGTGGATGTCGATCTTCAGGCCGTTCTCGCCGCTCAG |
| | CACGATCCTCTGGATGGGGGTCACGCTCACGCCCAGGTTCTGGAACAGGCTGCTCAC |
| | GCCGCCCTGCTCCAGCACCTGGTCCAGGTTGTAGCCGGCGGTCTGCCTCCAGTCGCC |
| | CACGAAGTCCTCCAGGGTGAACACGGCCTCCTCGAAGCTGCACTTCTCCTCCATGCA |
| | CTCCCTCTCCAGGTTGCCCTGCACGAACTCCTCCAGCTTGCCGCTGTTGTACCTCTT |
| | GGGCCTGTTCAGGATCTTGTTGGCGTTCTCGTGGTCCAGGAAaactgtggaaacagg |
| | gagagaaaaaccacacaacatatttaaagattgatgaagacaactaactgtaatatg |
| | ctgctttttgttcttctcttcactgaccta (SEQ ID NO: 1550) |

1.-97. (canceled)

98. A bidirectional nucleic acid construct comprising:
a) a first segment comprising a first alpha-1 antitrypsin (AAT) polypeptide coding sequence comprising the nucleic acid sequence of SEQ ID NO: 781; and
b) a second segment comprising a reverse complement of a second AAT polypeptide coding sequence comprising the nucleic acid sequence of SEQ ID NO: 782;
wherein the construct does not comprise a promoter that drives the expression of either the first AAT polypeptide coding sequence or the second AAT polypeptide coding sequence.

99. The bidirectional nucleic acid construct of claim 98, wherein the second segment is 3′ of the first segment.

100. The bidirectional nucleic acid construct of claim 98, wherein the construct does not comprise a homology arm.

101. The bidirectional nucleic acid construct of claim 98, wherein the first segment is linked to the second segment by a linker.

102. The bidirectional nucleic acid construct of claim 98, wherein each of the first and second segments comprises a polyadenylation tail sequence, a polyadenylation signal sequence, or a polyadenylation site.

103. The bidirectional nucleic acid construct of claim 98, wherein the construct comprises a splice acceptor site.

104. The bidirectional nucleic acid construct of claim 103, wherein the construct comprises a first splice acceptor site upstream of the first segment and a second (reverse) splice acceptor site downstream of the second segment.

105. The bidirectional nucleic acid construct of claim 98, wherein the construct is single-stranded.

106. The bidirectional nucleic acid construct of claim 98, wherein the construct comprises one or more of the following terminal structures: hairpin, loops, inverted terminal repeats (ITR), or toroid.

107. The bidirectional nucleic acid construct of claim 98, wherein the construct comprises one, two, or three inverted terminal repeats (ITR).

108. A method of treating alpha-1 antitrypsin deficiency (AATD) in a subject, the method comprising administering the bidirectional nucleic acid construct of claim 98.

109. The method of claim 108, comprising administering the bidirectional nucleic acid in combination with:
i) an RNA-guided DNA binding agent; and
ii) an albumin guide RNA (gRNA) comprising a sequence selected from:
a) a sequence that is at least 95% identical to a sequence selected from the group consisting of SEQ ID Nos: 2-33;
b) at least 17, 18, 19, or 20 contiguous nucleotides of a sequence selected from the group consisting of SEQ ID NOs: 2-33; and
c) a sequence selected from the group consisting of SEQ ID NOs: 2-33.

110. The method of claim 109, wherein the bidirectional nucleic acid is administered in combination with an endogenous SERPINA1 gene targeted nucleic acid agent that reduces expression of the endogenous SERPINA1 gene without significantly reducing expression of the AAT polypeptide coding sequences of the bidirectional nucleic acid construct.

111. The method of claim 108, wherein the bidirectional nucleic acid construct is administered in a nucleic acid vector or a lipid nanoparticle.

112. The method of claim 109, wherein
i) the RNA-guided DNA binding agent or the albumin gRNA is administered in a nucleic acid vector or lipid nanoparticle; and/or
ii) the RNA-guided DNA binding agent or the SERPINA1 gRNA is administered in a nucleic acid vector or lipid nanoparticle.

113. A vector comprising the bidirectional nucleic acid construct of claim 98.

114. The vector of claim 113, wherein the vector is an adeno-associated virus (AAV) vector.

115. A lipid nanoparticle comprising the bidirectional nucleic acid construct of claim 98.

116. A host cell comprising the bidirectional nucleic acid construct of claim 98.

117. The host cell of claim 116, wherein the cell expresses the AAT polypeptide encoded by the bidirectional nucleic acid construct.
